(19) United States
(12) Patent Application Publication
(10) Pub. No.: US 2021/0045385 A1
(43) Pub. Date: Feb. 18, 2021

(54) PYRIDAZINONE-SUBSTITUTED KETOXIMES AS HERBICIDES

(71) Applicant: FMC Corporation, Philadelphia, PA (US)

(72) Inventors: John Robbins DEBERGH, Middletown, DE (US); Eric Allen MARSHALL, Rising Sun, MD (US); Rachel Tran DAO, Newark, DE (US)

(21) Appl. No.: 16/963,810

(22) PCT Filed: Jan. 17, 2019
(86) PCT No.: PCT/US2019/013916

§ 371 Date: Jul. 21, 2020

Related U.S. Application Data
(60) Provisional application No. 62/619,801, filed on Jan. 21, 2018.

(51) Int. Cl. A01N 43/58; C07D 237/16

(52) U.S. Cl. CPC A01N 43/58; C07D 237/16

(57) ABSTRACT

Disclosed are compounds of Formula 1, including all stereoisomers, N-oxides, and salts thereof,

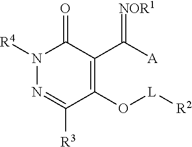

wherein R¹, A, L, R², R³ and R⁴ are as defined in the disclosure.

Also disclosed are compositions containing the compounds of Formula 1 and methods for controlling undesired vegetation comprising contacting the undesired vegetation or its environment with an effective amount of a compound or a composition of the invention.

### FIELD OF THE INVENTION

This invention relates to certain pyridazinone-substituted ketoximes, their N-oxides, salts and compositions, and methods of their use for controlling undesirable vegetation.

### BACKGROUND OF THE INVENTION

The control of undesired vegetation is extremely important in achieving high crop efficiency. Achievement of selective control of the growth of weeds especially in such useful crops as rice, soybean, sugar beet, maize, potato, wheat, barley, tomato and plantation crops, among others, is very desirable. Unchecked weed growth in such useful crops can cause significant reduction in productivity and thereby result in increased costs to the consumer. The control of undesired vegetation in noncrop areas is also important. Many products are commercially available for these purposes, but the need continues for new compounds that are more effective, less costly, less toxic, environmentally safer or have different sites of action.

### SUMMARY OF THE INVENTION

This disclosure relates, in part, to a compound of Formula 1, including all stereoisomers and N-oxides of such compounds, and salts of such compounds, stereoisomers and N-oxides and agricultural compositions containing them and their use as herbicides

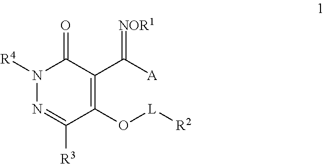

wherein
R¹ is H, C₁-C₇ alkyl, C₂-C₇ alkenyl, C₃-C₇ alkynyl, C₂-C₇ haloalkyl, C₂-C₇ haloalkenyl, C₄-C₈ alkylcycloalkyl, C₄-C₈ haloalkylcycloalkyl, C₃-C₇ cycloalkyl, C₃-C₇ halocycloalkyl, C₄-C₇ cycloalkylalkyl, C₂-C₇ cyanoalkyl, C₃-C₈ alkylcarbonylalkyl, C₃-C₈ alkoxycarbonylalkyl, C₁-C₄ nitroalkyl, C₂-C₇ haloalkoxyalkyl, C₂-C₇ alkoxyalkyl, C₇-C₇ hydroxyalkyl or C₃-C₇ alkylthioalkyl; or benzyl optionally substituted by halogen, C₁-C₄ alkyl or C₁-C₄ haloalkyl;
A is selected from the group consisting of

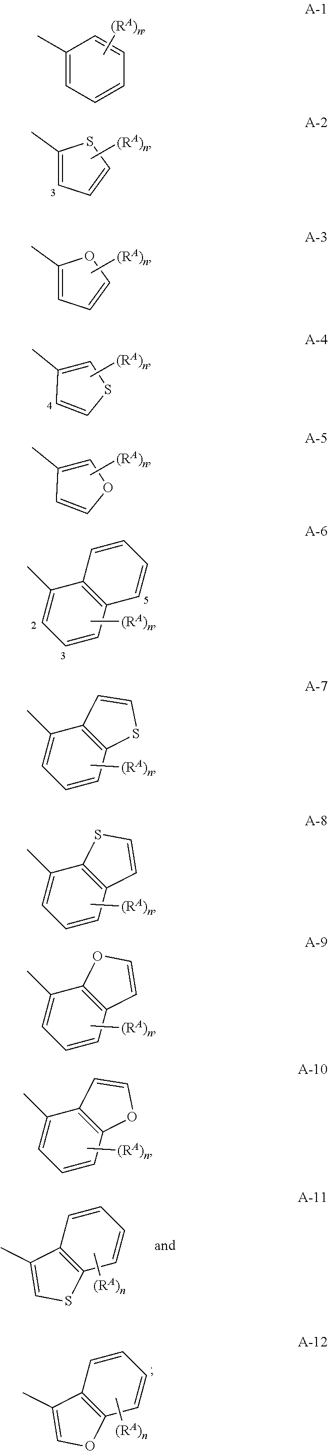

each R^{A} is independently halogen, nitro, cyano, C₁-C₅ alkyl, C₂-C₅ alkenyl, C₂-C₅ alkynyl, C₃-C₅ cycloalkyl, C₄-C₅ cycloalkylalkyl, C₁-C₅ haloalkyl, C₃-C₅ haloalkenyl, C₃-C₅ haloalkynyl, C₂-C₅ alkoxyalkyl, C₁-C₅ alkoxy, C₁-C₅ haloalkoxy, C₁-C₅ alkylthio, C₁-C₄ alkylsulfinyl, C₁-C₄ alkylsulfonyl. C₁-C₅ haloalkylthio or C₂-C₅ alkoxycarbonyl;
n is 0, 1 or 2;
L is a direct bond, C₁-C₄ alkanediyl or C₂-C₄ alkenediyl;
R² is H, C(═O)R⁵, C(═S)R⁵, CO₂R⁶, C(═O)SR⁶, S(O)₂R⁵, CONR⁷R⁸, S(O)₂N(R⁷)R⁸ or P(═O)(R⁹)R¹⁰; or C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₂-C₄ alkoxyalkyl, C₃-C₆ cycloalkyl or C₄-C₇ cycloalkylalkyl; or a 5- or 6-membered heterocyclic ring optionally substituted by halogen, C₁-C₄ alkyl or C₁-C₄ haloalkyl;
R³ is H, halogen, cyano, —CHO, C₁-C₇ alkyl, C₃-C₈ alkylcarbonylalkyl, C₃-C₈ alkoxycarbonylalkyl, C₁-C₄ alkylcarbonyl, C₂-C₇ alkylcarbonyloxy, C₄-C₇ alkylcycloalkyl, C₃-C₇ alkenyl, C₃-C₇ alkynyl, C₁-C₄ alkylsulfinyl, C₁-C₄ alkylsulfonyl, C₁-C₄ alkylamino, C₂-C₈ dialkylamino, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₃-C₇ haloalkenyl, C₂-C₇ alkoxyalkyl, C₁-C₇ alkoxy, C₁-C₅ alkylthio or C₂-C₃ alkoxycarbonyl;
R⁴ is H, C₁-C₇ alkyl, C₃-C₈ alkylcarbonylalkyl, C₃-C₈ alkoxycarbonylalkyl, C₄-C₇ alkylcycloalkyl, C₃-C₇ alkenyl, C₃-C₇ alkynyl, C₃-C₇ cycloalkyl, C₄-C₇ cycloalkylalkyl, C₂-C₃ cyanoalkyl, C₁-C₄ nitroalkyl. C₂-C₇ haloalkoxyalkyl, C₁-C₇ haloalkyl, C₃-C₇ haloalkenyl, C₂-C₇ alkoxyalkyl, C₃-C₇ alkylthioalkyl, C₁-C₇ alkoxy; or benzyl optionally substituted by halogen, C₁-C₄ alkyl or C₁-C₄ haloalkyl;
each R⁵ and R⁷ are independently H, C₁-C₇ alkyl, C₃-C₇ alkenyl, C₃-C₇ alkynyl, C₃-C₇ cycloalkyl, C₁-C₇ haloalkyl, C₃-C₇ haloalkenyl, C₂-C₇ alkoxyalkyl or C₄-C₇ cycloalkylalkyl; or phenyl, benzyl, or a 5- to 6-membered heterocyclic ring, each phenyl, benzyl or heterocyclic ring optionally substituted by halogen, C₁-C₄ alkyl or C₁-C₄ haloalkyl;
R⁶ is C₁-C₇ alkyl, C₃-C₇ alkenyl, C₃-C₇ alkynyl, C₃-C₇ cycloalkyl, C₂-C₇ haloalkyl, C₃-C₇ haloalkenyl, C₂-C₇ alkoxyalkyl or C₄-C₇ cycloalkylalkyl; or phenyl, benzyl or a 5- to 6-membered heterocyclic ring, each phenyl, benzyl or heterocyclic ring optionally substituted by halogen, C₁-C₄ alkyl or C₁-C₄ haloalkyl;
R⁸ is H, C₁-C₇ alkyl, C₂-C₇ alkenyl, C₂-C₇ alkynyl, C₃-C₇ cycloalkyl, C₄-C₇ cycloalkylalkyl, C₁-C₇ haloalkyl or C₂-C₇ alkoxyalkyl;
R⁹ is C₁-C₇ alkyl or C₁-C₇ alkoxy; and
R¹⁰ is C₁-C₇ alkyl or C₁-C₇ alkoxy.

This invention also relates to a herbicidal composition comprising a compound of the invention (i.e. in a herbicidally effective amount) and at least one component selected from the group consisting of surfactants, solid diluents and liquid diluents. This invention further relates to a method for controlling the growth of undesired vegetation comprising contacting the vegetation or its environment with a herbicidally effective amount of a compound of the invention (e.g., as a composition described herein).

This invention also relates to a herbicidal mixture comprising (a) a compound selected from Formula 1, N-oxides, and salts thereof, and (b) at least one additional active ingredient selected from (b1) through (b16); and salts of compounds of (b1) through (b16), as described below.

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause, other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to herein, the term “seedling”, used either alone or in a combination of words means a young plant developing from the embryo of a seed.

As referred to herein, the term “broadleaf” used either alone or in words such as “broadleaf weed” means dicot or dicotyledon, a term used to describe a group of angiosperms characterized by embryos having two cotyledons.

As used herein, the term “alkylating agent” refers to a chemical compound in which a carbon-containing radical is bound through a carbon atom to a leaving group such as halide or sulfonate, which is displaceable by bonding of a nucleophile to said carbon atom. Unless otherwise indicated, the term “alkylating” does not limit the carbon-containing radical to alkyl; the carbon-containing radicals in alkylating agents include the variety of carbon-bound substituent radicals specified, for example, for R³.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” also includes moieties comprised of multiple triple bonds such as 2,5-hexadiynyl. The term “alkanediyl” refers to a straight-chain or branched alkyl group with two points of attachment. Examples of “alkandiyl” include —CH₂—, —CH₂CH₂—, —CH(CH₃)—, —CH₂CH₂CH₂—, —CH₂CH(CH₃)— and the different butylene isomers. “Alkenediyl” denotes a straight-chain or branched alkene containing at lease one olefinic bond. Examples of “alkenediyl” include —CH═CH—, —CH₂CH═CH—, —CH═C(CH₃)— and the different butenylene isomers.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂—, CH₃CH₂CH₂—, CH₃CH₂OCH₂—CH₃CH₂CH₂CH₂OCH₂— and CH₃CH₂OCH₂CH₂—. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl isomers. “Alkylthioalkyl” denotes alkylthio substitution on alkyl. Examples of “alkylthioalkyl” include CH₃SCH₂—, CH₃SCH₂CH₂—, CH₃CH₂SCH₂—, CH₃CH₂CH₂CH₂SCH₂— and CH₃CH₂SCH₂CH₂—. “Cyanoalkyl” denotes an alkyl group substituted with one cyano group. Examples of “cyanoalkyl” include NCCH₂—, NCCH₂CH₂— and CH₃CH(CN)CH₂—. “Alkylamino”, “dialkylamino”, and the like, are defined analogously to the above examples.

“Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C—, ClCH₂—, CF₃CH₂ and CF₃CCl₂. The terms “haloalkoxy”, “haloalkylthio”, “haloalkenyl”, “haloalkynyl”, and the like, are defined analogously to the term “haloalkyl”. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—. Examples of “haloalkenyl” include (Cl)₂C═CHCH₂— and CF₃CH₂CH═CHCH₂—. Examples of “haloalkynyl” include HC≡CCHCl—, CF₃C≡C—, CCl₃C≡C— and FCH₂C≡CCH₂—.

“Alkylcarbonyl” denotes a straight-chain or branched alkyl moieties bonded to a C(═O) moiety. Examples of “alkylcarbonyl” include CH₃C(═O)—, CH₃CH₂CH₂C(═O)— and (CH₃)₂CHC(═O)—. Examples of “alkoxycarbonyl” include CH₃OC(═O)—, CH₃CH₂OC(═O)—, CH₃CH₂CH₂C(═O)—, (CH₃)₂CHOC(═O)— and the different butoxy- or pentoxycarbonyl isomers.

The total number of carbon atoms in a substituent group is indicated by the “Cᵢ-Cⱼ” prefix where i and j are numbers from 1 to 8. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

When a compound is substituted with a substituent bearing a subscript that indicates the number of said substituents can exceed 1, said substituents (when they exceed 1) are independently selected from the group of defined substituents (e.g., (R^{A})ₙ, n is 0, 1 or 2). When a group contains a substituent which can be hydrogen, for example R³, R⁴, R⁵ or R⁷, then when this substituent is taken as hydrogen, it is recognized that this is equivalent to said group being unsubstituted. When a variable group is shown to be optionally attached to a position, for example R^{A}ₙ wherein n may be 0, then hydrogen may be at the position even if not recited in the variable group definition. When one or more positions on a group are said to be “not substituted” or “unsubstituted”, then hydrogen atoms are attached to take up any free valency.

Unless otherwise indicated, a“ring” as a component of Formula 1 (e.g., substituent R², R⁴, R⁵, R⁶ or R⁷) is heterocyclic. The term “ring member” refers to an atom or other moiety (e.g., C(═O), C(═S), S(O) or S(O)₂) forming the backbone of a ring.

The terms “heterocyclic ring” or “heterocycle” denote a ring in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring” or “aromatic heterocyclic ring”. Unless otherwise indicated, heterocyclic rings can be attached through any available carbon or nitrogen by replacement of a hydrogen on said carbon or nitrogen. “Aromatic” indicates that each of the ring atoms is essentially in the same plane and has a p-orbital perpendicular to the ring plane, and that (4n+2) π electrons, where n is a positive integer, are associated with the ring to comply with Hückel's rule.

The term “optionally substituted” in connection with the heterocyclic rings refers to groups which are unsubstituted or have at least one non-hydrogen substituent that does not extinguish the biological activity possessed by the unsubstituted analog. As used herein, the following definitions shall apply unless otherwise indicated. The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

When R², R⁵, R⁶ or R⁷ is a 5- or 6-membered heterocyclic ring, it may be attached to the remainder of Formula 1 though any available carbon or nitrogen ring atom, unless otherwise described. As noted above, R², R⁵, R⁶ or R⁷ can be (among others) phenyl optionally substituted with one or more substituents selected from a group of substituents as defined in the Summary of the Invention. An example of phenyl optionally substituted with 0 to 4 substituents is the ring illustrated as U-1 in Exhibit 1, wherein R^{v} defined in the Summary of the Invention as halogen, C₁-C₄ alkyl or C₁-C₄ haloalkyl.

As noted above, R², R⁵, R⁶ or R⁷ can be (among others) a 5- or 6-membered heterocyclic ring, which may be saturated or unsaturated, optionally substituted with one or more substituents selected from a group of substituents as defined in the Summary of the Invention. Examples of a 5- or 6-membered unsaturated aromatic heterocyclic ring optionally substituted with from one or more substituents include the rings U-2 through U-61 illustrated in Exhibit 1 wherein R^{v} is any substituent as defined in the Summary of the Invention for R², R⁵, R⁶ or R⁷ (i.e. halogen, C₁-C₄ alkyl or C₁-C₄ haloalkyl) and r is an integer from 0 to 4, limited by the number of available positions on each U group. As U-29, U-30, U-36, U-37, U-38, U-39, U-40, U-41, U-42 and U-43 have only one available position, for these U groups r is limited to the integers 0 or 1, and r being 0 means that the U group is unsubstituted and a hydrogen is present at the position indicated by (R^{v})ᵣ.

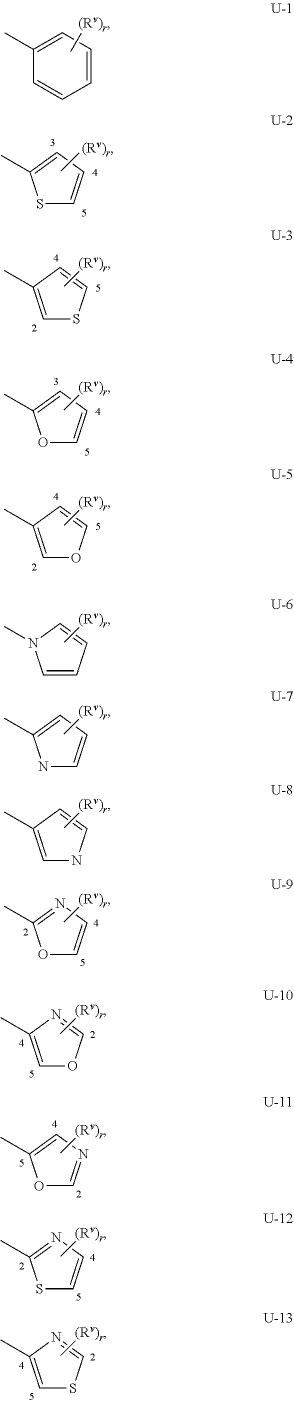

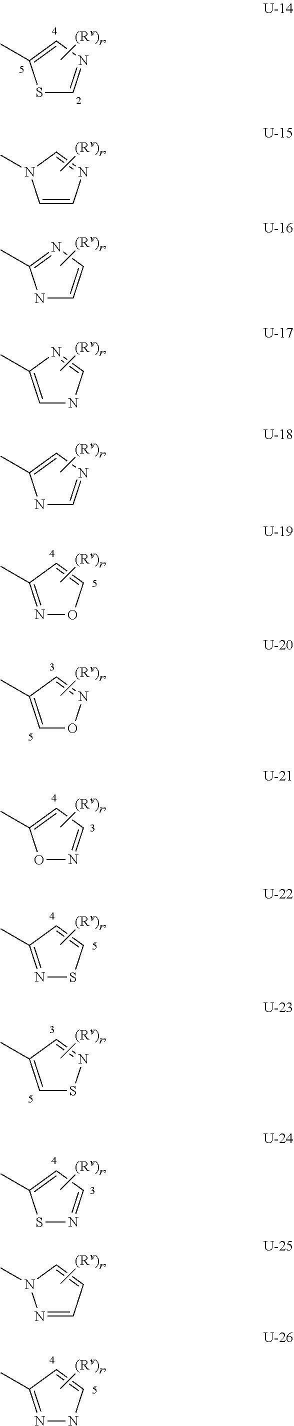

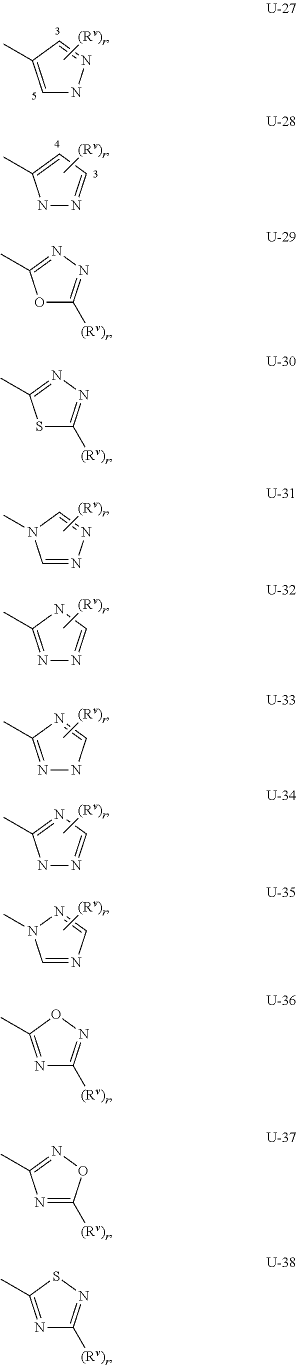

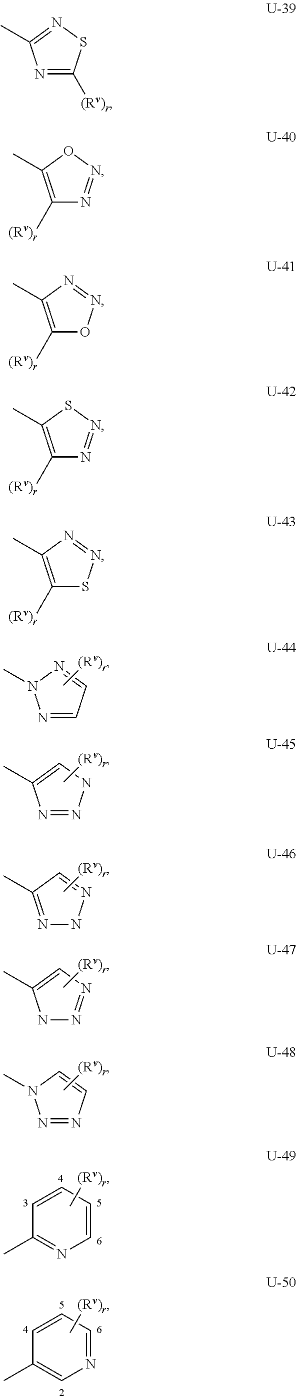

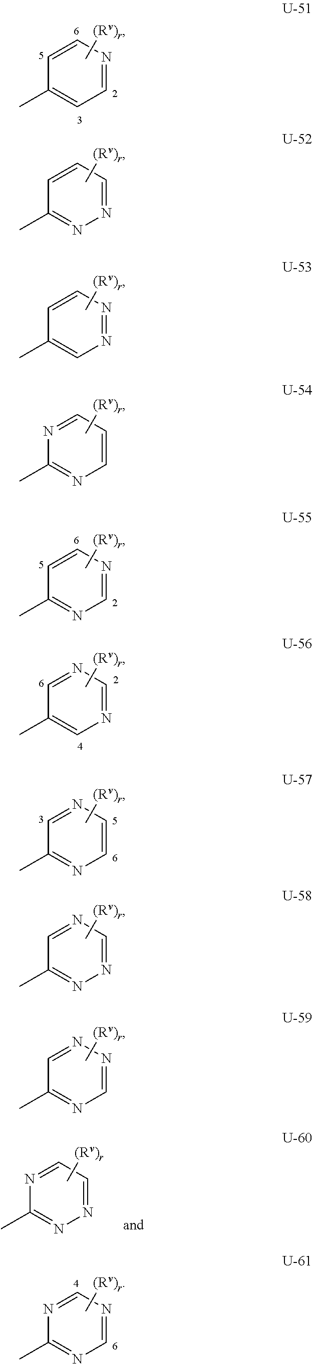

Note that when R², R⁵, R⁶ or R⁷ is a 5- or 6-membered saturated or unsaturated non-aromatic heterocyclic ring optionally substituted with one or four substituents selected from the group of substituents as defined in the Summary of the Invention (i.e. halogen, C₁-C₄ alkyl or C₁-C₄ haloalkyl), one or two carbon ring members of the heterocycle can optionally be in the oxidized form of a carbonyl moiety.

Examples of a 5- or 6-membered saturated or non-aromatic unsaturated heterocyclic ring containing ring members selected from up to two O atoms and up to two S atoms, and optionally substituted on carbon atom ring members with up to five halogen atoms includes the rings G-1 through G-35 as illustrated in Exhibit 2. Note that when the attachment point on the G group is illustrated as floating, the G group can be attached to the remainder of Formula 1 through any available carbon or nitrogen of the G group by replacement of a hydrogen atom. The optional substituents corresponding to R^{v} can be attached to any available carbon or nitrogen by replacing a hydrogen atom. For these G rings, r is typically an integer from 0 to 4, limited by the number of available positions on each G group.

Note that when R², R⁵, R⁶ or R⁷ comprises a ring selected from G-28 through G-35, G² is selected from O, S or N. Note that when G² is N, the nitrogen atom can complete its valence by substitution with either H or the substituents corresponding to R^{v} as defined in the Summary of the Invention (i.e. halogen, C₁-C₄ alkyl or C₁-C₄ haloalkyl).

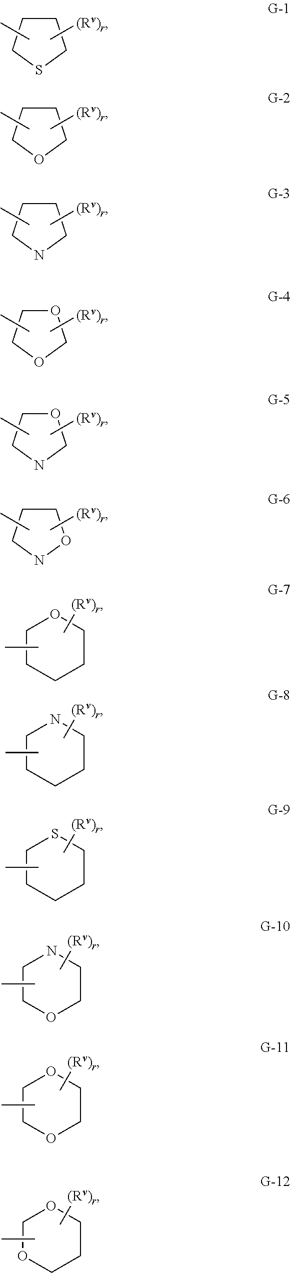

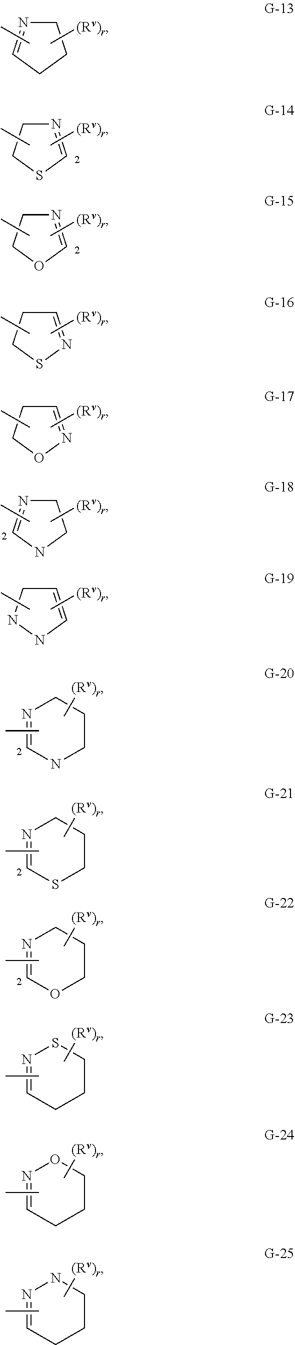

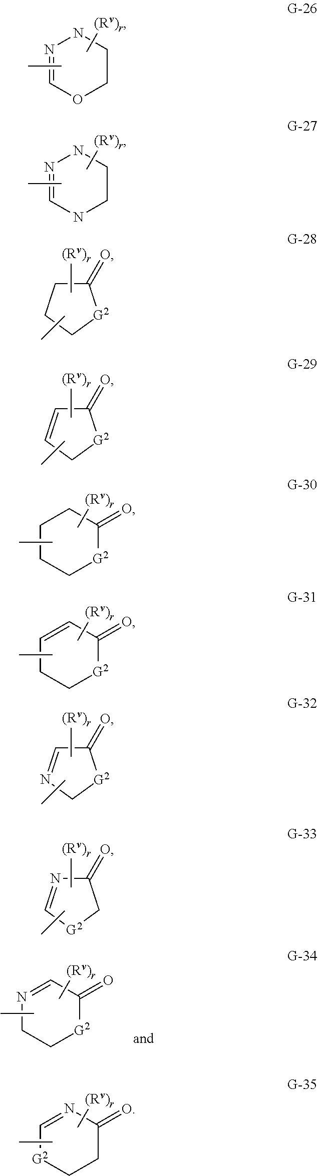

A wide variety of synthetic methods are known in the art to enable preparation of aromatic and nonaromatic heterocyclic rings; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as stereoisomers. The various stereoisomers include enantiomers, diastereomers, atropisomers and geometric isomers. Stereoisomers are isomers of identical constitution but differing in the arrangement of their atoms in space and include enantiomers, diastereomers, cis-trans isomers or Z/E isomers (also known as geometric isomers) and atropisomers.

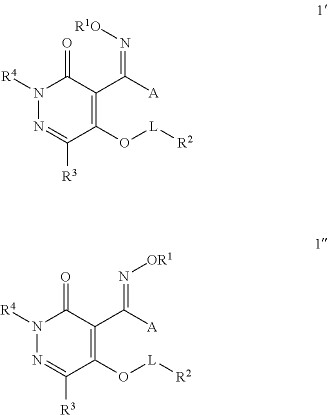

One skilled in the art will appreciate that one stereoisomer (i.e. Z/E isomer) may be more active and/or may exhibit beneficial effects when enriched relative to the other isomers or when separated from the other isomer. Additionally, the skilled artisan knows how to separate, enrich, and/or to selectively prepare said isomers. The compounds of the invention may be present as a mixture of isomers or individual isomers. Preferred for biological activity are compounds of Formula 1″, alternatively known as the E isomer. Conventions herein refer to the E and Z isomers about the C═N bond in Formula 1 irrespective of the priority of A. Compounds of Formula 1 can also comprise additional chiral centers. For example, substituents and other molecular constituents such as R² and R³ may themselves contain chiral centers. This invention comprises racemic mixtures as well as enriched and essentially pure stereoconfigurations at these additional chiral centers.

Compounds of Formula 1 typically exist in more than one form, and Formula 1 thus includes all crystalline and non-crystalline forms of the compounds they represent. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound of Formula 1 can exhibit beneficial effects (e.g., suitability for preparation of useful formulations, improved biological performance) relative to another polymorph or a mixture of polymorphs of the same compound of Formula 1. Preparation and isolation of a particular polymorph of a compound of Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750. S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of a compound of Formula 1 are useful for control of undesired vegetation (i.e. are agriculturally suitable). The salts of a compound of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Embodiments of the present invention as described in the Summary of the Invention include:

### Embodiment 1

A compound of Formula 1, including all isomers, stereoisomers and N-oxides of such compounds, and salts of such compounds, isomers, stereoisomers and N-oxides, and methods of their use for controlling undesired vegetation as described in the Summary of the Invention.

### Embodiment 2

A compound of Embodiment 1 wherein R¹ is H, C₁-C₇ alkyl, C₂-C₇ alkenyl, C₃-C₇ alkynyl, C₁-C₇ haloalkyl, C₂-C₇ haloalkenyl, C₄-C₈ alkylcycloalkyl or C₂-C₇ cyanoalkyl.

### Embodiment 3

A compound of Embodiment 2 wherein R¹ is H, C₁-C₇ alkyl, C₂-C₇ alkenyl, C₃-C₇ alkynyl, C₁-C₇ haloalkyl, C₂-C₇ haloalkenyl or C₄-C₈ alkylcycloalkyl.

### Embodiment 4

A compound of Embodiment 3 wherein R¹ is C₁-C₃ alkyl, C₂-C₃ alkenyl, C₂-C₃ alkynyl or C₂-C₃ haloalkenyl.

### Embodiment 5

A compound of Embodiment 4 wherein R¹ is CH₃, CH₂CH₃, i-Pr, —CH₂CH═CH₂ or —CH₂C═CH.

### Embodiment 6

A compound of Embodiment 5 wherein R¹ is CH₃, i-Pr or —CH₂C≡CH.

### Embodiment 7

A compound of Embodiment 6 wherein R¹ is CH₃ or i-Pr.

### Embodiment 8

A compound of Embodiment 6 wherein R¹ is —CH₂C≡CH.

### Embodiment 9

A compound of Embodiment 5 wherein R¹ is CH₂CH₃.

### Embodiment 10

A compound of Embodiment 5 wherein R¹ is CH₃.

### Embodiment 11

A compound of any one of Embodiments 1 through 10 wherein A is selected from the group consisting of A-1, A-2, A-3, A-4, A-6, A-7, A-8 and A-9.

### Embodiment 12

A compound of Embodiment 11 wherein A is selected from the group consisting of A-1, A-2, A-3, A-6, A-7 and A-8.

### Embodiment 13

A compound of Embodiment 12 wherein A is selected from the group consisting of A-1, A-6, A-7 and A-8.

### Embodiment 14

A compound of Embodiment 13 wherein A is selected from the group consisting of A-1 and A-6.

### Embodiment 15

A compound of Embodiment 14 wherein A is A-1.

### Embodiment 16

A compound of Embodiment 14 wherein A is A-6.

### Embodiment 17

A compound of any one of Embodiments 1 through 14 wherein A is other than A-1.

### Embodiment 18

A compound of any one of Embodiments 1 through 12 wherein A is selected from the group consisting of A-2 and A-3.

### Embodiment 19

A compound of any one of Embodiments 1 through 13 wherein A is selected from the group consisting of A-7 and A-8.

### Embodiment 20

A compound of any one of Embodiments 1 through 19 wherein each R^{A} is independently halogen, cyano, C₁-C₅ alkyl, C₃-C₅ cycloalkyl, C₄-C₅ cycloalkylalkyl, C₁-C₅ haloalkyl, C₂-C₅ alkoxyalkyl, C₁-C₅ alkoxy, C₁-C₅ alkylthio or C₁-C₄ alkylsulfonyl.

### Embodiment 21

A compound of Embodiment 20 wherein each R^{A} is independently halogen, C₁-C₅ alkyl, C₁-C₅ haloalkyl or C₁-C₅ alkoxy.

### Embodiment 22

A compound of Embodiment 21 wherein each R^{A} is independently F. Cl, Br, CH₃ or OCH₃.

### Embodiment 23

A compound of Embodiment 22 wherein each R^{A} is independently F, Cl, Br or CH₃.

### Embodiment 24

A compound of Embodiment 23 wherein each R^{A} is independently F. Cl or Br.

### Embodiment 25

A compound of any one of Embodiments 1 through 24 wherein n is 0, 1 or 2.

### Embodiment 26

A compound of Embodiment 25 wherein n is 0.

### Embodiment 27

A compound of Embodiment 25 wherein n is 1 or 2.

### Embodiment 28

A compound of Embodiment 27 wherein n is 1.

### Embodiment 29

A compound of Embodiment 27 wherein n is 2.

### Embodiment 30

A compound of any one of Embodiments 1 through 29 wherein L is a direct bond, C₁-C₂ alkanediyl or C₂-C₃ alkenediyl.

### Embodiment 31

A compound of any one of Embodiments 1 through 30 wherein L is a direct bond, —CH₂— or —CH═CH—.

### Embodiment 32

A compound of Embodiment 31 wherein L is a direct bond or —CH₂—.

### Embodiment 33

A compound of Embodiment 32 wherein L is a direct bond.

### Embodiment 34

A compound of Embodiment 30 wherein L is —CH₂— or —CH═CH—.

### Embodiment 35

A compound of Embodiment 34 wherein L is —CH₂—.

### Embodiment 36

A compound of any one of Embodiments 1 through 35 wherein R² is H, C(═O)R⁵, C(═S)R⁵, CO₂R⁶, C(═O)SR⁶, CON(R⁷)R⁸ or P(═O)(R⁹)R¹⁰; or C₁-C₄ alkyl, C₂-C₄ alkenyl, C₂-C₄ alkynyl, C₁-C₄ haloalkyl, C₂-C₄ haloalkenyl, C₂-C₄ haloalkynyl or C₂-C₄ alkoxyalkyl.

### Embodiment 37

A compound of Embodiment 36 wherein R² is H, C(═O)R⁵, CO₂R⁶, CON(R⁷)R⁸ or P(═O)(R⁹)R¹⁰; or C₁-C₄ alkyl, C₂-C₄ alkenyl, C₁-C₄ haloalkyl, C₂-C₄ haloalkenyl or C₂-C₄ alkoxyalkyl.

### Embodiment 38

A compound of Embodiment 37 wherein R² is H, C(═O)R⁵, CO₂R⁶ or P(═O)(R⁹)R¹⁰; or C₁-C₄ alkyl, C₁-C₄ haloalkyl or C₂-C₄ alkoxyalkyl.

### Embodiment 39

A compound of Embodiment 38 wherein R² is H, C(═O)R⁵ or CO₂R⁶; or C₂-C₄ alkoxyalkyl.

### Embodiment 40

A compound of Embodiment 39 wherein R² is H, C(═O)R⁵ or CO₂R⁶.

### Embodiment 41

A compound of Embodiment 39 wherein R² is H.

### Embodiment 42

A compound of Embodiment 39 wherein R² is C(═O)R⁵ or CO₂R⁶.

### Embodiment 43

A compound of Embodiment 39 wherein R² is C(═O)R⁵.

### Embodiment 44

A compound of any one of Embodiments 1 through 43 wherein R³ is H, halogen, cyano, —CHO, C₁-C₇ alkyl, C₃-C₈ alkylcarbonylalkyl, C₃-C₈ alkoxycarbonylalkyl, C₁-C₄ alkylcarbonyl, C₂-C₇ alkylcarbonyloxy, C₄-C₇ alkylcycloalkyl, C₃-C₇ alkenyl, C₃-C₇ alkynyl, C₁-C₄ alkylsulfinyl, C₁-C₄ alkylsulfonyl, C₁-C₄ alkylamino, C₂-C₈ dialkylamino, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₃-C₇ haloalkenyl, C₂-C₇ alkoxyalkyl, C₁-C₇ alkoxy or C₁-C₅ alkylthio.

### Embodiment 45

A compound of Embodiment 44 wherein R³ is H, halogen, cyano, —CHO, C₁-C₇ alkyl, C₁-C₄ alkylcarbonyl, C₂-C₇ alkylcarbonyloxy, C₄-C₇ alkylcycloalkyl, C₁-C₄ alkylsulfinyl, C₁-C₄ alkylsulfonyl, C₁-C₄ alkylamino, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₂-C₇ alkoxyalkyl or C₁-C₇ alkoxy.

### Embodiment 46

A compound of Embodiment 45 wherein R³ is H, halogen, cyano, C₁-C₄ alkyl, C₃-C₅ cycloalkyl, C₁-C₃ haloalkyl, C₂-C₄ alkoxyalkyl or C₁-C₃ alkoxy.

### Embodiment 47

A compound of Embodiment 46 wherein R³ is H, halogen, C₁-C₃ alkyl, cyclopropyl or C₁-C₂ haloalkyl.

### Embodiment 48

A compound of Embodiment 47 wherein R³ is H, Cl, Br, I, CH₃, CH₂CH₃ or cyclopropyl.

### Embodiment 49

A compound of Embodiment 48 wherein R³ is H, Cl, CH₃ or cyclopropyl.

### Embodiment 50

A compound of Embodiment 49 wherein R³ is Cl or CH₃.

### Embodiment 51

A compound of any one of Embodiments 1 through 50 wherein R³ is other than H.

### Embodiment 52

A compound of any one of Embodiments 1 through 51 wherein R⁴ is H, C₁-C₇ alkyl, C₃-C₈ alkylcarbonylalkyl, C₃-C₈ alkoxycarbonylalkyl, C₄-C₇ alkylcycloalkyl, C₃-C₇ alkenyl, C₃-C₇ alkynyl, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₃-C₇ haloalkenyl, C₂-C₇ alkoxyalkyl, C₃-C₇ alkylthioalkyl or C₁-C₇ alkoxy; or benzyl optionally substituted by halogen, C₁-C₄ alkyl or C₁-C₄ haloalkyl.

### Embodiment 53

A compound of Embodiment 52 wherein R⁴ is H, C₁-C₇ alkyl, C₃-C₈ alkoxycarbonylalkyl, C₄-C₇ alkylcycloalkyl, C₃-C₇ alkenyl, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₂-C₇ alkoxyalkyl or C₁-C₇ alkoxy; or benzyl optionally substituted by halogen, C₁-C₄ alkyl or C₁-C₄ haloalkyl.

### Embodiment 54

A compound of Embodiment 53 wherein R⁴ is C₁-C₄ alkyl, C₃-C₇ alkenyl, C₃-C₄ cycloalkyl, C₄-C₇ cycloalkylalkyl, C₂-C₃ cyanoalkyl, C₁-C₃ haloalkyl or C₂-C₄ alkoxyalkyl.

### Embodiment 55

A compound of Embodiment 54 wherein R⁴ is C₁-C₃ alkyl, C₃-C₄ cycloalkyl, —CH₂CH₂C═N, C₁-C₂ haloalkyl or 2-methoxyethyl.

### Embodiment 56

A compound of Embodiment 55 wherein R⁴ is CH₃, CH₂CH₃ or c-Pr.

### Embodiment 57

A compound of Embodiment 56 wherein R⁴ is CH₃, CH₂CH₃.

### Embodiment 58

A compound of Embodiment 57 wherein R⁴ is CH₃.

### Embodiment 59

A compound of Embodiment 52 or 53 wherein R⁴ is other than H.

### Embodiment 60

A compound of any one of Embodiments 1 through 69 wherein each R⁵ and R⁷ are independently H, C₁-C₇ alkyl, C₃-C₇ alkenyl, C₃-C₇ alkynyl, C₃-C₇ cycloalkyl, C₁-C₇ haloalkyl, C₃-C₇ haloalkenyl, C₂-C₇ alkoxyalkyl or C₄-C₇ cycloalkylalkyl; or phenyl or benzyl, each phenyl or benzyl optionally substituted by halogen, C₁-C₄ alkyl or C₁-C₄ haloalkyl.

### Embodiment 61

A compound of Embodiment 60 wherein each R⁵ and R⁷ are independently H, C₁-C₇ alkyl, C₃-C₇ cycloalkyl or C₂-C₇ alkoxyalkyl; or phenyl, optionally substituted by halogen, C₁-C₄ alkyl or C₁-C₄ haloalkyl.

### Embodiment 62

A compound of Embodiment 61 wherein R⁵ is H, C₁-C₇ alkyl, C₃-C₇ cycloalkyl or C₂-C₇ alkoxyalkyl.

### Embodiment 63

A compound of Embodiment 62 wherein R⁵ is C₁-C₇ alkyl.

### Embodiment 64

A compound of any one of Embodiments 1 through 59 wherein R⁶ is C₁-C₇ alkyl, C₃-C₇ alkenyl, C₃-C₇ alkynyl, C₃-C₇ cycloalkyl, C₂-C₇ haloalkyl, C₃-C₇ haloalkenyl, C₂-C₇ alkoxyalkyl or C₄-C₇ cycloalkylalkyl; or phenyl or benzyl, each phenyl or benzyl optionally substituted by halogen, C₁-C₄ alkyl or C₁-C₄ haloalkyl.

### Embodiment 65

A compound of Embodiment 64 wherein R⁶ is C₁-C₇ alkyl, C₂-C₇ haloalkyl or C₂-C₇ alkoxyalkyl; or phenyl optionally substituted by halogen, C₁-C₄ alkyl or C₁-C₄ haloalkyl.

### Embodiment 66

A compound of Embodiment 65 wherein R⁶ is C₁-C₇ alkyl; or phenyl optionally substituted by halogen or C₂-C₄ alkyl.

### Embodiment 67

A compound of Embodiment 66 wherein R⁶ is C₁-C₇ alkyl.

### Embodiment 68

A compound of any one of Embodiments 1 through 59 wherein R⁸ is H, C₁-C₇ alkyl, C₃-C₇ cycloalkyl, C₄-C₇ cycloalkylalkyl or C₁-C₇ haloalkyl.

### Embodiment 69

A compound of Embodiment 68 wherein R⁸ is H, C₁-C₇ alkyl or C₁-C₇ haloalkyl.

### Embodiment 70

A compound of any one of Embodiments 1 through 59 wherein R⁹ is C₁-C₄ alkyl or C₁-C₄ alkoxy.

### Embodiment 71

A compound of Embodiment 70 wherein R⁹ is CH₃ or OCH₃.

### Embodiment 72

A compound of Embodiment 70 wherein R⁹ is OCH₃.

### Embodiment 73

A compound of any one of Embodiments 1 through 59 wherein R¹⁰ is C₁-C₄ alkyl or C₁-C₄ alkoxy.

### Embodiment 74

A compound of any one of Embodiment 73 wherein R¹⁰ is CH₃ or OCH₃.

### Embodiment 75

A compound of any one of Embodiment 74 wherein R¹⁰ is OCH₃.

### Embodiment 76

A compound of any one of Embodiments 1 through 20 wherein each R^{A} is other than C₁-C₄ alkylsulfonyl.

### Embodiment 77

A compound of any one of Embodiments 1 through 20 wherein each R^{A} is other than C₁-C₅ alkylthio or C₁-C₄ alkylsulfonyl.

### Embodiment 78

A compound of any one of Embodiments 1 through 20 wherein each R^{A} is other than C₁-C₅ alkylthio, C₁-C₄ alkylsulfinyl, C₁-C₄ alkylsulfonyl, C₁-C₅ haloalkylthio.

### Embodiment 79

A compound of any one of Embodiments 1 through 20 wherein R^{A} is other than C₁-C₅ alkylthio.

### Embodiment 80

A compound of any one of Embodiments 1 through 20 wherein R^{A} is other than C₁-C₅ alkoxy.

### Embodiment 81

A compound of Embodiment 1 wherein when A is A-1, R^{A} is other than C₁-C₅ alkoxy.

### Embodiment 82

A compound of Embodiment 1 wherein R¹ is other than unsubstituted benzyl.

Embodiments of this invention, including Embodiments 1-82 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-82 above as well as any other embodiments described herein, and any combination thereof, pertain to the compositions and methods of the present invention.

### Embodiment A

A compound of the Summary of the Invention wherein
R¹ is H, C₁-C₇ alkyl, C₂-C₇ alkenyl, C₃-C₇ alkynyl, C₁-C₇ haloalkyl, C₂-C₇ haloalkenyl, C₄-C₈ alkylcycloalkyl or C₂-C₇ cyanoalkyl;
A is selected from the group consisting of A-1, A-2, A-3, A-4, A-6, A-7, A-8 and A-9;
each R^{A} is independently halogen, cyano, C₁-C₅ alkyl, C₃-C₅ cycloalkyl, C₄-C₅ cycloalkylalkyl, C₁-C₅ haloalkyl, C₂-C₅ alkoxyalkyl, C₁-C₅ alkoxy, C₁-C₅ alkylthio or C₁-C₄ alkylsulfonyl;
n is 0, 1 or 2;
L is a direct bond, C₁-C₂ alkanediyl or C₂-C₃ alkenediyl;
R² is H, C(═O)R⁵, C(═S)R⁵, CO₂R⁶, C(═O)SR⁶, CON(R⁷)R⁸ or P(═O)(R⁹)R¹⁰; or C₁-C₄ alkyl, C₂-C₄ alkenyl, C₂-C₄ alkynyl, C₁-C₄ haloalkyl, C₂-C₄ haloalkenyl, C₂-C₄ haloalkynyl or C₂-C₄ alkoxyalkyl;
R³ is H, halogen, cyano, —CHO, C₁-C₇ alkyl, C₃-C₈ alkylcarbonylalkyl, C₃-C₈ alkoxycarbonylalkyl, C₁-C₄ alkylcarbonyl, C₂-C₇ alkylcarbonyloxy, C₄-C₇ alkylcycloalkyl, C₃-C₇ alkenyl, C₃-C₇ alkynyl, C₁-C₄ alkylsulfinyl, C₁-C₄ alkylsulfonyl, C₁-C₄ alkylamino, C₂-C₈ dialkylamino, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₃-C₇ haloalkenyl, C₂-C₇ alkoxyalkyl, C₁-C₇ alkoxy or C₁-C₅ alkylthio;
R⁴ is H, C₁-C₇ alkyl, C₃-C₈ alkylcarbonylalkyl, C₃-C₈ alkoxycarbonylalkyl, C₄-C₇ alkylcycloalkyl, C₃-C₇ alkenyl, C₃-C₇ alkynyl, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₃-C₇ haloalkenyl, C₂-C₇ alkoxyalkyl, C₃-C₇ alkylthioalkyl or C₁-C₇ alkoxy; or benzyl optionally substituted by halogen, C₁-C₄ alkyl or C₁-C₄ haloalkyl;
each R⁵ and R⁷ are independently H, C₁-C₇ alkyl, C₃-C₇ alkenyl, C₃-C₇ alkynyl, C₃-C₇ cycloalkyl, C₁-C₇ haloalkyl, C₃-C₇ haloalkenyl, C₂-C₇ alkoxyalkyl or C₄-C₇ cycloalkylalkyl; or phenyl, benzyl, each phenyl, benzyl optionally substituted by halogen, C₁-C₄ alkyl or C₁-C₄ haloalkyl;
R⁶ is C₁-C₇ alkyl, C₃-C₇ alkenyl, C₃-C₇ alkynyl, C₃-C₇ cycloalkyl, C₂-C₇ haloalkyl, C₃-C₇ haloalkenyl, C₂-C₇ alkoxyalkyl or C₄-C₇ cycloalkylalkyl; or phenyl or benzyl, each phenyl or benzyl optionally substituted by halogen, C₁-C₄ alkyl or C₁-C₄ haloalkyl;
R⁸ is H, C₁-C₇ alkyl, C₃-C₇ cycloalkyl, C₄-C₇ cycloalkylalkyl or C₁-C₇ haloalkyl;
R⁹ is C₁-C₄ alkyl or C₁-C₄ alkoxy; and
R¹⁰ is C₁-C₄ alkyl or C₁-C₄ alkoxy.

### Embodiment B

A compound of Embodiment A wherein
R¹ is H, C₁-C₇ alkyl, C₂-C₇ alkenyl, C₃-C₇ alkynyl, C₁-C₇ haloalkyl, C₂-C₇ haloalkenyl or C₄-C₈ alkylcycloalkyl;
A is selected from the group consisting of A-1, A-2, A-3, A-6, A-7 and A-8;
each R^{A} is independently halogen, C₁-C₅ alkyl, C₁-C₅ haloalkyl or C₁-C₅ alkoxy;
n is 1 or 2;
L is a direct bond, —CH₂— or —CH═CH—;
R² is H, C(═O)R⁵, CO₂R⁶, CON(R⁷)R⁸ or P(═O)(R⁹)R¹⁰; or C₁-C₄ alkyl, C₂-C₄ alkenyl, C₁-C₄ haloalkyl, C₂-C₄ haloalkenyl or C₂-C₄ alkoxyalkyl;
R³ is H, halogen, cyano, —CHO, C₁-C₇ alkyl, C₁-C₄ alkylcarbonyl, C₂-C₇ alkylcarbonyloxy, C₄-C₇ alkylcycloalkyl, C₁-C₄ alkylsulfinyl, C₁-C₄ alkylsulfonyl, C₁-C₄ alkylamino, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₂-C₇ alkoxyalkyl or C₁-C₇ alkoxy;
R⁴ is H, C₁-C₇ alkyl, C₃-C₈ alkoxycarbonylalkyl, C₄-C₇ alkylcycloalkyl, C₃-C₇ alkenyl, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₂-C₇ alkoxyalkyl or C₁-C₇ alkoxy; or benzyl optionally substituted by halogen, C₁-C₄ alkyl or C₁-C₄ haloalkyl;
each R⁵ and R⁷ are independently H, C₁-C₇ alkyl, C₃-C₇ cycloalkyl or C₂-C₇ alkoxyalkyl; or phenyl, optionally substituted by halogen, C₁-C₄ alkyl or C₁-C₄ haloalkyl;
R⁶ is C₁-C₇ alkyl, C₂-C₇ haloalkyl or C₂-C₇ alkoxyalkyl; or phenyl optionally substituted by halogen, C₁-C₄ alkyl or C₁-C₄ haloalkyl;
R⁸ is H, C₁-C₇ alkyl or C₁-C₇ haloalkyl;
R⁹ is CH₃ or OCH₃; and
R¹⁰ is CH₃ or OCH₃.

### Embodiment C

A compound of the Embodiment B wherein
R¹ is C₁-C₃ alkyl, C₂-C₃ alkenyl, C₂-C₃ alkynyl or C₂-C₃ haloalkenyl;
A is selected from the group consisting of A-1, A-6, A-7 and A-8;
each R^{A} is independently F, Cl, Br, CH₃ or OCH₃;
R² is H, C(═O)R⁵, CO₂R⁶ or P(═O)(R⁹)R¹⁰; or C₁-C₄ alkyl, C₁-C₄ haloalkyl or C₂-C₄ alkoxyalkyl;
R³ is H, halogen, cyano, C₁-C₄ alkyl, C₃-C₅ cycloalkyl, C₁-C₃ haloalkyl, C₂-C₄ alkoxyalkyl or C₁-C₃ alkoxy;
R⁴ is C₁-C₄ alkyl, C₃-C₇ alkenyl, C₃-C₄ cycloalkyl, C₄-C₇ cycloalkylalkyl, C₂-C₃ cyanoalkyl, C₁-C₃ haloalkyl or C₂-C₄ alkoxyalkyl
R⁵ is C₁-C₇ alkyl;
R⁶ is C₁-C₇ alkyl; or phenyl optionally substituted by halogen or C₁-C₄ alkyl;
R⁹ is OCH₃; and
R¹⁰ is OCH₃.

### Embodiment D

A compound of Embodiment C wherein
R¹ is CH₃, CH₂CH₃, i-Pr, —CH₂CH═CH₂ or —CH₂C═CH;
A is selected from the group consisting of A-1 and A-6;
each R^{A} is independently F, Cl, Br or CH₃;
R² is H, C(═O)R⁵ or CO₂R⁶; or C₂-C₄ alkoxyalkyl;
R³ is H, halogen, C₁-C₃ alkyl, cyclopropyl or C₁-C₂ haloalkyl;
R⁴ is C₁-C₃ alkyl, —CH₂CH₂C≡N, C₁-C₂ haloalkyl or 2-methoxyethyl; and
R⁶ is C₁-C₇ alkyl.

### Embodiment E

A compound of Embodiment D wherein
R¹ is CH₃, i-Pr or —CH₂C═CH,
A is A-1;
each R^{A} is independently F, Cl or Br;
R² is H, C(═O)R⁵ or CO₂R⁶;
R³ is H, Cl, Br, I, CH₃, CH₂CH₃ or cyclopropyl; and
R⁴ is CH₃, CH₂CH₃ or c-Pr.

### Embodiment F

A compound of Embodiment D wherein
R¹ is CH₃ or i-Pr;
A is A-6;
each R^{A} is independently F, Cl or Br;
R² is H, C(═O)R⁵ or CO₂R⁶;
R³ is H, Cl, CH₃ or cyclopropyl; and
R⁴ is CH₃ or CH₂CH₃.

### Embodiment G

A compound of the Summary of the Invention selected from the group consisting of
4-[(E)-(3-bromo-1-naphthalenyl)(methoxyimino)methyl]-5-hydroxy-2,6-dimethyl-3(2H)-pyridazinone (Compound 99):
4-[(Z)-(3-bromo-1-naphthalenyl)(methoxyimino)methyl]-5-hydroxy-2,6-dimethyl-3(2H)-pyridazinone (Compound 91);
4-[(E)-(3-bromo-1-naphthalenyl)[(2-propyn-1-yloxy)imino]methyl]-5-hydroxy-2,6-dimethyl-3(2H)-pyridazinone (Compound 112):
4-[(E)-(3-bromo-1-naphthalenyl)(ethoxyimino)methyl]-5-hydroxy-2,6-dimethyl-3(2H)-pyridazinone (Compound 113)
4-[(Z)-(4-fluoro-1-naphthalenyl)(2-propyn-1-yloxy)imino]methyl-5-hydroxy-2,6-dimethyl-3(2H)-pyridazinone (Compound 108); and
4-[(E)-(4-fluoro-1-naphthalenyl)[(2-propyn-1-yloxy)imino]methyl]-5-hydroxy-2,6-dimethyl-3(2H)-pyridazinone (Compound 109).

### Embodiment H

A compound of the Summary of the Invention selected from the group consisting of
a mixture of Compound 129 and Compound 145 (i.e. a mixture of E and Z isomers wherein A is A-6; n=0; R¹ is CH₃; L is a direct bond; R² is H; R³ is Cl; and R⁴ is CH₃);
a mixture of Compound 147 and Compound 146 (a mixture of E and Z isomers wherein A is A-6; n=0; R¹ is CH₂CH₃; L is a direct bond; R² is H; R³ is Cl; and R⁴ is CH₃);
a mixture of Compound 99 and Compound 91 (a mixture of E and Z isomers wherein A is A-6; R^{A} is 3-Br; R¹ is CH₃; L is a direct bond; R² is H; R³ is CH₃; and R⁴ is CH₃);
a mixture of Compound 88 and Compound 89 (a mixture of E and Z isomers wherein A is A-6; R^{A} is 3-F; R¹ is CH(CH₃)₂; L is a direct bond; R² is H R³ is CH₃; and R⁴ is CH₃); and
a mixture of Compound 113 and Compound 114 (a mixture of E and Z isomers wherein A is A-6; R^{A} is 3-Br; R¹ is CH₂CH₃; L is a direct bond; R² is H; R³ is CH₃; and R⁴ is CH₃).

This invention also relates to a method for controlling undesired vegetation comprising applying to the locus of the vegetation herbicidally effective amounts of the compounds of the invention (e.g., as a composition described herein). Of note as embodiments relating to methods of use are those involving the compounds of embodiments described above. Compounds of the invention are particularly useful for selective control of weeds in cereal crops such as wheat, barley, maize, soybean, sunflower, cotton and oilseed rape, and specialty crops such as sugarcane, citrus, fruit and nut crops.

Also noteworthy as embodiments are herbicidal compositions of the present invention comprising the compounds of embodiments described above.

This invention also includes a herbicidal mixture comprising (a) a compound selected from Formula 1, N-oxides, and salts thereof, and (b) at least one additional active ingredient selected from (b1) photosystem II inhibitors, (b2) acetohydroxy acid synthase (AHAS) inhibitors, (b3) acetyl-CoA carboxylase (ACCase) inhibitors, (b4) auxin mimics, (b5) 5-enol-pyruvylshikimate-3-phosphate (EPSP) synthase inhibitors, (b6) photosystem I electron diverters, (b7) protoporphyrinogen oxidase (PPO) inhibitors, (b8) glutamine synthetase (GS) inhibitors, (b9) very long chain fatty acid (VLCFA) elongase inhibitors, (b10) auxin transport inhibitors, (b11) phytoene desaturase (PDS) inhibitors, (b12) 4-hydroxyphenyl-pyruvate dioxygenase (HPPD) inhibitors, (b13) homogentisate solenesyltransererase (HST) inhibitors, (b14) cellulose biosynthesis inhibitors, (b15) other herbicides including mitotic disruptors, organic arsenicals, asulam, bromobutide, cinmethylin, cumvluron, dazomet, difenzoquat, dymron, etobenzanid, flurenol, fosamine, fosamine-ammonium, hydantocidin, metam, methyldymron, oleic acid, oxaziclomefone, pelargonic acid and pyributicarb, and (b16) herbicide safeners; and salts of compounds of (b1) through (b16). Preferred is a herbicidal mixture comprising (a) a compound selected from Formula 1, N-oxides, and salts thereof, and (b) at least one additional active ingredient selected from (b2) acetohydroxy acid synthase (AHAS) inhibitors; and (b12) 4-hydroxyphenyl-pyruvate dioxygenase (HPPD) inhibitors.

“Photosystem II inhibitors” (b1) are chemical compounds that bind to the D-1 protein at the Q-binding niche and thus block electron transport from Q_{A} to Q_{B} in the chloroplast thylakoid membranes. The electrons blocked from passing through photosystem II are transferred through a series of reactions to form toxic compounds that disrupt cell membranes and cause chloroplast swelling, membrane leakage, and ultimately cellular destruction. The Q_{B}-binding niche has three different binding sites: binding site A binds the triazines such as atrazine, triazinones such as hexazinone, and uracils such as bromacil, binding site B binds the phenylureas such as diuron, and binding site C binds benzothiadiazoles such as bentazon, nitriles such as bromoxynil and phenyl-pyridazines such as pyridate. Examples of photosystem 1 inhibitors include ametryn, amicarbazone, atrazine, bentazon, bromacil, bromofenoxim, bromoxynil, chlorbromuron, chloridazon, chlorotoluron chloroxuron, cumyluron, cyanazine, daimuron, desmedipham, desmetryn, dimefuron, dimethametryn, diuron, ethidimuron, fenuron, fluometuron, hexazinone, ioxynil, isoproturon, isouron, lenacil, linuron, metamitron, methabenzthiazuron, metobromuron, metoxuron, metribuzin, monolinuron, neburon, pentanochlor, phenmedipham, prometon, prometryn, propanil, propazine, pyridafol, pyridate, siduron, simazine, simetryn, tebuthiuron, terbacil, terbumeton, terbuthylazine, terbutryn and trietazine.

“AHAS inhibitors” (b2) are chemical compounds that inhibit acetohydroxy acid synthase (AHAS), also known as acetolactate synthase (ALS), and thus kill plants by inhibiting the production of the branched-chain aliphatic amino acids such as valine, leucine and isoleucine, which are required for protein synthesis and cell growth. Examples of AHAS inhibitors include amidosulfuron, azimsulfuron, bensulfuron-methyl, bispyribac-sodium, cloransulam-methyl, chlorimuron-ethyl, chlorsulfuron, cinosulfuron, cyclosulfamuron, diclosulam, ethametsulfuron-methyl, ethoxysulfuron, flazasulfuron, florasulam, flucarbazone-sodium, flumetsulam, flupyrsulfuron-methyl, flupyrsulfuron-sodium, foramsulfuron, halosulfuron-methyl, imazamethabenz-methyl, imazamox, imazapic, imazapyr, imazaquin, imazethapyr, imazosulfuron, iodosulfuron-methyl (including sodium salt), iofensulfuron (2-iodo-N-[[(4-methoxy-6-methyl-1,3,5-triazin-2-yl)amino]carbonyl]benzenesulfonamide), mesosulfuron-methyl, metazosulfuron(3-chloro-4-(5,6-dihydro-5-methyl-1,4,2-dioxazin-3-yl)-N-[[(4,6-dimethoxy-2-pyrimidinyl)amino]carbonyl]-1-methyl-1H-pyrazole-5-sulfonamide), metosulam, metsulfuron-methyl, nicosulfuron, oxasulfuron, penoxsulam, primisulfuron-methyl, propoxycarbazone-sodium, propyrisulfuron (2-chloro-N-[[(4,6-dimethoxy-2-pyrimidinyl)amino]carbonyl]-6-propylimidazo[1,2-b]pyridazine-3-sulfonamide), prosulfuron, pyrazosulfuron-ethyl, pyribenzoxim, pyriftalid, pyriminobac-methyl, pyrithiobac-sodium, rimsulfuron, sulfometuron-methyl, sulfosulfuron, thiencarbazone, thifensulfuron-methyl, triafamone (N-[2-[(4,6-dimethoxy-1,3,5-triazin-2-yl)carbonyl]-6-fluorophenyl]-1,1-difluoro-N-methylmethanesulfonamide), triasulfuron, tribenuron-methyl, trifloxysulfuron (including sodium salt), triflusulfuron-methyl and tritosulfuron.

“ACCase inhibitors” (b3) are chemical compounds that inhibit the acetyl-CoA carboxylase enzyme, which is responsible for catalyzing an early step in lipid and fatty acid synthesis in plants. Lipids are essential components of cell membranes, and without them, new cells cannot be produced. The inhibition of acetyl CoA carboxylase and the subsequent lack of lipid production leads to losses in cell membrane integrity, especially in regions of active growth such as meristems. Eventually shoot and rhizome growth ceases, and shoot meristems and rhizome buds begin to die back. Examples of ACCase inhibitors include alloxydim, butroxydim, clethodim, clodinafop, cycloxydim, cyhalofop, diclofop, fenoxaprop, fluazifop, haloxyfop, pinoxaden, profoxydim, propaquizafop, quizalofop, sethoxydim, tepraloxydim and tralkoxydim, including resolved forms such as fenoxaprop-P, fluazifop-P, haloxyfop-P and quizalofop-P and ester forms such as clodinafop-propargyl, cyhalofop-butyl, diclofop-methyl and fenoxaprop-P-ethyl.

Auxin is a plant hormone that regulates growth in many plant tissues. “Auxin mimics” (b4) are chemical compounds mimicking the plant growth hormone auxin, thus causing uncontrolled and disorganized growth leading to plant death in susceptible species. Examples of auxin mimics include aminocyclopyrachlor (6-amino-5-chloro-2-cyclopropyl-4-pyrimidinecarboxylic acid) and its methyl and ethyl esters and its sodium and potassium salts, aminopyralid, benazolin-ethyl, chloramben, clacyfos, clomeprop, clopyralid, dicamba, 2,4-D, 2,4-DB, dichlorprop, fluroxypyr, halauxifen (4-amino-3-chloro-6-(4-chloro-2-fluoro-3-methoxyphenyl)-2-pyridinecarboxylic acid), halauxifen-methyl (methyl 4-amino-3-chloro-6-(4-chloro-2-fluoro-3-methoxyphenyl)-2-pyridinecarboxylate), MCPA, MCPB, mecoprop, picloram, quinclorac, quinmerac, 2,3,6-TBA, triclopyr, and methyl 4-amino-3-chloro-6-(4-chloro-2-fluoro-3-methoxyphenyl)-5-fluoro-2-pyridinecarboxylate.

“EPSP synthase inhibitors” (b5) are chemical compounds that inhibit the enzyme, 5-enol-pyruvylshikimate-3-phosphate synthase, which is involved in the synthesis of aromatic amino acids such as tyrosine, tryptophan and phenylalanine. EPSP inhibitor herbicides are readily absorbed through plant foliage and translocated in the phloem to the growing points. Glyphosate is a relatively nonselective postemergence herbicide that belongs to this group. Glyphosate includes esters and salts such as ammonium, isopropylammonium, potassium, sodium (including sesquisodium) and trimesium (alternatively named sulfosate).

“Photosystem I electron diverters” (b6) are chemical compounds that accept electrons from Photosvstem I, and after several cycles, generate hydroxyl radicals. These radicals are extremely reactive and readily destroy unsaturated lipids, including membrane fatty acids and chlorophyll. This destroys cell membrane integrity, so that cells and organelles “leak”, leading to rapid leaf wilting and desiccation, and eventually to plant death. Examples of this second type of photosynthesis inhibitor include diquat and paraquat.

“PPO inhibitors” (b7) are chemical compounds that inhibit the enzyme protoporphyrinogen oxidase, quickly resulting in formation of highly reactive compounds in plants that rupture cell membranes, causing cell fluids to leak out. Examples of PPO inhibitors include acifluorfen-sodium, azafenidin, benzfendizone, bifenox, butafenacil, carfentrazone, carfentrazone-ethyl, chlomethoxyfen, cinidon-ethyl, fluazolate, flufenpyr-ethyl, flumiclorac-pentyl, flumioxazin, fluoroglycofen-ethyl, fluthiacet-methyl, fomesafen, halosafen, lactofen, oxadiargyl, oxadiazon, oxyfluorfen, pentoxazone, profluazol, pyraclonil, pyraflufen-ethyl, saflufenacil, sulfentrazone, thidiazimin, trifludimoxazin (dihydro-1,5-dimehyl-6-thioxo-3-[2,2,7-trifluoro-3,4-dihydro-3-oxo-4-(2-propyn-1-yl)-2H-1,4-benzoxazin-6-yl]-1,3,5-triazine-2,4(1H,3H)-dione) and tiafenacil (methyl N-[2-[[2-chloro-5-[3,6-dihydro-3-methyl-2,6-dioxo-4-(trifluoromethyl)-1(2H)-pyrimidinyl]-4-fluorophenyl]thio]-1-oxopropyl]-β-alaninate).

“GS inhibitors” (b8) are chemical compounds that inhibit the activity of the glutamine synthetase enzyme, which plants use to convert ammonia into glutamine. Consequently, ammonia accumulates and glutamine levels decrease. Plant damage probably occurs due to the combined effects of ammonia toxicity and deficiency of amino acids required for other metabolic processes. The GS inhibitors include glufosinate and its esters and salts such as glufosinate-ammonium and other phosphinothricin derivatives, glufosinate-P ((2S)-2-amino-4-(hydroxymethylphosphinyl)butanoic acid) and bilanaphos.

“VLCFA elongase inhibitors” (b9) are herbicides having a wide variety of chemical structures, which inhibit the elongase. Elongase is one of the enzymes located in or near chloroplasts which are involved in biosynthesis of VLCFAs. In plants, very-long-chain fatty acids are the main constituents of hydrophobic polymers that prevent desiccation at the leaf surface and provide stability to pollen grains. Such herbicides include acetochlor, alachlor, anilofos, butachlor, cafenstrole, dimethachlor, dimethenamid, diphenamid, fenoxasulfone (3-[[(2,5-dichloro-4-ethoxyphenyl)methyl]sulfonyl]-4,5-dihydro-5,5-dimethylisoxazole), fentrazamide, flufenacet, indanofan, mefenacet, metazachlor, metolachlor, naproanilide, napropamide, napropamide-M ((2R)-N,N-diethyl-2-(1-naphthalenyloxy)propanamide), pethoxamid, piperophos, pretilachlor, propachlor, propisochlor, pyroxasulfone, and thenylchlor, including resolved forms such as S-metolachlor and chloroacetamides and oxyacetamides.

“Auxin transport inhibitors” (b10) are chemical substances that inhibit auxin transport in plants, such as by binding with an auxin-carrier protein. Examples of auxin transport inhibitors include diflufenzopyr, naptalam (also known as N-(1-naphthyl)phthalamic acid and 2-[(1-naphthalenylamino)carbonyl]benzoic acid).

“PDS inhibitors” (b11) are chemical compounds that inhibit carotenoid biosynthesis pathway at the phytoene desaturase step. Examples of PDS inhibitors include beflubutamid. S-beflubutamid, diflufenican, fluridone, flurochloridone, flurtamone norflurzon and picolinafen.

“HPPD inhibitors” (b12) are chemical substances that inhibit the biosynthesis of synthesis of 4-hydroxyphenyl-pyruvate dioxygenase. Examples of HPPD inhibitors include benzobicyclon, benzofenap, bicyclopyrone (4-hydroxy-3-[[2-[(2-methoxyethoxy)methyl]-6-(trifluoromethyl)-3-pyridinyl]carbonyl]bicyclo[3.2.1]oct-3-en-2-one), fenquinotrione (2-[[8-chloro-3,4-dihydro-4-(4-methoxyphenyl)-3-oxo-2-quinoxalinyl]carbonyl]-1,3-cyclohexanedione), isoxachlortole, isoxaflutole, mesotrione, pyrasulfotole, pyrazolynate, pyrazoxyfen, sulcotrione, tefuryltrione, tembotrione, tolpyralate (1-[[1-ethyl-4-[3-(2-methoxyethoxy)-2-methyl-4-(methylsulfonyl)benzoyl]-1H-pyrazol-5-yl]oxy]ethyl methyl carbonate), topramezone, 5-chloro-3-[(2-hydroxy-6-oxo-1-cyclohexen-1-yl)carbonyl]-1-(4-methoxyphenyl)-2(1H)-quinoxalinone, 4-(2,6-diethyl-4-methylphenyl)-5-hydroxy-2,6-dimethyl-3(2H)-pyridazinone, 4-(4-fluorophenyl)-6-[(2-hydroxy-6-oxo-1-cyclohexen-1-yl)carbonyl]-2-methyl-1,2,4-triazine-3,5(2H,4H)-dione, 5-[(2-hydroxy-6-oxo-1-cyclohexen-1-yl)carbonyl]-2-(3-methoxyphenyl)-3-(3-methoxypropyl)-4(3H)-pyrimidinone, 2-methyl-N-(4-methyl-1,2,5-oxadiazol-3-yl)-3-(methylsulfinyl)-4-(trifluoromethyl)benzamide and 2-methyl-3-(methylsulfonyl)-N-(1-methyl-1H-tetrazol-5-yl)-4-(trifluoromethyl)benzamide.

“HST inhibitors” (b3) disrupt a plant's ability to convert homogentisate to 2-methyl-6-solanyl-1,4-benzoquinone, thereby disrupting carotenoid biosynthesis. Examples of HST inhibitors include haloxydine, pyriclor, 3-(2-chloro-3,6-difluorophenyl)-4-hydroxy-1-methyl-1,5-naphthyridin-2(H)-one, 7-(3,5-dichloro-4-pyridinyl)-5-(2,2-difluoroethyl)-8-hydroxypyrido[2,3-b]pyrazin-6(5H)-one and 4-(2,6-diethyl-4-methylphenyl)-5-hydroxy-2,6-dimethyl-3(2H)-pyridazinone.

HST inhibitors also include compounds of Formulae A and B.

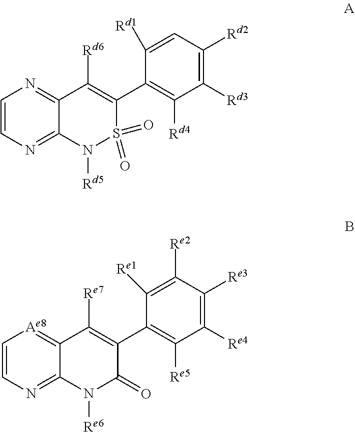

wherein R^{d1} is H, Cl or CF₃; R^{d2} is H, Cl or Br; R^{d3} is H or Cl; R^{d4} is H, Cl or CF₃; R^{d5} is CH₃, CH₂CH₃ or CH₂CHF₂; and R^{d6} is OH, or —OC(═O)-i-Pr; and R^{e1} is H, F, Cl, CH₃ or CH₂CH₃; R^{e2} is H or CF₃; R^{e3} is H, CH₃ or CH₂CH₃; R^{e4} is H, F or Br; R^{e5} is Cl, CH₃, CF₃, OCF₃ or CH₂CH₃; R^{e6} is H, CH₃, CH₂CHF₂ or C≡CH; R^{e7} is

OH, —OC(═O)Et, —OC(═O)-i-Pr or —OC(═O)-t-Bu; and A^{e8} is N or CH.

“Cellulose biosynthesis inhibitors” (b14) inhibit the biosynthesis of cellulose in certain plants. They are most effective when applied preemergence or early postemergence on young or rapidly growing plants. Examples of cellulose biosynthesis inhibitors include chlorthiamid, dichlobenil, flupoxam, indaziflam (N²-[(1R,2S)-2,3-dihydro-2,6-dimethyl-1H-inden-1-yl]-6-(1-fluoroethyl)-1,3,5-triazine-2,4-diamine), isoxaben and triaziflam.

“Other herbicides” (b15) include herbicides that act through a variety of different modes of action such as mitotic disruptors (e.g., flamprop-M-methyl and flamprop-M-isopropyl), organic arsenicals (e.g., DSMA, and MSMA), 7,8-dihydropteroate synthase inhibitors, chloroplast isoprenoid synthesis inhibitors and cell-wall biosynthesis inhibitors. Other herbicides include those herbicides having unknown modes of action or do not fall into a specific category listed in (b1) through (b14) or act through a combination of modes of action listed above. Examples of other herbicides include aclonifen, asulam, amitrole, bromobutide, cinmethylin, clomazone, cumyluron, cyclopyrimorate (6-chloro-3-(2-cyclopropyl-6-methylphenoxy)-4-pyridazinyl 4-morpholinecarboxylate), daimuron, difenzoquat, etobenzanid, fluometuron, flurenol, fosamine, fosamine-ammonium, dazomet, dymron, ipfencarbazone (1-(2,4-dichlorophenyl)-N-(2,4-difluorophenyl)-1,5-dihydro-N-(1-methylethyl)-5-oxo-4H-1,2,4-triazole-4-carboxamide), metam, methyldymron, oleic acid, oxaziclomefone, pelargonic acid, pyributicarb and 5-[[(2,6-difluorophenyl)methoxy]methyl]-4,5-dihydro-5-methyl-3-(3-methyl-2-thienyl)isoxazole.

“Other herbicides” (b15) also include a compound of Formula (b15A)

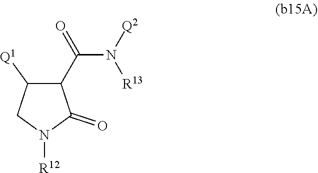

wherein
R¹² is H, C₁-C₆ alkyl, C₁-C₆ haloalkyl or C₄-C₈ cycloalkyl;
R¹³ is H, C₁-C₆ alkyl or C₁-C₆ alkoxy;
Q¹ is an optionally substituted ring system selected from the group consisting of phenyl, thienyl, pyridinyl, benzodioxolyl, naphthyl, naphthalenyl, benzofuranyl, furanyl, benzothiophenyl and pyrazolyl, wherein when substituted said ring system is substituted by 1 to 3 R¹⁴;
Q² is an optionally substituted ring system selected from the group consisting of phenyl, pyridinyl, benzodioxolyl, pyridinonyl, thiadiazolyl, thiazolyl, and oxazolyl, wherein when substituted said ring system is substituted by 1 to 3 R¹⁵;
each R¹⁴ is independently halogen, C₁-C₆ alkyl, C₁-C₆ haloalkyl, C₁-C₆ alkoxy, C₁-C₆ haloalkoxy, C₃-C₈ cyaloalkyl, cyano, C₁-C₆ alkylthio, C₁-C₆ alkylsulfinyl, C₁-C₆ alkylsulfonyl, SF₅, NHR¹⁷; or phenyl optionally substituted by 1 to 3 R¹⁶; or pyrazolyl optionally substituted by 1 to 3 R¹⁶;
each R¹⁵ is independently halogen, C₁-C₆ alkyl, C₁-C₆ haloalkyl, C₁-C₆ alkoxy, C₁-C₆ haloalkoxy, cyano, nitro, C₁-C₆ alkylthio, C₁-C₆ alkylsulfinyl, C₁-C₆ alkylsulfonyl;
each R¹⁶ is independently halogen, C₁-C₆ alkyl or C₁-C₆ haloalkyl;
R¹⁷ is C₁-C₄ alkoxycarbonyl.
   In one Embodiment wherein “other herbicides” (b15) also include a compound of Formula (b15A), it is preferred that R¹² is H or C₁-C₆ alkyl; more preferably R¹² is H or methyl. Preferrably R¹³ is H. Preferably Q¹ is either a phenyl ring or a pyridinyl ring, each ring substituted by I to 3 R¹⁴; more preferably Q¹ is a phenyl ring substituted by 1 to 2 R¹⁴. Preferably Q² is a phenyl ring substituted by 1 to 3 R¹⁵; more preferably Q² is a phenyl ring substituted by 1 to 2 R¹⁵. Preferably each R¹⁴ is independently halogen, C₁-C₄ alkyl, C₁-C₃ haloalkyl, C₁-C₃ alkoxy or C₁-C₃ haloalkoxy; more preferably each R¹⁴ is independently chloro, fluoro, bromo, C₁-C₂ haloalkyl, C₁-C₂ haloalkoxy or C₁-C₂ alkoxy. Preferrably each R¹⁵ is independently halogen, C₁-C₄ alkyl, C₁-C₃ haloalkoxy; more preferably each R¹⁵ is independently chloro, fluoro, bromo, C₁-C₂ haloalkyl, C₁-C₂ haloalkoxy or C₁-C₂ alkoxy. Specifically preferred as “other herbicides” (b15) include any one of the following (b15A-1) through (b5A-15) wherein the stereochemistry at the 3- and 4-positions of the pyrrolidinone ring are preferably in the trans configuration relative to each other:

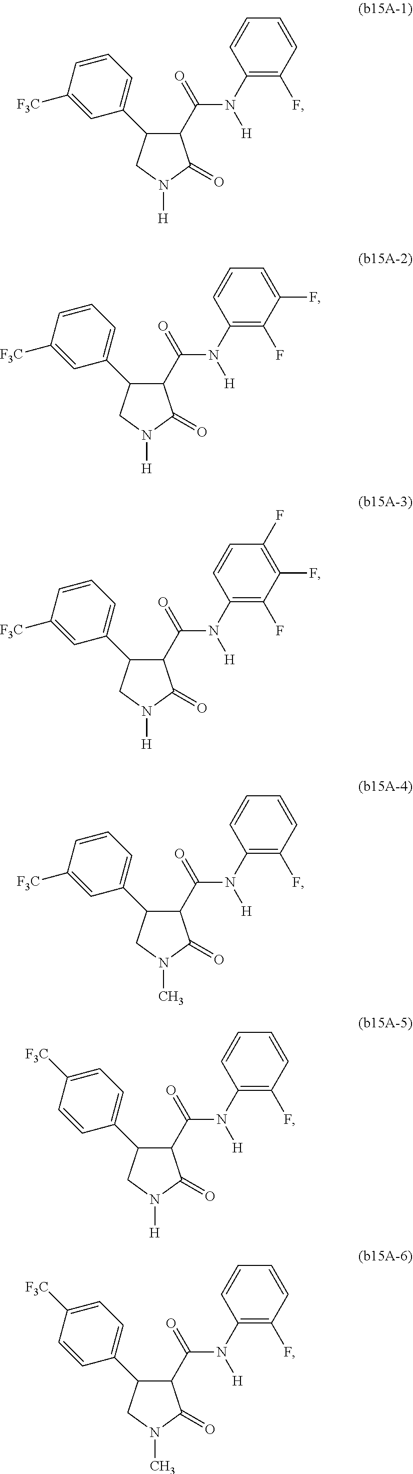 [SMILES: O=C1NCC(c2cccc(C(F)(F)F)c2)C1C(=O)Nc1ccccc1F ; O=C1NCC(c2cccc(C(F)(F)F)c2)C1C(=O)Nc1cccc(F)c1F ; O=C1NCC(c2cccc(C(F)(F)F)c2)C1C(=O)Nc1ccc(F)c(F)c1F ; CN1CC(c2cccc(C(F)(F)F)c2)C(C(=O)Nc2ccccc2F)C1=O ; O=C1NCC(c2ccc(C(F)(F)F)cc2)C1C(=O)Nc1ccccc1F ; CN1CC(c2ccc(C(F)(F)F)cc2)C(C(=O)Nc2ccccc2F)C1=O]

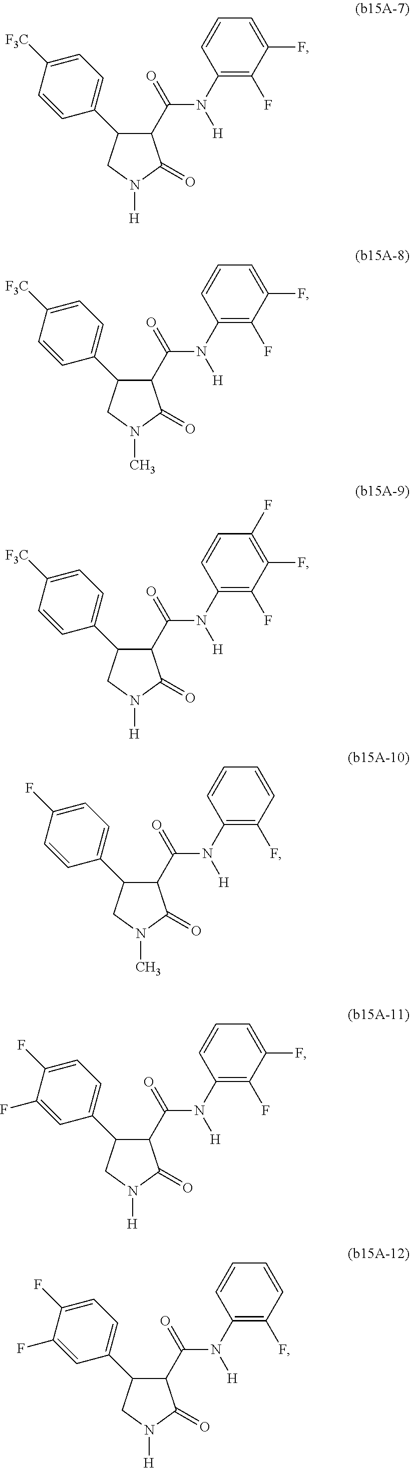 [SMILES: O=C1NCC(c2ccc(C(F)(F)F)cc2)C1C(=O)Nc1cccc(F)c1F ; CN1CC(c2ccc(C(F)(F)F)cc2)C(C(=O)Nc2cccc(F)c2F)C1=O ; O=C1NCC(c2ccc(C(F)(F)F)cc2)C1C(=O)Nc1ccc(F)c(F)c1F ; CN1CC(c2ccc(F)cc2)C(C(=O)Nc2ccccc2F)C1=O ; O=C1NCC(c2ccc(F)c(F)c2)C1C(=O)Nc1cccc(F)c1F ; O=C1NCC(c2ccc(F)c(F)c2)C1C(=O)Nc1ccccc1F]

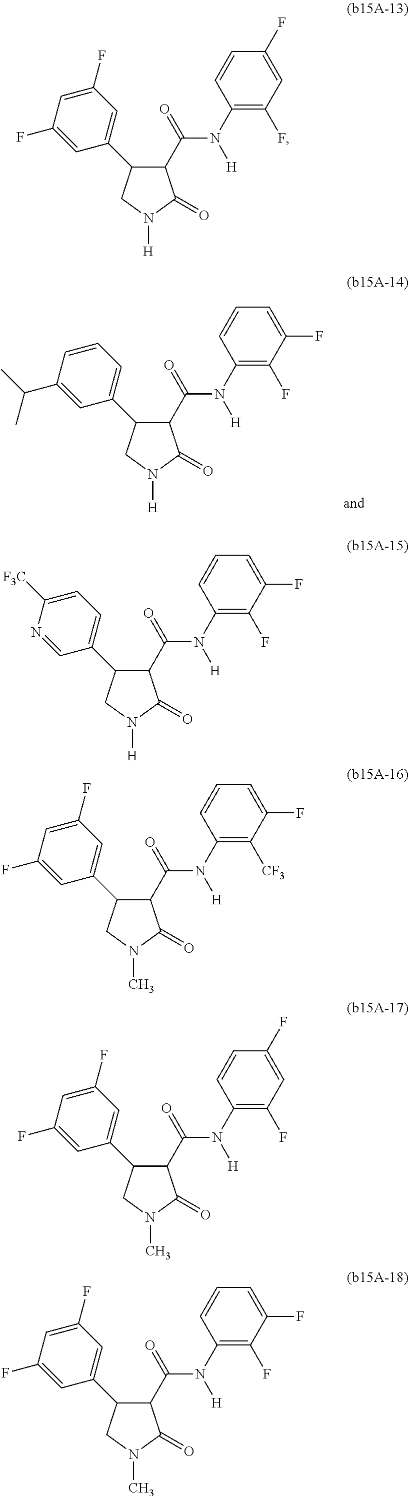 [SMILES: O=C1NCC(c2cc(F)cc(F)c2)C1C(=O)Nc1ccc(F)cc1F ; CC(C)c1cccc(C2CNC(=O)C2C(=O)Nc2cccc(F)c2F)c1 ; O=C1NCC(c2ccc(C(F)(F)F)nc2)C1C(=O)Nc1cccc(F)c1F ; CN1CC(c2cc(F)cc(F)c2)C(C(=O)Nc2cccc(F)c2C(F)(F)F)C1=O ; CN1CC(c2cc(F)cc(F)c2)C(C(=O)Nc2ccc(F)cc2F)C1=O ; CN1CC(c2cc(F)cc(F)c2)C(C(=O)Nc2cccc(F)c2F)C1=O]

“Other herbicides” (b15) also include a compound of Formula (b15B)

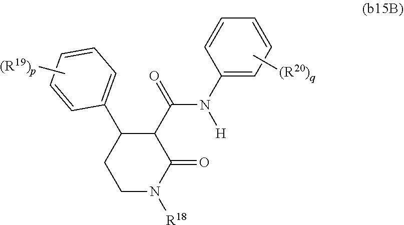

wherein
R¹⁸ is H, C₁-C₆ alkyl, C₁-C₆ haloalkyl or C₄-C₈ cycloalkyl;
each R¹⁹ is independently halogen, C₁-C₆ haloalkyl or C₁-C₆ haloalkoxy; p is an integer of 0, 1, 2 or 3;
each R²⁰ is independently halogen. C₁-C₆ haloalkyl or C₁-C₆ haloalkoxy; and
q is an integer of 0, 1, 2 or 3.
   In one Embodiment wherein “other herbicides” (b15) also include a compound of Formula (b15B), it is preferred that R¹⁸ is H, methyl, ethyl or propyl; more preferably R¹⁸ is H or methyl; most preferably R¹⁸ is H. Preferrably each R¹⁹ is independently chloro, fluoro, C₁-C₃ haloalkyl or C₁-C₃ haloalkoxy; more preferably each R¹⁹ is independently chloro, fluoro. C₁ fluoroalkyl (i.e. fluoromethyl, difluoromethyl or trifluoromethyl) or C₁ fluoroalkoxy (i.e. trifluoromethoxy, difluoromethoxy or fluoromethoxy). Preferably each R²⁰ is independently chloro, fluoro, C₁ haloalkyl or C₁ haloalkoxy; more preferably each R²⁰ is independently chloro, fluoro, C₁ fluoroalkyl (i.e. fluoromethyl, difluoromethyl or trifluromethyl) or C₁ fluoroalkoxy (i.e. trifluoromethoxy, difluoromethoxy or fluoromethoxy). Specifically preferred as “other herbicides” (b15) include any one of the following (b15B-1) through (b15B-19):
(b15B-1) 2-oxo-N-[2-(trifluoromethyl)phenyl]-4-(3,4-difluorophenyl)-3-piperidinecarboxamide,
(b15B-2)N-(2,3-difluorophenyl)-2-oxo-4-[3-(trifluoromethyl)phenyl]-3-piperidinecarboxamide,
(b15B-3) 2-oxo-N-[2-(trifluoromethyl)phenyl)]-4-[3-(trifluoromethyl)phenyl]-3-piperidinecarboxamide,
(b15B-4)N-(2-chlorophenyl)-2-oxo-4-[4-(trifluoromethyl)phenyl]-3-piperidinecarboxamide,
(b15B-5)N-(2-fluorophenyl)-2-oxo-4-[4-(trifluoromethyl)phenyl]-3-piperidinecarboxamide,
(b15B-6) (3R,4S)-N-(2,3-difluorophenyl)-2-oxo-4-[3-(trifluoromethyl)phenyl]-3-piperidinecarboxamide,
(b15B-7) (3R,4S)-N-(2,3-difluorophenyl)-2-oxo-4-[4-(trifluoromethyl)phenyl]-3-piperidinecarboxamide,
(b15B-8) (3R,4S)-N-(3-chloro-2-fluorophenyl)-2-oxo-4-[3-(trifluoromethyl)phenyl]-3-piperidinecarboxamide,
(b15B-9) (3R,4S)-2-oxo-4-[3-(trifluoromethyl)phenyl]-N-[2,3,4-trifluorophenyl]-3-piperidinecarboxamide,

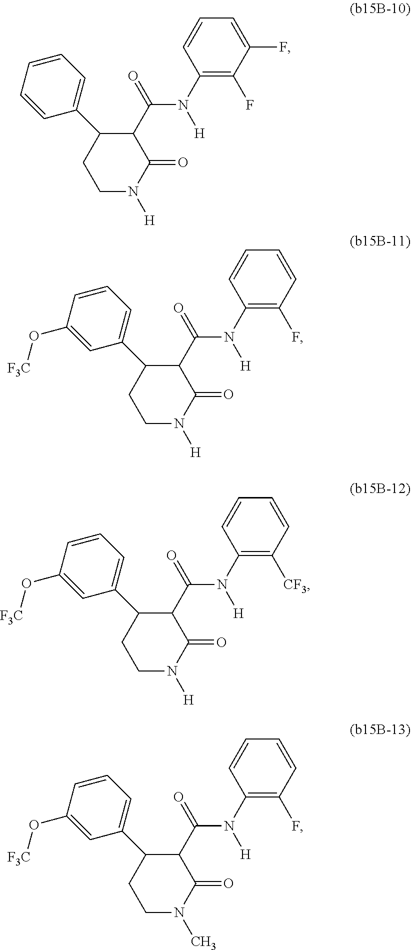 [SMILES: O=C1NCCC(c2ccccc2)C1C(=O)Nc1cccc(F)c1F ; O=C1NCCC(c2cccc(OC(F)(F)F)c2)C1C(=O)Nc1ccccc1F ; O=C1NCCC(c2cccc(OC(F)(F)F)c2)C1C(=O)Nc1ccccc1C(F)(F)F ; CN1CCC(c2cccc(OC(F)(F)F)c2)C(C(=O)Nc2ccccc2F)C1=O]

(b15B-14) (3R,4S)-4-(3-chlorophenyl)-N-(2,3-difluorophenyl)-2-oxo-3-piperidinecarboxamide,
(b15B-15) 4-[3-(difluoromethyl)phenyl]-N-(2,3,4-trifluorophenyl)-2-oxo-piperidinecarboxamide,
(b15B-16) 4-[3-(difluoromethyl)phenyl]-N-(2-fluorophenyl)-2-oxo-piperidinecarboxamide,
(b15B-17) 4-[3-(difluoromethyl)phenyl]-N-(2,3-difluorophenyl)-2-oxo-3-piperidinecarboxamide,
(b15B-18) (3S,4S)-N-(2,3-difluorophenyl)-4-(4-fluorophenyl)-1-methyl-2-oxo-3-piperidinecarboxamide and
(b15B-19) (3R,4S)-2-oxo-N-[2-(trifluoromethyl)phenyl]-4-(4-fluorophenyl)-3-piperidinecarboxamide.

“Other herbicides” (b15) also include a compound of Formula (b15C),

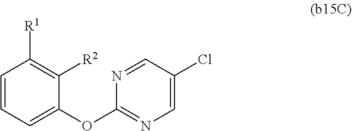

wherein R¹ is Cl, Br or CN; and R² is C(═O)CH₂CH₂CF₃, CH₂CH₂CH₂CH₂CF₃ or 3-CHF₂-isoxazol-5-yl.

“Other herbicides” (b15) also include a compound of Formula (b15D)

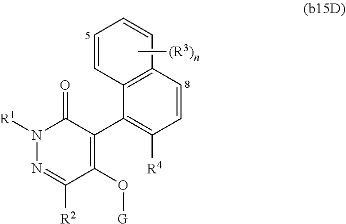

wherein R¹ is CH₃, R² is Me, R⁴ is OCHF₂, G is H, and n is 0; R¹ is CH₃, R² is Me, R³ is 5-F, R⁴ is Cl, G is H. and n is 1; R¹ is CH₃, R² is Cl, R⁴ is Me, G is H. and n is 0; R¹ is CH₃, R² is Me, R⁴ is Cl, G is H, and n is 0; R¹ is CH₃, R² is Me, R³ is 5-Me, R⁴ is OCHF₂, G is H, and n is 1; R¹ is CH₃, R² is Me R³ is 5-Br, R⁴ is OCHF₂, G is H, and n is 1; R¹ is CH₃, R² is Me, R³ is 5-Cl, R⁴ is Cl, G is H, and n is 1; and R¹ is CH₃, R² is CH₃, R⁴ is OCHF₂, G is C(O)Me, and n is 0.

“Other herbicides” (b15) also include a compound of Formula (b15E)

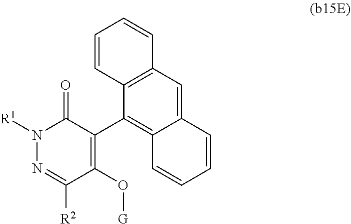

wherein

R¹ is CH₃, R² is Cl, and G is H; and

R¹ is CH₃, R² is Cl, and G is C(O)Me.

“Herbicide safeners” (b16) are substances added to a herbicide formulation to eliminate or reduce phytotoxic effects of the herbicide to certain crops. These compounds protect crops from injury by herbicides but typically do not prevent the herbicide from controlling undesired vegetation. Examples of herbicide safeners include but are not limited to benoxacor, cloquintocet-mexyl, cumyluron, cyometrinil, cyprosulfamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone, naphthalic anhydride, oxabetrinil, N-(aminocarbonyl)-2-methylbenzenesulfonamide and N-(aminocarbonyl)-2-fluorobenzenesulfonamide, 1-bromo-4-[(chloromethyl)sulfonyl]benzene, 2-(dichloromethyl)-2-methyl-1,3-dioxolane (MG 191), 4-(dichloroacetyl)-1-oxa-4-azospiro[4.5]decane (MON 4660), 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide.

One or more of the following methods as described in Schemes 1-10, or variations thereof can be used to prepare the compounds of Formula 1. The definitions of R¹, A, R², R³ and R⁴ in the compounds of Formulae 1-12 below are as defined above in the Summary of the Invention unless otherwise noted. Compounds of Formulae 1A-1D and 11A-11B are various subsets of the compounds of Formulae 1 and 11 and all substituents for Formulae 1A-1D and 11A-11B are as defined above for Formulae 1 and 11 unless otherwise noted.

As shown in Scheme 1, pyridazinones of Formula 1A (i.e. a subset of compounds of Formula 1 where L is other than a direct bond and R² is other than hydrogen) can be prepared by reacting substituted 5-hydroxy-3(2H)-pyridazinones of Formula 1B (i.e. a compound of Formula 1 wherein L is a direct bond and R² is H) with a suitable electrophilic reagent of Formula 2 (i.e. Z-L-R² where Z is a leaving group, alternatively known as a nucleofuge, such as a halogen) in the presence of base in an appropriate solvent. Some examples of reagent classes representing a compound of Formula 2 wherein Z is Cl and L is a direct bond include acid chlorides (R² is —(C═O)R⁵), chloroformates (R² is —CO₂R⁶), carbamoyl chlorides (R² is —CON(R⁷)R⁸), sulfonyl chlorides (R² is —S(O)₂R⁵) and sulfamoyl chlorides (R² is —S(O)₂N(R⁷)R⁸). Examples of suitable bases for this reaction include, but are not limited to, triethylamine, pyridine, N,N-diisopropylethylamine, potassium carbonate, sodium hydroxide, potassium hydroxide, sodium hydride or potassium tert-butoxide. Depending on the specific base used, appropriate solvents can be protic or aprotic and used anhydrous or as aqueous mixtures. Preferred solvents for this reaction include acetonitrile, methanol, ethanol, tetrahydrofuran, diethyl ether, 1,2-dimethoxyethane, dioxane, dichloromethane or N,N-dimethylformamide. The reaction can be performed at a range of temperatures, typically from 0° C. to the reflux temperature of the solvent.

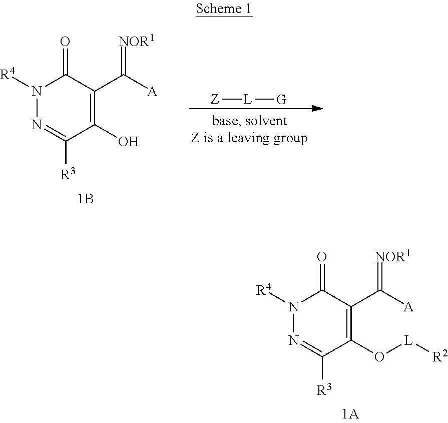

Pyridazinone-substituted ketoximes of Formula 1B can be prepared as outlined in Scheme 2 by condensation of a ketone of Formula 3 with hydroxylamine or an alkoxyamine of the formula H₂N—OR¹, or salt thereof, in the presence of base and solvent. Suitable bases for this reaction include but are not limited to sodium acetate, sodium bicarbonate, sodium carbonate, sodium hydroxide, potassium hydroxide, potassium carbonate, triethylamine, N,N-diisopropylethylamine, pyridine and 4-(dimethylamino)pyridine. Depending on the specific base used, appropriate solvents can be protic or aprotic and used anhydrous or as aqueous mixtures. Solvents for this condensation include acetonitrile, methanol, ethanol, water, tetrahydrofuran, diethyl ether, dioxane, 1,2-dimethoxyethane, dichloromethane or N,N-dimethylformamide. Temperatures for this condensation generally range from 0° C. to the reflux temperature of the solvent. Methods for the condensation of ketones with alkoxyamines to form the corresponding ketoximes are disclosed in U.S. Pat. Nos. 5,085,689 and 4,555,263.

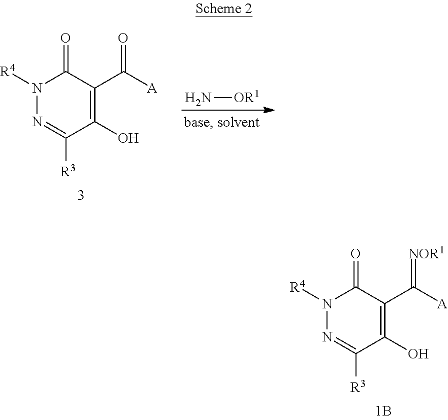

As shown in Scheme 3, pyridazinones of Formula 1D (i.e. a subset of a compound of Formula 1 where R¹ is other than H) can be synthesized by reacting substituted 5-hydroxy-3(2H)-pyridazinones of Formula 1C (i.e. Formula 1 wherein R¹ is H) with a suitable alkylating reagent of Formula 5 (i.e. Z¹—R¹, where Z¹ is a leaving group, alternatively known as a nucleofuge, such as a halogen) in the presence of base in an appropriate solvent. Some examples of reagent classes representing a compound of Formula 5 wherein Z¹ is I or Br include methyl iodide, ethyl iodide, ethyl bromide, 1-bromo-propane, allyl bromide and propargyl bromide. Examples of suitable bases for this reaction include, but are not limited to sodium carbonate, potassium carbonate, sodium hydroxide, potassium hydroxide, sodium hydride or potassium tert-butoxide. Preferred solvents for this reaction include acetonitrile, tetrahydrofuran, diethyl ether, 1,2-dimethoxyethane, dioxane, dichloromethane, dimethyl sulfoxide, acetone or N,N-dimethylformamide. The reaction can be performed at a range of temperatures, typically from 0° C. to the reflux temperature of the solvent.

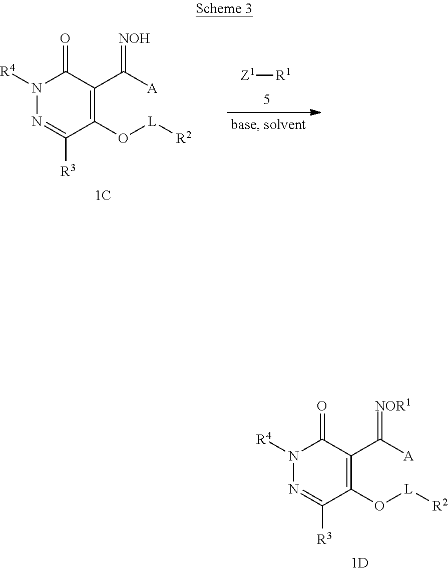

Hydrolysis of certain groups at the 5-position of the pyridazinone ring can be accomplished as shown in Scheme 4. When X is lower alkoxy, lower alkylsulfide (sulfoxide or sulfone), halide or N-linked azole, it can be removed by hydrolysis with basic reagents such as tetrabutylammonium hydroxide in solvents such as tetrahydrofuran, dimethoxyethane or dioxane at temperatures from 0 to 120° C. Other hydroxide reagents useful for this hydrolysis include potassium, lithium and sodium hydroxide (see, for example, WO 2009/086041). Alternatively, when X is lower alkoxy, dealkylation can be accomplished with dealkylation reagents such as boron tribromide, morpholine and inorganic salts, such as lithium chloride (as discussed in Boorg. & Med. Chem. 2013, 21(22), 6956).

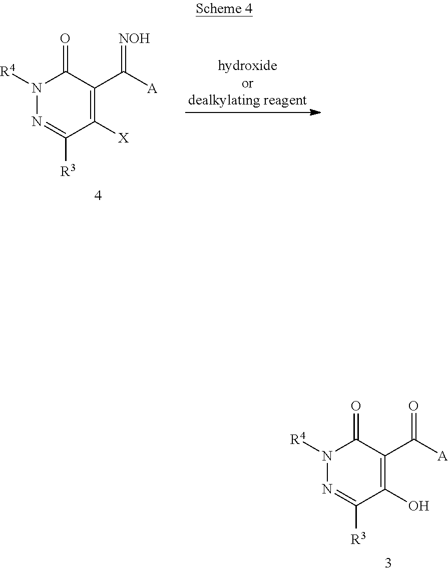

Zincation of the 4-position of a pyridazinone can be accomplished with zincation reagents such as 2,2,6,6-bis(tetramethylpiperidine)zinc, magnesium chloride, lithium chloride complex in toluene/tetrahydrofuran (i.e. Zn(TMP)-LiCl or Zn(TMP)₂-MgCl₂—LiCl). Magnesiation of this position can also be accomplished by treatment with Mg(TMP)-LiCl. See Verhelst, T., Ph.D. thesis, University of Antwerp, 2012 and J. Org. Chem. 2010, 76, 6670 for conditions for pyridazinone metallation and subsequent electrophilic trapping of 4-zincated and 4-magnesiated pyridazinones. The synthesis and cross-coupling conditions for 4-stannylpyridazinones is known from Stevenson et. al. J. Het. Chem. 2005, 42, 427.

Compounds of Formula 4 can be prepared by coupling reactions of organometallic pyridazinone coupling partners of Formula 5 (where Met is Zn, Mg or Sn; and X is hydroxy or lower alkoxy) with acetyl halides of Formula 6 as shown in Scheme 5. The organometallic coupling partner can be, for example, an organozinc, organomagnesium, organotin, or organoboron reagent. Copper reagents such as copper(I) cyanide di(lithium chloride) complex (see J. Org. Chem. 1988, 53, 2390) and copper(I) chloride—bis(lithium chloride) complex can be used in the coupling procedures. Alternatively, palladium catalysts such as palladium tetrakis (triphenylphosphine) and bis(triphenylphosphine)palladium(II) dichloride can be used in the coupling procedures (see Tetrahedron Letters 1983, 47, 5181). Nickel can also effect the coupling of organozinc reagents and acid chlorides as taught in J. Am. Chem. Soc. 2004, 126, 15964. The reaction can be carried out in solvents such as tetrahydrofuran, dimethoxyethane, N-Methyl-2-pyrrolidone, 1,4-dioxane and acetonitrile at temperatures from −40° C. to the reflux temperature of the solvent.

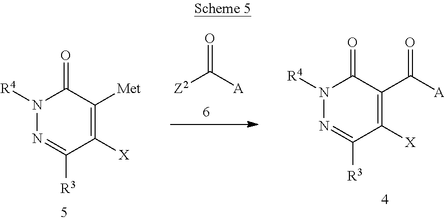

An alternative method for the preparation of an intermediate pyridazinone ketone of Formula 4 is outlined in Scheme 6, through oxidation of a secondary carbinol of Formula 7 where X is hydroxy or lower alkoxy. As taught by the method in J. Het. Chem. 2005, 42, 427, alcohols of Formula 7 can be oxidized with manganese(II) oxide in a solvent such as dichloromethane, hexanes, or acetonitrile at temperatures from 0° C. to the reflux temperature of the solvent. Other suitable oxidants include Jones reagent, pyridinium chlorochromate and Dess-Martin periodinane.

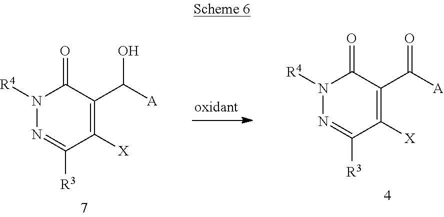

Pyridazinone compounds of Formula 7 can be prepared by the addition of an organometallic compound of Formula 5 (where Met is Li and Mg) with and aldehyde of Formula 8. Hydrolysis of leaving groups at the 5-position of the pyridazinone ring can be accomplished as shown in Scheme 7. When X is lower alkoxy, lower alkylsulfide (sulfoxide or sulfone), halide or N-linked azole, it can be removed by hydrolysis with basic reagents such as tetrabutylammonium hydroxide in solvents such as tetrahydrofuran, dimethoxyethane or dioxane at temperatures from 0-120° C. Other hydroxide reagents useful for this hydrolysis include potassium, lithium and sodium hydroxide (see, for example, WO 2009/086041). When X is lower alkoxy, hydrolysis of X can alternatively be accomplished with dealkylation reagents such as boron tribromide or morpholine (see, for example WO 2013/160126 and WO 2013/050421).

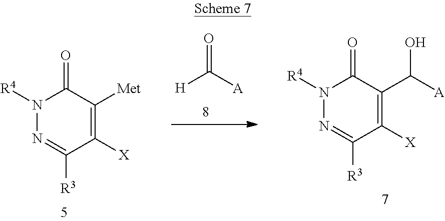

Introduction of a halogen at the 6-position of the pyridazinone can be accomplished by zincation followed by halogenation. For conditions, reagents and examples of zincation of pyridazinones see Verhelst, T., Ph. D. thesis, University of Antwerp, 2012. Typically, the pyridazinone of Formula 9 is treated in tetrahydrofuran with a solution of Zn(TMP)-LiCl or Zn(TMP)₂—MgCl₂—LiCl (i.e. 2,2,66-Bis(tetramethylpiperidine)zinc, magnesium chloride, lithium chloride complex in toluene/tetraydrofuran) at −20 to 30° C. to form a zinc reagent. Subsequent addition of bromine, N-bromosuccinimide or iodine provides compounds of Formula 1D (wherein R² is Br or I, respectively). Reagents such as trichloroisocyanuric acid or 1,3-dichloro-5,5-dimethylhydantoin give a compound of Formula 1D (wherein R² is Cl). This method is shown in Scheme 8. For preparation of a variety of appropriate zincation reagents, see Wunderlich. S. Ph.D. thesis, University of Munich, 2010 and references cited therein, as well as WO2008/138946 and WO2010/092096.

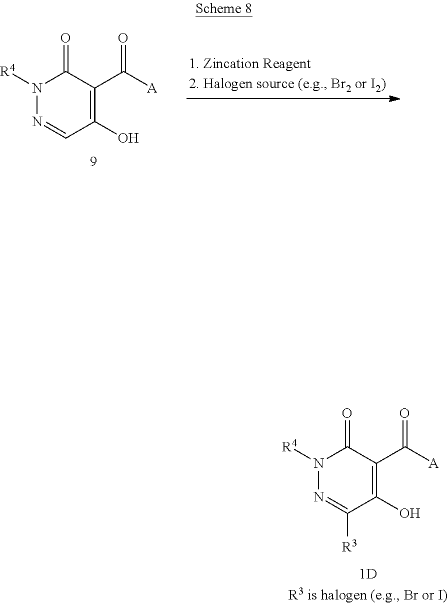

The R³ substituent of compounds of Formula 12 (wherein R³ is defined in Scheme 9; L is a direct bond and R² is H) can be further transformed into other functional groups. Compounds wherein R³ is alkyl, cycloalkyl or substituted alkyl can be prepared by transition metal catalyzed reactions of compounds of Formula 11 (wherein R³ is halogen or sulfonate; L is a direct bond and R² is H) as shown in Scheme 9. For reviews of these types of reactions, see: E. Negishi, Handbook of Organopalladium Chemistry for Organic Synthesis, John Wiley and Sons, Inc., New York, 2002 or N. Miyaura, Cross-Coupling Reactions: A Practical Guide, Springer, New York, 2002. For a review of Buchwald-Hartwig chemistry see Yudin and Hartwig, Catalyzed Carbon-Heteroatom Bond Formation, 2010, Wiley, New York. For iron-catalyzed cross coupling reactions see Furstner, Alois, J. Am. Chem Soc. 2002, 124, 13856.

Related synthetic methods for the introduction of other functional groups at the R³ position of Formula 12 are known in the art. Copper-catalyzed reactions are useful for introducing the CF₃ group. For a comprehensive recent review of reagents for this reaction see Wu, Neumann and Beller in Chemistry: An Asian Journal, 2012, ASAP, and references cited therein. For introduction of a sulfur containing substituent at this position, see methods disclosed in WO 2013/160126. For introduction of a cyano group, see WO 2014/031971, Org. Lett., 2005, 17, 202 and Angew. Chem. Int. Ed 2013, 52, 10035. For introduction of a fluoro substituent, see J. Am. Chem. Soc. 2014, 3792. For introduction of a halogen, see Org. Lett. 2011, 13, 4974. And for a review of palladium-catalyzed carbon-nitrogen bond formation, see Buchwald and Ruiz-Castillo, Chem. Rev. 2016, 116, 125(4 and Sury and Buchwald, Acc. Chem. Res. 2008, 41, 1461.

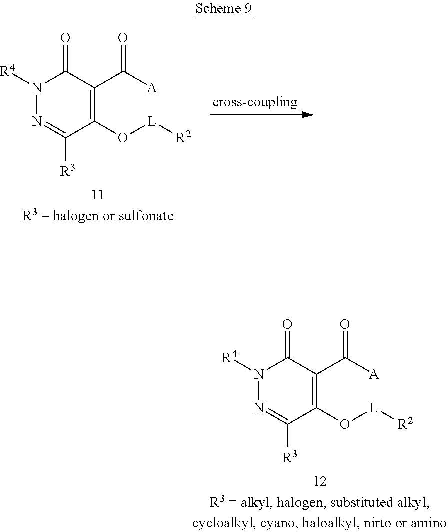

Compounds of Formula 11B can be prepared by the alkylation of compounds of Formula 11A (where R⁴ is H). Typical bases useful in this method include potassium, sodium or cesium carbonate. Typical solvents include acetonitrile, tetrahydrofuran or N,N-dimethylformamide as shown in Scheme 10.

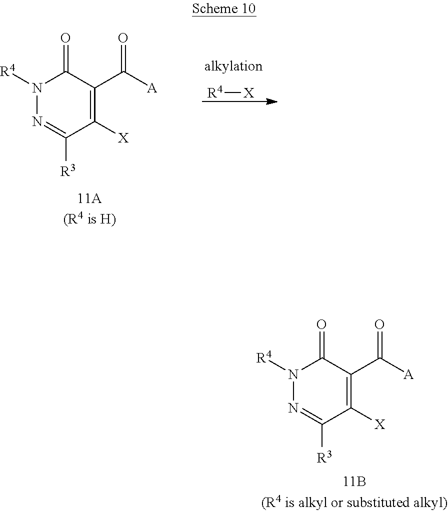

It is recognized by one skilled in the art that various functional groups can be converted into others to provide different compounds of Formula 1. For a valuable resource that illustrates the interconversion of functional groups in a simple and straightforward fashion, see Larock, R C., Comprehensive Organic Transformations: A Guide to Functional Group Preparations, 2nd Ed., Wiley-VCH, New York, 1999. It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following non-limiting Examples are illustrative of the invention. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane in CDCl₃; “s” means singlet, “d” means doublet, “m” means multiplet and “br s” means broad singlet.

### Synthesis Example 1

### Preparation of 6-chloro-5-hydroxy-4-[(Z)-(methoxyimino)-1-naphthalenylmethyl]-2-methyl-3(2H)-pyridazinone (Compound 129) and 6-chloro-5-hydroxy-4-[(E)-(methoxyimino)-1-naphthalenylmethyl]-2-methyl-3(2H)-pyridazinone (Compound 145)

### Step A: Preparation of 6-chloro-5-methoxy-2-methyl-4-(1-naphthalenylcarbonyl)-3(2H)-pyridazinone

To a solution of 6-chloro-5-methoxy-2-methyl-3(2H)-pyridazinone (1.00 g, 5.66 mmol, 1.0 eq) in anhydrous tetrahydrofuran (18 mL) was added 2,2,6,6-tetramethylpiperidinyl zinc chloride lithium chloride complex (0.7 M in tetrahydrofuran, 11.3 mL, 1.4 eq) at ambient temperature. After stirring for 30 min, the reaction mixture was treated with copper(I) cyanide di(lithium chloride) complex (I M in tetrahydrofuran, 8.49 mL, 1.5 eq), followed by a solution of 1-naphthoyl chloride (1.27 mL, 8.49 mmol, 1.5 eq) in 2 mL anhydrous tetrahydrofuran. The reaction was stirred for 18 h. The mixture was quenched with 1 N aqueous hydrochloric acid and extracted with portions of ethyl acetate. The combined organic layers were dried and concentrated onto Celite® diatomaceous earth filter aid and purified with chromatography, eluting with 0 to 50% ethyl acetate in hexanes to afford 1.86 g of the title compound.

¹H NMR δ 9.17-9.29 (m, 1H), 8.06-8.14 (m, 1H), 7.87-7.95 (m, 2H), 7.70-7.74 (m, 1H), 7.59-7.62 (m, 1H), 7.48-7.53 (m, 1H), 3.90 (s, 3H), 3.70 (s, 3H).

### Step B: Preparation of 6-chloro-5-hydroxy-2-methyl-4-(1-naphthalenylcarbonyl)-3(2H)-pyridazinone

To a solution of 6-chloro-5-methoxy-2-methyl-4-(1-naphthalenylcarbonyl)-3(2H)-pyridazinone (i.e. the product of Step A) (0.200 g, 0.608 mmol, 1.0 eq) in dichloromethane (5 mL) was added boron tribromide (1.0 M in dichloromethane, 1.82 mL, 3.0 eq). The resulting solution was stirred at ambient temperature for 18 h. The reaction mixture was concentrated in vacuo and the residue was stirred in 1 N hydrochloric acid for 1 h. The solid was filtered, washed with water and dried to afford 0.178 g of the title compound.

¹H NMR δ 7.98-8.04 (m, 1H), 7.89-7.94 (m, 1H), 7.79-7.85 (m, 1H), 7.46-7.56 (m, 4H), 3.61 (s, 3H).

### Step C: Preparation of 6-chloro-5-hydroxy-4-[(Z)-(methoxyimino)-1-naphthalenylmethyl]-2-methyl-3(2H)-pyridazinone and 6-chloro-5-hydroxy-4-[(E)-(methoxyimino)-1-naphthalenylmethyl]-2-methyl-3(2H)-pyridazinone

A suspension of 6-chloro-5-hydroxy-2-methyl-4-(1-naphthalenylcarbonyl)-3(2H)-pyridazinone (i.e. the product of Step B) (0.300 g, 0.954 mmol, 1.0 eq), methoxyamine hydrochloride (0.158 g, 1.90 mmol, 2.0 eq) and sodium bicarbonate (0.176 g, 2.10 mmol, 2.2 eq) in methanol (5 mL) was heated at 60° C. for 18 h. The reaction mixture was cooled to ambient temperature and concentrated under reduced pressure. The resulting residue was dissolved in ethyl acetate and washed with 1 N aqueous hydrochloric acid. The organic phase was dried and concentrated onto Celite® diatomaceous earth filter aid and purified by reverse-phase chromatography, eluting with 10% to 100% acetonitrile in water with 0.05% trifluoroacetic acid to afford 0.100 g of the Z-isomer and 0.120 g of the E-isomer.

Z-isomer: ¹H NMR δ 8.15-8.21 (m, 1H), 7.84-7.91 (m, 2H), 7.73-7.83 (br s, 1H), 7.47-7.54 (m, 2H), 7.39-7.47 (m, 2H), 4.22 (s, 3H), 3.57 (m, 3H).

E-isomer: ¹H NMR δ 13.51 (br s, 1H), 7.82-8.01 (m, 2H), 7.56-7.61 (m, 1H), 7.43-7.55 (m, 3H), 7.20-7.31 (m, 1H), 3.92 (s, 3H), 3.49 (s, 3H).

### Synthesis Example 2

### 5-hydroxy-2,6-dimethyl-4-[(E)-[(2-propyn-1-yloxy)imino]-1-naphthalenylmethyl]-3(2H)-pyridazinone (Compound 82) and 5-hydroxy-2,6-dimethyl-4-[(Z)-[(2-propyn-1-yloxy)imino]-1-naphthalenylmethyl]-3(2H)-pyridazinone (Compound 83)

### Step A: Preparation of 5-methoxy-2,6-dimethyl-3(2H)-pyridazinone

A reaction vessel was charged with 6-chloro-5-methoxy-2-methyl-3(2H)-pyridazinone (5.0 g, 28.6 mmol), potassium carbonate (9.9 g, 71.6 mmol), and [1,1′-bis(diphenylphosphino)ferrocene]dichloropalladium(II) (1.05 g, 1.43 mmol). The reaction was evacuated and purged with nitrogen five times, then 100 mL of dioxane and trimethylboroxine (8 mL, 57.2 mmol) were added via syringe. The reaction mixture was stirred at room temperature for 15 min, heated to the reflux temperature of the solvent for 4 h, and partitioned between ethyl acetate and water. The organic phase was separated and the aqueous phase was extracted with dichloromethane. The two organic phases were combined, dried over magnesium sulfate, filtered through a pad of Celite® diatomaceous earth filter aid, and concentrated. The crude material was purified via silica gel chromatography (dichloromethane:ethyl acetate gradient) to provide 3.5 g of the title compound.

¹H NMR δ 6.12 (s, 1H), 3.81 (s, 3H), 3.68 (s, 3H), 2.22 (s, 3H).

### Step B: Preparation of 5-methoxy-2,6-dimethyl-4-(1-naphthalenylcarbonyl)-3(2H)-pyridazinone

To a solution of 5-methoxy-2,6-dimethyl-3(2H)-pyridazinone (i.e. the product of Step A) (1.1 g, 7.2 mmol) in 12 mL of tetrahydrofuran was added 2,2,6,6-tetramethylpiperidinylzinc chloride lithium chloride complex solution (0.7 M in tetrahydrofuran, 14.2 mL, 9.94 mmol). The resulting solution was stirred at room temperature for 30 min, then copper(I) cyanide di(lithium chloride) complex (1.0 M in tetrahydrofuran, 10.65 mL, 10.65 mmol and 1-naphthoyl chloride (2.03 g, 10.65 mmol) were added. The resulting mixture was stirred overnight, concentrated onto a mixture of Celite® diatomaceous earth filter aid and silica, and purified via silica gel chromatography using dichloromethane and ethyl acetate as the solvent gradient to provide 2.03 g of the title compound.

¹H NMR δ 9.21 (m, 1H), 8.06 (d, 1H), 7.87-7.98 (m, 2H), 7.65-7.76 (m, 1H), 7.55-7.63 (m, 1H), 7.49 (m, 1H), 3.84 (s, 3H), 3.66 (s, 3H), 2.31 (s, 3H).

### Step C: Preparation of 5-hydroxy-2,6-dimethyl-4-(1-naphthalenylcarbonyl)-3(2H)-pyridazinone

To a solution of 5-methoxy-2,6-dimethyl-4-(1-naphthalenylcarbonyl)-3(2H)-pyridazinone (i.e. the product from Step B) (6.0 g, 19.48 mmol) in 100 mL of dichloromethane at 0° C. was added boron tribromide (1.0 M in dichloromethane, 58.44 mL, 58.44 mmol). The solution was allowed to warm to room temperature and stirred for 3 h. Additional boron tribromide (1.0 M in dichloromethane, 19.48 mL, 19.48 mmol) was added and the reaction mixture was stirred overnight. Water (100 mL, ice-cold) was added and the reaction mixture was stirred for 30 min. The organic phase was separated and the aqueous phase was extracted with additional dichloromethane. The organic phases were combined, washed with brine, dried over magnesium sulfate, filtered, and concentrated under vacuum to provide 5.8 g of the title compound.

¹H NMR δ 14.66 (s, 1H), 7.95-8.00 (m, 1H), 7.88-7.91 (m 1H), 7.82-7.86 (m, 1H), 7.49 (s, 4H), 3.55 (s, 3H), 2.37-2.41 (m, 3H).

### Step D: Preparation of 5-hydroxy-2,6-dimethyl-4-[(E)-[(2-propyn-1-yloxy)imino]-1-naphthalenylmethyl]-3(2H)-pyridazinone and 5-hydroxy-2,6-dimethyl-4-[(Z)-[(2-propyn-1-yloxy)imino]-1-naphthalenylmethyl]-3(2H)-pyridazinone

To a solution of 5-hydroxy-2,6-dimethyl-4-(1-naphthalenylcarbonyl)-3(2H)-pyridazinone (i.e. the product from Step C) (5.8 g, 19.71 mmol) and sodium bicarbonate (2.48 g, 29.56 mmol) in 50 mL of methanol was added O-2-propargylhydroxylamine hydrochloride (4.24 g, 39.42 mmol). The reaction mixture was heated at 45° C. over the weekend and partitioned between water and dichloromethane. The aqueous phase was extracted with additional dichloromethane and the combined organic phases were washed with brine. The organic phase was dried over magnesium sulfate, filtered, and concentrated under vacuum. The crude material was purified via silica gel chromatography using ethyl acetate in dichloromethane as the solvent gradient to provide 2.3 g the E-isomer and 3.1 g of the Z-isomer.

E-isomer ¹H NMR δ 12.37 (s, 1H), 7.85-7.92 (m, 2H), 7.62-7.69 (m, 1H), 7.41-7.54 (m, 3H), 7.26-7.29 (m, 1H), 4.61 (m, 2H), 3.47 (s, 3H), 2.54-2.60 (m, 1H), 2.35-2.42 (m, 3H).

Z-isomer ¹H NMR δ 8.25-8.28 (m, 1H), 7.83-7.90 (m, 2H), 7.38-7.54 (m, 4H), 4.96-5.00 (m, 2H), 3.53-3.56 (m 3H), 2.62-2.65 (m, 1H), 2.39-2.43 (m 3H).

### Synthesis Example 3

### Preparation of 4-[(Z)-(3-chlorophenyl)(methoxyimino)methyl]-5-hydroxy-2,6-dimethyl-3(2H)-pyridazinone (Compound 11) and 4-[(E)-(3-chlorophenyl)(methoxyimino)methyl]-5-hydroxy-2,6-dimethyl-3(2H)-pyridazinone (Compound 10)

### Step A: Preparation 4-(3-chlorobenzoyl)-5-methoxy-2,6-dimethyl-3(2H)-pyridazinone

An oven-dried flask containing a stirbar was charged with 5-methoxy-2,6-dimethyl-3(2H)-pyridazinone (0.60 g, 3.89 mmol, 1.0 eq), and the flask was evacuated and backfilled with nitrogen three times. Anhydrous tetrahydrofuran (1.5 mL) was added and the resulting solution was cooled to 0° C. and treated with a solution of 2,2,6,6-tetramethylpiperidinylzinc chloride lithium chloride complex solution (0.7 M in tetrahydrofuran, 8.04 mL, 1.4 eq). After stirring for 25 min at 0° C., the reaction mixture was warmed to ambient temperature and allowed to stir at this temperature for 15 min. The reaction mixture was then cooled to −40° C. and a solution of copper(I) cyanide di(lithium chloride) complex (1 M in toluene/tetrahydrofuran, 6.03 mL, 1.5 eq) was added. After 5 min of additional stirring at −40° C., neat 3-chlorobenzoyl chloride (0.796 mL, 6.03 mmol, 1.5 eq) was added, and the reaction mixture was stirred for an additional 10 min at −40° C. The solution was allowed to warm and stir for 1 h at ambient temperature, and then quenched at 0° C. with a 1:1 mixture of saturated aqueous ammonium chloride/10% ammonium hydroxide. This mixture was stirred for 60 h at ambient temperature and extracted with ethyl acetate. The organic portion was combined and dried with sodium sulfate and concentrated, and the resulting crude reaction material was purified via chromatography (0-80% ethyl acetate in hexanes) to provide 1.0 g of the title product.

¹H NMR δ 7.90 (m, 1H), 7.81 (m, 1H), 7.57 (m, 1H), 7.38-7.50 (m, 1H), 3.72 (s, 3H), 3.67 (s, 3H), 2.29 (s, 3H).

### Step B: Preparation of 4-(3-chlorobenzoyl)-5-hydroxy-2,6-dimethyl-3(2H)-pyridazinone

To a flask containing a magnetic stirbar, 5-hydroxy-2,6-dimethyl-4-(1-naphthalenylcarbonyl)-3(2H)-pyridazinone (i.e. the product from Step A) (0.35 g, 0.854 mmol, 1.0 eq) and lithium chloride (0.36 g, 8.54 mmol, 10 eq) was added 1,4-dioxane (3 mL) and N,N-dimethylacetamide (2 mL). The solution was heated to 130° C. and allowed to stir at this temperature for 40 min. The reaction mixture was then cooled to ambient temperature and diluted with I N hydrochloric acid, and the resulting solids were filtered and washed with water to afford 0.287 g of the title compound.

¹H NMR δ 13.74 (s, 1H), 7.62 (m, 1H), 7.47-7.57 (m, 2H), 7.34-7.41 (m, 1H), 3.67 (s, 3H), 2.36 (s, 3H).

### Step C: Preparation of 4-[(Z)-(3-chlorophenyl)(methoxyimino)methyl]-5-hydroxy-5-2,6-dimethyl-3(2H)-pyridazinone and 4-[(E)-(3-chlorophenyl)(methoxyimino)methyl]-5-hydroxy-2,6-dimethyl-3(2H)-pyridazinone

Methanol (1.0 mL) was added to a sealed vial containing 4-(3-chlorobenzoyl)-5-hydroxy-2,6-dimethyl-3(2H)-pyridazinone (i.e. the product from Step B) (0.1 g, 0.359 mmol, 1.0 eq), methoxyamine hydrochloride (46 mg, 0.539 mmol, 1.5 eq) and sodium bicarbonate (45 mg, 0.539 mmol, 1.5 eq), and the resulting suspension was stirred overnight at ambient temperature. The solution was then quenched with 1 N aqueous hydrochloric acid and extracted with ethyl acetate. The organic portions were combined, dried with sodium sulfate and concentrated. The resulting residue was purified by chromatography to afford 81.8 mg of the Z-isomer and 24.3 mg of the E-isomer.

Z-isomer: ¹H NMR 68.27 (s, 1H), 7.44 (m, 11H), 7.25-7.30 (m, 2H), 7.18-7.22 (m, 1H), 4.01 (s, 3H), 3.55 (s, 3H), 2.27 (s, 3H).

E-isomer ¹H NMR δ 12.17 (s, 1H), 7.33-7.38 (m, 2H), 7.23-7.27 (m, 1H), 7.11-7.17 (m, 1H), 3.97 (s, 3H), 3.57 (s, 3H), 2.34 (s, 3H).

By the procedures described herein together with the methods known in the art, the following compounds of Tables 1-6 can be prepared, where both the E and Z isomers, or a mixture thereof are disclosed. The following abbreviations are used in the Tables which follow: Me means methyl, Et means ethyl, i-Pr means isopropyl, CN means cyano, and NO₂ means nitro.

TABLE 1

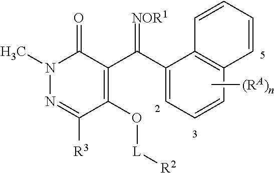

L is a direct bond; and R² is H

| R¹ | R³ | R^{A} |
|---|---|---|
| Me | Me | 2-Me |
| Me | Me | 2-Et |
| Me | Me | 2-F |
| Me | Me | 2-Cl |
| Me | Me | 2-Br |
| Me | Me | 2-CF₃ |
| Me | Me | 2-OCHF₂ |
| Me | Me | 2-CN |
| Me | Me | 3-Me |
| Me | Me | 3-Et |
| Me | Me | 3-F |
| Me | Me | 3-CF₃ |
| Me | Me | 3-OCHF₂ |
| Me | Me | 3-CN |
| Me | Me | 3-SO₂Me |
| Me | Me | 3-SO₂Et |
| Me | Me | 3-NO₂ |
| Me | Me | 4-CN |
| Me | Me | 5-Me |
| Me | Me | 5-Et |
| Me | Me | 5-F |
| Me | Me | 5-Br |
| Me | Me | 5-CF₃ |
| Me | Me | 5-OCHF₂ |
| Me | Me | 5-CN |
| Me | Me | 6-Me |
| Me | Me | 6-Et |
| Me | Me | 6-Cl |
| Me | Me | 6-CF₃ |
| Me | Me | 6-OCHF₂ |
| Me | Me | 6-CN |
| Me | Me | 6-SO₂Me |
| Me | Me | 6-SO₂Et |
| Me | Me | 6-NO₂ |
| Me | Me | 7-Me |
| Me | Me | 7-Et |
| Me | Me | 7-F |
| Me | Me | 7-Cl |
| Me | Me | 7-Br |
| Me | Me | 7-CF₃ |
| Me | Me | 7-OCHF₂ |
| Me | Me | 7-CN |
| Me | Me | 7-NO₂ |
| Me | Me | 8-Me |
| Me | Me | 8-Et |
| Me | Me | 8-F |
| Me | Me | 8-Cl |
| Me | Me | 8-Br |
| Me | Me | 8-CF₃ |
| Me | Me | 8-OCHF₂ |
| Me | Me | 8-CN |
| Me | Me | 8-NO₂ |
| Me | Cl | 2-Me |
| Me | Cl | 2-Et |
| Me | Cl | 2-F |
| Me | Cl | 2-Cl |
| Me | Cl | 2-Br |
| Me | Cl | 2-CF₃ |
| Me | Cl | 2-OCHF₂ |
| Me | Cl | 2-CN |
| Me | Cl | 3-Me |
| Me | Cl | 3-Et |
| Me | Cl | 3-F |
| Me | Cl | 3-CF₃ |
| Me | Cl | 3-OCHF₂ |
| Me | Cl | 3-CN |
| Me | Cl | 3-SO₂Me |
| Me | Cl | 3-SO₂Et |
| Me | Cl | 3-NO₂ |
| Me | Cl | 4-CN |
| Me | Cl | 5-Me |
| Me | Cl | 5-Et |
| Me | Cl | 5-F |
| Me | Cl | 5-Br |
| Me | Cl | 5-CF₃ |
| Me | Cl | 5-OCHF₂ |
| Me | Cl | 5-CN |
| Me | Cl | 6-Me |
| Me | Cl | 6-Et |
| Me | Cl | 6-Cl |
| Me | Cl | 6-CF₃ |
| Me | Cl | 6-OCHF₂ |
| Me | Cl | 6-CN |
| Me | Cl | 6-SO₂Me |
| Me | Cl | 6-SO₂Et |
| Me | Cl | 6-NO₂ |
| Me | Cl | 7-Me |
| Me | Cl | 7-Et |
| Me | Cl | 7-F |
| Me | Cl | 7-Cl |
| Me | Cl | 7-Br |
| Me | Cl | 7-CF₃ |
| Me | Cl | 7-OCHF₂ |
| Me | Cl | 7-CN |
| Me | Cl | 7-NO₂ |
| Me | Cl | 8-Me |
| Me | Cl | 8-Et |
| Me | Cl | 8-F |
| Me | Cl | 8-Cl |
| Me | Cl | 8-Br |
| Me | Cl | 8-CF₃ |
| Me | Cl | 8-OCHF₂ |
| Me | Cl | 8-CN |
| Me | Cl | 8-NO₂ |
| Et | Me | 2-Me |
| Et | Me | 2-Et |
| Et | Me | 2-F |
| Et | Me | 2-Cl |
| Et | Me | 2-Br |
| Et | Me | 2-CF₃ |
| Et | Me | 2-OCHF₂ |
| Et | Me | 2-CN |
| Et | Me | 3-Me |
| Et | Me | 3-Et |
| Et | Me | 3-F |
| Et | Me | 3-CF₃ |
| Et | Me | 3-OCHF₂ |
| Et | Me | 3-CN |
| Et | Me | 3-SO₂Me |
| Et | Me | 3-SO₂Et |
| Et | Me | 3-NO₂ |
| Et | Me | 4-CN |
| Et | Me | 5-Me |
| Et | Me | 5-Et |
| Et | Me | 5-F |
| Et | Me | 5-Br |
| Et | Me | 5-CF₃ |
| Et | Me | 5-OCHF₂ |
| Et | Me | 5-CN |
| Et | Me | 6-Me |
| Et | Me | 6-Et |
| Et | Me | 6-Cl |
| Et | Me | 6-CF₃ |
| Et | Me | 6-OCHF₂ |
| Et | Me | 6-CN |
| Et | Me | 6-SO₂Me |
| Et | Me | 6-SO₂Et |
| Et | Me | 6-NO₂ |
| Et | Me | 7-Me |
| Et | Me | 7-Et |
| Et | Me | 7-F |
| Et | Me | 7-Cl |
| Et | Me | 7-Br |
| Et | Me | 7-CF₃ |
| Et | Me | 7-OCHF₂ |
| Et | Me | 7-CN |
| Et | Me | 7-NO₂ |
| Et | Me | 8-Me |
| Et | Me | 8-Et |
| Et | Me | 8-F |
| Et | Me | 8-Cl |
| Et | Me | 8-Br |
| Et | Me | 8-CF₃ |
| Et | Me | 8-OCHF₂ |
| Et | Me | 8-CN |
| Et | Me | 8-NO₂ |
| Et | Cl | 2-Me |
| Et | Cl | 2-Et |
| Et | Cl | 2-F |
| Et | Cl | 2-Cl |
| Et | Cl | 2-Br |
| Et | Cl | 2-CF₃ |
| Et | Cl | 2-OCHF₂ |
| Et | Cl | 2-CN |
| Et | Cl | 3-Me |
| Et | Cl | 3-Et |
| Et | Cl | 3-F |
| Et | Cl | 3-CF₃ |
| Et | Cl | 3-OCHF₂ |
| Et | Cl | 3-CN |
| Et | Cl | 3-SO₂Me |
| Et | Cl | 3-SO₂Et |
| Et | Cl | 3-NO₂ |
| Et | Cl | 4-CN |
| Et | Cl | 5-Me |
| Et | Cl | 5-Et |
| Et | Cl | 5-F |
| Et | Cl | 5-Br |
| Et | Cl | 5-CF₃ |
| Et | Cl | 5-OCHF₂ |
| Et | Cl | 5-CN |
| Et | Cl | 6-Me |
| Et | Cl | 6-Et |
| Et | Cl | 6-Cl |
| Et | Cl | 6-CF₃ |
| Et | Cl | 6-OCHF₂ |
| Et | Cl | 6-CN |
| Et | Cl | 6-SO₂Me |
| Et | Cl | 6-SO₂Et |
| Et | Cl | 6-NO₂ |
| Et | Cl | 7-Me |
| Et | Cl | 7-Et |
| Et | Cl | 7-F |
| Et | Cl | 7-Cl |
| Et | Cl | 7-Br |
| Et | Cl | 7-CF₃ |
| Et | Cl | 7-OCHF₂ |
| Et | Cl | 7-CN |
| Et | Cl | 7-NO₂ |
| Et | Cl | 8-Me |
| Et | Cl | 8-Et |
| Et | Cl | 8-F |
| Et | Cl | 8-Cl |
| Et | Cl | 8-Br |
| Et | Cl | 8-CF₃ |
| Et | Cl | 8-OCHF₂ |
| Et | Cl | 8-CN |
| Et | Cl | 8-NO₂ |
| i-Pr | Me | 2-Me |
| i-Pr | Me | 2-Et |
| i-Pr | Me | 2-F |
| i-Pr | Me | 2-Cl |
| i-Pr | Me | 2-Br |
| i-Pr | Me | 2-CF₃ |
| i-Pr | Me | 2-OCHF₂ |
| i-Pr | Me | 2-CN |
| i-Pr | Me | 3-Me |
| i-Pr | Me | 3-Et |
| i-Pr | Me | 3-F |
| i-Pr | Me | 3-CF₃ |
| i-Pr | Me | 3-OCHF₂ |
| i-Pr | Me | 3-CN |
| i-Pr | Me | 3-SO₂Me |
| i-Pr | Me | 3-SO₂Et |
| i-Pr | Me | 3-NO₂ |
| i-Pr | Me | 4-CN |
| i-Pr | Me | 5-Me |
| i-Pr | Me | 5-Et |
| i-Pr | Me | 5-F |
| i-Pr | Me | 5-Br |
| i-Pr | Me | 5-CF₃ |
| i-Pr | Me | 5-OCHF₂ |
| i-Pr | Me | 5-CN |
| i-Pr | Me | 6-Me |
| i-Pr | Me | 6-Et |
| i-Pr | Me | 6-Cl |
| i-Pr | Me | 6-CF₃ |
| i-Pr | Me | 6-OCHF₂ |
| i-Pr | Me | 6-CN |
| i-Pr | Me | 6-SO₂Me |
| i-Pr | Me | 6-SO₂Et |
| i-Pr | Me | 6-NO₂ |
| i-Pr | Me | 7-Me |
| i-Pr | Me | 7-Et |
| i-Pr | Me | 7-F |
| i-Pr | Me | 7-Cl |
| i-Pr | Me | 7-Br |
| i-Pr | Me | 7-CF₃ |
| i-Pr | Me | 7-OCHF₂ |
| i-Pr | Me | 7-CN |
| i-Pr | Me | 7-NO₂ |
| i-Pr | Me | 8-Me |
| i-Pr | Me | 8-Et |
| i-Pr | Me | 8-F |
| i-Pr | Me | 8-Cl |
| i-Pr | Me | 8-Br |
| i-Pr | Me | 8-CF₃ |
| i-Pr | Me | 8-OCHF₂ |
| i-Pr | Me | 8-CN |
| i-Pr | Me | 8-NO₂ |
| i-Pr | Cl | 2-Me |
| i-Pr | Cl | 2-Et |
| i-Pr | Cl | 2-F |
| i-Pr | Cl | 2-Cl |
| i-Pr | Cl | 2-Br |
| i-Pr | Cl | 2-CF₃ |
| i-Pr | Cl | 2-OCHF₂ |
| i-Pr | Cl | 2-CN |
| i-Pr | Cl | 3-Me |
| i-Pr | Cl | 3-Et |
| i-Pr | Cl | 3-F |
| i-Pr | Cl | 3-CF₃ |
| i-Pr | Cl | 3-OCHF₂ |
| i-Pr | Cl | 3-CN |
| i-Pr | Cl | 3-SO₂Me |
| i-Pr | Cl | 3-SO₂Et |
| i-Pr | Cl | 3-NO₂ |
| i-Pr | Cl | 4-CN |
| i-Pr | Cl | 5-Me |
| i-Pr | Cl | 5-Et |
| i-Pr | Cl | 5-F |
| i-Pr | Cl | 5-Br |
| i-Pr | Cl | 5-CF₃ |
| i-Pr | Cl | 5-OCHF₂ |
| i-Pr | Cl | 5-CN |
| i-Pr | Cl | 6-Me |
| i-Pr | Cl | 6-Et |
| i-Pr | Cl | 6-Cl |
| i-Pr | Cl | 6-CF₃ |
| i-Pr | Cl | 6-OCHF₂ |
| i-Pr | Cl | 6-CN |
| i-Pr | Cl | 6-SO₂Me |
| i-Pr | Cl | 6-SO₂Et |
| i-Pr | Cl | 6-NO₂ |
| i-Pr | Cl | 7-Me |
| i-Pr | Cl | 7-Et |
| i-Pr | Cl | 7-F |
| i-Pr | Cl | 7-Cl |
| i-Pr | Cl | 7-Br |
| i-Pr | Cl | 7-CF₃ |
| i-Pr | Cl | 7-OCHF₂ |
| i-Pr | Cl | 7-CN |
| i-Pr | Cl | 7-NO₂ |
| i-Pr | Cl | 8-Me |
| i-Pr | Cl | 8-Et |
| i-Pr | Cl | 8-F |
| i-Pr | Cl | 8-Cl |
| i-Pr | Cl | 8-Br |
| i-Pr | Cl | 8-CF₃ |
| i-Pr | Cl | 8-OCHF₂ |
| i-Pr | Cl | 8-CN |
| i-Pr | Cl | 8-NO₂ |
| -CH₂C≡CH | Me | 2-Me |
| -CH₂C≡CH | Me | 2-Et |
| -CH₂C≡CH | Me | 2-F |
| -CH₂C≡CH | Me | 2-Cl |
| -CH₂C≡CH | Me | 2-Br |
| -CH₂C≡CH | Me | 2-CF₃ |
| -CH₂C≡CH | Me | 2-OCHF₂ |
| -CH₂C≡CH | Me | 2-CN |
| -CH₂C≡CH | Me | 3-Me |
| -CH₂C≡CH | Me | 3-Et |
| -CH₂C≡CH | Me | 3-F |
| -CH₂C≡CH | Me | 3-CF₃ |
| -CH₂C≡CH | Me | 3-OCHF₂ |
| -CH₂C≡CH | Me | 3-CN |
| -CH₂C≡CH | Me | 3-SO₂Me |
| -CH₂C≡CH | Me | 3-SO₂Et |
| -CH₂C≡CH | Me | 3-NO₂ |
| -CH₂C≡CH | Me | 4-CN |
| -CH₂C≡CH | Me | 5-Me |
| -CH₂C≡CH | Me | 5-Et |
| -CH₂C≡CH | Me | 5-F |
| -CH₂C≡CH | Me | 5-Br |
| -CH₂C≡CH | Me | 5-CF₃ |
| -CH₂C≡CH | Me | 5-OCHF₂ |
| -CH₂C≡CH | Me | 5-CN |
| -CH₂C≡CH | Me | 6-Me |
| -CH₂C≡CH | Me | 6-Et |
| -CH₂C≡CH | Me | 6-Cl |
| -CH₂C≡CH | Me | 6-CF₃ |
| -CH₂C≡CH | Me | 6-OCHF₂ |
| -CH₂C≡CH | Me | 6-CN |
| -CH₂C≡CH | Me | 6-SO₂Me |
| -CH₂C≡CH | Me | 6-SO₂Et |
| -CH₂C≡CH | Me | 6-NO₂ |
| -CH₂C≡CH | Me | 7-Me |
| -CH₂C≡CH | Me | 7-Et |
| -CH₂C≡CH | Me | 7-F |
| -CH₂C≡CH | Me | 7-Cl |
| -CH₂C≡CH | Me | 7-Br |
| -CH₂C≡CH | Me | 7-CF₃ |
| -CH₂C≡CH | Me | 7-OCHF₂ |
| -CH₂C≡CH | Me | 7-CN |
| -CH₂C≡CH | Me | 7-NO₂ |
| -CH₂C≡CH | Me | 8-Me |
| -CH₂C≡CH | Me | 8-Et |
| -CH₂C≡CH | Me | 8-F |
| -CH₂C≡CH | Me | 8-Cl |
| -CH₂C≡CH | Me | 8-Br |
| -CH₂C≡CH | Me | 8-CF₃ |
| -CH₂C≡CH | Me | 8-OCHF₂ |
| -CH₂C≡CH | Me | 8-CN |
| -CH₂C≡CH | Me | 8-NO₂ |
| -CH₂C≡CH | Cl | 2-Me |
| -CH₂C≡CH | Cl | 2-Et |
| -CH₂C≡CH | Cl | 2-F |
| -CH₂C≡CH | Cl | 2-Cl |
| -CH₂C≡CH | Cl | 2-Br |
| -CH₂C≡CH | Cl | 2-CF₃ |
| -CH₂C≡CH | Cl | 2-OCHF₂ |
| -CH₂C≡CH | Cl | 2-CN |
| -CH₂C≡CH | Cl | 3-Me |
| -CH₂C≡CH | Cl | 3-Et |
| -CH₂C≡CH | Cl | 3-F |
| -CH₂C≡CH | Cl | 3-CF₃ |
| -CH₂C≡CH | Cl | 3-OCHF₂ |
| -CH₂C≡CH | Cl | 3-CN |
| -CH₂C≡CH | Cl | 3-SO₂Me |
| -CH₂C≡CH | Cl | 3-SO₂Et |
| -CH₂C≡CH | Cl | 3-NO₂ |
| -CH₂C≡CH | Cl | 4-CN |
| -CH₂C≡CH | Cl | 5-Me |
| -CH₂C≡CH | Cl | 5-Et |
| -CH₂C≡CH | Cl | 5-F |
| -CH₂C≡CH | Cl | 5-Br |
| -CH₂C≡CH | Cl | 5-CF₃ |
| -CH₂C≡CH | Cl | 5-OCHF₂ |
| -CH₂C≡CH | Cl | 5-CN |
| -CH₂C≡CH | Cl | 6-Me |
| -CH₂C≡CH | Cl | 6-Et |
| -CH₂C≡CH | Cl | 6-Cl |
| -CH₂C≡CH | Cl | 6-CF₃ |
| -CH₂C≡CH | Cl | 6-OCHF₂ |
| -CH₂C≡CH | Cl | 6-CN |
| -CH₂C≡CH | Cl | 6-SO₂Me |
| -CH₂C≡CH | Cl | 6-SO₂Et |
| -CH₂C≡CH | Cl | 6-NO₂ |
| -CH₂C≡CH | Cl | 7-Me |
| -CH₂C≡CH | Cl | 7-Et |
| -CH₂C≡CH | Cl | 7-F |
| -CH₂C≡CH | Cl | 7-Cl |
| -CH₂C≡CH | Cl | 7-Br |
| -CH₂C≡CH | Cl | 7-CF₃ |
| -CH₂C≡CH | Cl | 7-OCHF₂ |
| -CH₂C≡CH | Cl | 7-CN |
| -CH₂C≡CH | Cl | 7-NO₂ |
| -CH₂C≡CH | Cl | 8-Me |
| -CH₂C≡CH | Cl | 8-Et |
| -CH₂C≡CH | Cl | 8-F |
| -CH₂C≡CH | Cl | 8-Cl |
| -CH₂C≡CH | Cl | 8-Br |
| -CH₂C≡CH | Cl | 8-CF₃ |
| -CH₂C≡CH | Cl | 8-OCHF₂ |
| -CH₂C≡CH | Cl | 8-CN |
| -CH₂C≡CH | Cl | 8-NO₂ |
| Me | Me | 3,6-(Br)₂ |
| Me | Me | 3,6-(Cl)₂ |
| Me | Me | 3,6-(F)₂ |
| Me | Me | 3,6-(Me)₂ |
| Et | Me | 3,6-(Br)₂ |
| Et | Me | 3,6-(Cl)₂ |
| Et | Me | 3,6-(F)₂ |
| Et | Me | 3,6-(Me)₂ |
| i-Pr | Me | 3,6-(Br)₂ |
| i-Pr | Me | 3,6-(Cl)₂ |
| i-Pr | Me | 3,6-(F)₂ |
| i-Pr | Me | 3,6-(Me)₂ |
| -CH₂C≡CH | Me | 3,6-(Br)₂ |
| -CH₂C≡CH | Me | 3,6-(Cl)₂ |
| -CH₂C≡CH | Me | 3,6-(F)₂ |
| -CH₂C≡CH | Me | 3,6-(Me)₂ |
| Me | Cl | 3-Br |
| Me | Cl | 4-F |
| Me | Cl | 6-Br |
| Et | Cl | 3-Br |
| Et | Cl | 4-F |
| Et | Cl | 6-Br |
| i-Pr | Cl | 3-Br |
| i-Pr | Cl | 4-F |
| i-Pr | Cl | 6-Br |
| -CH₂C≡CH | Cl | 3-Br |
| -CH₂C≡CH | Cl | 4-F |
| -CH₂C≡CH | Cl | 6-Br |
| Me | Me | H (n = 0) |
| Me | Cl | H (n = 0) |
| Et | Me | H (n = 0) |
| Et | Cl | H (n = 0) |
| i-Pr | Me | H (n = 0) |
| i-Pr | Cl | H (n = 0) |
| -CH₂C≡CH | Me | H (n = 0) |
| -CH₂C≡CH | Cl | H (n = 0) |

Tables 2 through 6 are constructed in the same fashion as Table 1 except the header row “L is a direct bond; and R² is H” is replaced with the listed header row.

| Table | Header Row |
|---|---|
| 2 | L is a direct bond; and R² is C(═O)Me |
| 3 | L is a direct bond; and R² is C(═O)Et |
| 4 | L is a direct bond; and R² is C(═O)i-Pr |
| 5 | L is a direct bond; and R² is CO₂Me |
| 6 | L is a direct bond; and R² is CO₂Et |

A compound of this invention will generally be used as a herbicidal active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil-in-water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil-in-water emulsion, flowable concentrate and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation: alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water-soluble | 0.001-90 | 0-99.999 | 0-15 |
| Granules, Tablets and Powders | | | |
| Oil Dispersions, Suspensions, | 1-50 | 40-99 | 0-50 |
| Emulsions, Solutions (including | | | |
| Emulsifiable Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methy-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils: alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters: ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides: amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition. John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”. Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96: Hance et al., Weed Control Handbook, 8th Ed., Blackwell Scientific Publications, Oxford, 1989; and Developments in formulation technology, PJB Publications. Richmond, U K, 2000.

In the following Examples, all percentages are by weight and all formulations are prepared in conventional ways. Compound numbers refer to compounds in Index Table A. Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except where otherwise indicated.

### Example A

High Strength Concentrate

| | |
|---|---|
| Compound 1 | 98.5% |
| silica aerogel | 0.5% |
| synthetic amorphous fine silica | 1.0% |

### Example B

Wettable Powder

| | |
|---|---|
| Compound 1 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 1 | 10.0% |
| attapulgite granules (low volatile matter, | 90.0% |
| 0.71/0.30 mm; U.S.S. No. 25-50 sieves) | |

### Example D

Extruded Pellet

| | |
|---|---|
| Compound 1 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 1 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 1 | 5.0% |
| polyvinylpyrrolidone-vinyl acetate copolymer | 30.0% |
| alkylpolyglycoside | 30.0% |
| glyceryl monooleate | 15.0% |
| Water | 20.0% |

### Example G

Suspension Concentrate

| | |
|---|---|
| Compound 1 | 35% |
| butyl polyoxyethylene/polypropylene block | 4.0% |
| copolymer | |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| Water | 53.7% |

### Example H

Emulsion in Water

| | |
|---|---|
| Compound 1 | 10.0% |
| butyl polyoxyethylene/polypropylene block | 4.0% |
| copolymer | |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| Water | 58.7% |

### Example I

Oil Dispersion

| | |
|---|---|
| Compound 1 | 25% |
| polyoxyethylene sorbitol hexaoleate | 15% |
| organically modified bentonite clay | 2.5% |
| fatty acid methyl ester | 57.5% |

Test results indicate that the compounds of the present invention are highly active preemergent and/or postemergent herbicides and/or plant growth regulants. The compounds of the invention generally show highest activity for postemergence weed control (i.e. applied after weed seedlings emerge from the soil) and preemergence weed control (i.e. applied before weed seedlings emerge from the soil). Many of them have utility for broad-spectrum pre- and/or postemergence weed control in areas where complete control of all vegetation is desired such as around fuel storage tanks, industrial storage areas, parking lots, drive-in theaters, air fields, river banks, irrigation and other waterways, around billboards and highway and railroad structures. Many of the compounds of this invention, by virtue of selective metabolism in crops versus weeds, or by selective activity at the locus of physiological inhibition in crops and weeds, or by selective placement on or within the environment of a mixture of crops and weeds, are useful for the selective control of grass and broadleaf weeds within a crop/weed mixture. One skilled in the art will recognize that the preferred combination of these selectivity factors within a compound or group of compounds can readily be determined by performing routine biological and/or biochemical assays. Compounds of this invention may show tolerance to important agronomic crops including, but is not limited to, alfalfa, barley, cotton, wheat, rape, sugar beets, corn (maize), sorghum, soybeans, rice, oats, peanuts, vegetables, tomato, potato, perennial plantation crops including coffee, cocoa, oil palm, rubber, sugarcane, citrus, grapes, fruit trees, nut trees, banana, plantain, pineapple, hops, tea and forests such as eucalyptus and conifers (e.g., loblolly pine), and turf species (e.g., Kentucky bluegrass, St. Augustine grass, Kentucky fescue and Bermuda grass). Compounds of this invention can be used in crops genetically transformed or bred to incorporate resistance to herbicides, express proteins toxic to invertebrate pests (such as Bacillus thuringiensis toxin), and/or express other useful traits. Those skilled in the art will appreciate that not all compounds are equally effective against all weeds. Alternatively, the subject compounds are useful to modify plant growth.

As the compounds of the invention have both preemergent and postemergent herbicidal activity, to control undesired vegetation by killing or injuring the vegetation or reducing its growth, the compounds can be usefully applied by a variety of methods involving contacting a herbicidally effective amount of a compound of the invention, or a composition comprising said compound and at least one of a surfactant, a solid diluent or a liquid diluent, to the foliage or other part of the undesired vegetation or to the environment of the undesired vegetation such as the soil or water in which the undesired vegetation is growing or which surrounds the seed or other propagule of the undesired vegetation. Of note is the control of undesired vegetation selected from the group consisting of ragweed, gallium, wild oats, kochia, giant foxtail, green foxtail and blackgrass. Of particular note is the control of kochia.

A herbicidally effective amount of the compounds of this invention is determined by a number of factors. These factors include: formulation selected, method of application, amount and type of vegetation present, growing conditions, etc. In general, a herbicidally effective amount of compounds of this invention is about 0.001 to 20 kg/ha with a preferred range of about 0.004 to I kg/ha. One skilled in the art can easily determine the herbicidally effective amount necessary for the desired level of weed control.

In one common embodiment, a compound of the invention is applied, typically in a formulated composition, to a locus comprising desired vegetation (e.g., crops) and undesired vegetation (i.e. weeds), both of which may be seeds, seedlings and/or larger plants, in contact with a growth medium (e.g., soil). In this locus, a composition comprising a compound of the invention can be directly applied to a plant or a part thereof, particularly of the undesired vegetation, and/or to the growth medium in contact with the plant.

Plant varieties and cultivars of the desired vegetation in the locus treated with a compound of the invention can be obtained by conventional propagation and breeding methods or by genetic engineering methods. Genetically modified plants (transgenic plants) are those in which a heterologous gene (transgene) has been stably integrated into the plant's genome. A transgene that is defined by its particular location in the plant genome is called a transformation or transgenic event.

Genetically modified plant cultivars in the locus which can be treated according to the invention include those that are resistant against one or more biotic stresses (pests such as nematodes, insects, mites, fungi, etc.) or abiotic stresses (drought, cold temperature, soil salinity, etc.), or that contain other desirable characteristics. Plants can be genetically modified to exhibit traits of, for example, herbicide tolerance, insect-resistance, modified oil profiles or drought tolerance. Useful genetically modified plants containing single gene transformation events or combinations of transformation events are listed in Exhibit C. Additional information for the genetic modifications listed in Exhibit C can be obtained from publicly available databases maintained, for example, by the U.S. Department of Agriculture.

Compounds of this invention can also be mixed with one or more other biologically active compounds or agents including herbicides, herbicide safeners, fungicides, insecticides, nematocides, bactericides, acaricides, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Mixtures of the compounds of the invention with other herbicides can broaden the spectrum of activity against additional weed species, and suppress the proliferation of any resistant biotypes. Thus the present invention also pertains to a composition comprising a compound of Formula 1 (in a herbicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

A mixture of one or more of the following herbicides with a compound of this invention may be particularly useful for weed control: acetochlor, acifluorfen and its sodium salt, aclonifen, acrolein (2-propenal), alachlor, alloxydim, ametryn, amicarbazone, amidosulfuron, aminocyclopyrachlor and its esters (e.g., methyl, ethyl) and salts (e.g., sodium, potassium), aminopyralid, amitrole, ammonium sulfamate, anilofos, asulam, atrazine, azimsulfuron, beflubutamid, S-beflubutamid, benazolin, benazolin-ethyl, bencarbazone, benfluralin, benfuresate, bensulfuron-methyl, bensulide, bentazone, benzobicyclon, benzofenap, bicyclopyrone, bifenox, bilanafos, bispyribac and its sodium salt, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinmethylin, cinosulfuron, clacyfos, clefoxydim, clethodim, clodinafop-propargyl, clomazone, clomeprop, clopyralid, clopyralid-olamine, cloransulam-methyl, cumyluron, cyanazine, cycloate, cyclopyrimorate, cyclosulfamuron, cycloxydim, cyhalofop-butyl, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimethachlor, dimethametryn, dimethenamid, dimethenamid-P, dimethipin, dimethylarsinic acid and its sodium salt, dinitramine, dinoterb, diphenamid, diquat dibromide, dithiopyr, diuron, DNOC, endothal, EPTC, esprocarb, ethalfluralin, ethametsulfuron-methyl, ethiozin, ethofumesate, ethoxyfen, ethoxysulfuron, etobenzanid, fenoxaprop-ethyl, fenoxaprop-P-ethyl, fenoxasulfone, fenquinotrione, fentrazamide, fenuron, fenuron-TCA, flamprop-methyl, flamprop-M-isopropyl, flamprop-M-methyl, flazasulfuron, florasulam, fluazifop-butyl, fluazifop-P-butyl, fluazolate, flucarbazone, flucetosulfuron, fluchloralin, flufenacet, flufenpyr, flufenpyr-ethyl, flumetsulam, flumiclorac-pentyl, flumioxazin, fluometuron, fluoroglycofen-ethyl, flupoxam, flupyrsulfuron-methyl and its sodium salt, flurenol, flurenol-butyl, fluridone, flurochloridone, fluroxypyr, flurtamone, fluthiacet-methyl, fomesafen, foramsulfuron, fosamine-ammonium, glufosinate, glufosinate-ammonium, glufosinate-P, glyphosate and its salts such as ammonium, isopropylammonium, potassium, sodium (including sesquisodium) and trimesium (alternatively named sulfosate), halauxifen, halauxifen-methyl, halosulfuron-methyl, haloxyfop-etotyl, haloxyfop-methyl, hexazinone, hydantocidin, imazamethabenz-methyl, imazamox, imazapic, imazapyr, imazaquin, imazaquin-ammonium, imazethapyr, imazethapyr-ammonium, imazosulfuron, indanofan, indaziflam, iofensulfuron, iodosulfuron-methyl, ioxynil, ioxynil octanoate, ioxynil-sodium, ipfencarbazone, isoproturon, isouron, isoxaben, isoxaflutole, isoxachlortole, lactofen, lenacil, linuron, maleic hydrazide, MCPA and its salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, mesotrione, metam-sodium, metamifop, metamitron, metazachlor, metazosulfuron, methabenzthiazuron, methylarsonic acid and its calcium, monoammonium, monosodium and disodium salts, methyldymron, metobenzuron, metobromuron, metolachlor, S-metolachlor, metosulam, metoxuron, metribuzin, metsulfuron-methyl, molinate, monolinuron, naproanilide, napropamide, napropamide-M, naptalam, neburon, nicosulfuron, norflurazon, orbencarb, orthosulfamuron, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxamid, pethoxyamid, phenmedipham, picloram, picloram-potassium, picolinafen, pinoxaden, piperophos, pretilachlor, primisulfuron-methyl, prodiamine, profoxydim, prometon, prometryn, propachlor, propanil, propaquizafop, propazine, propham, propisochlor, propoxycarbazone, propyrisulfuron, propyzamide, prosulfocarb, prosulfuron, pyraclonil, pyraflufen-ethyl, pyrasulfotole, pyrazogyl, pyrazolynate, pyrazoxyfen, pyrazosulfuron-ethyl, pyribenzoxim, pyributicarb, pyridate, pyriftalid, pyriminobac-methyl, pyrimisulfan, pyrithiobac, pyrithiobac-sodium, pyroxasulfone, pyroxsulam, quinclorac, quinmerac, quinoclamine, quizalofop-ethyl, quizalofop-P-ethyl, quizalofop-P-tefuryl, rimsulfuron, saflufenacil, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tefuryltrione, tembotrione, tepraloxydim, terbacil, terbumeton, terbuthylazine, terbutryn, thenylchlor, thiazopyr, thiencarbazone, thifensulfuron-methyl, thiobencarb, tiafenacil, tiocarbazil, tolpyralate, topramezone, tralkoxydim, tri-allate, triafamone, triasulfuron, triaziflam, tribenuron-methyl, triclopyr, triclopyr-butotyl, triclopyr-triethylammonium, tridiphane, trietazine, trifloxysulfuron, trifludimoxazin, trifluralin, triflusulfuron-methyl, tritosulfuron, vernolate, 3-(2-chloro-3.6-difluorophenyl)-4-hydroxy-1-methyl-1,5-naphthyridin-2(11)-one, 5-chloro-3-[(2-hydroxy-6-oxo-1-cyclohexen-1-yl)carbonyl]-1-(4-methoxyphenyl)-2(1H)-quinoxalinone, 2-chloro-N-(1-methyl-1H-tetrazol-5-yl)-6-(trifluoromethyl)-3-pyridinecarboxamide, 7-(3,5-dichloro-4-pyridinyl)-5-(2,2-difluoroethyl)-8-hydroxypyrido[2,3-b]pyrazin-6(5H)-one), 4-(2,6-diethyl-4-methylphenyl)-5-hydroxy-2,6-dimethyl-3(2H)-pyridazinone), 5-[[(2,6-difluorophenyl)methoxy]methyl]-4,5-dihydro-5-methyl-3-(3-methyl-2-thienyl)isoxazole (previously methioxolin), 4-(4-fluorophenyl)-6-[(2-hydroxy-6-oxo-1-cyclohexen-1-yl)carbonyl]-2-methyl-1,2,4-triazine-3,5(2H,4H)-dione, methyl 4-amino-3-chloro-6-(4-chloro-2-fluoro-3-methoxyphenyl)-5-fluoro-2-pyridinecarboxylate, 2-methyl-3-(methylsulfonyl)-N-(1-methyl-1H-tetrazol-5-yl)-4-(trifluoromethyl)benzamide and 2-methyl-N-(4-methyl-1,2,5-oxadiazol-3-yl)-3-(methylsulfinyl)-4-(trifluoromethyl)benzamide. Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palmivora (Butl.) Butl. and Puccinia thlaspeos Schub.

Compounds of this invention can also be used in combination with plant growth regulators such as aviglycine, N-(phenylmethyl)-1H-purin-6-amine, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

General references for agricultural protectants (i.e. herbicides, herbicide safeners, insecticides, fungicides, nematocides, acaricides and biological agents) include The Pesticide Manual, 13th Edition, C. D. S. Tomlin, Ed., British Crop Protection Council, Farnham, Surrey, U. K., 2003 and The BioPesticide Manual, 2nd Edition, L. G. Copping, Ed., British Crop Protection Council, Farnham, Surrey, U. K., 2001.

For embodiments where one or more of these various mixing partners are used, the mixing partners are typically used in the amounts similar to amounts customary when the mixture partners are used alone. More particularly in mixtures, active ingredients are often applied at an application rate between one-half and the full application rate specified on product labels for use of active ingredient alone. These amounts are listed in references such as The Pesticide Manual and The BioPesticide Manual. The weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of weeds controlled beyond the spectrum controlled by the compound of Formula 1 alone.

In certain instances, combinations of a compound of this invention with other biologically active (particularly herbicidal) compounds or agents (i.e. active ingredients) can result in a greater-than-additive (i.e. synergistic) effect on weeds and/or a less-than-additive effect (i.e. safening) on crops or other desirable plants. Reducing the quantity of active ingredients released in the environment while ensuring effective pest control is always desirable. Ability to use greater amounts of active ingredients to provide more effective weed control without excessive crop injury is also desirable. When synergism of herbicidal active ingredients occurs on weeds at application rates giving agronomically satisfactory levels of weed control, such combinations can be advantageous for reducing crop production cost and decreasing environmental load. When safening of herbicidal active ingredients occurs on crops, such combinations can be advantageous for increasing crop protection by reducing weed competition.

Of note is a combination of a compound of the invention with at least one other herbicidal active ingredient. Of particular note is such a combination where the other herbicidal active ingredient has different site of action from the compound of the invention. In certain instances, a combination with at least one other herbicidal active ingredient having a similar spectrum of control but a different site of action will be particularly advantageous for resistance management. Thus, a composition of the present invention can further comprise (in a herbicidally effective amount) at least one additional herbicidal active ingredient having a similar spectrum of control but a different site of action.

Compounds of this invention can also be used in combination with herbicide safeners such as allidochlor, benoxacor, cloquintocet-mexyl, cumvluron, cyometrinil, cyprosulfonamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone naphthalic anhydride (1,8-naphthalic anhydride), oxabetrinil, N-(aminocarbonyl)-2-methylbenzenesulfonamide, N-(aminocarbonyl)-2-fluorobenzenesulfonamide, 1-bromo-4-[(chloromethyl)sulfonyl]benzene (BCS), 4-(dichloroacetyl)-1-oxa-4-azospiro[4.5]decane (MON 4660), 2-(dichloromethyl)-2-methyl-1,3-dioxolane (MG 191), ethyl 1,6-dihydro-l-(2-methoxyphenyl)-6-oxo-2-phenyl-5-pyrimidinecarboxylate, 2-hydroxy-N,N-dimethyl-6-(trifluoromethyl)pyridine-3-carboxamide, and 3-oxo-1-cyclohexen-1-yl 1-(3,4-dimethylphenyl)-1,6-dihydro-6-oxo-2-phenyl-5-pyrimidinecarboxylate, 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide to increase safety to certain crops. Antidotally effective amounts of the herbicide safeners can be applied at the same time as the compounds of this invention, or applied as seed treatments. Therefore an aspect of the present invention relates to a herbicidal mixture comprising a compound of this invention and an antidotally effective amount of a herbicide safener. Seed treatment is particularly useful for selective weed control, because it physically restricts antidoting to the crop plants. Therefore a particularly useful embodiment of the present invention is a method for selectively controlling the growth of undesired vegetation in a crop comprising contacting the locus of the crop with a herbicidally effective amount of a compound of this invention wherein seed from which the crop is grown is treated with an antidotally effective amount of safener. Antidotally effective amounts of safeners can be easily determined by one skilled in the art through simple experimentation.

Compounds of the invention cans also be mixed with: (1) polynucleotides including but not limited to DNA, RNA, and/or chemically modified nucleotides influencing the amount of a particular target through down regulation, interference, suppression or silencing of the genetically derived transcript that render a herbicidal effect; or (2) polynucleotides including but not limited to DNA, RNA, and/or chemically modified nucleotides influencing the amount of a particular target through down regulation, interference, suppression or silencing of the genetically derived transcript that render a safening effect.

Of note is a composition comprising a compound of the invention (in a herbicidally effective amount), at least one additional active ingredient selected from the group consisting of other herbicides and herbicide safeners (in an effective amount), and at least one component selected from the group consisting of surfactants, solid diluents and liquid diluents.

Table A1 lists specific combinations of a Component (a) with Component (b) illustrative of the mixtures, compositions and methods of the present invention. Compound 1 in the Component (a) column is identified in Index Table A. The second column of Table A1 lists the specific Component (b) compound (e.g., “2,4-D” in the first line). The third, fourth and fifth columns of Table A1 lists ranges of weight ratios for rates at which the Component (a) compound is typically applied to a field-grown crop relative to Component (b) (i.e. (a):(b)). Thus, for example, the first line of Table A1 specifically discloses the combination of Component (a) (i.e. Compound 1 in Index Table A) with 2,4-D is typically applied in a weight ratio between 1:192-6:1. The remaining lines of Table A1 are to be construed similarly.

TABLE A1

| Component (a) | | Typical | More Typical | Most Typical |
|---|---|---|---|---|
| (Compound #) | Component (b) | Weight Ratio | Weight Ratio | Weight Ratio |
| 1 | 2,4-D | 1:192-6:1 | 1:64-2:1 | 1:24-1:3 |
| 1 | Acetochlor | 1:768-2:1 | 1:256-1:2 | 1:96-1:11 |
| 1 | Acifluorfen | 1:96-12:1 | 1:32-4:1 | 1:12-1:2 |
| 1 | Aclonifen | 1:857-2:1 | 1:285-1:3 | 1:107-1:12 |
| 1 | Alachlor | 1:768-2:1 | 1:256-1:2 | 1:96-1:11 |
| 1 | Ametryn | 1:384-3:1 | 1:128-1:1 | 1:48-1:6 |
| 1 | Amicarbazone | 1:192-6:1 | 1:64-2:1 | 1:24-1:3 |
| 1 | Amidosulfuron | 1:6-168:1 | 1:2-56:1 | 1:1-11:1 |
| 1 | Aminocyclopyrachlor | 1:48-24:1 | 1:16-8:1 | 1:6-2:1 |
| 1 | Aminopyralid | 1:20-56:1 | 1:6-19:1 | 1:2-4:1 |
| 1 | Amitrole | 1:768-2:1 | 1:256-1:2 | 1:96-1:11 |
| 1 | Anilofos | 1:96-12:1 | 1:32-4:1 | 1:12-1:2 |
| 1 | Asulam | 1:960-2:1 | 1:320-1:3 | 1:120-1:14 |
| 1 | Atrazine | 1:192-6:1 | 1:64-2:1 | 1:24-1:3 |
| 1 | Azimsulfuron | 1:6-168:1 | 1:2-56:1 | 1:1-11:1 |
| 1 | Beflubutamid | 1:342-4:1 | 1:114-2:1 | 1:42-1:5 |
| 1 | S-Beflubutamid | 1:175-2:1 | 1:65-1:1 | 1:18-1:3 |
| 1 | Benfuresate | 1:617-2:1 | 1:205-1:2 | 1:77-1:9 |
| 1 | Bensulfuron-methyl | 1:25-45:1 | 1:8-15:1 | 1:3-3:1 |
| 1 | Bentazone | 1:192-6:1 | 1:64-2:1 | 1:24-1:3 |
| 1 | Benzobicyclon | 1:85-14:1 | 1:28-5:1 | 1:10-1:2 |
| 1 | Benzofenap | 1:257-5:1 | 1:85-2:1 | 1:32-1:4 |
| 1 | Bicyclopyrone | 1:42-27:1 | 1:14-9:1 | 1:5-2:1 |
| 1 | Bifenox | 1:257-5:1 | 1:85-2:1 | 1:32-1:4 |
| 1 | Bispyribac-sodium | 1:10-112:1 | 1:3-38:1 | 1:1-7:1 |
| 1 | Bromacil | 1:384-3:1 | 1:128-1:1 | 1:48-1:6 |
| 1 | Bromobutide | 1:384-3:1 | 1:128-1:1 | 1:48-1:6 |
| 1 | Bromoxynil | 1:96-12:1 | 1:32-4:1 | 1:12-1:2 |
| 1 | Butachlor | 1:768-2:1 | 1:256-1:2 | 1:96-1:11 |
| 1 | Butafenacil | 1:42-27:1 | 1:14-9:1 | 1:5-2:1 |
| 1 | Butylate | 1:1542-1:2 | 1:514-1:5 | 1:192-1:22 |
| 1 | Carfenstrole | 1:192-6:1 | 1:64-2:1 | 1:24-1:3 |
| 1 | Carfentrazone-ethyl | 1:128-9:1 | 1:42-3:1 | 1:16-1:2 |
| 1 | Chlorimuron-ethyl | 1:8-135:1 | 1:2-45:1 | 1:1-9:1 |
| 1 | Chlorotoluron | 1:768-2:1 | 1:256-1:2 | 1:96-1:11 |
| 1 | Chlorsulfuron | 1:6-168:1 | 1:2-56:1 | 1:1-11:1 |
| 1 | Cincosulfuron | 1:17-68:1 | 1:5-23:1 | 1:2-5:1 |
| 1 | Cinidon-ethyl | 1:384-3:1 | 1:128-1:1 | 1:48-1:6 |
| 1 | Cinmethylin | 1:34-34:1 | 1:11-12:1 | 1:4-3:1 |
| 1 | Clacyfos | 1:34-34:1 | 1:11-12:1 | 1:4-3:1 |
| 1 | Clethodim | 1:48-24:1 | 1:16-8:1 | 1:6-2:1 |
| 1 | Clodinafop-propargyl | 1:20-56:1 | 1:6-19:1 | 1:2-4:1 |
| 1 | Clomazone | 1:384-3:1 | 1:128-1:1 | 1:48-1:6 |
| 1 | Clomeprop | 1:171-7:1 | 1:57-3:1 | 1:21-1:3 |
| 1 | Clopyralid | 1:192-6:1 | 1:64-2:1 | 1:24-1:3 |
| 1 | Cloransulam-methyl | 1:12-96:1 | 1:4-32:1 | 1:1-6:1 |
| 1 | Cumyluron | 1:384-3:1 | 1:128-1:1 | 1:48-1:6 |
| 1 | Cyanazine | 1:384-3:1 | 1:128-1:1 | 1:48-1:6 |
| 1 | Cyclopyrimorate | 1:17-68:1 | 1:5-23:1 | 1:2-5:1 |
| 1 | Cyclosulfamuron | 1:17-68:1 | 1:5-23:1 | 1:2-5:1 |
| 1 | Cycloxydim | 1:96-12:1 | 1:32-4:1 | 1:12-1:2 |
| 1 | Cyhalofop | 1:25-45:1 | 1:8-15:1 | 1:3-3:1 |
| 1 | Daimuron | 1:192-6:1 | 1:64-2:1 | 1:24-1:3 |
| 1 | Desmedipham | 1:322-4:1 | 1:107-2:1 | 1:40-1:5 |
| 1 | Dicamba | 1:192-6:1 | 1:64-2:1 | 1:24-1:3 |
| 1 | Dichlobenil | 1:1371-1:2 | 1:457-1:4 | 1:171-1:20 |
| 1 | Dichlorprop | 1:925-2:1 | 1:308-1:3 | 1:115-1:13 |
| 1 | Diclofop-methyl | 1:384-3:1 | 1:128-1:1 | 1:48-1:6 |
| 1 | Diclosulam | 1:10-112:1 | 1:3-38:1 | 1:1-7:1 |
| 1 | Difenzoquat | 1:288-4:1 | 1:96-2:1 | 1:36-1:4 |
| 1 | Diflufenican | 1:857-2:1 | 1:285-1:3 | 1:107-1:12 |
| 1 | Diflufenzopyr | 1:12-96:1 | 1:4-32:1 | 1:1-6:1 |
| 1 | Dimethachlor | 1:768-2:1 | 1:256-1:2 | 1:96-1:11 |
| 1 | Dimethametryn | 1:192-6:1 | 1:64-2:1 | 1:24-1:3 |
| 1 | Dimethenamid-P | 1:384-3:1 | 1:128-1:1 | 1:48-1:6 |
| 1 | Dithiopyr | 1:192-6:1 | 1:64-2:1 | 1:24-1:3 |
| 1 | Diuron | 1:384-3:1 | 1:128-1:1 | 1:48-1:6 |
| 1 | EPTC | 1:768-2:1 | 1:256-1:2 | 1:96-1:11 |
| 1 | Esprocarb | 1:1371-1:2 | 1:457-1:4 | 1:171-1:20 |
| 1 | Ethalfluralin | 1:384-3:1 | 1:128-1:1 | 1:48-1:6 |
| 1 | Ethametsulfuron-methyl | 1:17-68:1 | 1:5-23:1 | 1:2-5:1 |
| 1 | Ethoxyfen | 1:8-135:1 | 1:2-45:1 | 1:1-9:1 |
| 1 | Ethoxysulfuron | 1:20-56:1 | 1:6-19:1 | 1:2-4:1 |
| 1 | Etobenzanid | 1:257-5:1 | 1:85-2:1 | 1:32-1:4 |
| 1 | Fenoxaprop-ethyl | 1:120-10:1 | 1:40-4:1 | 1:15-1:2 |
| 1 | Fenoxasulfone | 1:85-14:1 | 1:28-5:1 | 1:10-1:2 |
| 1 | Fenquinotrione | 1:17-68:1 | 1:5-23:1 | 1:2-5:1 |
| 1 | Fentrazamide | 1:17-68:1 | 1:5-23:1 | 1:2-5:1 |
| 1 | Flazasulfuron | 1:17-68:1 | 1:5-23:1 | 1:2-5:1 |
| 1 | Florasulam | 1:2-420:1 | 1:1-140:1 | 2:1-27:1 |
| 1 | Fluazifop-butyl | 1:192-6:1 | 1:64-2:1 | 1:24-1:3 |
| 1 | Flucarbazone | 1:8-135:1 | 1:2-45:1 | 1:1-9:1 |
| 1 | Flucetosulfuron | 1:8-135:1 | 1:2-45:1 | 1:1-9:1 |
| 1 | Flufenacet | 1:257-5:1 | 1:85-2:1 | 1:32-1:4 |
| 1 | Flumetsulam | 1:24-48:1 | 1:8-16:1 | 1:3-3:1 |
| 1 | Flumiclorac-pentyl | 1:10-112:1 | 1:3-38:1 | 1:1-7:1 |
| 1 | Flumioxazin | 1:25-45:1 | 1:8-15:1 | 1:3-3:1 |
| 1 | Fluometuron | 1:384-3:1 | 1:128-1:1 | 1:48-1:6 |
| 1 | Flupyrsulfuron-methyl | 1:3-336:1 | 1:1-112:1 | 2:1-21:1 |
| 1 | Fluridone | 1:384-3:1 | 1:128-1:1 | 1:48-1:6 |
| 1 | Fluroxypyr | 1:96-12:1 | 1:32-4:1 | 1:12-1:2 |
| 1 | Flurtamone | 1:857-2:1 | 1:285-1:3 | 1:107-1:12 |
| 1 | Fluthiacet-methyl | 1:48-42:1 | 1:16-14:1 | 1:3-3:1 |
| 1 | Fomesafen | 1:96-12:1 | 1:32-4:1 | 1:12-1:2 |
| 1 | Foramsulfuron | 1:13-84:1 | 1:4-28:1 | 1:1-6:1 |
| 1 | Glufosinate | 1:288-4:1 | 1:96-2:1 | 1:36-1:4 |
| 1 | Glyphosate | 1:288-4:1 | 1:96-2:1 | 1:36-1:4 |
| 1 | Halosulfuron-methyl | 1:17-68:1 | 1:5-23:1 | 1:2-5:1 |
| 1 | Halauxifen | 1:20-56:1 | 1:6-19:1 | 1:2-4:1 |
| 1 | Halauxifen methyl | 1:20-56:1 | 1:6-19:1 | 1:2-4:1 |
| 1 | Haloxyfop-methyl | 1:34-34:1 | 1:11-12:1 | 1:4-3:1 |
| 1 | Hexazinone | 1:192-6:1 | 1:64-2:1 | 1:24-1:3 |
| 1 | Hydantocidin | 1:1100-16:1 | 1:385-8:1 | 1:144-4:1 |
| 1 | Imazamox | 1:13-84:1 | 1:4-28:1 | 1:1-6:1 |
| 1 | Imazapic | 1:20-56:1 | 1:6-19:1 | 1:2-4:1 |
| 1 | Imazapyr | 1:85-14:1 | 1:28-5:1 | 1:10-1:2 |
| 1 | Imazaquin | 1:34-34:1 | 1:11-12:1 | 1:4-3:1 |
| 1 | Imazethabenz-methyl | 1:171-7:1 | 1:57-3:1 | 1:21-1:3 |
| 1 | Imazethapyr | 1:24-48:1 | 1:8-16:1 | 1:3-3:1 |
| 1 | Imazosulfuron | 1:27-42:1 | 1:9-14:1 | 1:3-3:1 |
| 1 | Indanofan | 1:342-4:1 | 1:114-2:1 | 1:42-1:5 |
| 1 | Indaziflam | 1:25-45:1 | 1:8-15:1 | 1:3-3:1 |
| 1 | Iodosulfuron-methyl | 1:3-336:1 | 1:1-112:1 | 2:1-21:1 |
| 1 | Ioxynil | 1:192-6:1 | 1:64-2:1 | 1:24-1:3 |
| 1 | Ipfencarbazone | 1:85-14:1 | 1:28-5:1 | 1:10-1:2 |
| 1 | Isoproturon | 1:384-3:1 | 1:128-1:1 | 1:48-1:6 |
| 1 | Isoxaben | 1:288-4:1 | 1:96-2:1 | 1:36-1:4 |
| 1 | Isoxaflutole | 1:60-20:1 | 1:20-7:1 | 1:7-2:1 |
| 1 | Lactofen | 1:42-27:1 | 1:14-9:1 | 1:5-2:1 |
| 1 | Lenacil | 1:384-3:1 | 1:128-1:1 | 1:48-1:6 |
| 1 | Linuron | 1:384-3:1 | 1:128-1:1 | 1:48-1:6 |
| 1 | MCPA | 1:192-6:1 | 1:64-2:1 | 1:24-1:3 |
| 1 | MCPB | 1:288-4:1 | 1:96-2:1 | 1:36-1:4 |
| 1 | Mecoprop | 1:768-2:1 | 1:256-1:2 | 1:96-1:11 |
| 1 | Mefenacet | 1:384-3:1 | 1:128-1:1 | 1:48-1:6 |
| 1 | Mefluidide | 1:192-6:1 | 1:64-2:1 | 1:24-1:3 |
| 1 | Mesosulfuron-methyl | 1:5-224:1 | 1:1-75:1 | 1:1-14:1 |
| 1 | Mesotrione | 1:42-27:1 | 1:14-9:1 | 1:5-2:1 |
| 1 | Metamifop | 1:42-27:1 | 1:14-9:1 | 1:5-2:1 |
| 1 | Metazachlor | 1:384-3:1 | 1:128-1:1 | 1:48-1:6 |
| 1 | Metazosulfuron | 1:25-45:1 | 1:8-15:1 | 1:3-3:1 |
| 1 | Methabenzthiazuron | 1:768-2:1 | 1:256-1:2 | 1:96-1:11 |
| 1 | Metolachlor | 1:768-2:1 | 1:256-1:2 | 1:96-1:11 |
| 1 | Metosulam | 1:8-135:1 | 1:2-45:1 | 1:1-9:1 |
| 1 | Metribuzin | 1:192-6:1 | 1:64-2:1 | 1:24-1:3 |
| 1 | Metsulfuron-methyl | 1:2-560:1 | 1:1-187:1 | 3:1-35:1 |
| 1 | Molinate | 1:1028-2:1 | 1:342-1:3 | 1:128-1:15 |
| 1 | Napropamide | 1:384-3:1 | 1:128-1:1 | 1:48-1:6 |
| 1 | Napropamide-M | 1:192-6:1 | 1:64-2:1 | 1:24-1:3 |
| 1 | Naptalam | 1:192-6:1 | 1:64-2:1 | 1:24-1:3 |
| 1 | Nicosulfuron | 1:12-96:1 | 1:4-32:1 | 1:1-6:1 |
| 1 | Norflurazon | 1:1152-1:1 | 1:384-1:3 | 1:144-1:16 |
| 1 | Orbencarb | 1:1371-1:2 | 1:457-1:4 | 1:171-1:20 |
| 1 | Orthosulfamuron | 1:20-56:1 | 1:6-19:1 | 1:2-4:1 |
| 1 | Oryzalin | 1:514-3:1 | 1:171-1:2 | 1:64-1:8 |
| 1 | Oxadiargyl | 1:384-3:1 | 1:128-1:1 | 1:48-1:6 |
| 1 | Oxadiazon | 1:548-3:1 | 1:182-1:2 | 1:68-1:8 |
| 1 | Oxasulfuron | 1:27-42:1 | 1:9-14:1 | 1:3-3:1 |
| 1 | Oxaziclomefone | 1:42-27:1 | 1:14-9:1 | 1:5-2:1 |
| 1 | Oxyfluorfen | 1:384-3:1 | 1:128-1:1 | 1:48-1:6 |
| 1 | Paraquat | 1:192-6:1 | 1:64-2:1 | 1:24-1:3 |
| 1 | Pendimethalin | 1:384-3:1 | 1:128-1:1 | 1:48-1:6 |
| 1 | Penoxsulam | 1:10-112:1 | 1:3-38:1 | 1:1-7:1 |
| 1 | Penthoxamid | 1:384-3:1 | 1:128-1:1 | 1:48-1:6 |
| 1 | Pentoxazone | 1:102-12:1 | 1:34-4:1 | 1:12-1:2 |
| 1 | Phenmedipham | 1:102-12:1 | 1:34-4:1 | 1:12-1:2 |
| 1 | Picloram | 1:96-12:1 | 1:32-4:1 | 1:12-1:2 |
| 1 | Picolinafen | 1:34-34:1 | 1:11-12:1 | 1:4-3:1 |
| 1 | Pinoxaden | 1:25-45:1 | 1:8-15:1 | 1:3-3:1 |
| 1 | Pretilachlor | 1:192-6:1 | 1:64-2:1 | 1:24-1:3 |
| 1 | Primisulfuron-methyl | 1:8-135:1 | 1:2-45:1 | 1:1-9:1 |
| 1 | Prodiamine | 1:384-3:1 | 1:128-1:1 | 1:48-1:6 |
| 1 | Profoxydim | 1:42-27:1 | 1:14-9:1 | 1:5-2:1 |
| 1 | Prometryn | 1:384-3:1 | 1:128-1:1 | 1:48-1:6 |
| 1 | Propachlor | 1:1152-1:1 | 1:384-1:3 | 1:144-1:16 |
| 1 | Propanil | 1:384-3:1 | 1:128-1:1 | 1:48-1:6 |
| 1 | Propaquizafop | 1:48-24:1 | 1:16-8:1 | 1:6-2:1 |
| 1 | Propoxycarbazone | 1:17-68:1 | 1:5-23:1 | 1:2-5:1 |
| 1 | Propyrisulfuron | 1:17-68:1 | 1:5-23:1 | 1:2-5:1 |
| 1 | Propyzamide | 1:384-3:1 | 1:128-1:1 | 1:48-1:6 |
| 1 | Prosulfocarb | 1:1200-1:2 | 1:400-1:4 | 1:150-1:17 |
| 1 | Prosulfuron | 1:6-168:1 | 1:2-56:1 | 1:1-11:1 |
| 1 | Pyraclonil | 1:42-27:1 | 1:14-9:1 | 1:5-2:1 |
| 1 | Pyraflufen-ethyl | 1:5-224:1 | 1:1-75:1 | 1:1-14:1 |
| 1 | Pyrasulfotole | 1:13-84:1 | 1:4-28:1 | 1:1-6:1 |
| 1 | Pyrazolynate | 1:857-2:1 | 1:285-1:3 | 1:107-1:12 |
| 1 | Pyrazosulfuron-ethyl | 1:10-112:1 | 1:3-38:1 | 1:1-7:1 |
| 1 | Pyrazoxyfen | 1:5-224:1 | 1:1-75:1 | 1:1-14:1 |
| 1 | Pyribenzoxim | 1:10-112:1 | 1:3-38:1 | 1:1-7:1 |
| 1 | Pyributicarb | 1:384-3:1 | 1:128-1:1 | 1:48-1:6 |
| 1 | Pyridate | 1:288-4:1 | 1:96-2:1 | 1:36-1:4 |
| 1 | Pyriftalid | 1:10-112:1 | 1:3-38:1 | 1:1-7:1 |
| 1 | Pyriminobac-methyl | 1:20-56:1 | 1:6-19:1 | 1:2-4:1 |
| 1 | Pyrimisulfan | 1:17-68:1 | 1:5-23:1 | 1:2-5:1 |
| 1 | Pyrithiobac | 1:24-48:1 | 1:8-16:1 | 1:3-3:1 |
| 1 | Pyroxasulfone | 1:85-14:1 | 1:28-5:1 | 1:10-1:2 |
| 1 | Pyroxsulam | 1:5-224:1 | 1:1-75:1 | 1:1-14:1 |
| 1 | Quinclorac | 1:192-6:1 | 1:64-2:1 | 1:24-1:3 |
| 1 | Quizalofop-ethyl | 1:42-27:1 | 1:14-9:1 | 1:5-2:1 |
| 1 | Rimsulfuron | 1:13-84:1 | 1:4-28:1 | 1:1-6:1 |
| 1 | Saflufenacil | 1:25-45:1 | 1:8-15:1 | 1:3-3:1 |
| 1 | Sethoxydim | 1:96-12:1 | 1:32-4:1 | 1:12-1:2 |
| 1 | Simazine | 1:384-3:1 | 1:128-1:1 | 1:48-1:6 |
| 1 | Sulcotrione | 1:120-10:1 | 1:40-4:1 | 1:15-1:2 |
| 1 | Sulfentrazone | 1:147-8:1 | 1:49-3:1 | 1:18-1:3 |
| 1 | Sulfometuron-methyl | 1:34-34:1 | 1:11-12:1 | 1:4-3:1 |
| 1 | Sulfosulfuron | 1:8-135:1 | 1:2-45:1 | 1:1-9:1 |
| 1 | Tebuthiuron | 1:384-3:1 | 1:128-1:1 | 1:48-1:6 |
| 1 | Tefuryltrione | 1:42-27:1 | 1:14-9:1 | 1:5-2:1 |
| 1 | Tembotrione | 1:31-37:1 | 1:10-13:1 | 1:3-3:1 |
| 1 | Tepraloxydim | 1:25-45:1 | 1:8-15:1 | 1:3-3:1 |
| 1 | Terbacil | 1:288-4:1 | 1:96-2:1 | 1:36-1:4 |
| 1 | Terbuthylazine | 1:857-2:1 | 1:285-1:3 | 1:107-1:12 |
| 1 | Terbutyrn | 1:192-6:1 | 1:64-2:1 | 1:24-1:3 |
| 1 | Thenylchlor | 1:85-14:1 | 1:28-5:1 | 1:10-1:2 |
| 1 | Thiazopyr | 1:384-3:1 | 1:128-1:1 | 1:48-1:6 |
| 1 | Thiencarbazone | 1:3-336:1 | 1:1-112:1 | 2:1-21:1 |
| 1 | Thifensulfuron-methyl | 1:5-224:1 | 1:1-75:1 | 1:1-14:1 |
| 1 | Tiafenacil | 1:17-68:1 | 1:5-23:1 | 1:2-5:1 |
| 1 | Thiobencarb | 1:768-2:1 | 1:256-1:2 | 1:96-1:11 |
| 1 | Tolpyralate | 1:31-37:1 | 1:10-13:1 | 1:3-3:1 |
| 1 | Topramzone | 1:6-168:1 | 1:2-56:1 | 1:1-11:1 |
| 1 | Tralkoxydim | 1:68-17:1 | 1:22-6:1 | 1:8-2:1 |
| 1 | Triafamone | 1:2-420:1 | 1:1-140:1 | 2:1-27:1 |
| 1 | Triallate | 1:768-2:1 | 1:256-1:2 | 1:96-1:11 |
| 1 | Triasulfuron | 1:5-224:1 | 1:1-75:1 | 1:1-14:1 |
| 1 | Triaziflam | 1:171-7:1 | 1:57-3:1 | 1:21-1:3 |
| 1 | Tribenuron-methyl | 1:3-336:1 | 1:1-112:1 | 2:1-21:1 |
| 1 | Triclopyr | 1:192-6:1 | 1:64-2:1 | 1:24-1:3 |
| 1 | Trifloxysulfuron | 1:2-420:1 | 1:1-140:1 | 2:1-27:1 |
| 1 | Trifludimoxazin | 1:25-45:1 | 1:8-15:1 | 1:3-3:1 |
| 1 | Trifluralin | 1:288-4:1 | 1:96-2:1 | 1:36-1:4 |
| 1 | Triflusulfuron-methyl | 1:17-68:1 | 1:5-23:1 | 1:2-5:1 |
| 1 | Tritosulfuron | 1:13-84:1 | 1:4-28:1 | 1:1-6:1 |
| 1 | (4-(4-fluorophenyl)-6-[(2- | 1:42-27:1 | 1:14-9:1 | 1:5-2:1 |
| | hydroxy-6-oxo-1-cyclohexen-1- | | | |
| | yl)carbonyl]-2-methyl-1,2,4- | | | |
| | triazine-3,5(2H,4H)-dione, | | | |

Table A2 is constructed the same as Table A1 above except that entries below the “Component (a)” column heading are replaced with the respective Component (a) Column Entry shown below. Compound 1 in the Component (a) column is identified in Index Table A. Thus, for example, in Table A2 the entries below the “Component (a)” column heading all recite “Compound 1” (i.e. Compound 1 identified in Index Table A), and the first line below the column headings in Table A2 specifically discloses a mixture of Compound 1 with 2,4-D. Tables A3 through A148 are constructed similarly.

| Table | Component (a) |
|---|---|
| Number | Column Entries |
| A2 | Compound 2 |
| A3 | Compound 3 |
| A4 | Compound 4 |
| A5 | Compound 5 |
| A6 | Compound 6 |
| A7 | Compound 7 |
| A8 | Compound 8 |
| A9 | Compound 9 |
| A10 | Compound 10 |
| A11 | Compound 11 |
| A12 | Compound 12 |
| A13 | Compound 13 |
| A14 | Compound 14 |
| A15 | Compound 15 |
| A16 | Compound 16 |
| A17 | Compound 17 |
| A18 | Compound 18 |
| A19 | Compound 19 |
| A20 | Compound 20 |
| A21 | Compound 21 |
| A22 | Compound 22 |
| A23 | Compound 23 |
| A24 | Compound 24 |
| A25 | Compound 25 |
| A26 | Compound 26 |
| A27 | Compound 27 |
| A28 | Compound 28 |
| A29 | Compound 29 |
| A30 | Compound 30 |
| A31 | Compound 31 |
| A32 | Compound 32 |
| A33 | Compound 33 |
| A34 | Compound 34 |
| A35 | Compound 35 |
| A36 | Compound 36 |
| A37 | Compound 37 |
| A38 | Compound 38 |
| A39 | Compound 39 |
| A40 | Compound 40 |
| A41 | Compound 41 |
| A42 | Compound 42 |
| A43 | Compound 43 |
| A44 | Compound 44 |
| A45 | Compound 45 |
| A46 | Compound 46 |
| A47 | Compound 47 |
| A48 | Compound 48 |
| A49 | Compound 49 |
| A50 | Compound 50 |
| A51 | Compound 51 |
| A52 | Compound 52 |
| A53 | Compound 53 |
| A54 | Compound 54 |
| A55 | Compound 55 |
| A56 | Compound 56 |
| A57 | Compound 57 |
| A58 | Compound 58 |
| A59 | Compound 59 |
| A60 | Compound 60 |
| A61 | Compound 61 |
| A62 | Compound 62 |
| A63 | Compound 63 |
| A64 | Compound 64 |
| A65 | Compound 65 |
| A66 | Compound 66 |
| A67 | Compound 67 |
| A68 | Compound 68 |
| A69 | Compound 69 |
| A70 | Compound 70 |
| A71 | Compound 71 |
| A72 | Compound 72 |
| A73 | Compound 73 |
| A74 | Compound 74 |
| A75 | Compound 75 |
| A76 | Compound 76 |
| A77 | Compound 77 |
| A78 | Compound 78 |
| A79 | Compound 79 |
| A80 | Compound 80 |
| A81 | Compound 81 |
| A82 | Compound 82 |
| A83 | Compound 83 |
| A84 | Compound 84 |
| A85 | Compound 85 |
| A86 | Compound 86 |
| A87 | Compound 87 |
| A88 | Compound 88 |
| A89 | Compound 89 |
| A90 | Compound 90 |
| A91 | Compound 91 |
| A92 | Compound 92 |
| A93 | Compound 93 |
| A94 | Compound 94 |
| A95 | Compound 95 |
| A96 | Compound 96 |
| A97 | Compound 97 |
| A98 | Compound 98 |
| A99 | Compound 99 |
| A100 | Compound 100 |
| A101 | Compound 101 |
| A102 | Compound 102 |
| A103 | Compound 103 |
| A104 | Compound 104 |
| A105 | Compound 105 |
| A106 | Compound 106 |
| A107 | Compound 107 |
| A108 | Compound 108 |
| A109 | Compound 109 |
| A110 | Compound 110 |
| A111 | Compound 111 |
| A112 | Compound 112 |
| A113 | Compound 113 |
| A114 | Compound 114 |
| A115 | Compound 115 |
| A116 | Compound 116 |
| A117 | Compound 117 |
| A118 | Compound 118 |
| A119 | Compound 119 |
| A120 | Compound 120 |
| A121 | Compound 121 |
| A122 | Compound 122 |
| A123 | Compound 123 |
| A124 | Compound 124 |
| A125 | Compound 125 |
| A126 | Compound 126 |
| A127 | Compound 127 |
| A128 | Compound 128 |
| A129 | Compound 129 |
| A130 | Compound 130 |
| A131 | Compound 131 |
| A132 | Compound 132 |
| A133 | Compound 133 |
| A134 | Compound 134 |
| A135 | Compound 135 |
| A136 | Compound 136 |
| A137 | Compound 137 |
| A138 | Compound 138 |
| A139 | Compound 139 |
| A140 | Compound 140 |
| A141 | Compound 141 |
| A142 | Compound 142 |
| A143 | Compound 143 |
| A144 | Compound 144 |
| A145 | Compound 145 |
| A146 | Compound 146 |
| A147 | Compound 147 |
| A148 | Compound 148 |
| A149 | Compound 149 |
| A150 | Compound 150 |
| A151 | Compound 151 |
| A152 | Compound 152 |
| A153 | Compound 153 |
| A154 | Compound 154 |
| A155 | Compound 155 |
| A156 | Compound 156 |
| A157 | Compound 157 |
| A158 | Compound 158 |
| A159 | Compound 159 |
| A160 | Compound 160 |
| A161 | Compound 161 |
| A162 | Compoutid 162 |
| A163 | Compound 163 |
| A164 | Compound 164 |
| A165 | Compound 165 |
| A166 | Compound 166 |
| A167 | Compound 167 |
| A168 | Compound 168 |
| A169 | Compound 169 |
| A170 | Compound 170 |
| A171 | Compound 171 |
| A172 | Compound 172 |
| A173 | Compound 173 |
| A174 | Compound 174 |
| A175 | Compound 175 |
| A176 | Compoutid 176 |
| A177 | Compoutid 177 |
| A178 | Compound 178 |
| A179 | Compound 179 |
| A180 | Compound 180 |
| A181 | Compound 181 |
| A182 | Compound 182 |
| A183 | Compound 183 |
| A184 | Compound 184 |
| A185 | Compound 185 |
| A186 | Compound 186 |
| A187 | Compound 187 |
| A188 | Compound 188 |
| A189 | Compound 189 |
| A190 | Compound 190 |
| A191 | Compound 191 |
| A192 | Compound 192 |
| A193 | Compound 193 |
| A194 | Compound 194 |
| A195 | Compound 195 |
| A196 | Compound 196 |
| A197 | Compound 197 |
| A198 | Compound 198 |
| A199 | Compound 199 |
| A200 | Compound 200 |
| A201 | Compound 201 |
| A202 | Compound 202 |
| A203 | Compound 203 |
| A204 | Compound 204 |
| A205 | Compound 205 |
| A206 | Compound 206 |
| A207 | Compound 207 |
| A208 | Compound 208 |
| A209 | Compound 209 |
| A210 | Compound 210 |
| A211 | Compound 211 |
| A212 | Compoutid 212 |
| A213 | Compound 213 |
| A214 | Compound 214 |
| A215 | Compound 215 |
| A216 | Compound 216 |
| A217 | Compound 217 |
| A218 | Compound 218 |
| A219 | Compound 219 |
| A220 | Compound 220 |
| A221 | Compound 221 |
| A222 | Compound 222 |
| A223 | Compound 223 |
| A224 | Compound 224 |
| A225 | Compound 225 |
| A226 | Compound 226 |
| A227 | Compound 227 |
| A228 | Compound 228 |
| A229 | Compound 229 |
| A230 | Compound 230 |
| A231 | Compound 231 |
| A232 | Compound 232 |
| A233 | Compound 233 |
| A234 | Compound 234 |
| A235 | Compound 235 |
| A236 | Compound 236 |
| A237 | Compound 237 |
| A238 | Compound 238 |
| A239 | Compound 239 |
| A240 | Compound 240 |
| A241 | Compound 241 |
| A242 | Compound 242 |
| A243 | Compound 243 |
| A244 | Compound 244 |
| A245 | Compound 245 |
| A246 | Compound 246 |
| A247 | Compound 247 |
| A248 | Compound 248 |
| A249 | Compound 249 |
| A250 | Compound 250 |
| A251 | Compound 251 |
| A252 | Compound 252 |
| A253 | Compound 253 |
| A254 | Compound 254 |
| A255 | Compound 255 |
| A256 | Compound 256 |
| A257 | Compound 257 |
| A258 | Compound 258 |
| A259 | Compound 259 |
| A260 | Compound 260 |
| A261 | Compound 261 |
| A262 | Compound 262 |
| A263 | Compound 263 |
| A264 | Compound 264 |
| A265 | Compound 265 |
| A266 | Compound 266 |
| A267 | Compound 267 |
| A268 | Compound 268 |
| A269 | Compound 269 |
| A270 | Compound 270 |
| A271 | Compound 271 |
| A272 | Compound 272 |
| A273 | Compound 273 |
| A274 | Compound 274 |
| A275 | Compound 275 |
| A276 | Compound 276 |
| A277 | Compound 277 |
| A278 | Compound 278 |
| A279 | Compound 279 |
| A280 | Compound 280 |
| A281 | Compound 281 |
| A282 | Compound 282 |
| A283 | Compound 283 |
| A284 | Compound 284 |
| A285 | Compound 285 |
| A286 | Compound 286 |
| A287 | Compound 287 |
| A288 | Compound 288 |
| A289 | Compound 289 |
| A290 | Compound 290 |
| A291 | Compound 291 |
| A292 | Compound 292 |
| A293 | Compound 293 |
| A294 | Compound 294 |
| A295 | Compound 295 |
| A296 | Compound 296 |
| A297 | Compound 297 |
| A298 | Compound 298 |
| A299 | Compound 299 |
| A300 | Compound 300 |
| A301 | Compound 301 |
| A302 | Compound 302 |
| A303 | Compound 303 |
| A304 | Compound 304 |
| A305 | Compound 305 |

Preferred for better control of undesired vegetation (e.g., lower use rate such as from synergism, broader spectrum of weeds controlled, or enhanced crop safety) or for preventing the development of resistant weeds are mixtures of a compound of this invention with a herbicide selected from the group consisting of atrazine, azimsulfuron, S-beflubutamid, benzisothiazolinone, carfentrazone-ethyl, chlorimuron-ethyl, chlorsulfuron-methyl, clomazone, clopyralid potassium, cloransulam-methyl, 2-[(2,4-dichlorophenyl)methyl]-4,4-dimethyl-isoxazolidinone, ethametsulfuron-methyl, flumetsulam, 4-(4-fluorophenyl)-6-[(2-hydroxy-6-oxo-1-cyclohexen-1-yl)carbonyl]-2-methyl-1,24-triazine-3,5-(2H,4H)-dione, flupyrsulfuron-methyl, fluthiacet-methyl, fomesafen, imazethapyr, lenacil, mesotrione, metribuzin, metsulfuron-methyl, pethoxamid, picloram, pyroxasulfone, quinclorac, rimsulfuron, S-metolachlor, sulfentrazone, thifensulfuron-methyl, triflusulfuron-methyl and tribenuron-methyl. The following Tests demonstrate the control efficacy of the compounds of this invention against specific weeds. The weed control afforded by the compounds is not limited, however, to these species. See Index Tables A for compound descriptions. The following abbreviations are used in the Index Table A which follows: i is iso, c is cyclo, i-Pr is isopropyl, c-Pr is cyclopropyl, n-Pr is n-propyl, n-Bu is n-butyl, Me is methyl, Et is ethyl, Ph is phenyl, OMe is methoxy, OEt is ethoxy, “3-CPL” is (E) 3-chloropropenyl (e.g., —CH₂CH═CHC), “2-PNL” is 2-propenyl (i.e. —CH₂CH═CH₂), CN is cyano, —NO₂ is nitro. The abbreviation “Cmpd. No.” stands for “Compound Number”, “Maj.” stands for major, and “Min” stands for minor. The abbreviation “Ex.” stands for “Example” and is followed by a number indicating in which example the compound is prepared. Mass spectra (MS) are reported as the molecular weight of the highest isotopic abundance parent ion (M+1) formed by addition of H+ (molecular weight of 1) to the molecule, or (M−1) formed by the loss of H+ (molecular weight of 1) from the molecule, observed by using liquid chromatography coupled to a mass spectrometer (LCMS) using either atmospheric pressure chemical ionization (AP+) where “amu” stands for unified atomic mass units.

INDEX TABLE A

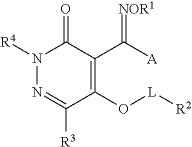

| Cmpd | | | | | | | | M.S. or |
|---|---|---|---|---|---|---|---|---|
| No. | E/Z | R¹ | A | R^{A} | L-R² | R³ | R⁴ | M.P. (°C.) |
| 1 | E | CH₃ | A-1 | 3-CH₃ | H | CH₃ | CH₂CH₃ | 302 |
| 2 | E/Z | CH₃ | A-1 | 3-Cl | H | CH₃ | CH₂CH₃ | 138-142 |
| 3 | E/Z | CH₃ | A-1 | 3-Cl | H | CH₃ | CH₂C=CH | 150-154 |
| 4 | E/Z | CH₃ | A-1 | 3-Cl | H | CH₃ | CH₂-c-Pr | 200-204 |
| 5 | E/Z | CH₃ | A-1 | 3-Cl | H | CH₃ | 2-PNL | 147-151 |
| 6 | E/Z | CH₂CH₃ | A-1 | 3-CH₃ | H | CH₃ | CH₃ | 302 |
| 7 | E | i-Pr | A-1 | 3-CH₃ | H | CH₃ | CH₃ | 316 |
| 8 | E | CH₂CH₃ | A-1 | 3-Cl | H | CH₃ | CH₃ | 322 |
| 9 | Z | CH₂CH₃ | A-1 | 3-Cl | H | CH₃ | CH₃ | 322 |
| 10 | E | CH₃ | A-1 | 3-Cl | H | CH₃ | CH₃ | 308 |
| Ex 3 | | | | | | | | |
| 11 | Z | CH₃ | A-1 | 3-Cl | H | CH₃ | CH₃ | 308 |
| Ex 3 | | | | | | | | |
| 12 | E | CH₃ | A-1 | 2-SO₂CH₃,4-CF₃ | H | CH₃ | CH₃ | 434 |
| 13 | E | CH₃ | A-1 | 3-CH₃ | H | CH₃ | CCH₃H3 | 288 |
| 14 | E | CH₂CH₃ | A-1 | 2-CH₃ | H | CH₃ | CH₃ | 302 |
| 15 | Z | CH₂CH₃ | A-1 | 2-CH₃ | H | CH₃ | CH₃ | 302 |
| 16 | Z | CH₂CH₃ | A-1 | 4-CH₃ | H | CH₃ | CH | 302 |
| 17 | E | CH₂CH₃ | A-1 | 4-CH₃ | H | CH₃ | CH₃ | 302 |
| 18 | Z | i-Pr | A-1 | 3-CH₃ | H | CH₃ | CH₃ | 316 |
| 19 | Z | i-Pr | A-1 | 2-CH₃ | H | CH₃ | CH₃ | 316 |
| 20 | E | i-Pr | A-1 | 2-CH₃ | H | CH₃ | CH₃ | 316 |
| 21 | E | H | A-1 | 3-CH₃ | H | CH₃ | CH₃ | 274 |
| 22 | E | CH₃ | A-1 | 2,3-di-CH₃ | H | CH₃ | CH₃ | 302 |
| 23 | Z | CH₃ | A-1 | 2,3-di-CH₃ | H | CH₃ | CH₃ | 302 |
| 24 | Z | CH₂CH₃ | A-1 | 2,3-di-CH₃ | H | CH₃ | CH₃ | 316 |
| 25 | E | CH₂CH₃ | A-1 | 2,3-di-CH₃ | H | CH₃ | CH₃ | 316 |
| 26 | Z | CH₃ | A-6 | (n = 0) | H | CH₃ | CH₃ | 106-110 |
| 27 | E | CH₃ | A-1 | 3-CH₃ | H | c-Pr | CH₃ | 314 |
| 28 | Z | CH₃ | A-1 | 3-CH₃ | H | c-Pr | CH₃ | 314 |
| 29 | Z | CH₃ | A-1 | 2-Cl | H | CH₃ | CH₃ | 177-181 |
| 30 | Z | CH₃ | A-1 | 2,5-di-CH₃ | H | CH₃ | CH₃ | 80-84 |
| 31 | E | CH₃ | A-6 | (n = 0) | H | CH₃ | CH₃ | 204-208 |
| 32 | Z | CH₃ | A-1 | 3-F | H | CH₃ | CH₃ | 186-190 |
| 33 | E/Z | CH₃ | A-1 | 3-Cl | H | CH₃ | c-Pr | 210-214 |
| 34 | Z | CH₃ | A-1 | 2-F | H | CH₃ | CH₃ | 179-183 |
| 35 | E | H | A-1 | 5-Cl,2-CH₃ | H | CH₃ | CH₃ | 308 |
| 36 | Z | CH₃ | A-1 | 3-CH₂CH₃ | H | CH₃ | CH₃ | 76-80 |
| 37 | Z | CH₃ | A-1 | 2-CH₂CH₃ | H | CH₃ | CH₃ | 125-129 |
| 38 | Z | CH₃ | A-1 | 2-Cl,5-CH₃ | H | CH₃ | CH₃ | 156-160 |
| 39 | Z | CH₃ | A-1 | 2-F,6-CH₃ | H | CH₃ | CH₃ | 163-167 |
| 40 | Z | CH₃ | A-6 | 4-Cl | H | CH₃ | CH₃ | 168-171 |
| 41 | Z | CH₃ | A-1 | 4-F,2-CH₃ | H | CH₃ | CH₃ | 133-137 |
| 42 | Z | CH₃ | A-2 | 3-CH₃ | H | CH₃ | CH₃ | 150.1-162.9 |
| 43 | Z | CH₃ | A-4 | 2-CH₃ | H | CH₃ | CH₃ | 56.3-76.9 |
| 44 | Z | CH₃ | A-4 | 5-CH₃ | H | CH₃ | CH₃ | 294 |
| 45 | Maj. | CH₃ | A-11 | (n = 0) | H | CH₃ | CH₃ | 330 |
| 46 | Min | CH₃ | A-11 | (n = 0) | H | CH₃ | CH₃ | 330 |
| 47 | Maj. | CH₃ | A-8 | (n = 0) | H | CH₃ | CH₃ | 330 |
| 48 | E | CH₃ | A-6 | (n = 0) | C(═O)Me | CH₃ | CH₃ | 365 |
| 49 | E | CH₃ | A-6 | (n = 0) | C(═O)Et | CH₃ | CH₃ | 380 |
| 50 | Z | CH₃ | A-1 | 2-F,3-CH₃ | H | CH₃ | CH₃ | 158-162 |
| 51 | Z | CH₃ | A-6 | 4-F | H | CH₃ | CH₃ | 342 |
| 52 | Z | CH₃ | A-4 | 4-CH₃ | H | CH₃ | CH₃ | 58.8-70.5 |
| 53 | Z | CH₃ | A-3 | 5-CH₃ | H | CH₃ | CH₃ | 276 (M-1) |
| 54 | Z | CH₃ | A-1 | 2-Cl,5-CF₃ | H | CH₃ | CH₃ | 376 |
| 55 | Z | CH₃ | A-6 | 4-OCH₃ | H | CH₃ | CH₃ | 144-148 |
| 56 | Z | CH₃ | A-1 | 3-CF₃ | H | CH₃ | CH₃ | 166-170 |
| 57 | Z | CH₃ | A-1 | 3-CN | H | CH₃ | CH₃ | 219-223 |
| 58 | Z | CH₃ | A-7 | (n = 0) | H | CH₃ | CH₃ | 49.8-81.8 |
| 59 | E | CH₃ | A-7 | (n = 0) | H | CH₃ | CH₃ | 116-139 |
| 60 | Z | CH₃ | A-9 | (n = 0) | H | CH₃ | CH₃ | 66.8-104.5 |
| 61 | E | CH₃ | A-9 | (n = 0) | H | CH₃ | CH₃ | 140.3-148.1 |
| 62 | E | CH₃ | A-1 | 2-F | H | CH₃ | CH₃ | 144-148 |
| 63 | E | CH₃ | A-1 | 2-Cl | H | CH₃ | CH₃ | 150-154 |
| 64 | E | CH₃ | A-1 | 3-F | H | CH₃ | CH₃ | 128-132 |
| 65 | E | CH₃ | A-1 | 5-Cl,2-CH₃ | H | CH₃ | CH₃ | 144-148 |
| 66 | E | CH₃ | A-1 | 2,5-di-CH₃ | H | CH₃ | CH₃ | 150-154 |
| 67 | E | CH₃ | A-1 | 2-Cl,5-CH₃ | H | CH₃ | CH₃ | 168-172 |
| 68 | E | CH₃ | A-1 | 3-CH₂CH₃ | H | CH₃ | CH₃ | 136-140 |
| 69 | E | CH₃ | A-1 | 2-CH₂CH₃ | H | CH₃ | CH₃ | 115-119 |
| 70 | E | CH₃ | A-1 | 2-F,3-CH₃ | H | CH₃ | CH₃ | 125-129 |
| 71 | E | CH₃ | A-1 | 3-CF₃ | H | CH₃ | CH₃ | 162-166 |
| 72 | E | CH₃ | A-1 | 4-F,2-CH₃ | H | CH₃ | CH₃ | 106-110 |
| 73 | E | CH₃ | A-1 | 2-Cl,5-CF₃ | H | CH₃ | CH₃ | 144-148 |
| 74 | E | CH₃ | A-1 | 3-CN | H | CH₃ | CH₃ | 172-176 |
| 75 | E | CH₃ | A-6 | 4-F | H | CH₃ | CH₃ | 200-204 |
| 76 | E | CH₃ | A-1 | 2-CN | H | CH₃ | CH₃ | 150-154 |
| 77 | Z | CH₃ | A-1 | 2-CN | H | CH₃ | CH₃ | 186-190 |
| 78 | E | CH₂CH₃ | A-6 | (n = 0) | H | CH₃ | CH₃ | 338 |
| 79 | Z | CH₂CH₃ | A-6 | (n = 0) | H | CH₃ | CH₃ | 338 |
| 80 | Z | n-Pr | A-6 | (n = 0) | H | CH₃ | CH₃ | 352 |
| 81 | E | n-Pr | A-6 | (n = 0) | H | CH₃ | CH₃ | 352 |
| 82 | E | CH₂C≡CH | A-6 | (n = 0) | H | CH₃ | CH₃ | 348 |
| Ex. 2 | | | | | | | | |
| 83 | Z | CH₂C≡CH | A-6 | (n = 0) | H | CH₃ | CH₃ | 348 |
| Ex. 2 | | | | | | | | |
| 84 | E | CH₂CH₃ | A-6 | 4-F | H | CH₃ | CH₃ | 356 |
| 85 | Z | CH₂CH₃ | A-6 | 4-F | H | CH₃ | CH₃ | 356 |
| 86 | Z | 2-PNL | A-6 | 4-F | H | CH3 | CH3 | 368 |
| 87 | E | 2-PNL | A-6 | 4-F | H | CH3 | CH3 | 368 |
| 88 | E | i-Pr | A-6 | 4-F | H | CH3 | CH3 | 370 |
| 89 | Z | i-Pr | A-6 | 4-F | H | C143 | C143 | 370 |
| 90 | Z | CH₃ | A-1 | 2,5-di-Cl | H | CH₃ | CH₃ | 189-193 |
| 91 | Z | CH₃ | A-6 | 3-Br | H | CH₃ | CH₃ | 129-133 |
| 92 | E | CH₃ | A-1 | 2-n-Pr | H | CH₃ | CH₃ | 316 |
| 93 | Z | CH₃ | A-1 | 2-i-Pr | H | CH₃ | CH₃ | 316 |
| 94 | E | CH₃ | A-1 | 2-i-Pr | H | CH₃ | CH₃ | 170.2-172.1 |
| 95 | Z | CH₃ | A-1 | 5-Cl,2-CF₃ | H | CH₃ | CH₃ | 142-146 |
| 96 | Z | CH₃ | A-6 | 6-Br | H | CH₃ | CH₃ | 200-204 |
| 97 | E | CH₃ | A-1 | 5-Cl,2-CF₃ | H | CH₃ | CH₃ | 170-174 |
| 98 | E | CH₃ | A-1 | 2,5-di-Cl | H | CH₃ | CH₃ | 157-161 |
| 99 | E | CH₃ | A-6 | 3-Br | H | CH₃ | CH₃ | 194-198 |
| 100 | E | CH₃ | A-6 | 6-Br | H | CH₃ | CH₃ | 199-203 |
| 101 | Z | CH₃ | A-1 | 6-Cl,2-F,3-CH₃ | H | CH₃ | CH₃ | 142-146 |
| 102 | E | CH₃ | A-1 | 2-Cl,3-CF₃ | H | CH₃ | CH₃ | 134-138 |
| 103 | Z | CH₃ | A-1 | 2-Cl,3-CF₃ | H | CH₃ | CH₃ | 163-167 |
| 104 | Z | CH₃ | A-1 | 2-c-Pr | H | CH₃ | CH₃ | 312 (M-1) |
| 105 | E | CH₃ | A-1 | 2-c-Pr | H | CH₃ | CH₃ | 138.2-140.5 |
| 106 | Z | 3-CPL | A-6 | 4-F | H | CH₃ | CH₃ | 402 |
| 107 | E | 3-CPL | A-6 | 4-F | H | CH₃ | CH₃ | 402 |
| 108 | Z | CH₂C≡CH | A-6 | 4-F | H | CH₃ | CH₃ | 366 |
| 109 | E | CH₂C≡CH | A-6 | 4-F | H | CH₃ | CH₃ | 366 |
| 110 | Z | CH₃ | A-6 | 7-Cl | H | CH₃ | CH₃ | 221-225 |
| 111 | E | CH₃ | A-6 | 7-Cl | H | CH₃ | CH₃ | 227-231 |
| 112 | E | CH₂C≡CH | A-6 | 3-Br | H | CH₃ | CH₃ | 426 |
| 113 | E | CH₂CH₃ | A-6 | 3-Br | H | CH₃ | CH₃ | 416 |
| 114 | Z | 2-PNL | A-6 | 3-Br | H | CH₃ | CH₃ | 428 |
| 115 | E | 2-PNL | A-6 | 3-Br | H | CH₃ | CH₃ | 428 |
| 116 | Z | CH₂C≡CH | A-6 | 3-Br | H | CH₃ | CH₃ | 426 |
| 117 | E | CH₂CH₃ | A-6 | 3-Br | H | CH₃ | CH₃ | 426 |
| 118 | E/Z | CH₂C≡CH | A-6 | (n = 0) | H | CH₃ | CH₃ | 348 |
| 119 | Z | CH₃ | A-6 | 6-F | H | CH₃ | CH₃ | 139-143 |
| 120 | Z | CH₃ | A-6 | (n +32 0) | C(═O)Me | CH₃ | CH₃ | * |
| 121 | Z | CH₃ | A-1 | 2-(2-PNL) | H | CH₃ | CH₃ | 146.1-150.6 |
| 122 | E | CH₃ | A-1 | 2-(2-PNL) | H | CH₃ | CH₃ | 107.5-109 |
| 123 | Z | CH₃ | A-1 | 3-c-Pr | H | CH₃ | CH₃ | 96-100 |
| 124 | E/Z | CH₂CH₃ | A-6 | 3-Br | H | CH₃ | CH₃ | |
| 125 | E | CH₃ | A-6 | 5-Cl | H | CH₃ | CH₃ | 150-154 |
| 126 | Z | CH₃ | A-6 | 5-Cl | H | CH₃ | CH₃ | 172-176 |
| 127 | E | CH₃ | A-6 | 6-F | H | CH₃ | CH₃ | 174-178 |
| 128 | E | CH₃ | A-1 | 3-CH₃ | H | Cl | CH₃ | 308 |
| 129 | Z | CH₃ | A-6 | n = 0 | H | Cl | CH₃ | 344 |
| Ex. 1 | | | | | | | | |
| 130 | E | CH₃ | A-6 | 4-Cl | H | CH₃ | CH₃ | 189-193 |
| 131 | E | CH₃ | A-6 | 4-CH₃ | H | CH₃ | CH₃ | 192-196 |
| 132 | E | CH₃ | A-6 | n = 0 | C(═O)Me | Cl | CH₃ | * |
| 133 | E/Z | CH₃ | A-2 | 4-CH₃ | H | CH₃ | CH₃ | 169-174.5 |
| 134 | E | CH₃ | A-2 | 3-CH₃ | H | CH₃ | CH₃ | 124-158.7 |
| 135 | E | CH₃ | A-4 | 2-CH₃ | H | CH₃ | CH₃ | 144.5-148.3 |
| 136 | E | CH₂C≡CH | A-6 | n = 0 | H | Cl | CH₃ | 368 |
| 137 | Z | CH₂C≡CH | A-6 | n = 0 | H | Cl | CH₃ | 368 |
| 138 | E | CH₂CH₃ | A-6 | 3-Cl | H | CH₃ | CH₃ | 372 |
| 139 | Z | CH₂CH₃ | A-6 | 3-Cl | H | CH₃ | CH₃ | 372 |
| 140 | E | CH₃ | A-4 | 4-CH₃ | H | CH₃ | CH₃ | 128.1-132.6 |
| 141 | E | CH₃ | A-3 | 5-CH₃ | H | CH₃ | CH₃ | 278 |
| 142 | E | CH₃ | A-6 | 4-OCH₃ | H | CH₃ | CH₃ | 162-166 |
| 143 | E/Z | CH₃ | A-1 | 3-Cl | H | CH₃ | H | 176-180 |
| 144 | E/Z | CH₃ | A-2 | 5-CH₃ | H | CH₃ | CH₃ | 174.4-195.1 |
| 145 | E | CH₃ | A-6 | n = 0 | H | Cl | CH₃ | 344 |
| Ex. 1 | | | | | | | | |
| 146 | Z | CH₂CH₃ | A-6 | n = 0 | H | Cl | CH₃ | 358 |
| 147 | E | CH₂CH₃ | A-6 | n = 0 | H | Cl | CH₃ | 358 |
| 148 | E | CH₃ | A-1 | 3-SO₂CH₃ | H | CH₃ | CH₃ | 352 |
| 149 | Z | CH₂Ph | A-1 | 3-Cl | H | CH₃ | CH₃ | |
| 150 | E | CH₂Ph | A-1 | 3-Cl | H | CH₃ | CH₃ | |
| 151 | E | CH₃ | A-4 | 5-CH₃ | H | CH₃ | CH₃ | |
| 152 | E | CH₃ | A-1 | 3,5-di-F,2-CH₃ | H | CH₃ | CH₃ | 155-159 |
| 153 | Z | CH₃ | A-1 | 3,5-di-Cl,2-F | H | CH₃ | CH₃ | 182-186 |
| 154 | Z | CH₂C≡CH | A-1 | 3-Cl,5-CH₃ | H | CH₃ | CH₃ | 121-125 |
| 155 | E | CH₂C≡CH | A-1 | 3-Cl,5-CH₃ | H | CH₃ | CH₃ | 183-187 |
| 156 | Z | CH₃ | A-1 | 3-Br,5-Cl | H | CH₃ | CH₃ | 150.7-166.7 |
| 157 | E | CH₃ | A-1 | 3-Br,5-Cl | H | CH₃ | CH₃ | 135-214 |
| 158 | E | CH₂Ph | A-6 | 4-F | H | CH₃ | CH₃ | 418 |
| 159 | E | CH₃ | A-6 | 6-Cl | H | CH₃ | CH₃ | 358 |
| 160 | E/Z* | CH₃ | A-1 | 2,5-di-Cl | H | CH₃ | CH₃ | |
| 161 | Z | CH₂CH₃ | A-6 | n = 0 | H | I | CH₃ | 448 (M-1) |
| 162 | E | CH₂CH₃ | A-6 | n = 0 | H | I | CH₃ | 450 |
| 163 | Z | CH₂C≡CH | A-1 | 3-Br,4-F | H | CH₃ | CH₃ | 191-195 |
| 164 | E. | CH₂C≡CH | A-1 | 3-Br,4-F | H | CH₃ | CH₃ | 143-147 |
| 165 | Z | CH₃ | A-1 | 3-Cl,5-OCH₃ | H | CH₃ | CH₃ | 167-171 |
| 166 | E | CH₂C≡CH | A-1 | 3,4-di-Cl | H | CH₃ | CH₃ | 158-162 |
| 167 | E | i -Pr | A-1 | 3,5-di-Cl,2-F | H | CH₃ | CH₃ | 155-159 |
| 168 | E/Z | CH₂C≡CH | A-6 | n = 0 | H | OCH₃ | CH₃ | 364 |
| 169 | Z | i-Pr | A-6 | 3-Br | H | H | CH₃ | 170-174 |
| 170 | Z | CH₂CH₃ | A-6 | 3-Br | H | H | CH₃ | 173-177 |
| 171 | E | CH₂CH₃ | A-6 | 3-Br | H | H | CH₃ | 197-201 |
| 172 | E | CH₂CH₃ | A-6 | n = 0 | H | Br | CH₃ | 403 |
| 173 | Z | CH₃ | A-1 | 3-Cl,5-CH₃ | H | CH₃ | CH₃ | 171-175 |
| 174 | E | CH₃ | A-1 | 3-Cl,5-CH₃ | H | CH₃ | CH₃ | 185-189 |
| 175 | E | CH₃ | A-1 | 3-Cl,5-OCH₃ | H | CH₃ | CH₃ | 165-169 |
| 176 | Z | CH₃ | A- 1 | 3-Br,4-F | H | CH₃ | CH₃ | 174-178 |
| 177 | E | CH₃ | A-1 | 3-Br,4-F | H | CH₃ | CH₃ | 114-118 |
| 178 | Z | CH₂C≡CH | A-1 | 3,4-di-Cl | H | CH₃ | CH₃ | 141-145 |
| 179 | Z | i-Pr | A-1 | 3,5-di-Cl,2-F | H | CH₃ | CH₃ | 174-178 |
| 180 | Z | CH₂CH₃ | A-1 | 3-Br,5-Cl | H | CH₃ | CH₃ | 52.5-178.9 |
| 181 | E | CH₂CH₃ | A-1 | 3-Br,5-Cl | H | CH₃ | CH₃ | 131.6-270.2 |
| 182 | Z | CH₂-c-Pr | A-1 | 2,3,5-tri-Cl | H | CH₃ | CH₃ | 154.7-157.7 |
| 183 | Z | n-Bu | A-1 | 2,3,5-tri-Cl | H | CH₃ | CH₃ | 101.5-108.2 |
| 184 | Z | CH₂C≡CH | A-1 | 2,3,5-tri-Cl | H | CH₃ | CH₃ | 90.5-123.7 |
| 185 | Z | i-Pr | A-1 | 2,3,5-tri-Cl | H | CH₃ | CH₃ | 144.3-147.9 |
| 186 | Z | CH₂CH₃ | A-1 | 2,3,5-tri-Cl | H | CH₃ | CH₃ | 130.9-148.5 |
| 187 | Z | CH₂C≡CH | A-1 | 2,3,5-tri-Cl | H | CH₃ | CH₃ | 113.4-142.3 |
| 188 | Z | CH₃ | A-1 | 3,5-di-Cl | H | CH₃ | CH₃ | 342 |
| 189 | E | CH₃ | A-1 | 3,5-di-Cl | H | CH₃ | CH₃ | 342 |
| 190 | Z | CH₃ | A-6 | 4-F | C(↑O)Me | Cl | CH₃ | 177-182 |
| 191 | Z | CH₂C≡CH | A-6 | 3-Cl | H | CH₃ | CH₃ | 382 |
| 192 | E | CH₂C≡CH | A-6 | 3-Cl | H | CH₃ | CH₃ | 382 |
| 193 | Z | CH₃ | A-1 | 2,5-di-F,3-CH₃ | H | CH₃ | CH₃ | 199-203 |
| 194 | E | i-Pr | A-1 | 3,5-di-Cl,2-F | H | H | CH₃ | 189-193 |
| 195 | E | CH₂CH₃ | A-1 | 3,5-di-Cl,2-F | H | H | CH | 130-134 |
| 196 | Z | CH₂C≡CH | A-6 | 3-Br | H | H | CH₃ | 161-165 |
| 197 | Z | CH₂C≡CH | A-1 | 3-OCH₂CF₃ | H | CH₃ | CH₃ | |
| 198 | Z | CH₃ | A-1 | 3-OCH₂CF₃ | H | CH₃ | CH₃ | |
| 199 | Z | CH₂C≡CH | A-1 | 3-OCH₂CH₃ | H | CH₃ | CH₃ | |
| 200 | Z | CH₃ | A-1 | 3-OCH₂CH₃ | H | CH₃ | CH₃ | |
| 201 | Z | CH₂C≡CH | A-1 | 3-OCHF₂ | H | CH₃ | CH₃ | |
| 202 | Z | CH₃ | A-1 | 3-OCHF_{2+L} | H | CH₃ | CH₃ | |
| 203 | Z | CH₂C≡CH | A-1 | 3-OCH₃ | H | CH₃ | CH₃ | |
| 204 | Z | CH₃ | A-1 | 3-OCH₃ | H | CH₃ | CH₃ | |
| 205 | E | CH₂C≡CH | A-6 | 4-F | H | Cl | CH₃ | 165-169 |
| 206 | E | CH₂C≡CH | A-6 | n = 0 | H | OCH₃ | CH₂C≡CH | 364 |
| 207 | E | CH₃ | A-6 | 4-F | H | Cl | CH₃ | 158-163 |
| 208 | Z | CH₃ | A-6 | 4-F | H | Cl | CH₃ | 120-124 |
| 209 | E | i-Pr | A-6 | 4-F | H | Cl | CH₃ | 155-160 |
| 210 | Z | i-Pr | A-6 | 4-F | H | Cl | CH₃ | 150-155 |
| 211 | E | CH₂CH₃ | A-6 | 4-F | H | Cl | CH₃ | 133-138 |
| 212 | Z | CH₂CH₃ | A-6 | 4-F | H | Cl | CH₃ | 149-154 |
| 213 | Z | CH₃ | A-1 | 2,3,5-tri-Cl | H | CH₃ | CH₃ | 169-173 |
| 214 | E | CH₃ | A-1 | 2,3,5-tri-Cl | H | CH₃ | CH₃ | 170-174 |
| 215 | E | CH₂C≡CH | A-1 | 3,5-di-Cl,2-F | H | H | CH₃ | 149-153 |
| 216 | Z | CH₃ | A-1 | 3,4-di-Cl | H | CH₃ | CH₃ | 180-184 |
| 217 | E | CH₃ | A-1 | 3,4-di-Cl | H | CH₃ | CH₃ | 148-152 |
| 218 | Z | CH₃ | A-1 | 3-Cl,4-F | H | CH₃ | CH₃ | 168-172 |
| 219 | E | CH₃ | A-1 | 3-Cl,4-F | H | CH₃ | CH₃ | 165-169 |
| 220 | Z | CH₂C≡CH | A-6 | 4-F | H | Cl | CH₃ | 187-191 |
| 221 | E | CH₂C≡CH | A-6 | n = 0 | H | H | CH₃ | 334 |
| 222 | Z | i-Pr | A-6 | n = 0 | H | H | CH₃ | 338 |
| 223 | E | i-Pr | A-6 | n = 0 | H | H | CH₃ | 338 |
| 224 | Z | CH₂CH₃ | A-6 | n = 0 | H | H | CH₃ | 324 |
| 225 | E | CH₂CH₃ | A-6 | n = 0 | H | H | CH₃ | 324 |
| 226 | Z | CH₃ | A-1 | 2,3,5-tri-F | H | CH₃ | CH₃ | 170-174 |
| 227 | E | CH₃ | A-1 | 2,3,5-tri-F | H | CH₃ | CH₃ | 139-143 |
| 228 | E | CH₂CH₃ | A-6 | n = 0 | H | CN | CH₃ | 349 |
| 229 | Z | CH₂C≡CH | A-1 | 3-Cl,4-F | H | CH₃ | CH₃ | 141-145 |
| 230 | E | CH₂C≡CH | A-1 | 3-Cl,4-F | H | CH₃ | CH₃ | 127-131 |
| 231 | Z | CH₃ | A-6 | 3-Br | C(═O)Me | Cl | CH₃ | 182-186 |
| 232 | E | i-Pr | A-6 | 3-Br | H | Cl | CH₃ | 180-185 |
| 233 | Z | i-Pr | A-6 | 3-Br | H | Cl | CH₃ | 248-253 |
| 234 | Z | CH₃ | A-1 | 3-Br | H | CH₃ | CH₃ | 159-163 |
| 235 | Z | CH₂CH₃ | A-6 | 3-Br | H | Cl | CH₃ | 183-187 |
| 236 | E | CH₂CH₃ | A-6 | 3-Br | H | Cl | CH₃ | 124-128 |
| 237 | E | CH₃ | A-6 | n = 0 | H | H | CH₃ | 310 |
| 238 | Z | CH₃ | A-6 | n = 0 | H | H | CH₃ | 310 |
| 239 | E | CH₃ | A-1 | 3-Br | H | CH₃ | CH₃ | 200-204 |
| 240 | E | CH₂CH₂OH | A-6 | 6-Cl | H | CH₃ | CH₃ | 221-225 |
| 241 | Z | i-Pr | A-6 | 6-Cl | H | CH₃ | CH₃ | 286-291 |
| 242 | E | CH₂C≡CH | A-6 | 6-Cl | H | CH₃ | CH₃ | 181-186 |
| 243 | Z | CH₂CH═CH₂ | A-6 | 6-Cl | H | CH₃ | CH₃ | 200-204 |
| 244 | E | CH₂CH₃ | A-6 | 6-Cl | H | CH₃ | CH₃ | 205-209 |
| 245 | E | i-Pr | A-6 | 6-Cl | H | CH₃ | CH₃ | 170-175 |
| 246 | E | CH₂CH═CH₂ | A-6 | 6-Cl | H | CH₃ | CH₃ | 269-273 |
| 247 | Z | CH₂C≡CH | A-1 | 3-CH₂OEt | H | CH₃ | CH₃ | |
| 248 | Z | CH₃ | A-1 | 3-CH₂OEt | H | CH₃ | CH₃ | |
| 249 | E | CH₃ | A-1 | 3-CH₂OEt | H | CH₃ | CH₃ | |
| 250 | Z | CH₃ | A-6 | 3-Br | H | Cl | CH₃ | |
| 251 | E | CH₃ | A-1 | 3,5-di-Cl,2-F | H | H | CH₃ | |
| 252 | E | CH₂CH₃ | A-6 | n = 0 | H | c-Pr | CH₃ | 364 |
| 253 | Z | CH₂C≡CH | A-1 | 3-CH═CHCl(E) | H | CH₃ | CH₃ | |
| 254 | E | CH₂C≡CH | A-1 | 3-CH═CHCl(E) | H | CH₃ | CH₃ | |
| 255 | E/Z | CH₂C≡CH | A-6 | 3-Br | H | CH₃ | CH₃ | |
| 256 | E/Z | i-Pr | A-6 | 4-F | H | CH₃ | CH₃ | 370 |
| 257 | E/Z | CH₃ | A-6 | 3-Br | H | CH₃ | CH₃ | |
| 258 | E/Z | CH₂CH₃ | A-6 | n = 0 | H | Cl | CH₃ | 358 |
| 259 | E/Z | CH₃ | A-6 | n = 0 | H | Cl | CH₃ | 344 |
| 260 | Z | CH₃ | A-6 | 3-Br | H | H | CH₃ | 194-198 |
| 261 | Z | CH₂C≡CH | A-1 | 3-CH═CHCl(Z) | H | CH₃ | CH₃ | |
| 262 | E | CH₂C≡CH | A-1 | 2,3,5-tri-Cl | H | H | CH₃ | 107-111 |
| 263 | E | i-Pr | A-1 | 2,3,5-tri-Cl | H | H | CH₃ | 153-157 |
| 264 | E | CH₂C≡CH | A-1 | 3-CH═CHCl(Z) | H | CH₃ | CH₃ | |
| 265 | E | CH₂CH₃ | A-1 | 2,3,5-tri-Cl | H | H | CH₃ | 154-158 |
| 266 | E/Z* | CH₃ | A-1 | 3-Cl | H | CH₃ | CH₃ | 308 |
| 267 | E/Z* | CH₃ | A-1 | 2-F,3-CH₃ | H | CH₃ | CH₃ | 306 |
| 268 | E | n-Bu | A-1 | 2,3,5-tri-Cl | H | CH₃ | CH₃ | |
| 269 | Z | CH₂CH₃ | A-6 | 4-F | H | H | CH₃ | 191-195 |
| 270 | E | CH₂CH₃ | A-6 | 4-F | H | H | CH₃ | 152-156 |
| 271 | Z | CH₃ | A-1 | 2,5-di-Cl,3-F | H | CH₃ | CH₃ | 162-166 |
| 272 | E | CH₃ | A-1 | 2,5-di-Cl,3-F | H | CH₃ | CH₃ | 167-171 |
| 273 | Z | CH₂CH₃ | A-6 | 6-Cl | H | CH₃ | CH₃ | 168-171 |
| 274 | E | CH₃ | A-1 | 2,3,5-tri-Cl | H | H | CH₃ | 175-179 |
| 275 | Z | CH₂C≡CH | A-1 | 3-Cl,5-OCH₃ | H | CH₃ | CH₃ | 115-119 |
| 276 | E | CH₂C≡CH | A-1 | 3-Cl,5-OCH₃ | H | CH₃ | CH₃ | 182-186 |
| 277 | Z | CH₂C≡CH | A-1 | 3-Br | H | CH₃ | CH₃ | 191-195 |
| 278 | E | CH₂C≡CH | A-1 | 3-Br | H | CH₃ | CH₃ | 128-132 |
| 279 | Z | CH₃ | A-6 | 3-SCH₃ | H | CH₃ | CH₃ | 242-247 |
| 280 | Z | CH₂C≡CH | A-1 | 3-O-i-Pr | H | CH₃ | CH₃ | |
| 281 | Z | CH₃ | A-1 | 3-O-i-Pr | H | CH₃ | CH₃ | |
| 282 | E | CH₂CH₃ | A-6 | n = 0 | H | CF₃ | CH₃ | 392 |
| 283 | Z | CH₂-c-Pr | A-1 | 3,5-di-Cl,2-F | H | CH₃ | CH₃ | 123-127 |
| 284 | E | CH₂-c-Pr | A-1 | 3,5-di-Cl,2-F | H | CH₃ | CH₃ | 141-145 |
| 285 | Z | CH₃ | A-1 | 3-CH═CHCl(E) | H | CH₃ | CH₃ | |
| 286 | E | CH₃ | A-1 | 3-CH═CHCl(E) | H | CH₃ | CH₃ | |
| 287 | Z | CH₃ | A-1 | 3-CH═CHCl(Z) | H | CH₃ | CH₃ | 184.5-195.9 |
| 288 | E | CH₃ | A-1 | 3-CH═CHCl(Z) | H | CH₃ | CH₃ | 88.4-178 |
| 289 | Z | CH₃ | A-1 | 3,5-di-Cl,4-F | H | CH₃ | CH₃ | 159.8-164.2 |
| 290 | E | CH₃ | A-1 | 3,5-di-Cl,4-F | H | CH₃ | CH₃ | 179.2-193.8 |
| 291 | Z | CH₂C≡CH | A-1 | 3-Br,5-Cl | H | CH₃ | CH₃ | 87.8-110 |
| 292 | E | CH₂C≡CH | A-1 | 3-Br,5-Cl | H | CH₃ | CH₃ | 72-149.1 |
| 293 | E | CH₂-c-Pr | A-1 | 2,3,5-tri-Cl | H | CH₃ | CH₃ | 81.9-129.1 |
| 294 | E | CH₂CH═CH₂ | A-1 | 2,3,5-tri-Cl | H | CH₃ | CH₃ | 81-91.4 |
| 295 | E | i-Pr | A-1 | 2,3,5-tri-Cl | H | CH₃ | CH₃ | 110.9-119.4 |
| 296 | E | CH₂C≡CH | A-1 | 2,3,5-tri-Cl | H | CH₃ | CH₃ | 41.7-57.9 |
| 297 | E | CH₃ | A-6 | 6-SCH₃ | H | CH₃ | CH₃ | 184-188 |
| 298 | E | CH₃ | A-6 | 3-SCH₃ | H | CH₃ | CH₃ | 195-199 |
| 299 | E | CH₃ | A-1 | 3,5-di-Cl,2-F | H | CH₃ | CH₃ | 193-197 |
| 300 | Z | CH₃ | A-6 | 6-Cl | H | CH₃ | CH₃ | 217-221 |
| 301 | E | CH₃ | A-6 | 6-Cl | H | CH₃ | CH₃ | 220-224 |
| 302 | E | i-Pr | A-6 | 4-F | H | H | CH₃ | 139-143 |
| 303 | Z | CH₃ | A-6 | 4-F | H | H | CH₃ | 204-208 |
| 304 | E | CH₃ | A-6 | 4-F | H | H | CH₃ | 161-165 |
| 305 | Z | CH₃ | A-1 | 3,5-di-F,2-CH₃ | H | CH₃ | CH₃ | 170-174 |

*See Index Table B for ¹H NMR data and E/Z ratios.

INDEX TABLE B

| Cmpd. | ¹H NMR Data (CDCl₃ solution unless |
|---|---|
| No. | indicated otherwise)^{a} |
| 120 | δ 7.82-7.98 (m, 3H), 7.56-7.61 (m, 1H), 7.50-7.55 |
| | (m, 1H), 7.41-7.48 (m, 2H), 4.72 (s, 2H), 3.69 |
| | (s, 3H), 2.12-2.32 (m, 6H). |
| 132 | δ 7.84-7.88 (m, 2H), 7.80-7.84 (m, 1H), 7.48-7.57 |
| | (m, 2H), 7.41-7.45 (m, 1H), 7.31-7.37 (m, 1H), |
| | 3.93 (s, 3H), 3.71 (s, 3H), 2.02 (s, 3H). |
| 160 | 4:1.5 MIXTURE OF E:Z ISOMERS |
| 266 | 1:4 MIXTURE OF E:Z |
| 267 | 1:4 MIXTURE OF E:Z |

^{a1}H NMR data are in ppm downfield from tetramethylsilane at 500 MHz.
Couplings are designated by (s)-singlet and (m)-multiplet.

### Biological Examples of the Invention

### Test A

Seeds of plant species selected from barnyardgrass (Echinochloa crus-galli), kochia (Kochia scoparia), ragweed (common ragweed, Ambrosia elatior), Italian ryegrass (Lolium multiflorum), foxtail, giant (giant foxtail, Setaria faberii), foxtail, green (green foxtail, Setaria viridis), and pigweed (Amaranthus retroflexus) were planted into a blend of loam soil and sand and treated preemergence with a directed soil spray using test chemicals formulated in a non-phytotoxic solvent mixture which included a surfactant.

At the same time, plants selected from these weed species and also wheat (Triticum aestivum), corn (Zea mays), blackgrass (Alopecurus myosuroides), and galium (catchweed bedstraw, Galium aparine) were planted in pots containing the same blend of loam soil and sand and treated with postemergence applications of test chemicals formulated in the same manner. Plants ranged in height from 2 to 10 cm and were in the one- to two-leaf stage for the postemergence treatment. Treated plants and untreated controls were maintained in a greenhouse for approximately 10 days, after which time all treated plants were compared to untreated controls and visually evaluated for injury. Plant response ratings, summarized in Table A, are based on a 0 to 100 scale where 0 is no effect and 100 is complete control. A dash (-) response means no test result.

TABLE A

| 500 g ai/ha | Compounds | | | | | | | | | | | | | |
|---|---|---|---|---|---|---|---|---|---|---|---|---|---|---|
| Postemergence | 1 | 2 | 3 | 4 | 5 | 8 | 9 | 10 | 11 | 12 | 13 | 14 | 15 | 16 |
| Barnyardgrass | 10 | 30 | 40 | 0 | 30 | 10 | 20 | 10 | 10 | 10 | 50 | 40 | 30 | 10 |
| Blackgrass | 20 | 20 | 50 | 40 | 50 | 20 | 20 | 30 | 70 | 10 | 50 | 60 | 60 | 0 |
| Corn | 0 | 0 | 20 | 0 | 0 | 0 | 10 | 0 | 10 | 0 | 10 | 10 | 10 | 0 |
| Foxtail, Giant | — | — | 40 | 0 | 10 | — | — | — | — | — | — | — | — | — |
| Foxtail, Green | 10 | 20 | — | — | — | 20 | 30 | 20 | 40 | 10 | 60 | 40 | 30 | 10 |
| Galium | 30 | 30 | 70 | 0 | 0 | 90 | 90 | — | — | 70 | 100 | 90 | 90 | 0 |
| Kochia | 0 | 20 | 50 | 0 | 0 | 70 | 60 | 80 | 80 | 10 | 60 | 50 | 20 | 10 |
| Pigweed | 0 | 10 | 50 | 0 | 0 | 20 | 20 | 60 | 80 | 30 | 90 | 80 | 30 | 0 |
| Ragweed | 0 | 20 | 60 | 0 | 0 | 80 | 80 | 100 | 100 | 30 | 70 | 70 | 80 | 30 |
| Ryegrass, | 20 | 80 | 80 | 0 | 30 | 60 | 60 | 90 | 80 | 0 | 90 | 60 | 80 | 0 |
| Italian | | | | | | | | | | | | | | |
| Wheat | 0 | 10 | 10 | 0 | 0 | 0 | 0 | 20 | 10 | 0 | 0 | 0 | 0 | 0 |
| 500 g ai/ha | Compounds | | | | | | | | | | | | | |
| Postemergence | 19 | 20 | 21 | 22 | 23 | 24 | 25 | 26 | 27 | 28 | 29 | 30 | 31 | 32 |
| Barnyardgrass | 60 | 50 | 0 | 10 | 0 | 30 | 30 | 60 | 0 | 30 | 20 | 40 | 90 | 20 |
| Blackgrass | 50 | 60 | 0 | 40 | 50 | 40 | 40 | 90 | 10 | 40 | 70 | 80 | 90 | 20 |
| Corn | 0 | 0 | 0 | 0 | 0 | 0 | 0 | 30 | 0 | 0 | 10 | 0 | 50 | 0 |
| Foxtail, Giant | — | — | — | — | — | — | — | — | — | — | — | — | — | 20 |
| Foxtail, Green | 50 | 60 | 0 | 0 | 0 | 20 | 10 | 80 | 0 | 10 | 20 | 40 | 80 | — |
| Galium | 90 | 90 | 80 | 60 | 60 | 60 | 20 | 100 | 80 | 60 | 80 | 80 | 90 | 80 |
| Kochia | 30 | 50 | 10 | 30 | 30 | 20 | 10 | 90 | 0 | 0 | 30 | 30 | 80 | 70 |
| Pigweed | 80 | 70 | 60 | 50 | 80 | 60 | 50 | 90 | 30 | 40 | 50 | 50 | 90 | 40 |
| Ragweed | 80 | 80 | 20 | 50 | 50 | 60 | 40 | 100 | 40 | 40 | 70 | 60 | 90 | 80 |
| Ryegrass, | 30 | 50 | 20 | 30 | 50 | 40 | 40 | 70 | 20 | 10 | 90 | 70 | 90 | 60 |
| Italian | | | | | | | | | | | | | | |
| Wheat | 0 | 0 | 0 | 0 | 0 | 0 | 0 | 10 | 20 | 0 | 20 | 30 | 20 | 10 |
| 500 g ai/ha | Compounds | | | | | | | | | | | | | |
| Postemergence | 33 | 34 | 35 | 36 | 37 | 38 | 39 | 40 | 41 | 42 | 43 | 44 | 45 | 46 |
| Barnyardgrass | 0 | 20 | 0 | 0 | 20 | 20 | 10 | 30 | 0 | 10 | 0 | 30 | 0 | 0 |
| Blackgrass | 0 | 10 | 20 | 20 | 80 | 30 | 0 | 80 | 0 | 0 | 20 | 30 | 20 | 0 |
| Corn | 0 | 0 | 0 | 0 | 30 | 20 | 20 | 10 | 0 | 0 | 0 | 20 | 0 | 0 |
| Foxtail, Giant | 10 | 10 | 0 | 10 | 70 | 30 | 0 | 50 | 0 | 10 | 0 | 50 | 0 | 0 |
| Foxtail, Green | — | — | — | — | — | — | — | — | — | — | — | — | — | — |
| Galium | 70 | 70 | 70 | 70 | 90 | 90 | 70 | 100 | 70 | 60 | 50 | 50 | 70 | 60 |
| Kochia | 40 | 20 | 10 | 70 | 80 | 50 | 0 | 40 | 60 | 50 | 20 | 0 | 0 | 0 |
| Pigweed | 30 | 20 | 20 | 70 | 90 | 60 | 10 | 40 | 90 | 80 | 60 | 10 | 10 | 20 |
| Ragweed | 60 | 50 | 20 | 80 | 80 | 70 | 60 | 80 | 60 | 50 | 30 | 40 | 70 | 70 |
| Ryegrass, | 60 | 60 | 20 | 50 | 90 | 20 | 0 | 90 | 60 | 70 | 30 | 0 | 20 | 10 |
| Italian | | | | | | | | | | | | | | |
| Wheat | 20 | 20 | 0 | 0 | 10 | 0 | 10 | 50 | 20 | 0 | 0 | 0 | 0 | 0 |
| 500 g ai/ha | Compounds | | | | | | | | | | | | | |
| Postemergence | 47 | 48 | 49 | 50 | 51 | 52 | 53 | 55 | 56 | 57 | 58 | 59 | 60 | 61 |
| Barnyardgrass | 20 | 30 | 50 | 40 | 80 | 30 | 70 | 30 | 10 | 0 | 50 | 50 | 0 | 20 |
| Blackgrass | 30 | 90 | 90 | 30 | 100 | 30 | 40 | 80 | 40 | 0 | 90 | 80 | 10 | 20 |
| Corn | 20 | 80 | 50 | 0 | 20 | 0 | 30 | 30 | 0 | 0 | 0 | 20 | 0 | 0 |
| Foxtail, Giant | 70 | 80 | 80 | 70 | 90 | 20 | 90 | 50 | 0 | 0 | 80 | 60 | 30 | 20 |
| Foxtail, Green | — | — | — | — | — | — | — | — | — | — | — | — | — | — |
| Galium | 100 | 90 | 90 | 70 | 100 | 80 | 0 | 70 | 80 | 50 | 90 | 90 | 70 | 80 |
| Kochia | 30 | 80 | 90 | 80 | 90 | 60 | 0 | 30 | 50 | 50 | 70 | 80 | 50 | 50 |
| Pigweed | 90 | 90 | 90 | 90 | 90 | 90 | 0 | 20 | 70 | 30 | 90 | 90 | 90 | 80 |
| Ragweed | 90 | 90 | 90 | 80 | 100 | 90 | 0 | 30 | 50 | 10 | 100 | 80 | 40 | 30 |
| Ryegrass, | 70 | 80 | 80 | 80 | 100 | 80 | 90 | 70 | 50 | 20 | 90 | 100 | 20 | 70 |
| Italian | | | | | | | | | | | | | | |
| Wheat | 0 | 10 | 10 | 0 | 90 | 0 | 0 | 20 | 0 | 10 | 80 | 0 | 40 | 40 |
| 500 g ai/ha | Compounds | | | | | | | | | | | | | |
| Postemergence | 62 | 63 | 64 | 65 | 66 | 67 | 68 | 69 | 70 | 71 | 72 | 73 | 74 | 75 |
| Barnyardgrass | 0 | 0 | 20 | 40 | 30 | 0 | 0 | 30 | 30 | 30 | 0 | 60 | 0 | 70 |
| Blackgrass | 10 | 80 | 20 | 30 | 50 | 30 | 30 | 60 | 30 | 50 | 0 | 90 | 20 | 100 |
| Corn | 0 | 0 | 0 | 0 | 0 | 0 | 0 | 20 | 0 | 10 | 20 | 20 | 0 | 20 |
| Foxtail, Giant | 10 | — | 10 | 70 | — | 10 | 10 | 40 | 40 | 20 | 10 | 60 | 0 | 80 |
| Foxtail, Green | — | 10 | — | — | 20 | — | — | — | — | — | — | — | — | — |
| Galium | 70 | 80 | 80 | 90 | 80 | 80 | 70 | 80 | 80 | 80 | 70 | 70 | 50 | 100 |
| Kochia | 20 | 30 | 60 | 80 | 40 | 20 | 80 | 70 | 80 | 60 | 60 | 80 | 30 | 90 |
| Pigweed | 30 | 50 | 40 | 90 | 50 | 30 | 80 | 70 | 90 | 70 | 80 | 90 | 30 | 90 |
| Ragweed | 40 | 50 | 50 | 80 | 50 | 70 | 90 | 70 | 80 | 60 | 70 | 80 | 20 | 100 |
| Ryegrass, | 20 | 70 | 70 | 90 | 80 | 20 | 30 | 80 | 70 | 40 | 40 | 90 | 20 | 100 |
| Italian | | | | | | | | | | | | | | |
| Wheat | 0 | 20 | 0 | 20 | 10 | 0 | 0 | 0 | 0 | 40 | 20 | 40 | 0 | 80 |
| 500 g ai/ha | Compounds | | | | | | | | | | | | | |
| Postemergence | 76 | 77 | 78 | 79 | 80 | 81 | 82 | 83 | 84 | 85 | 86 | 87 | 88 | 89 |
| Barnyardgrass | 20 | 0 | 80 | 90 | 60 | 70 | 80 | 80 | 80 | 80 | 80 | 80 | 80 | 60 |
| Blackgrass | 0 | 0 | 80 | 80 | 90 | 80 | 100 | 100 | 100 | 100 | 90 | 90 | 90 | 100 |
| Corn | 0 | 0 | 30 | 20 | 20 | 20 | 30 | 40 | 30 | 30 | 20 | 30 | 30 | 30 |
| Foxtail, Giant | 0 | 10 | 80 | 80 | 80 | 80 | 80 | 80 | 80 | 80 | 80 | 80 | 90 | 80 |
| Foxtail, Green | — | — | — | — | — | — | — | — | — | — | — | — | — | — |
| Galium | 70 | 40 | 100 | 100 | 90 | 90 | 100 | 100 | 100 | 90 | 100 | 100 | 90 | 90 |
| Kochia | 0 | 30 | 70 | 80 | 60 | 60 | 90 | 90 | 80 | 90 | 50 | 70 | 70 | 80 |
| Pigweed | 70 | 70 | 90 | 90 | 80 | 90 | 90 | 90 | 90 | 90 | 80 | 90 | 100 | 100 |
| Ragweed | 60 | 30 | 90 | 100 | 90 | 90 | 100 | 100 | 100 | 100 | 100 | 90 | 90 | 90 |
| Ryegrass, | 30 | 20 | 40 | 30 | 70 | 20 | 80 | 100 | 100 | 100 | 100 | 100 | 100 | 100 |
| Italian | | | | | | | | | | | | | | |
| Wheat | 0 | 0 | 0 | 0 | 0 | 0 | 80 | 60 | 50 | 50 | 80 | 60 | 50 | 70 |
| 500 g ai/ha | Compounds | | | | | | | | | | | | | |
| Postemergence | 90 | 91 | 92 | 93 | 94 | 95 | 96 | 97 | 98 | 99 | 100 | 101 | 102 | 103 |
| Barnyardgrass | 60 | 80 | 40 | 20 | 30 | 60 | 30 | 60 | 80 | 90 | 70 | 30 | 30 | 30 |
| Blackgrass | 90 | 100 | 70 | 70 | 80 | 100 | 100 | 90 | 90 | 100 | 90 | 30 | 30 | 40 |
| Corn | 30 | 70 | 10 | 10 | 20 | 40 | 30 | 40 | 20 | 60 | 40 | 0 | 0 | 0 |
| Foxtail, Giant | 80 | 90 | 60 | 40 | 70 | 80 | 70 | 80 | 80 | 80 | 80 | 20 | 30 | 30 |
| Foxtail, Green | — | — | — | — | — | — | — | — | — | — | — | — | — | — |
| Galium | 90 | 100 | 70 | 70 | 70 | 90 | 90 | 90 | 100 | 100 | 90 | 80 | 70 | 70 |
| Kochia | 80 | 80 | 60 | 30 | 60 | 80 | 80 | 70 | 80 | 70 | 70 | 30 | 20 | 20 |
| Pigweed | 90 | 90 | 80 | 80 | 80 | 80 | 90 | 80 | 80 | 100 | 90 | 40 | 40 | 30 |
| Ragweed | 90 | 100 | 70 | 50 | 70 | 90 | 100 | 80 | 90 | 90 | 100 | 60 | 30 | 20 |
| Ryegrass, | 90 | 100 | 60 | 80 | 80 | 100 | 80 | 100 | 80 | 90 | 90 | 10 | 30 | 50 |
| Italian | | | | | | | | | | | | | | |
| Wheat | 40 | 80 | 0 | 10 | 40 | 70 | 50 | 50 | 20 | 80 | 50 | 0 | 30 | 0 |
| 500 g ai/ha | Compounds | | | | | | | | | | | | | |
| Postemergence | 104 | 105 | 106 | 107 | 108 | 109 | 110 | 111 | 112 | 113 | 114 | 115 | 116 | 117 |
| Barnyardgrass | 80 | 80 | 90 | 90 | 90 | 90 | 70 | 80 | 90 | 90 | 90 | 90 | 90 | 90 |
| Blackgrass | 90 | 90 | 90 | 80 | 100 | 100 | 90 | 100 | 100 | 100 | 90 | 90 | 100 | 100 |
| Corn | 30 | 30 | 30 | 20 | 80 | 70 | 30 | 30 | 90 | 80 | 80 | 70 | 80 | 80 |
| Foxtail, Giant | 80 | 80 | 70 | 80 | 90 | 90 | 90 | 70 | 90 | 90 | 90 | 90 | 90 | 90 |
| Foxtail, Green | — | — | — | — | — | — | — | — | — | — | — | — | — | — |
| Galium | 100 | 90 | 90 | 90 | 100 | 100 | 100 | 90 | 100 | 100 | 100 | 100 | 100 | 100 |
| Kochia | 80 | 80 | 50 | 60 | 80 | 90 | 60 | 60 | 90 | 90 | 40 | 60 | 80 | 70 |
| Pigweed | 90 | 70 | 80 | 100 | 100 | 100 | 100 | 90 | 100 | 100 | 90 | 90 | 100 | 100 |
| Ragweed | 90 | 90 | 90 | 90 | 100 | 90 | 90 | 80 | 100 | 100 | 100 | 100 | 100 | 100 |
| Ryegrass, | 100 | 90 | 100 | 90 | 100 | 100 | 80 | 90 | 100 | 90 | 90 | 90 | 100 | 90 |
| Italian | | | | | | | | | | | | | | |
| Wheat | 80 | 80 | 50 | 40 | 80 | 90 | 0 | 60 | 70 | 60 | 50 | 40 | 60 | 60 |
| 500 g ai/ha | Compounds | | | | | | | | | | | | | |
| Postemergence | 118 | 119 | 121 | 122 | 123 | 128 | 129 | 130 | 131 | 132 | 133 | 134 | 135 | 140 |
| Barnyardgrass | 90 | 80 | 70 | 60 | 40 | 30 | 50 | 30 | 50 | 60 | 0 | 20 | 0 | 20 |
| Blackgrass | 100 | 90 | 70 | 40 | 30 | 10 | 70 | 70 | 50 | 60 | 0 | 10 | 0 | 0 |
| Corn | 70 | 40 | 10 | 10 | 0 | 0 | 0 | 30 | 20 | 10 | 0 | 0 | 0 | 20 |
| Foxtail, Giant | 90 | 80 | 60 | 70 | 60 | — | 60 | 70 | 50 | 60 | 10 | 20 | 0 | 30 |
| Foxtail, Green | — | — | — | — | — | 30 | — | — | — | — | — | — | — | — |
| Galium | 100 | 100 | 90 | 80 | 80 | 90 | 90 | 90 | 80 | 100 | 60 | 60 | 40 | 70 |
| Kochia | 100 | 70 | 60 | 50 | 70 | 70 | 80 | 70 | 30 | 80 | 60 | 70 | 10 | 60 |
| Pigweed | 100 | 90 | 70 | 70 | 80 | 60 | 70 | 80 | 10 | 70 | 80 | 80 | 30 | 90 |
| Ragweed | 90 | 90 | 70 | 60 | 90 | 80 | 100 | 80 | 80 | 90 | 30 | 70 | 20 | 70 |
| Ryegrass, | 100 | 90 | 90 | 90 | 80 | 10 | 10 | 90 | 10 | 20 | 0 | 80 | 30 | 60 |
| Italian | | | | | | | | | | | | | | |
| Wheat | 90 | 0 | 40 | 30 | 10 | 10 | 0 | 40 | 0 | 0 | 0 | 0 | 0 | 0 |

| 500 g ai/ha | Compounds | | | | | | | |
|---|---|---|---|---|---|---|---|---|
| Postemergence | 141 | 142 | 143 | 144 | 145 | 146 | 147 | 148 |
| Barnyardgrass | 0 | 10 | 40 | 0 | 60 | 60 | 60 | 0 |
| Blackgrass | 30 | 80 | 40 | 0 | 70 | 60 | 60 | 0 |
| Corn | 0 | 30 | 20 | 0 | 0 | 20 | 30 | 0 |
| Foxtail, Giant | 0 | 30 | — | 0 | 60 | 70 | 70 | — |
| Foxtail, Green | — | — | 40 | — | — | — | — | 0 |
| Galium | 10 | 70 | 0 | 30 | 100 | 90 | 90 | 0 |
| Kochia | 0 | 30 | 20 | 0 | 90 | 80 | 90 | 0 |
| Pigweed | 0 | 10 | 20 | 10 | 80 | 90 | 90 | 0 |
| Ragweed | 0 | 30 | 0 | 30 | 100 | 100 | 90 | 0 |
| Ryegrass, | 0 | 70 | 60 | 0 | 10 | 0 | 0 | 0 |
| Italian | | | | | | | | |
| Wheat | 0 | 10 | 20 | 0 | 0 | 0 | 0 | 0 |

| 125 g ai/ha | Compounds | | | | | | | | | | | | | |
|---|---|---|---|---|---|---|---|---|---|---|---|---|---|---|
| Postemergence | 1 | 2 | 3 | 4 | 5 | 8 | 9 | 10 | 11 | 12 | 13 | 14 | 15 | 16 |
| Barnyardgrass | 0 | 0 | 0 | 0 | 10 | 0 | 0 | 0 | 10 | 0 | 10 | 20 | 10 | 0 |
| Blackgrass | 0 | 0 | 10 | 0 | 10 | 0 | 0 | 10 | 30 | 0 | 10 | 30 | 30 | 0 |
| Corn | 0 | 0 | 0 | 0 | 0 | 0 | 0 | 0 | 0 | 0 | 0 | 0 | 0 | 0 |
| Foxtail, Giant | — | — | 10 | 0 | 0 | — | — | — | — | — | — | — | — | — |
| Foxtail, Green | 0 | 0 | — | — | — | 10 | 10 | 0 | 10 | 0 | 10 | 20 | 10 | 0 |
| Galium | 0 | 10 | 30 | 0 | 0 | 80 | 80 | — | — | 30 | 90 | 90 | 80 | 0 |
| Kochia | 0 | 10 | 10 | 0 | 0 | 10 | 10 | 70 | 70 | 0 | 20 | 20 | 0 | 0 |
| Pigweed | 0 | 0 | 20 | 0 | 0 | 0 | 0 | 20 | 40 | 0 | 40 | 20 | 20 | 0 |
| Ragweed | 0 | 10 | 20 | 0 | 0 | 20 | 30 | 90 | 80 | 0 | 70 | 60 | 60 | 0 |
| Ryegrass, | 0 | 20 | 20 | 0 | 0 | 30 | 40 | 60 | 60 | 0 | 80 | 60 | 60 | 0 |
| Italian | | | | | | | | | | | | | | |
| Wheat | 0 | 10 | 0 | 0 | 0 | 0 | 0 | 0 | 0 | 0 | 0 | 0 | 0 | 0 |
| 125 g ai/ha | Compounds | | | | | | | | | | | | | |
| Postemergence | 19 | 20 | 21 | 22 | 23 | 24 | 25 | 26 | 28 | 29 | 30 | 31 | 32 | 33 |
| Barnyardgrass | 20 | 30 | 0 | 0 | 0 | 0 | 0 | 50 | 0 | 0 | 20 | 40 | 10 | 0 |
| Blackgrass | 10 | 20 | 0 | 10 | 20 | 10 | 0 | 80 | 0 | 20 | 10 | 80 | 0 | 0 |
| Corn | 0 | 0 | 0 | 0 | 0 | 0 | 0 | 0 | 0 | 0 | 0 | 20 | 0 | 0 |
| Foxtail, Giant | — | — | — | — | — | — | — | — | — | — | — | — | 10 | 0 |
| Foxtail, Green | 10 | 30 | 0 | 0 | 0 | 0 | 0 | 60 | 0 | 0 | 0 | 50 | — | — |
| Galium | 80 | 80 | 0 | 60 | 50 | 50 | 20 | 90 | 20 | 70 | 60 | 90 | 70 | 40 |
| Kochia | 10 | 0 | 0 | 20 | 10 | 0 | 0 | 80 | 0 | 10 | 10 | 70 | 20 | 20 |
| Pigweed | 70 | 60 | 0 | 30 | 20 | 30 | 40 | 60 | 30 | 40 | 30 | 60 | 40 | 10 |
| Ragweed | 70 | 60 | 0 | 40 | 40 | 30 | 20 | 90 | 0 | 60 | 40 | 70 | 50 | 10 |
| Ryegrass, | 20 | 20 | 0 | 10 | 30 | 10 | 10 | 70 | 0 | 30 | 40 | 60 | 40 | 10 |
| Italian | | | | | | | | | | | | | | |
| Wheat | 0 | 0 | 0 | 0 | 0 | 0 | 0 | 0 | 0 | 20 | 10 | 0 | 0 | 0 |
| 125 g ai/ha | Compounds | | | | | | | | | | | | | |
| Postemergence | 34 | 35 | 36 | 37 | 38 | 39 | 40 | 41 | 42 | 43 | 44 | 45 | 46 | 47 |
| Barnyardgrass | 0 | 0 | 0 | 10 | 20 | 0 | 30 | 0 | 0 | 0 | 0 | 0 | 0 | 30 |
| Blackgrass | 0 | 0 | 10 | 60 | 10 | 0 | 70 | 0 | 0 | 0 | 0 | 0 | 0 | 20 |
| Corn | 0 | 0 | 20 | 0 | 10 | 0 | 0 | 0 | 0 | 0 | 0 | 0 | 0 | 0 |
| Foxtail, Giant | 0 | 0 | 0 | 30 | 10 | 0 | 10 | 0 | 0 | 0 | 0 | 0 | 0 | 30 |
| Foxtail, Green | — | — | — | — | — | — | — | — | — | — | — | — | — | — |
| Galium | 40 | 20 | 50 | 80 | 80 | 40 | 70 | 70 | 70 | 20 | 20 | 50 | 30 | 70 |
| Kochia | 10 | 0 | 60 | 60 | 10 | 0 | 10 | 20 | 20 | 0 | 0 | 0 | 0 | 10 |
| Pigweed | 20 | 10 | 20 | 70 | 20 | 0 | 10 | 40 | 60 | 20 | 10 | 0 | 10 | 60 |
| Ragweed | 40 | 10 | 70 | 60 | 60 | 0 | 70 | 30 | 40 | 20 | 0 | 30 | 40 | 80 |
| Ryegrass, | 20 | 0 | 30 | 80 | 10 | 0 | 50 | 0 | 50 | 0 | 20 | 0 | 10 | 0 |
| Italian | | | | | | | | | | | | | | |
| Wheat | 20 | 0 | 0 | 0 | 0 | 0 | 20 | 0 | 0 | 0 | 0 | 0 | 0 | 0 |
| 125 g ai/ha | Compounds | | | | | | | | | | | | | |
| Postemergence | 48 | 49 | 50 | 51 | 52 | 53 | 54 | 55 | 56 | 57 | 58 | 59 | 60 | 61 |
| Barnyardgrass | 20 | 20 | 10 | 30 | 10 | 10 | 30 | 20 | 0 | 0 | 20 | 30 | 0 | 0 |
| Blackgrass | 70 | 70 | 0 | 90 | 0 | 10 | 40 | 70 | 30 | 0 | 80 | 70 | 0 | 10 |
| Corn | 10 | 0 | 0 | 10 | 0 | 0 | 10 | 0 | 0 | 20 | 0 | 0 | 0 | 0 |
| Foxtail, Giant | 30 | 40 | 20 | 50 | 10 | 10 | 40 | 20 | 0 | 0 | 40 | 40 | 0 | 0 |
| Foxtail, Green | — | — | — | — | — | — | — | — | — | — | — | — | — | — |
| Galium | 80 | 80 | 70 | 100 | 70 | 0 | 70 | 50 | 70 | 10 | 80 | 70 | 60 | 60 |
| Kochia | 70 | 70 | 60 | 80 | 20 | 0 | 50 | 10 | 20 | 0 | 60 | 80 | 30 | 30 |
| Pigweed | 80 | 80 | 90 | 90 | 70 | 0 | 60 | 0 | 50 | 30 | 70 | 80 | 50 | 50 |
| Ragweed | 80 | 80 | 70 | 80 | 60 | 0 | 60 | 0 | 10 | 0 | 50 | 60 | 20 | 10 |
| Ryegrass, | 70 | 40 | 20 | 90 | 10 | 20 | 40 | 0 | 20 | 10 | 80 | 80 | 10 | 10 |
| Italian | | | | | | | | | | | | | | |
| Wheat | 0 | 0 | 0 | 70 | 0 | 0 | 30 | 0 | 0 | 0 | 40 | 0 | — | 30 |
| 125 g ai/ha | Compounds | | | | | | | | | | | | | |
| Postemergence | 62 | 63 | 64 | 65 | 66 | 67 | 68 | 69 | 70 | 71 | 72 | 73 | 74 | 75 |
| Barnyardgrass | 0 | 0 | 20 | 30 | 10 | 0 | 0 | 20 | 0 | 10 | 0 | 20 | 0 | 20 |
| Blackgrass | 10 | 10 | 0 | 10 | 0 | 0 | 30 | 10 | 0 | 20 | 20 | 30 | 0 | 90 |
| Corn | 0 | 0 | 0 | 0 | 0 | 0 | 0 | 0 | 0 | 0 | 0 | 0 | 0 | 10 |
| Foxtail, Giant | 0 | — | 0 | 30 | — | 0 | 0 | 10 | 0 | 10 | 0 | 30 | 0 | 50 |
| Foxtail, Green | — | 0 | — | — | 0 | — | — | — | — | — | — | — | — | — |
| Galium | 20 | 60 | 70 | 70 | 60 | 70 | 60 | 70 | 70 | 60 | 70 | 70 | 10 | 100 |
| Kochia | 0 | 10 | 20 | 70 | 20 | 20 | 70 | 50 | 70 | 50 | 20 | 60 | 0 | 70 |
| Pigweed | 10 | 30 | 20 | 90 | 50 | 0 | 30 | 40 | 80 | 60 | 30 | 80 | 20 | 70 |
| Ragweed | 20 | 30 | 20 | 60 | 40 | 50 | 70 | 70 | 80 | 20 | 40 | 50 | 0 | 90 |
| Ryegrass, | 0 | 30 | 50 | 50 | 30 | 10 | 20 | 80 | 0 | 40 | 0 | 40 | 10 | 90 |
| Italian | | | | | | | | | | | | | | |
| Wheat | 0 | 0 | 0 | 0 | 0 | 0 | 0 | 0 | 0 | 20 | 0 | 20 | 0 | 60 |
| 125 g ai/ha | Compounds | | | | | | | | | | | | | |
| Postemergence | 76 | 77 | 78 | 79 | 80 | 81 | 82 | 83 | 84 | 85 | 86 | 87 | 88 | 89 |
| Barnyardgrass | 20 | 0 | 40 | 40 | 50 | 40 | 50 | 60 | 60 | 50 | 50 | 60 | 50 | 40 |
| Blackgrass | 0 | 0 | 70 | 70 | 70 | 70 | 90 | 90 | 90 | 90 | 90 | 80 | 80 | 90 |
| Corn | 0 | 0 | 20 | 10 | 10 | 20 | 0 | 20 | 20 | 10 | 0 | 0 | 10 | 20 |
| Foxtail, Giant | 0 | 0 | 70 | 60 | 60 | 60 | 70 | 70 | 70 | 70 | 70 | 70 | 80 | 70 |
| Foxtail, Green | — | — | — | — | — | — | — | — | — | — | — | — | — | — |
| Galium | 40 | 40 | 90 | 90 | 80 | 80 | 90 | 90 | 90 | 80 | 70 | 80 | 90 | 90 |
| Kochia | 0 | 0 | 60 | 70 | 30 | 40 | 80 | 80 | 70 | 70 | 40 | 40 | 60 | 70 |
| Pigweed | 40 | 30 | 80 | 80 | 70 | 60 | 70 | 80 | 80 | 80 | 70 | 70 | 90 | 80 |
| Ragweed | 10 | 0 | 80 | 90 | 80 | 80 | 90 | 90 | 90 | 90 | 100 | 100 | 100 | 90 |
| Ryegrass, | 20 | 0 | 30 | 30 | 20 | 30 | 80 | 90 | 90 | 90 | 90 | 80 | 100 | 100 |
| Italian | | | | | | | | | | | | | | |
| Wheat | 0 | 0 | 0 | 0 | 0 | 0 | 50 | 40 | 40 | 40 | 50 | 40 | 10 | 50 |
| 125 g ai/ha | Compounds | | | | | | | | | | | | | |
| Postemergence | 90 | 91 | 92 | 93 | 94 | 95 | 96 | 97 | 98 | 99 | 100 | 101 | 102 | 103 |
| Barnyardgrass | 20 | 80 | 20 | 10 | 20 | 50 | 20 | 40 | 50 | 80 | 30 | 30 | 20 | 20 |
| Blackgrass | 80 | 100 | 20 | 0 | 20 | 60 | 90 | 70 | 80 | 90 | 90 | 0 | 0 | 20 |
| Corn | 20 | 30 | 0 | 10 | 10 | 0 | 10 | 0 | 0 | 30 | 10 | 0 | 0 | 0 |
| Foxtail, Giant | 30 | 80 | 50 | 30 | 40 | 60 | 20 | 70 | 40 | 80 | 70 | 0 | 10 | 0 |
| Foxtail, Green | — | — | — | — | — | — | — | — | — | — | — | — | — | — |
| Galium | 90 | 100 | 70 | 60 | 60 | 80 | 90 | 70 | 80 | 90 | 90 | 80 | 40 | 40 |
| Kochia | 60 | 60 | 30 | 0 | 20 | 60 | 60 | 60 | 60 | 50 | 70 | 0 | 20 | 0 |
| Pigweed | 60 | 80 | 60 | 50 | 50 | 70 | 80 | 80 | 70 | 60 | 90 | 10 | 20 | 10 |
| Ragweed | 70 | 100 | 60 | 10 | 50 | 80 | 90 | 70 | 80 | 90 | 90 | 40 | 10 | 10 |
| Ryegrass, | 80 | 100 | 50 | 40 | 30 | 80 | 80 | 90 | 80 | 90 | 80 | 0 | 0 | 0 |
| Italian | | | | | | | | | | | | | | |
| Wheat | 30 | 50 | 0 | 0 | 0 | 40 | 0 | 30 | 0 | 50 | 0 | 0 | 0 | 0 |
| 125 g ai/ha | Compounds | | | | | | | | | | | | | |
| Postemergence | 104 | 105 | 106 | 107 | 108 | 109 | 110 | 111 | 112 | 113 | 114 | 115 | 116 | 117 |
| Barnyardgrass | 40 | 40 | 80 | 80 | 90 | 90 | 50 | 50 | 80 | 90 | 80 | 80 | 90 | 90 |
| Blackgrass | 80 | 80 | 80 | 80 | 100 | 90 | 90 | 80 | 100 | 90 | 80 | 80 | 100 | 100 |
| Corn | 30 | 10 | 20 | 0 | 30 | 20 | 10 | 20 | 20 | 40 | 20 | 20 | 30 | 40 |
| Foxtail, Giant | 70 | 70 | 60 | 70 | 80 | 80 | 50 | 60 | 90 | 90 | 90 | 90 | 90 | 90 |
| Foxtail, Green | — | — | — | — | — | — | — | — | — | — | — | — | — | — |
| Galium | 90 | 90 | 80 | 70 | 100 | 100 | 90 | 80 | 100 | 100 | 100 | 100 | 100 | 100 |
| Kochia | 70 | 70 | 20 | 30 | 60 | 70 | 50 | 40 | 70 | 60 | 30 | 30 | 60 | 50 |
| Pigweed | 70 | 60 | 70 | 80 | 90 | 90 | 90 | 60 | 90 | 100 | 70 | 60 | 90 | 80 |
| Ragweed | 80 | 90 | 70 | 80 | 80 | 80 | 60 | 80 | 100 | 90 | 90 | 90 | 100 | 100 |
| Ryegrass, | 90 | 90 | 70 | 70 | 100 | 100 | 30 | 70 | 90 | 80 | 80 | 80 | 90 | 80 |
| Italian | | | | | | | | | | | | | | |
| Wheat | 40 | 60 | 0 | 0 | 70 | 90 | 0 | 0 | 50 | 50 | 0 | 20 | 50 | 50 |
| 125 g ai/ha | Compounds | | | | | | | | | | | | | |
| Postemergence | 118 | 119 | 120 | 121 | 122 | 123 | 128 | 129 | 130 | 131 | 132 | 133 | 134 | 135 |
| Barnyardgrass | 90 | 70 | 20 | 0 | 20 | 0 | 10 | 30 | 30 | 20 | 20 | 0 | 10 | 0 |
| Blackgrass | 90 | 80 | 20 | 30 | 20 | 20 | 0 | 30 | 50 | 30 | 40 | 0 | 0 | 0 |
| Corn | 30 | 10 | 0 | 0 | 0 | 0 | 0 | 0 | 10 | 0 | 10 | 0 | 0 | 0 |
| Foxtail, Giant | 80 | 70 | 40 | 20 | 50 | 20 | — | 40 | 30 | 10 | 40 | 0 | 10 | 0 |
| Foxtail, Green | — | — | — | — | — | — | 20 | — | — | — | — | — | — | — |
| Galium | 90 | 80 | 80 | 60 | 60 | 70 | 70 | 80 | 80 | 80 | 90 | 20 | 60 | 30 |
| Kochia | 70 | 70 | 30 | 40 | 40 | 50 | 50 | 70 | 50 | 10 | 60 | 20 | 20 | 0 |
| Pigweed | 100 | 80 | 50 | 50 | 60 | 70 | 30 | 40 | 70 | 0 | 40 | 50 | 40 | 20 |
| Ragweed | 90 | 90 | 20 | 40 | 50 | 80 | 50 | 90 | 60 | 80 | 90 | 10 | 60 | 0 |
| Ryegrass, | 90 | 80 | 30 | 60 | 70 | 60 | 0 | 0 | 30 | 0 | 10 | 0 | 20 | 0 |
| Italian | | | | | | | | | | | | | | |
| Wheat | 40 | 0 | 0 | 30 | 0 | 0 | 0 | 0 | 10 | 0 | 0 | 0 | 0 | 0 |

| 125 g ai/ha | Compounds | | | | | | | | |
|---|---|---|---|---|---|---|---|---|---|
| Postemergence | 140 | 141 | 142 | 143 | 144 | 145 | 146 | 147 | 148 |
| Barnyardgrass | 0 | 0 | 10 | 10 | 0 | 30 | 30 | 20 | 0 |
| Blackgrass | 0 | 10 | 30 | 10 | 0 | 30 | 30 | 30 | 0 |
| Corn | 10 | 0 | 0 | 0 | 0 | 0 | 30 | 0 | 0 |
| Foxtail, Giant | 10 | 0 | 20 | — | 0 | 30 | 60 | 30 | — |
| Foxtail, Green | — | — | — | 20 | — | — | — | — | 0 |
| Galium | 70 | 0 | 50 | 0 | 0 | 90 | 70 | 90 | 0 |
| Kochia | 20 | 0 | 0 | 0 | 0 | 80 | 60 | 40 | 0 |
| Pigweed | 80 | 0 | 10 | 0 | 0 | 30 | 70 | 40 | 0 |
| Ragweed | 50 | 0 | 0 | 0 | 0 | 100 | 90 | 100 | 0 |
| Ryegrass, | 10 | 0 | 0 | 20 | 0 | 20 | 0 | 0 | 0 |
| Italian | | | | | | | | | |
| Wheat | 0 | 0 | 0 | 0 | 0 | 0 | 0 | 0 | 0 |

| 31 g ai/ha | Compound | 1000 g ai/ha | Compounds | |
|---|---|---|---|---|
| Postemergence | 120 | Postemergence | 6 | 18 |
| Barnyardgrass | 0 | Barnyardgrass | 30 | 10 |
| Blackgrass | 0 | Blackgrass | 10 | 10 |
| Corn | 0 | Corn | 0 | 0 |
| Foxtail, Giant | 0 | Foxtail, Giant | 50 | 40 |
| Galium | 40 | Galium | 90 | 90 |
| Kochia | 10 | Kochia | 80 | 30 |
| Pigweed | 30 | Pigweed | 80 | 90 |
| Ragweed | 0 | Ragweed | 90 | 100 |
| Ryegrass, Italian | 0 | Ryegrass, Italian | 70 | 60 |
| Wheat | 0 | Wheat | 0 | 20 |

| 500 g ai/ha | Compounds | | | | | | | | | | | | | |
|---|---|---|---|---|---|---|---|---|---|---|---|---|---|---|
| Preemergence | 1 | 2 | 3 | 4 | 5 | 8 | 9 | 10 | 11 | 12 | 13 | 14 | 15 | 16 |
| Barnyardgrass | 0 | 50 | 20 | 70 | 70 | 20 | 50 | 10 | 40 | 50 | 80 | 80 | 80 | 0 |
| Foxtail, Giant | — | — | 10 | 50 | 10 | — | — | — | — | — | — | — | — | — |
| Foxtail, Green | 30 | 50 | — | — | — | 30 | 40 | 20 | 70 | 20 | 20 | 70 | 60 | 0 |
| Kochia | 0 | 0 | 10 | 0 | 0 | 60 | 60 | 90 | 80 | 0 | 30 | 20 | 10 | 0 |
| Pigweed | 0 | 0 | 10 | 0 | 0 | 90 | 90 | 100 | 100 | 70 | 80 | 50 | 70 | 0 |
| Ragweed | 10 | 0 | 10 | 0 | 0 | 100 | 90 | 90 | 50 | 30 | 100 | 80 | 90 | 20 |
| Ryegrass, | 40 | 60 | 70 | 30 | 20 | 50 | 40 | 90 | 80 | 70 | 100 | 90 | 80 | 50 |
| Italian | | | | | | | | | | | | | | |
| 500 g ai/ha | Compounds | | | | | | | | | | | | | |
| Preemergence | 19 | 20 | 21 | 22 | 23 | 24 | 25 | 26 | 27 | 28 | 29 | 30 | 31 | 32 |
| Barnyardgrass | 60 | 70 | 0 | 0 | 20 | 10 | 0 | 80 | 10 | 0 | 50 | 50 | 70 | 0 |
| Foxtail, Giant | — | — | — | — | — | — | — | — | — | — | — | — | — | 0 |
| Foxtail, Green | 40 | 60 | 0 | 0 | 50 | 60 | 60 | 100 | 0 | 30 | 50 | 60 | 90 | — |
| Kochia | 0 | 0 | 0 | 10 | 20 | 0 | 0 | 80 | 0 | 0 | 10 | 30 | 90 | 10 |
| Pigweed | 80 | 100 | 0 | 90 | 80 | 40 | 40 | 100 | 0 | 10 | 20 | 80 | 90 | 50 |
| Ragweed | 80 | 90 | 0 | 90 | 90 | 60 | 90 | 100 | 10 | 10 | 90 | 70 | 80 | 40 |
| Ryegrass, | 50 | 70 | 0 | 30 | 90 | 50 | 30 | 100 | 10 | 0 | 70 | 50 | 70 | 50 |
| Italian | | | | | | | | | | | | | | |
| 500 g ai/ha | Compounds | | | | | | | | | | | | | |
| Preemergence | 33 | 34 | 35 | 36 | 37 | 38 | 39 | 40 | 41 | 42 | 43 | 44 | 45 | 46 |
| Barnyardgrass | 0 | 0 | 0 | 20 | 20 | 0 | 0 | 60 | 10 | 0 | 0 | 10 | 0 | 0 |
| Foxtail, Giant | 0 | 40 | 0 | 50 | 80 | 10 | 0 | 90 | 0 | 0 | 0 | 10 | 0 | 0 |
| Foxtail, Green | — | — | — | — | — | — | — | — | — | — | — | — | — | — |
| Kochia | 10 | 0 | 0 | 50 | 80 | 20 | 0 | 10 | 0 | 10 | 0 | 0 | 50 | 30 |
| Pigweed | 70 | 10 | 0 | 30 | 90 | 0 | 0 | 60 | 80 | 20 | 30 | 0 | 0 | 10 |
| Ragweed | 20 | 50 | 10 | 70 | 80 | 30 | 30 | 80 | 80 | 80 | 20 | 0 | 40 | 50 |
| Ryegrass, | 60 | 60 | 10 | 50 | 60 | 0 | 0 | 60 | 20 | 20 | 0 | 50 | 10 | 0 |
| Italian | | | | | | | | | | | | | | |
| 500 g ai/ha | Compounds | | | | | | | | | | | | | |
| Preemergence | 47 | 48 | 49 | 50 | 51 | 52 | 53 | 55 | 56 | 57 | 58 | 59 | 60 | 61 |
| Barnyardgrass | 50 | 70 | 90 | 60 | 80 | 10 | 0 | 50 | 50 | 0 | 60 | 30 | 0 | 0 |
| Foxtail, Giant | 90 | 90 | 80 | 40 | 100 | 10 | 0 | 90 | 40 | 30 | 100 | 70 | 10 | 10 |
| Foxtail, Green | — | — | — | — | — | — | — | — | — | — | — | — | — | — |
| Kochia | 90 | 100 | 70 | 70 | 80 | 20 | 0 | 0 | 60 | 10 | 60 | 60 | 0 | 0 |
| Pigweed | 80 | 100 | 100 | 90 | 90 | 80 | 0 | 0 | 90 | 0 | 50 | 70 | 30 | 0 |
| Ragweed | 100 | 100 | 100 | 90 | 100 | 30 | 0 | 80 | 50 | 0 | 80 | 100 | 30 | 70 |
| Ryegrass, | 70 | 70 | 70 | 30 | 100 | 40 | 0 | 30 | 40 | 0 | 100 | 90 | 40 | 80 |
| Italian | | | | | | | | | | | | | | |
| 500 g ai/ha | Compounds | | | | | | | | | | | | | |
| Preemergence | 62 | 63 | 64 | 65 | 66 | 67 | 68 | 69 | 70 | 71 | 72 | 73 | 74 | 75 |
| Barnyardgrass | 0 | 0 | 0 | 50 | 20 | 0 | 0 | 10 | 10 | 0 | 0 | 80 | 0 | 90 |
| Foxtail, Giant | 0 | — | 0 | 40 | — | 0 | 20 | 30 | 10 | 0 | 10 | 80 | 0 | 100 |
| Foxtail, Green | — | 20 | — | — | 60 | — | — | — | — | — | — | — | — | — |
| Kochia | 0 | 20 | 0 | 30 | 20 | 10 | 40 | 10 | 50 | 0 | 0 | 20 | 0 | 90 |
| Pigweed | 0 | 80 | 10 | 100 | 80 | 0 | 80 | 70 | 40 | 60 | 20 | 100 | 0 | 90 |
| Ragweed | 30 | 70 | 20 | 100 | 50 | 10 | 50 | 40 | 80 | 30 | 20 | 90 | 0 | 100 |
| Ryegrass, | 40 | 50 | 60 | 30 | 50 | 10 | 60 | 80 | 10 | 20 | 40 | 50 | 0 | 100 |
| Italian | | | | | | | | | | | | | | |
| 500 g ai/ha | Compounds | | | | | | | | | | | | | |
| Preemergence | 76 | 77 | 78 | 79 | 80 | 81 | 82 | 83 | 84 | 85 | 86 | 87 | 88 | 89 |
| Barnyardgrass | 0 | 0 | 80 | 80 | 80 | 70 | 100 | 100 | 100 | 90 | 90 | 90 | 90 | 100 |
| Foxtail, Giant | 0 | 0 | 90 | 90 | 100 | 100 | 100 | 100 | 100 | 100 | 100 | 100 | 100 | 100 |
| Foxtail, Green | — | — | — | — | — | — | — | — | — | — | — | — | — | — |
| Kochia | 0 | 0 | 30 | 70 | 10 | 10 | 80 | 100 | 60 | 50 | 30 | 30 | 90 | 90 |
| Pigweed | 10 | 0 | 100 | 100 | 100 | 100 | 100 | 100 | 100 | 100 | 100 | 100 | 100 | 100 |
| Ragweed | 0 | 30 | 90 | 100 | 100 | 100 | 100 | 100 | 100 | 100 | 100 | 90 | — | — |
| Ryegrass, | 0 | 0 | 50 | 60 | 30 | 50 | 100 | 100 | 100 | 100 | 100 | 100 | 100 | 100 |
| Italian | | | | | | | | | | | | | | |
| 500 g ai/ha | Compounds | | | | | | | | | | | | | |
| Preemergence | 90 | 91 | 92 | 93 | 94 | 95 | 96 | 97 | 98 | 99 | 100 | 101 | 102 | 103 |
| Barnyardgrass | 80 | 100 | 60 | 50 | 50 | 60 | 80 | 90 | 60 | 100 | 80 | 10 | 40 | 40 |
| Foxtail, Giant | 90 | 100 | 80 | 30 | 60 | 100 | 100 | 90 | 70 | 100 | 100 | 10 | 30 | 20 |
| Foxtail, Green | — | — | — | — | — | — | — | — | — | — | — | — | — | — |
| Kochia | 70 | 60 | 10 | 0 | 10 | 70 | 100 | 50 | 60 | 90 | 60 | 10 | 0 | 0 |
| Pigweed | 100 | 100 | 90 | 20 | 70 | 100 | 100 | 90 | 100 | 100 | 100 | 10 | 10 | 50 |
| Ragweed | — | — | 30 | 0 | 70 | 100 | 100 | 100 | — | — | 100 | 70 | 40 | 50 |
| Ryegrass, | 90 | 100 | 80 | 70 | 90 | 100 | 100 | 100 | 90 | 100 | 100 | 40 | 50 | 70 |
| Italian | | | | | | | | | | | | | | |
| 500 g ai/ha | Compounds | | | | | | | | | | | | | |
| Preemergence | 104 | 105 | 106 | 107 | 108 | 109 | 110 | 111 | 112 | 113 | 114 | 115 | 116 | 117 |
| Barnyardgrass | 80 | 80 | 90 | 90 | 100 | 100 | 80 | 70 | 100 | 100 | 100 | 100 | 100 | 100 |
| Foxtail, Giant | 100 | 90 | 100 | 100 | 100 | 100 | 100 | 90 | 100 | 100 | 100 | 100 | 100 | 100 |
| Foxtail, Green | — | — | — | — | — | — | — | — | — | — | — | — | — | — |
| Kochia | 70 | 70 | 20 | 30 | 100 | 50 | 10 | 100 | 80 | 90 | 20 | 10 | 100 | 70 |
| Pigweed | 70 | 70 | 80 | 80 | 100 | 100 | 100 | 100 | 100 | 100 | 80 | 90 | 100 | 100 |
| Ragweed | 80 | 60 | 80 | 80 | 100 | 100 | 90 | 100 | 90 | 90 | 90 | 90 | 90 | 90 |
| Ryegrass, | 100 | 100 | 90 | 90 | 100 | 100 | 70 | 70 | 100 | 100 | 100 | 90 | 100 | 100 |
| Italian | | | | | | | | | | | | | | |
| 500 g ai/ha | Compounds | | | | | | | | | | | | | |
| Preemergence | 118 | 119 | 121 | 122 | 123 | 128 | 129 | 130 | 131 | 132 | 133 | 134 | 135 | 140 |
| Barnyardgrass | 100 | 80 | 20 | 30 | 50 | 30 | 80 | 70 | 60 | 80 | 0 | 10 | 0 | 0 |
| Foxtail, Giant | 100 | 100 | 80 | 80 | 40 | — | 80 | 60 | 70 | 90 | 0 | 10 | 0 | 10 |
| Foxtail, Green | — | — | — | — | — | 10 | — | — | — | — | — | — | — | — |
| Kochia | 60 | 80 | 30 | 10 | 50 | 80 | 50 | 0 | 20 | 90 | 20 | 50 | 10 | 0 |
| Pigweed | 100 | 100 | 90 | 90 | 60 | 80 | 90 | 50 | 0 | 80 | 0 | 60 | 20 | 90 |
| Ragweed | 100 | 90 | 50 | 50 | 80 | 80 | 100 | 50 | 80 | — | 40 | 70 | 10 | 50 |
| Ryegrass, | 100 | 100 | 100 | 60 | 90 | 20 | 0 | 50 | 0 | 0 | 0 | 30 | 0 | 50 |
| Italian | | | | | | | | | | | | | | |

| 500 g ai/ha | Compounds | | | | | | | |
|---|---|---|---|---|---|---|---|---|
| Preemergence | 141 | 142 | 143 | 144 | 145 | 146 | 147 | 148 |
| Barnyardgrass | 0 | 50 | 30 | 0 | 80 | 60 | 60 | 0 |
| Foxtail, Giant | 0 | 60 | — | 0 | 80 | 80 | 80 | — |
| Foxtail, Green | — | — | 80 | — | — | — | — | 0 |
| Kochia | 0 | 0 | 10 | 0 | 90 | 100 | 100 | 0 |
| Pigweed | 0 | 0 | 0 | 0 | 90 | 100 | 100 | 0 |
| Ragweed | 0 | 20 | 0 | 0 | 100 | 100 | 100 | 0 |
| Ryegrass, | 20 | 20 | 50 | 0 | 10 | 0 | 0 | 0 |
| Italian | | | | | | | | |

| 125 g ai/ha | Compounds | | | | | | | | | | | | | |
|---|---|---|---|---|---|---|---|---|---|---|---|---|---|---|
| Preemergence | 1 | 2 | 3 | 4 | 5 | 8 | 9 | 10 | 11 | 12 | 13 | 14 | 15 | 16 |
| Barnyardgrass | 0 | 0 | 0 | 20 | 20 | 0 | 0 | 0 | 0 | 0 | 0 | 40 | 30 | 0 |
| Foxtail, Giant | — | — | 0 | 0 | 0 | — | — | — | — | — | — | — | — | — |
| Foxtail, Green | 10 | 0 | — | — | — | 10 | 20 | 20 | 0 | 0 | 0 | 20 | 0 | 0 |
| Kochia | 0 | 0 | 10 | 0 | 0 | 10 | 10 | 30 | 10 | 0 | 30 | 0 | 0 | 0 |
| Pigweed | 0 | 0 | 0 | 0 | 0 | 30 | 40 | 20 | 40 | 0 | 90 | 0 | 60 | 0 |
| Ragweed | 0 | 0 | 10 | 0 | 0 | 0 | 80 | 70 | 50 | 60 | 30 | 80 | 30 | 0 |
| Ryegrass, | 0 | 20 | 30 | 0 | 0 | 50 | 20 | 30 | 60 | 0 | 50 | 20 | 70 | 0 |
| Italian | | | | | | | | | | | | | | |
| 125 g ai/ha | Compounds | | | | | | | | | | | | | |
| Preemergence | 19 | 20 | 21 | 22 | 23 | 24 | 25 | 26 | 28 | 29 | 30 | 31 | 32 | 33 |
| Barnyardgrass | 50 | 20 | 0 | 0 | 0 | 0 | 0 | 40 | 0 | 30 | 0 | 10 | 0 | 0 |
| Foxtail, Giant | — | — | — | — | — | — | — | — | — | — | — | — | 0 | 0 |
| Foxtail, Green | 0 | 0 | 0 | 0 | 0 | 20 | 0 | 70 | 0 | 30 | 0 | 30 | — | — |
| Kochia | 0 | 0 | 0 | 10 | 0 | 0 | 0 | 50 | 0 | 0 | 0 | 10 | 0 | 0 |
| Pigweed | 0 | 80 | 0 | 30 | 30 | 20 | 90 | 100 | 0 | 0 | 30 | 40 | 0 | 0 |
| Ragweed | 50 | 30 | 0 | 50 | 60 | 30 | 30 | 90 | 0 | 30 | 20 | 30 | 20 | 0 |
| Ryegrass, | 0 | 30 | 0 | 10 | 40 | 10 | 10 | 70 | 0 | 20 | 20 | 40 | 20 | 20 |
| Italian | | | | | | | | | | | | | | |
| 125 g ai/ha | Compounds | | | | | | | | | | | | | |
| Preemergence | 34 | 35 | 36 | 37 | 38 | 39 | 40 | 41 | 42 | 43 | 44 | 45 | 46 | 47 |
| Barnyardgrass | 0 | 0 | 10 | 0 | 0 | 0 | 30 | 0 | 0 | 0 | 0 | 0 | 0 | 0 |
| Foxtail, Giant | 0 | 0 | 0 | 30 | 0 | 0 | 50 | 0 | 0 | 0 | 0 | 0 | 0 | 20 |
| Foxtail, Green | — | — | — | — | — | — | — | — | — | — | — | — | — | — |
| Kochia | 0 | 0 | 10 | 0 | 0 | 0 | 0 | 0 | 0 | 0 | 0 | 0 | 0 | 0 |
| Pigweed | 0 | 0 | 10 | 40 | 0 | 0 | 30 | 0 | 0 | 0 | 0 | 0 | 0 | 20 |
| Ragweed | 20 | 0 | 10 | 10 | 30 | 0 | 40 | 0 | 60 | 0 | 0 | 10 | 20 | 100 |
| Ryegrass, | 10 | 0 | 30 | 40 | 0 | 0 | 20 | 0 | 0 | 0 | 0 | 0 | 0 | 10 |
| Italian | | | | | | | | | | | | | | |
| 125 g ai/ha | Compounds | | | | | | | | | | | | | |
| Preemergence | 48 | 49 | 50 | 51 | 52 | 53 | 54 | 55 | 56 | 57 | 58 | 59 | 60 | 61 |
| Barnyardgrass | 20 | 30 | 10 | 60 | 0 | 0 | 40 | 0 | 0 | 0 | 10 | 0 | 0 | 0 |
| Foxtail, Giant | 90 | 50 | 0 | 70 | 0 | 0 | 40 | 10 | 0 | 0 | 60 | 30 | 10 | 0 |
| Foxtail, Green | — | — | — | — | — | — | — | — | — | — | — | — | — | — |
| Kochia | 40 | 50 | 10 | 10 | 0 | 0 | 0 | 0 | 0 | 0 | 30 | 10 | 0 | 0 |
| Pigweed | 30 | 90 | 30 | 90 | 60 | 0 | 80 | 0 | 30 | 0 | 10 | 0 | 0 | 0 |
| Ragweed | 100 | 100 | 50 | 90 | 0 | 0 | 70 | 10 | 70 | 0 | 60 | 30 | 0 | 0 |
| Ryegrass, | 40 | 40 | 30 | 70 | 0 | 0 | 70 | 0 | 0 | 0 | 70 | 30 | 0 | 30 |
| Italian | | | | | | | | | | | | | | |
| 125 g ai/ha | Compounds | | | | | | | | | | | | | |
| Preemergence | 62 | 63 | 64 | 65 | 66 | 67 | 68 | 69 | 70 | 71 | 72 | 73 | 74 | 75 |
| Barnyardgrass | 0 | 0 | 0 | 20 | 0 | 0 | 0 | 0 | 0 | 0 | 0 | 20 | 0 | 30 |
| Foxtail, Giant | 0 | — | 0 | 20 | — | 0 | 0 | 10 | 0 | 0 | 0 | 20 | 0 | 70 |
| Foxtail, Green | — | 0 | — | — | 30 | — | — | — | — | — | — | — | — | — |
| Kochia | 0 | 0 | 0 | 0 | 0 | 0 | 10 | 0 | 0 | 0 | 0 | 0 | 0 | 10 |
| Pigweed | 0 | 0 | 0 | 50 | 30 | 0 | 50 | 10 | 0 | 20 | 0 | 70 | 0 | 60 |
| Ragweed | 0 | 30 | 10 | 20 | 20 | 10 | 20 | 10 | 70 | 10 | 0 | 30 | 0 | 80 |
| Ryegrass, | 10 | 10 | 30 | 0 | 30 | 0 | 30 | 10 | 0 | 0 | 0 | 20 | 0 | 100 |
| Italian | | | | | | | | | | | | | | |
| 125 g ai/ha | Compounds | | | | | | | | | | | | | |
| Preemergence | 76 | 77 | 78 | 79 | 80 | 81 | 82 | 83 | 84 | 85 | 86 | 87 | 88 | 89 |
| Barnyardgrass | 0 | 0 | 80 | 70 | 60 | 60 | 60 | 80 | 60 | 70 | 90 | 70 | 70 | 70 |
| Foxtail, Giant | 0 | 0 | 80 | 60 | 60 | 60 | 100 | 90 | 100 | 80 | 100 | 90 | 90 | 90 |
| Foxtail, Green | — | — | — | — | — | — | — | — | — | — | — | — | — | — |
| Kochia | 0 | 0 | 10 | 10 | 0 | 10 | 10 | 20 | 20 | 10 | 20 | 0 | 10 | 60 |
| Pigweed | 0 | 0 | 20 | 30 | 20 | 20 | 100 | 100 | 100 | 100 | 90 | 30 | 100 | 100 |
| Ragweed | 0 | 10 | 90 | 90 | 50 | 90 | 90 | 100 | 80 | 90 | 80 | 90 | — | — |
| Ryegrass, | 0 | 0 | 30 | 30 | 10 | 20 | 90 | 80 | 100 | 100 | 100 | 90 | 90 | 90 |
| Italian | | | | | | | | | | | | | | |
| 125 g ai/ha | Compounds | | | | | | | | | | | | | |
| Preemergence | 90 | 91 | 92 | 93 | 94 | 95 | 96 | 97 | 98 | 99 | 100 | 101 | 102 | 103 |
| Barnyardgrass | 60 | 100 | 30 | 0 | 10 | 60 | 60 | 70 | 50 | 90 | 50 | 0 | 0 | 0 |
| Foxtail, Giant | 50 | 100 | 70 | 10 | 20 | 80 | 70 | 80 | 60 | 100 | 60 | 0 | 0 | 0 |
| Foxtail, Green | — | — | — | — | — | — | — | — | — | — | — | — | — | — |
| Kochia | 50 | 60 | 0 | 0 | 0 | 10 | 20 | 20 | 60 | 70 | 10 | 0 | 0 | 0 |
| Pigweed | 90 | 100 | 30 | 0 | 10 | 50 | 80 | 40 | 90 | 90 | 90 | 0 | 0 | 0 |
| Ragweed | — | — | 20 | 0 | 10 | 30 | 90 | 60 | — | — | 100 | 20 | 20 | — |
| Ryegrass, | 60 | 80 | 40 | 20 | 50 | 90 | 30 | 80 | 80 | 90 | 50 | 0 | 20 | 30 |
| Italian | | | | | | | | | | | | | | |
| 125 g ai/ha | Compounds | | | | | | | | | | | | | |
| Preemergence | 104 | 105 | 106 | 107 | 108 | 109 | 110 | 111 | 112 | 113 | 114 | 115 | 116 | 117 |
| Barnyardgrass | 60 | 60 | 50 | 70 | 90 | 90 | 70 | 60 | 100 | 90 | 90 | 90 | 100 | 100 |
| Foxtail, Giant | 80 | 60 | 100 | 80 | 100 | 100 | 60 | 50 | 100 | 100 | 100 | 90 | 90 | 100 |
| Foxtail, Green | — | — | — | — | — | — | — | — | — | — | — | — | — | — |
| Kochia | 20 | 10 | 0 | 0 | 50 | 10 | 0 | 20 | 50 | 30 | 10 | — | 10 | 10 |
| Pigweed | 70 | 70 | 50 | 60 | 100 | 100 | 50 | 100 | 100 | 80 | 10 | 30 | 100 | 100 |
| Ragweed | 50 | 50 | 40 | 20 | 90 | 90 | 40 | 70 | 90 | 90 | 80 | 90 | 90 | 90 |
| Ryegrass, | 90 | 90 | 60 | 70 | 100 | 100 | 60 | 30 | 100 | 80 | 70 | 80 | 100 | 100 |
| Italian | | | | | | | | | | | | | | |
| 125 g ai/ha | Compounds | | | | | | | | | | | | | |
| Preemergence | 118 | 119 | 120 | 121 | 122 | 123 | 128 | 129 | 130 | 131 | 132 | 133 | 134 | 135 |
| Barnyardgrass | 60 | 50 | 10 | 0 | 0 | 0 | 0 | 60 | 0 | 0 | 70 | 0 | 0 | 0 |
| Foxtail, Giant | 80 | 80 | 50 | 10 | 10 | 10 | — | 60 | 30 | 60 | 40 | 0 | 0 | 0 |
| Foxtail, Green | — | — | — | — | — | — | 0 | — | — | — | — | — | — | — |
| Kochia | 40 | 30 | 10 | 0 | 0 | 10 | 30 | 10 | 0 | 0 | 90 | 0 | 10 | 0 |
| Pigweed | 100 | 80 | 100 | 10 | 20 | 40 | 100 | 10 | 50 | 0 | 80 | 0 | 20 | 0 |
| Ragweed | 80 | 90 | 10 | 10 | 10 | 50 | 60 | 70 | 20 | 60 | — | 0 | — | 0 |
| Ryegrass, | 100 | 30 | 10 | 50 | 50 | — | 0 | 0 | 30 | 0 | 0 | 0 | 10 | 0 |
| Italian | | | | | | | | | | | | | | |

| 125 g ai/ha | Compounds | | | | | | | | |
|---|---|---|---|---|---|---|---|---|---|
| Preemergence | 140 | 141 | 142 | 143 | 144 | 145 | 146 | 147 | 148 |
| Barnyardgrass | 0 | 0 | 0 | 0 | 0 | 60 | 60 | 50 | 0 |
| Foxtail, Giant | 0 | 0 | 10 | — | 0 | 50 | 70 | 50 | — |
| Foxtail, Green | — | — | — | 30 | — | — | — | — | 0 |
| Kochia | 0 | 0 | 0 | 0 | 0 | 70 | 30 | 100 | 0 |
| Pigweed | 0 | 0 | 0 | 0 | 0 | 50 | 80 | 60 | 0 |
| Ragweed | 10 | 0 | 0 | 0 | 0 | 100 | 90 | 90 | 0 |
| Ryegrass, | 20 | 0 | 0 | 20 | 0 | 0 | 0 | 0 | 0 |
| Italian | | | | | | | | | |

| 1000 g ai/ha | Compounds | | 31 g ai/ha | Compound |
|---|---|---|---|---|
| Preemergence | 6 | 18 | Preemergence | 120 |
| Barnyardgrass | 30 | 30 | Barnyardgrass | 0 |
| Foxtail, Giant | 30 | 20 | Foxtail, Giant | 20 |
| Kochia | 20 | 50 | Kochia | 0 |
| Pigweed | 70 | 100 | Pigweed | 60 |
| Ragweed | 50 | 80 | Ragweed | 0 |
| Ryegrass, Italian | 80 | 60 | Ryegrass, Italian | 0 |

### Test B

Plant species in the flooded paddy test selected from rice (Oryza sativa), sedge, umbrella (small-flower umbrella sedge, Cyperus difformis), ducksalad (Heteranhera limosa), and barnyardgrass (Echinochloa crus-galli) were grown to the 2-leaf stage for testing. At time of treatment, test pots were flooded to 3 cm above the soil surface treated by application of test compounds directly to the paddy water, and then maintained at that water depth for the duration of the test. Treated plants and controls were maintained in a greenhouse for 13 to 15 days, after which time all species were compared to controls and visually evaluated. Plant response ratings, summarized in Table B, are based on a scale of 0 to 100 where 0 is no effect and 100 is complete control. A dash (-) response means no test result.

TABLE B

| | Compounds | | | | | | | | | | | | | |
|---|---|---|---|---|---|---|---|---|---|---|---|---|---|---|
| 250 g ai/ha | 1 | 2 | 3 | 4 | 5 | 6 | 8 | 9 | 10 | 11 | 12 | 13 | 14 | 15 |
| Flood | | | | | | | | | | | | | | |
| Barnyardgrass | 0 | 0 | 0 | 0 | 0 | 0 | 0 | 0 | 0 | 0 | 0 | 0 | 0 | 0 |
| Ducksalad | 0 | 0 | 0 | 0 | 0 | 0 | 0 | 0 | 0 | 0 | 0 | 0 | 0 | 0 |
| Rice | 0 | 0 | 0 | 0 | 0 | 0 | 0 | 0 | 0 | 0 | 0 | 0 | 0 | 0 |
| Sedge, Umbrella | 0 | 0 | 0 | 0 | 0 | 0 | 0 | 0 | 0 | 0 | 0 | 0 | 0 | 0 |
| | Compounds | | | | | | | | | | | | | |
| 250 g ai/ha | 16 | 18 | 19 | 20 | 21 | 22 | 23 | 24 | 25 | 26 | 28 | 29 | 30 | 31 |
| Flood | | | | | | | | | | | | | | |
| Barnyardgrass | 0 | 0 | 0 | 0 | 0 | 0 | 0 | 0 | 0 | 0 | 0 | 0 | 0 | 25 |
| Ducksalad | 0 | 0 | 0 | 0 | 0 | 0 | 0 | 0 | 0 | 0 | 0 | 0 | 0 | 0 |
| Rice | 0 | 0 | 0 | 0 | 0 | 0 | 0 | 0 | 0 | 0 | 0 | 0 | 0 | 40 |
| Sedge, Umbrella | 0 | 0 | 0 | 0 | 0 | 0 | 0 | 0 | 0 | 0 | 0 | 0 | 0 | 0 |
| | Compounds | | | | | | | | | | | | | |
| 250 g ai/ha | 32 | 33 | 34 | 35 | 36 | 37 | 38 | 39 | 40 | 41 | 42 | 43 | 44 | 45 |
| Flood | | | | | | | | | | | | | | |
| Barnyardgrass | 0 | 0 | 0 | 0 | 0 | 0 | 0 | 0 | 0 | 0 | 15 | 0 | 0 | 0 |
| Ducksalad | 0 | 0 | 0 | 0 | 0 | 0 | 0 | 0 | 0 | 0 | 20 | 0 | 0 | 0 |
| Rice | 0 | 0 | 0 | 0 | 0 | 0 | 0 | 0 | 0 | 0 | 10 | 0 | 0 | 0 |
| Sedge, Umbrella | 0 | 0 | 0 | 0 | 0 | 0 | 0 | 0 | 0 | 0 | 20 | 0 | 0 | 0 |
| | Compounds | | | | | | | | | | | | | |
| 250 g ai/ha | 46 | 47 | 48 | 49 | 50 | 51 | 52 | 53 | 55 | 56 | 57 | 58 | 59 | 60 |
| Flood | | | | | | | | | | | | | | |
| Barnyardgrass | 0 | 0 | 0 | 0 | 0 | 15 | 0 | 0 | 0 | 0 | 15 | 0 | 0 | 0 |
| Ducksalad | 0 | 0 | 0 | 0 | 0 | 75 | 0 | 0 | 0 | 30 | 0 | 0 | 0 | 0 |
| Rice | 0 | 0 | 0 | 0 | 0 | 15 | 0 | 0 | 0 | 20 | 15 | 0 | 0 | 0 |
| Sedge, Umbrella | 0 | 0 | 0 | 0 | 0 | 65 | 0 | 0 | 0 | 15 | 0 | 0 | 0 | 0 |
| | Compounds | | | | | | | | | | | | | |
| 250 g ai/ha | 61 | 62 | 63 | 64 | 65 | 66 | 67 | 68 | 69 | 70 | 71 | 72 | 73 | 74 |
| Flood | | | | | | | | | | | | | | |
| Barnyardgrass | 0 | 0 | 0 | 0 | 0 | 0 | 0 | 0 | 0 | 0 | 0 | 0 | 0 | 0 |
| Ducksalad | 0 | 0 | 0 | 0 | 0 | 0 | 0 | 0 | 0 | 0 | 0 | 0 | 0 | 25 |
| Rice | 0 | 0 | 0 | 0 | 0 | 0 | 0 | 0 | 0 | 0 | 10 | 0 | 0 | 15 |
| Sedge, Umbrella | 0 | 0 | 0 | 0 | 0 | 0 | 0 | 0 | 0 | 0 | 0 | 0 | 0 | 10 |
| | Compounds | | | | | | | | | | | | | |
| 250 g ai/ha | 75 | 76 | 77 | 78 | 79 | 80 | 81 | 82 | 83 | 84 | 85 | 86 | 87 | 88 |
| Flood | | | | | | | | | | | | | | |
| Barnyardgrass | 0 | 0 | 0 | 0 | 0 | 0 | 0 | 0 | 0 | 0 | 0 | 0 | 0 | 0 |
| Ducksalad | 0 | 0 | 0 | 0 | 0 | 0 | 0 | 0 | 75 | 0 | 0 | 0 | 0 | 0 |
| Rice | 0 | 0 | 0 | 0 | 0 | 0 | 0 | 0 | 0 | 0 | 0 | 0 | 0 | 0 |
| Sedge, Umbrella | 0 | 0 | 0 | 0 | 0 | 0 | 0 | 0 | 70 | 0 | 0 | 0 | 0 | 0 |
| | Compounds | | | | | | | | | | | | | |
| 250 g ai/ha | 89 | 90 | 91 | 92 | 93 | 94 | 95 | 96 | 97 | 98 | 99 | 100 | 101 | 102 |
| Flood | | | | | | | | | | | | | | |
| Barnyardgrass | 0 | 0 | 65 | 0 | 0 | 0 | 0 | 0 | 0 | 0 | 25 | 0 | 0 | 0 |
| Ducksalad | 0 | 0 | 60 | 0 | 65 | 0 | 0 | 0 | 0 | 0 | 70 | 0 | 0 | 0 |
| Rice | 0 | 0 | 20 | 0 | 0 | 0 | 0 | 0 | 0 | 0 | 15 | 0 | 0 | 0 |
| Sedge, Umbrella | 0 | 0 | 60 | 0 | 0 | 0 | 0 | 0 | 0 | 0 | 70 | 0 | 0 | 0 |
| | Compounds | | | | | | | | | | | | | |
| 250 g ai/ha | 103 | 104 | 105 | 106 | 107 | 108 | 109 | 110 | 111 | 112 | 113 | 114 | 115 | 116 |
| Flood | | | | | | | | | | | | | | |
| Barnyardgrass | 0 | 0 | 0 | 0 | 0 | 0 | 50 | 0 | 0 | 55 | 85 | 70 | 75 | 55 |
| Ducksalad | 0 | 0 | 0 | 0 | 0 | 0 | 40 | 0 | 0 | 60 | 75 | 85 | 80 | 60 |
| Rice | 0 | 0 | 0 | 0 | 0 | 0 | 45 | 0 | 0 | 0 | 60 | 35 | 35 | 5 |
| Sedge, Umbrella | 0 | 0 | 0 | 0 | 0 | 0 | 60 | 0 | 0 | 60 | 50 | 85 | 85 | 30 |
| | Compounds | | | | | | | | | | | | | |
| 250 g ai/ha | 117 | 118 | 119 | 120 | 121 | 122 | 123 | 128 | 129 | 130 | 131 | 132 | 133 | 134 |
| Flood | | | | | | | | | | | | | | |
| Barnyardgrass | 85 | 0 | 0 | 0 | 0 | 0 | 0 | 0 | 0 | 0 | 0 | 0 | 0 | 0 |
| Ducksalad | 80 | 55 | 0 | 0 | 0 | 0 | 0 | 40 | 0 | 0 | 0 | 25 | 0 | 0 |
| Rice | 75 | 0 | 0 | 0 | 0 | 0 | 0 | 30 | 0 | 0 | 0 | 0 | 0 | 0 |
| Sedge, Umbrella | 75 | 35 | 0 | 0 | 0 | 0 | 0 | 0 | 0 | 35 | 0 | 15 | 0 | 0 |

| | Compounds | | | | | | | | | |
|---|---|---|---|---|---|---|---|---|---|---|
| 250 g ai/ha | 135 | 140 | 141 | 142 | 143 | 144 | 145 | 146 | 147 | 148 |
| Flood | | | | | | | | | | |
| Barnyardgrass | 0 | 0 | 0 | 0 | 0 | 0 | 0 | 0 | 0 | 0 |
| Ducksalad | 0 | 0 | 0 | 0 | 0 | 0 | 0 | 0 | 0 | 0 |
| Rice | 0 | 0 | 0 | 0 | 0 | 0 | 0 | 0 | 0 | 0 |
| Sedge, Umbrella | 0 | 0 | 0 | 0 | 0 | 0 | 0 | 0 | 0 | 0 |

### Test C

Seeds of plant species selected from barnyardgrass (Echinochloa crus-galli), kochia (Bassia scoparia), ragweed (common ragweed, Ambrosia artemisiifolia), Italian ryegrass (Lolium mutiflorum), foxtail, giant (giant foxtail, Setaria faberi), foxtail, green (green foxtail, Setaria viridis), and pigweed (Amaranthus retroflexus) were planted into a blend of loam soil and sand and treated preemergence with a directed soil spray using test chemicals formulated in anon-phytotoxic solvent mixture which included a surfactant.

At the same time, plants selected from these weed species and also wheat (Triticum aestivum), corn (Zea mays), blackgrass (Alopecurus myosuroides), and galium (catchweed bedstraw, Galium aparine) were planted in pots containing the same blend of loam soil and sand and treated with post emergence applications of test chemicals formulated in the same manner. Plants ranged in height from 2 to 10 cm and were in the one- to two-leaf stage for the postemergence treatment. Treated plants and untreated controls were maintained in a greenhouse for approximately 10 d, after which time all treated plants were compared to untreated controls and visually evaluated for injury. Plant response ratings, summarized in Table C, are based on a 0 to 100 scale where 0 is no effect and 100 is complete control. A dash (-)response means no test result.

TABLE C

| | Compounds | | | | | | |
|---|---|---|---|---|---|---|---|
| 1000 g ai/ha | 221 | 222 | 223 | 224 | 225 | 237 | 238 |
| Postemergence | | | | | | | |
| Barnyardgrass | 70 | 10 | 20 | 10 | 10 | 0 | 20 |
| Blackgrass | 90 | 0 | 0 | 10 | 20 | 10 | 20 |
| Corn | 20 | 0 | 0 | 0 | 0 | 0 | 0 |
| Foxtail, Giant | 60 | 20 | 30 | 50 | 20 | 0 | 40 |
| Galium | 100 | 90 | 90 | 90 | 90 | 100 | 90 |
| Kochia | 70 | 0 | 0 | 0 | 10 | 80 | 80 |
| Pigweed | 90 | 90 | 40 | 50 | 60 | 90 | 90 |
| Ragweed | 80 | 50 | 50 | 50 | 70 | 30 | 30 |
| Ryegrass, Italian | 100 | 0 | 0 | 0 | 0 | 10 | 0 |
| Wheat | 60 | 0 | 0 | 0 | 0 | 0 | 0 |

| | Compounds | | | | | | Compound |
|---|---|---|---|---|---|---|---|
| 500 g ai/ha | 149 | 150 | 151 | 168 | 206 | 125 g ai/ha | 305 |
| Postemergence | | | | | | Postemergence | |
| Barnyardgrass | 0 | 0 | 10 | 60 | 80 | Barnyardgrass | 30 |
| Blackgrass | 0 | 0 | 30 | 80 | 90 | Blackgrass | 20 |
| Corn | 0 | 0 | 0 | 20 | 20 | Corn | 0 |
| Foxtail, Giant | — | — | 10 | 90 | 90 | Foxtail, Giant | 50 |
| Foxtail, Green | 0 | 0 | — | — | — | Foxtail, Green | — |
| Galium | — | — | 50 | 90 | 100 | Galium | 70 |
| Kochia | 0 | 0 | 20 | 80 | 90 | Kochia | 70 |
| Pigweed | 0 | 0 | 10 | 100 | 100 | Pigweed | 90 |
| Ragweed | 0 | 20 | 30 | 90 | 100 | Ragweed | 70 |
| Ryegrass, Italian | 10 | 10 | 30 | 90 | 100 | Ryegrass, Italian | 80 |
| Wheat | 0 | 0 | 0 | 30 | 90 | Wheat | 0 |

| | Compounds | | | | | | | | | | |
|---|---|---|---|---|---|---|---|---|---|---|---|
| 125 g ai/ha | 149 | 151 | | 152 | | 153 | | 154 | 155 | | 156 |
| Postemergence | | | | | | | | | | | |
| Barnyardgrass | 0 | 0 | | 20 | | 90 | | 90 | 80 | | 70 |
| Blackgrass | 0 | 0 | | 10 | | 80 | | 90 | 90 | | 30 |
| Corn | 0 | 0 | | 0 | | 50 | | 40 | 20 | | 20 |
| Foxtail, Giant | — | 0 | | 50 | | 100 | | 90 | 90 | | 90 |
| Foxtail, Green | 0 | — | | — | | — | | — | — | | — |
| Galium | — | 40 | | 80 | | 100 | | 100 | 90 | | 100 |
| Kochia | 0 | 0 | | 80 | | 100 | | 70 | 80 | | 100 |
| Pigweed | 0 | 10 | | 70 | | 100 | | 100 | 100 | | 80 |
| Ragweed | 0 | 0 | | 90 | | 100 | | 90 | 100 | | 100 |
| Ryegrass, Italian | 0 | 20 | | 80 | | 100 | | 100 | 100 | | 100 |
| Wheat | 0 | 0 | | 0 | | 80 | | 90 | 60 | | 50 |
| | Compounds | | | | | | | | | | |
| 125 g ai/ha | 157 | 158 | | 159 | | 163 | | 164 | 165 | | 166 |
| Postemergence | | | | | | | | | | | |
| Barnyardgrass | 80 | 70 | | 60 | | 50 | | 60 | 80 | | 30 |
| Blackgrass | 20 | 10 | | 30 | | 60 | | 50 | 40 | | 20 |
| Corn | 30 | 10 | | 30 | | 30 | | 30 | 20 | | 20 |
| Foxtail, Giant | 90 | 70 | | 70 | | 70 | | 70 | 70 | | 30 |
| Foxtail, Green | — | — | | — | | — | | — | — | | — |
| Galium | 100 | 0 | | 80 | | 90 | | 70 | 100 | | 80 |
| Kochia | 100 | 0 | | 80 | | 50 | | 70 | 90 | | 40 |
| Pigweed | 60 | 50 | | 100 | | 70 | | 70 | 70 | | 50 |
| Ragweed | 100 | 60 | | 90 | | 60 | | 50 | 100 | | 30 |
| Ryegrass, Italian | 100 | 20 | | 80 | | 90 | | 70 | 100 | | 80 |
| Wheat | 60 | 0 | | 10 | | 80 | | 80 | 30 | | 60 |
| | Compounds | | | | | | | | | | |
| 125 g ai/ha | 167 | 168 | | 173 | | 174 | | 175 | 176 | | 177 |
| Postemergence | | | | | | | | | | | |
| Barnyardgrass | 90 | 40 | | 80 | | 90 | | 70 | 40 | | 20 |
| Blackgrass | 90 | 20 | | 40 | | 40 | | 30 | 30 | | 0 |
| Corn | 70 | 0 | | 20 | | 20 | | 20 | 0 | | 0 |
| Foxtail, Giant | 90 | 50 | | 80 | | 90 | | 80 | 20 | | 10 |
| Foxtail, Green | — | — | | — | | — | | — | — | | — |
| Galium | 100 | 70 | | 100 | | 100 | | 100 | 90 | | 80 |
| kochia | 100 | 70 | | 90 | | 90 | | 100 | 70 | | 70 |
| Pigweed | 100 | 60 | | 90 | | 80 | | 70 | 80 | | 60 |
| Ragweed | 100 | 60 | | 100 | | 90 | | 90 | 70 | | 60 |
| Ryegrass, Italian | 100 | 40 | | 100 | | 100 | | 70 | 90 | | 60 |
| Wheat | 100 | 0 | | 0 | | 0 | | 0 | 0 | | 0 |
| | Compounds | | | | | | | | | | |
| 125 g ai/ha | 178 | 179 | | 180 | | 181 | | 182 | 183 | | 184 |
| Postemergence | | | | | | | | | | | |
| Barnyardgrass | 50 | 80 | | 60 | | 60 | | 60 | 40 | | 60 |
| Blackgrass | 30 | 90 | | 30 | | 20 | | 10 | 0 | | 0 |
| Corn | 10 | 80 | | 20 | | 30 | | 30 | 0 | | 10 |
| Foxtail, Giant | 30 | 100 | | 90 | | 80 | | 90 | 80 | | 80 |
| Foxtail, Green | — | — | | — | | — | | — | — | | — |
| Galium | 80 | 100 | | 100 | | 100 | | 100 | 30 | | 90 |
| kochia | 70 | 100 | | 90 | | 80 | | 70 | 10 | | 10 |
| Pigweed | 40 | 100 | | 70 | | 90 | | 50 | 10 | | 20 |
| Ragweed | 40 | 100 | | 100 | | 100 | | 100 | 100 | | 100 |
| Ryegrass, Italian | 90 | 100 | | 100 | | 100 | | 100 | 60 | | 80 |
| Wheat | 80 | 100 | | 50 | | 50 | | 70 | 0 | | 0 |
| | Compounds | | | | | | | | | | |
| 125 g ai/ha | 185 | 186 | | 187 | | 188 | | 189 | 190 | | 191 |
| Postemergence | | | | | | | | | | | |
| Barnyardgrass | 60 | 70 | | 70 | | 60 | | 50 | 20 | | 90 |
| Blackgrass | 30 | 20 | | 0 | | 30 | | 30 | 20 | | 70 |
| Corn | 20 | 20 | | 20 | | 30 | | 10 | 0 | | 60 |
| Foxtail, Giant | 90 | 90 | | 80 | | 90 | | 80 | 30 | | 80 |
| Foxtail, Green | — | — | | — | | — | | — | — | | — |
| Galium | 100 | 100 | | 100 | | 100 | | 100 | 100 | | 100 |
| Kochia | 50 | 50 | | 70 | | 100 | | 90 | 20 | | 80 |
| Pigweed | 100 | 90 | | 70 | | 90 | | 80 | 30 | | 100 |
| Ragweed | 100 | 90 | | 100 | | 100 | | 90 | 60 | | 80 |
| Ryegrass, Italian | 100 | 100 | | 100 | | 100 | | 100 | 20 | | 80 |
| Wheat | 80 | 70 | | 50 | | 50 | | 50 | 0 | | 70 |
| | Compounds | | | | | | | | | | |
| 125 g ai/ha | 192 | 193 | | 197 | | 198 | | 199 | 200 | | 201 |
| Postemergence | | | | | | | | | | | |
| Barnyardgrass | 90 | 50 | | 10 | | 80 | | 20 | 30 | | 40 |
| Blackgrass | 70 | 50 | | 30 | | 40 | | 20 | 0 | | 50 |
| Corn | 30 | 10 | | 0 | | 20 | | 0 | 0 | | 0 |
| Foxtail, Giant | 80 | 50 | | 20 | | 70 | | 20 | 20 | | 40 |
| Foxtail, Green | — | — | | — | | — | | — | — | | — |
| Galium | 100 | 90 | | 30 | | 20 | | 30 | 40 | | 80 |
| Kochia | 60 | 70 | | 0 | | 0 | | 0 | 0 | | 70 |
| Pigweed | 80 | 80 | | 30 | | 10 | | 0 | 0 | | 70 |
| Ragweed | 90 | 70 | | 0 | | 0 | | 0 | 0 | | 30 |
| Ryegrass, Italian | 70 | 70 | | 20 | | 60 | | 0 | 0 | | 60 |
| Wheat | 50 | 0 | | 0 | | 0 | | 0 | 0 | | 40 |
| | Compounds | | | | | | | | | | |
| 125 g ai/ha | 202 | 203 | | 204 | | 205 | | 206 | 207 | | 208 |
| Postemergence | | | | | | | | | | | |
| Barnyardgrass | 10 | 20 | | 0 | | 90 | | 30 | 60 | | 60 |
| Blackgrass | 0 | 0 | | 0 | | 80 | | 30 | 20 | | 30 |
| Corn | 0 | 0 | | 0 | | 40 | | 0 | 10 | | 10 |
| Foxtail, Giant | 0 | 30 | | 0 | | 100 | | 60 | 70 | | 60 |
| Foxtail, Green | — | — | | — | | — | | — | — | | — |
| Galium | 70 | 30 | | 40 | | 90 | | 80 | 100 | | 100 |
| Kochia | 40 | 50 | | 10 | | 70 | | 70 | 70 | | 70 |
| Pigweed | 60 | 20 | | 10 | | 100 | | 60 | 80 | | 40 |
| Ragweed | 20 | 10 | | 0 | | 100 | | 60 | 90 | | 100 |
| Ryegrass, Italian | 30 | 30 | | 0 | | 100 | | 100 | 80 | | 80 |
| Wheat | 0 | 0 | | 0 | | 70 | | 60 | 0 | | 0 |
| | Compounds | | | | | | | | | | |
| 125 g ai/ha | 209 | 210 | | 211 | | 212 | | 213 | 214 | | 216 |
| Postemergence | | | | | | | | | | | |
| Barnyardgrass | 80 | 70 | | 70 | | 70 | | 80 | 80 | | 10 |
| Blackgrass | 30 | 30 | | 30 | | 0 | | 80 | 80 | | 0 |
| Corn | 20 | 20 | | 20 | | 0 | | 40 | 40 | | 0 |
| Foxtail, Giant | 90 | 90 | | 90 | | 80 | | 80 | 80 | | 0 |
| Foxtail, Green | — | — | | — | | — | | — | — | | — |
| Galium | 100 | 100 | | 100 | | 100 | | 90 | 90 | | 80 |
| Kochia | 40 | 40 | | 90 | | 50 | | 70 | 80 | | 50 |
| Pigweed | 90 | 100 | | 100 | | 90 | | 90 | 90 | | 20 |
| Ragweed | 90 | 90 | | 80 | | 90 | | 80 | 90 | | 40 |
| Ryegrass, Italian | 40 | 20 | | 80 | | 80 | | 100 | 100 | | 0 |
| Wheat | 0 | 0 | | 40 | | 0 | | 60 | 70 | | 0 |
| | Compounds | | | | | | | | | | |
| 125 g ai/ha | 217 | 218 | | 219 | | 220 | | 226 | 227 | | 229 |
| Postemergence | | | | | | | | | | | |
| Barnyardgrass | 20 | 10 | | 30 | | 80 | | 20 | 20 | | 30 |
| Blackgrass | 0 | 0 | | 0 | | 80 | | 0 | 0 | | 0 |
| Corn | 0 | 0 | | 0 | | 40 | | 0 | 0 | | 40 |
| Foxtail, Giant | 0 | 0 | | 0 | | 100 | | 40 | 0 | | 40 |
| Foxtail, Green | — | — | | — | | — | | — | — | | — |
| Galium | 80 | 80 | | 60 | | 100 | | 90 | 80 | | 80 |
| Kochia | 60 | 30 | | 50 | | 80 | | 60 | 70 | | 0 |
| Pigweed | 20 | 60 | | 40 | | 100 | | 50 | 50 | | 50 |
| Ragweed | 20 | 10 | | 10 | | 100 | | 70 | 50 | | 20 |
| Ryegrass, Italian | 0 | 60 | | 40 | | 100 | | 50 | 40 | | 70 |
| Wheat | 0 | 0 | | 0 | | 90 | | 0 | 0 | | 60 |
| | Compounds | | | | | | | | | | |
| 125 g ai/ha | 230 | 231 | | 232 | | 233 | | 234 | 235 | | 236 |
| Postemergence | | | | | | | | | | | |
| Barnyardgrass | 20 | 70 | | 90 | | 60 | | 30 | 90 | | 90 |
| Blackgrass | 10 | 20 | | 50 | | 30 | | 0 | 20 | | 30 |
| Corn | 0 | 20 | | 10 | | 10 | | 0 | 10 | | 30 |
| Foxtail, Giant | 30 | 70 | | 80 | | 80 | | 30 | 80 | | 90 |
| Foxtail, Green | — | — | | — | | — | | — | — | | — |
| Galium | 70 | 100 | | 100 | | 90 | | 90 | 90 | | 100 |
| Kochia | 0 | 20 | | 50 | | 10 | | 70 | 60 | | 50 |
| Pigweed | 20 | 60 | | 90 | | 90 | | 30 | 90 | | 60 |
| Ragweed | 20 | 100 | | 100 | | 100 | | 80 | 100 | | 100 |
| Ryegrass, Italian | 40 | 10 | | 0 | | 0 | | 70 | 0 | | 20 |
| Wheat | 60 | 0 | | 0 | | 0 | | 0 | 0 | | 0 |
| | Compounds | | | | | | | | | | |
| 125 g ai/ha | 239 | 240 | | 241 | | 242 | | 243 | 244 | | 245 |
| Postemergence | | | | | | | | | | | |
| Barnyardgrass | 30 | 20 | | 60 | | 90 | | 80 | 50 | | 50 |
| Blackgrass | 0 | 10 | | 40 | | 90 | | 50 | 70 | | 0 |
| Corn | 0 | 0 | | 0 | | 20 | | 30 | 30 | | 30 |
| Foxtail, Giant | 20 | 0 | | 80 | | 80 | | 80 | 80 | | 80 |
| Foxtail, Green | — | — | | — | | — | | — | — | | — |
| Galium | 90 | 50 | | 100 | | 100 | | 90 | 90 | | 90 |
| Kochia | 60 | 0 | | 20 | | 50 | | 20 | 20 | | 20 |
| Piqweed | 60 | 80 | | 90 | | 100 | | 90 | 90 | | 90 |
| Ragweed | 70 | 0 | | 100 | | 90 | | 90 | 40 | | 70 |
| Ryegrass, Italian | 60 | 0 | | 0 | | 90 | | 40 | 10 | | 0 |
| Wheat | 0 | 0 | | 0 | | 60 | | 0 | 0 | | 0 |
| | Compounds | | | | | | | | | | |
| 125 g ai/ha | 246 | 250 | | 253 | | 254 | | 261 | 264 | | 268 |
| Postemergence | | | | | | | | | | | |
| Barnyardgrass | 70 | 80 | | 40 | | 50 | | 40 | 50 | | 60 |
| Blackgrass | 70 | 30 | | 60 | | 70 | | 60 | 60 | | 20 |
| Corn | 10 | 10 | | 0 | | 10 | | 10 | 0 | | 30 |
| Foxtail, Giant | 70 | 80 | | 50 | | 60 | | 50 | 60 | | 90 |
| Foxtail, Green | — | — | | — | | — | | — | — | | — |
| Galium | 90 | 100 | | 50 | | 40 | | 80 | 70 | | 50 |
| Kochia | 20 | 60 | | 60 | | 70 | | 60 | 70 | | 20 |
| Piqweed | 80 | 70 | | 70 | | 70 | | 60 | 60 | | 20 |
| Ragweed | 100 | 100 | | 40 | | 50 | | 60 | 60 | | 100 |
| Ryegrass, Italian | 30 | 0 | | 60 | | 70 | | 60 | 70 | | 100 |
| Wheat | 0 | 0 | | 30 | | 60 | | 50 | 50 | | 20 |
| | Compounds | | | | | | | | | | |
| 125 g ai/ha | 269 | 270 | | 271 | | 272 | | 273 | 277 | | 278 |
| Postemergence | | | | | | | | | | | |
| Barnyardgrass | 0 | 0 | | 80 | | 60 | | 80 | 60 | | 50 |
| Blackgrass | 30 | 30 | | 40 | | 80 | | 90 | 80 | | 90 |
| Corn | 20 | 0 | | 30 | | 20 | | 30 | 30 | | 20 |
| Foxtail, Giant | 40 | 10 | | 80 | | 90 | | 100 | 60 | | 70 |
| Foxtail, Green | — | — | | — | | — | | — | — | | — |
| Galium | 80 | 80 | | 100 | | 100 | | 100 | 80 | | 90 |
| Kochia | 30 | 30 | | 80 | | 80 | | 60 | 70 | | 80 |
| Pigweed | 60 | 50 | | 90 | | 90 | | 100 | 60 | | 70 |
| Ragweed | 40 | 70 | | 90 | | 80 | | 100 | 90 | | 80 |
| Ryegrass, Italian | 60 | 10 | | 100 | | 100 | | 60 | 70 | | 80 |
| Wheat | 0 | 0 | | 70 | | 60 | | 0 | 70 | | 80 |
| | Compounds | | | | | | | | | | |
| 125 g ai/ha | 279 | 285 | | 286 | | 287 | | 288 | 289 | | 290 |
| Postemergence | | | | | | | | | | | |
| Barnyardgrass | 0 | 30 | | 30 | | 60 | | 100 | 10 | | 20 |
| Blackgrass | 0 | 10 | | 0 | | 30 | | 40 | 0 | | 0 |
| Corn | 0 | 0 | | 0 | | 10 | | 20 | 0 | | 0 |
| Foxtail, Giant | 0 | 20 | | 20 | | 70 | | 90 | 0 | | 0 |
| Foxtail, Green | — | — | | — | | — | | — | — | | — |
| Galium | 0 | 60 | | 60 | | 70 | | 80 | 100 | | 100 |
| Kochia | 30 | 20 | | 30 | | 10 | | 30 | 40 | | 20 |
| Pigweed | 10 | 70 | | 50 | | 20 | | 30 | 0 | | 0 |
| Ragweed | 10 | 60 | | 50 | | 50 | | 60 | 90 | | 50 |
| Ryegrass, Italian | 0 | 0 | | 0 | | 80 | | 100 | 60 | | 60 |
| Wheat | 0 | 0 | | 30 | | 0 | | 0 | 0 | | 0 |
| | Compounds | | | | | | | | | | |
| 125 g ai/ha | 291 | 292 | | 293 | | 294 | | 295 | 296 | | 297 |
| Postemergence | | | | | | | | | | | |
| Barnyardgrass | 70 | 70 | | 50 | | 60 | | 70 | 70 | | 0 |
| Blackgrass | 70 | 80 | | 0 | | 0 | | 0 | 70 | | 0 |
| Corn | 30 | 40 | | 10 | | 10 | | 30 | 20 | | 0 |
| Foxtail, Giant | 80 | 100 | | 80 | | 60 | | 90 | 90 | | 0 |
| Foxtail, Green | — | — | | — | | — | | — | — | | — |
| Galium | 100 | 100 | | 100 | | 100 | | 100 | 100 | | 0 |
| Kochia | 100 | 100 | | 30 | | 30 | | 50 | 50 | | 0 |
| Pigweed | 60 | 90 | | 40 | | 20 | | 90 | 90 | | 0 |
| Ragweed | 100 | 100 | | 100 | | 100 | | 100 | 100 | | 0 |
| Ryegrass, Italian | 100 | 100 | | 100 | | 100 | | 100 | 100 | | 0 |
| Wheat | 90 | 100 | | 70 | | 60 | | 100 | 100 | | 0 |
| | Compounds | | | | | | | | | | |
| 125 g ai/ha | 298 | 299 | | 300 | | 301 | | 302 | 303 | | 304 |
| Postemergence | | | | | | | | | | | |
| Barnyardgrass | 0 | 90 | | 70 | | 40 | | 0 | 10 | | 0 |
| Blackgrass | 20 | 80 | | 80 | | 60 | | 0 | 0 | | 0 |
| Corn | 0 | 30 | | 30 | | 20 | | 0 | 0 | | 20 |
| Foxtail, Giant | 0 | 100 | | 80 | | 70 | | 30 | 20 | | 20 |
| Foxtail, Green | — | — | | — | | — | | — | — | | — |
| Galium | 30 | 100 | | 100 | | 90 | | 60 | 70 | | 80 |
| Kochia | 10 | 100 | | 50 | | 30 | | 20 | 50 | | 50 |
| Pigweed | 40 | 100 | | 80 | | 70 | | 60 | 60 | | 80 |
| Ragweed | 10 | 100 | | 90 | | 30 | | 20 | 20 | | 10 |
| Ryegrass, Italian | 0 | 100 | | 40 | | 10 | | 30 | 40 | | 60 |
| Wheat | 0 | 70 | | 0 | | 0 | | 0 | 0 | | 0 |
| | Compounds | | | | | | | | | | |
| 31 g ai/ha | 152 | 153 | | 154 | | 155 | | 156 | 157 | | 158 |
| Postemergence | | | | | | | | | | | |
| Barnyardgrass | 0 | 80 | | 50 | | 50 | | 40 | 30 | | 50 |
| Blackgrass | 0 | 50 | | 50 | | 30 | | 30 | 10 | | 0 |
| Corn | 0 | 20 | | 0 | | 0 | | 20 | 10 | | 0 |
| Foxtail, Giant | 10 | 90 | | 80 | | 80 | | 60 | 60 | | 30 |
| Galium | 80 | 100 | | 90 | | 90 | | 100 | 90 | | 90 |
| Kochia | 50 | 90 | | 60 | | 70 | | 80 | 80 | | 0 |
| Pigweed | 60 | 100 | | 30 | | 50 | | 70 | 20 | | 50 |
| Ragweed | 40 | 90 | | 70 | | 90 | | 90 | 70 | | 40 |
| Ryegrass, Italian | 20 | 90 | | 70 | | 90 | | 100 | 70 | | 0 |
| Wheat | 0 | 40 | | 30 | | 0 | | 0 | 0 | | 0 |
| | Compounds | | | | | | | | | | |
| 31 g ai/ha | 159 | 163 | | 164 | | 165 | | 166 | 167 | | 173 |
| Postemergence | | | | | | | | | | | |
| Barnyardgrass | 40 | 20 | | 30 | | 40 | | 20 | 80 | | 50 |
| Blackgrass | 10 | 20 | | 30 | | 20 | | 0 | 80 | | 20 |
| Corn | 10 | 0 | | 10 | | 0 | | 0 | 30 | | 0 |
| Foxtail, Giant | 50 | 20 | | 10 | | 20 | | 10 | 90 | | 30 |
| Galium | 60 | 70 | | 60 | | 100 | | 50 | 80 | | 90 |
| Kochia | 70 | 30 | | 30 | | 90 | | 30 | 80 | | 60 |
| Pigweed | 90 | 40 | | 50 | | 40 | | 30 | 100 | | 70 |
| Ragweed | 70 | 30 | | 20 | | 80 | | 20 | 100 | | 80 |
| Ryegrass, Italian | 20 | 50 | | 0 | | 70 | | 0 | 100 | | 80 |
| Wheat | 0 | 60 | | 20 | | 0 | | 0 | 70 | | 0 |
| | Compounds | | | | | | | | | | |
| 31 g ai/ha | 174 | 175 | | 176 | | 177 | | 178 | 179 | | 180 |
| Postemergence | | | | | | | | | | | |
| Barnyardgrass | 60 | 50 | | 30 | | 0 | | 20 | 90 | | 30 |
| Blackgrass | 30 | 20 | | 0 | | 0 | | 0 | 80 | | 0 |
| Corn | 10 | 10 | | 0 | | 0 | | 0 | 30 | | 0 |
| Foxtail, Giant | 50 | 50 | | 10 | | 10 | | 10 | 90 | | 70 |
| Galium | 100 | 90 | | 60 | | 40 | | 40 | 100 | | 90 |
| Kochia | 70 | 80 | | 20 | | 10 | | 40 | 80 | | 80 |
| Pigweed | 60 | 50 | | 70 | | 40 | | 20 | 100 | | 10 |
| Ragweed | 80 | 80 | | 30 | | 20 | | 0 | 100 | | 100 |
| Ryegrass, Italian | 60 | 50 | | 60 | | 30 | | 20 | 90 | | 70 |
| Wheat | 0 | 0 | | 0 | | 0 | | 0 | 80 | | 30 |
| | Compounds | | | | | | | | | | |
| 31 g ai/ha | 181 | 182 | | 183 | | 184 | | 185 | 186 | | 187 |
| Postemergence | | | | | | | | | | | |
| Barnyardgrass | 40 | 50 | | 0 | | 50 | | 10 | 30 | | 30 |
| Blackgrass | 0 | 10 | | 0 | | 0 | | 10 | 10 | | 0 |
| Corn | 0 | 0 | | 0 | | 0 | | 0 | 0 | | 0 |
| Foxtail, Giant | 50 | 70 | | 30 | | 60 | | 60 | 60 | | 50 |
| Galium | 100 | 70 | | 10 | | 60 | | 70 | 90 | | 80 |
| Kochia | 60 | 10 | | 0 | | 10 | | 10 | 50 | | 40 |
| Pigweed | 40 | 10 | | 0 | | 10 | | 40 | 20 | | 20 |
| Ragweed | 100 | 40 | | 20 | | 60 | | 90 | 50 | | 90 |
| Ryegrass, Italian | 70 | 70 | | 40 | | 0 | | 70 | 80 | | 70 |
| Wheat | 0 | 0 | | 0 | | 0 | | 50 | 20 | | 0 |
| | Compounds | | | | | | | | | | |
| 31 g ai/ha | 188 | 189 | | 190 | | 191 | | 192 | 193 | | 197 |
| Postemergence | | | | | | | | | | | |
| Barnyardgrass | 30 | 30 | | 30 | | 80 | | 80 | 20 | | 0 |
| Blackgrass | 40 | 10 | | 0 | | 80 | | 40 | 20 | | 0 |
| Corn | 0 | 0 | | 0 | | 10 | | 20 | 0 | | 0 |
| Foxtail, Giant | 40 | 50 | | 10 | | 70 | | 70 | 40 | | 0 |
| Galium | 100 | 100 | | 90 | | 70 | | 80 | 80 | | 0 |
| Kochia | 80 | 70 | | 10 | | 50 | | 30 | 60 | | 0 |
| Pigweed | 80 | 50 | | 30 | | 90 | | 50 | 40 | | 10 |
| Ragweed | 60 | 70 | | 10 | | 70 | | 80 | 70 | | 0 |
| Ryegrass, Italian | 80 | 90 | | 10 | | 0 | | 20 | 40 | | 0 |
| Wheat | 10 | 10 | | 0 | | 0 | | 0 | 0 | | 0 |
| | Compounds | | | | | | | | | | |
| 31 g ai/ha | 198 | 199 | | 200 | | 201 | | 202 | 203 | | 204 |
| Postemergence | | | | | | | | | | | |
| Barnyardgrass | 20 | 0 | | 20 | | 10 | | 0 | 0 | | 0 |
| Blackgrass | 0 | 0 | | 0 | | 10 | | 0 | 0 | | 0 |
| Corn | 0 | 0 | | 0 | | 0 | | 0 | 0 | | 0 |
| Foxtail, Giant | 20 | 0 | | 10 | | 20 | | 0 | 0 | | 0 |
| Galium | 0 | 20 | | 30 | | 50 | | 40 | 30 | | 10 |
| Kochia | 0 | 0 | | 0 | | 40 | | 10 | 20 | | 0 |
| Pigweed | 0 | 0 | | 0 | | 20 | | 20 | 10 | | 0 |
| Ragweed | 0 | 0 | | 0 | | 10 | | 0 | 0 | | 0 |
| Ryegrass, Italian | 0 | 0 | | 0 | | 30 | | 10 | 30 | | 0 |
| Wheat | 0 | 0 | | 0 | | 0 | | 0 | 0 | | 0 |
| | Compounds | | | | | | | | | | |
| 31 g ai/ha | 205 | 207 | | 208 | | 209 | | 210 | 211 | | 212 |
| Postemergence | | | | | | | | | | | |
| Barnyardgrass | 70 | 20 | | 30 | | 50 | | 40 | 30 | | 50 |
| Blackgrass | 20 | 30 | | 0 | | 10 | | 0 | 30 | | 0 |
| Corn | 0 | 30 | | 0 | | 10 | | 20 | 30 | | 0 |
| Foxtail, Giant | 80 | 30 | | 20 | | 70 | | 70 | 60 | | 50 |
| Galium | 80 | 90 | | 90 | | 90 | | 90 | 100 | | 90 |
| Kochia | 60 | 60 | | 50 | | 10 | | 30 | 50 | | 50 |
| Pigweed | 90 | 50 | | 30 | | 50 | | 40 | 70 | | 50 |
| Ragweed | 100 | 30 | | 100 | | 90 | | 90 | 80 | | 100 |
| Ryegrass, Italian | 70 | 20 | | 30 | | 10 | | 0 | 30 | | 10 |
| Wheat | 20 | 0 | | 0 | | 0 | | 0 | 0 | | 0 |
| | Compounds | | | | | | | | | | |
| 31 g ai/ha | 213 | 214 | | 216 | | 217 | | 218 | 219 | | 220 |
| Postemergence | | | | | | | | | | | |
| Barnyardgrass | 80 | 70 | | 0 | | 0 | | 0 | 20 | | 40 |
| Blackgrass | 30 | 30 | | 0 | | 0 | | 0 | 0 | | 30 |
| Corn | 20 | 30 | | 0 | | 0 | | 0 | 0 | | 10 |
| Foxtail, Giant | 70 | 80 | | 0 | | 0 | | 0 | 0 | | 60 |
| Galium | 90 | 90 | | 30 | | 40 | | 50 | 50 | | 70 |
| Kochia | 60 | 70 | | 20 | | 30 | | 10 | 10 | | 50 |
| Pigweed | 80 | 80 | | 0 | | 10 | | 20 | 20 | | 80 |
| Ragweed | 70 | 80 | | 10 | | 0 | | 0 | 0 | | 60 |
| Ryegrass, Italian | 90 | 80 | | 0 | | 0 | | 40 | 0 | | 70 |
| Wheat | 40 | 40 | | 0 | | 0 | | 0 | 0 | | 40 |
| | Compounds | | | | | | | | | | |
| 31 g ai/ha | 226 | 227 | | 229 | | 230 | | 231 | 232 | | 233 |
| Postemergence | | | | | | | | | | | |
| Barnyardgrass | 10 | 0 | | 0 | | 0 | | 30 | 40 | | 40 |
| Blackgrass | 0 | 0 | | 50 | | 0 | | 10 | 20 | | 0 |
| Corn | 0 | 0 | | 0 | | 0 | | 0 | 0 | | 0 |
| Foxtail, Giant | 30 | 0 | | 10 | | 10 | | 30 | 60 | | 30 |
| Galium | 50 | 60 | | 60 | | 50 | | 70 | 80 | | 60 |
| Kochia | 20 | 30 | | 0 | | 0 | | 10 | 20 | | 0 |
| Pigweed | 30 | 30 | | 20 | | 0 | | 20 | 70 | | 60 |
| Ragweed | 30 | 20 | | 10 | | 10 | | 100 | 100 | | 100 |
| Ryegrass, Italian | 20 | 20 | | 40 | | 0 | | 0 | 0 | | 0 |
| Wheat | 0 | 0 | | 30 | | 0 | | 0 | 0 | | 0 |
| | Compounds | | | | | | | | | | |
| 31 g ai/ha | 234 | 235 | | 236 | | 239 | | 240 | 241 | | 242 |
| Postemergence | | | | | | | | | | | |
| Barnyardgrass | 10 | 60 | | 70 | | 10 | | 0 | 30 | | 80 |
| Blackgrass | 10 | 20 | | 0 | | 0 | | 0 | 20 | | 80 |
| Corn | 0 | 0 | | 20 | | 0 | | 0 | 10 | | 0 |
| Foxtail, Giant | 10 | 40 | | 60 | | 0 | | 0 | 70 | | 70 |
| Galium | 80 | 90 | | 90 | | 70 | | 20 | 90 | | 80 |
| Kochia | 30 | 50 | | 30 | | 40 | | 0 | 0 | | 40 |
| Pigweed | 10 | 50 | | 20 | | 20 | | 70 | 80 | | 90 |
| Ragweed | 40 | 100 | | 100 | | 50 | | 0 | 80 | | 50 |
| Ryegrass, Italian | 50 | 0 | | 0 | | 50 | | 0 | 0 | | 40 |
| Wheat | 0 | 0 | | 0 | | 0 | | 0 | 0 | | 0 |
| | Compounds | | | | | | | | | | |
| 31 g ai/ha | 243 | 244 | | 245 | | 246 | | 250 | 253 | | 254 |
| Postemergence | | | | | | | | | | | |
| Barnyardgrass | 70 | 30 | | 30 | | 40 | | 50 | 10 | | 30 |
| Blackgrass | 30 | 40 | | 0 | | 50 | | 10 | 20 | | 10 |
| Corn | 0 | 10 | | 30 | | 20 | | 0 | 0 | | 0 |
| Foxtail, Giant | 70 | 80 | | 30 | | 70 | | 20 | 20 | | 20 |
| Galium | 80 | 80 | | 70 | | 80 | | 90 | 30 | | 0 |
| Kochia | 20 | 20 | | 0 | | 0 | | 40 | 20 | | 50 |
| Pigweed | 80 | 80 | | 70 | | 70 | | 50 | 50 | | 20 |
| Ragweed | 60 | 90 | | 20 | | 80 | | 100 | 30 | | 30 |
| Ryegrass, Italian | 10 | 0 | | 0 | | 0 | | 0 | 10 | | 20 |
| Wheat | 0 | 0 | | 0 | | 0 | | 0 | 0 | | 30 |
| | Compounds | | | | | | | | | | |
| 31 g ai/ha | 261 | 264 | | 268 | | 269 | | 270 | 271 | | 272 |
| Postemergence | | | | | | | | | | | |
| Barnyardgrass | 20 | 10 | | 50 | | 0 | | 0 | 40 | | 40 |
| Blackgrass | 0 | 30 | | 0 | | 0 | | 0 | 20 | | 30 |
| Corn | 0 | 10 | | 0 | | 0 | | 0 | 10 | | 10 |
| Foxtail, Giant | 10 | 10 | | 60 | | 10 | | 0 | 70 | | 60 |
| Galium | 40 | 50 | | 20 | | 50 | | 50 | 100 | | 90 |
| Kochia | 30 | 40 | | 10 | | 0 | | 20 | 60 | | 70 |
| Pigweed | 50 | 40 | | 10 | | 50 | | 80 | 80 | | 70 |
| Ragweed | 40 | 40 | | 70 | | 10 | | 20 | 70 | | 60 |
| Ryegrass, Italian | 0 | 0 | | 60 | | 0 | | 0 | 80 | | 90 |
| Wheat | 0 | 20 | | 0 | | 0 | | 0 | 30 | | 30 |
| | Compounds | | | | | | | | | | |
| 31 g ai/ha | 273 | 277 | | 278 | | 279 | | 285 | 286 | | 287 |
| Postemergence | | | | | | | | | | | |
| Barnyardgrass | 40 | 20 | | 40 | | 0 | | 20 | 0 | | 0 |
| Blackgrass | 80 | 30 | | 50 | | 0 | | 0 | 20 | | 10 |
| Corn | 0 | 10 | | 0 | | 0 | | 0 | 0 | | 0 |
| Foxtail, Giant | 100 | 10 | | 30 | | 0 | | 10 | 0 | | 10 |
| Galium | 100 | 70 | | 80 | | 0 | | 50 | 50 | | 50 |
| Kochia | 40 | 50 | | 40 | | 0 | | 10 | 10 | | 0 |
| Pigweed | 90 | 40 | | 30 | | 0 | | 0 | 20 | | 10 |
| Ragweed | 90 | 40 | | 60 | | 0 | | 30 | 30 | | 20 |
| Ryegrass, Italian | 10 | 40 | | 70 | | 0 | | 0 | 0 | | 20 |
| Wheat | 0 | 30 | | 40 | | 0 | | 0 | 30 | | 0 |
| | Compounds | | | | | | | | | | |
| 31 g ai/ha | 288 | 289 | | 290 | | 291 | | 292 | 293 | | 294 |
| Postemergence | | | | | | | | | | | |
| Barnyardgrass | 70 | 0 | | 0 | | 20 | | 30 | 20 | | 20 |
| Blackgrass | 40 | 0 | | 0 | | 0 | | 0 | 0 | | 0 |
| Corn | 10 | 0 | | 0 | | 20 | | 0 | 0 | | 0 |
| Foxtail, Giant | 80 | 0 | | 0 | | 40 | | 60 | 40 | | 40 |
| Galium | 60 | 70 | | 70 | | 100 | | 100 | 100 | | 80 |
| Kochia | 10 | 20 | | 0 | | 50 | | 60 | 0 | | 20 |
| Pigweed | 0 | 0 | | 0 | | 30 | | 60 | 20 | | 20 |
| Ragweed | 30 | 40 | | 20 | | 100 | | 100 | 100 | | 80 |
| Ryegrass, Italian | 40 | 0 | | 0 | | 90 | | 100 | 100 | | 50 |
| Wheat | 0 | 0 | | 0 | | 30 | | 60 | 40 | | 0 |
| | Compounds | | | | | | | | | | |
| 31 g ai/ha | 295 | 296 | 297 | 298 | 299 | 300 | 301 | 302 | 303 | 304 | 305 |
| Postemergence | | | | | | | | | | | |
| Barnyardgrass | 50 | 20 | 0 | 0 | 70 | 30 | 20 | 0 | 0 | 0 | 20 |
| Blackgrass | 0 | 0 | 0 | 0 | 80 | 30 | 0 | 0 | 0 | 0 | 10 |
| Corn | 0 | 0 | 0 | 0 | 10 | 10 | 0 | 0 | 0 | 10 | 0 |
| Foxtail, Giant | 70 | 30 | 0 | 0 | 90 | 60 | 40 | 10 | 0 | 0 | 30 |
| Galium | 100 | 100 | 0 | 0 | 100 | 90 | 70 | 20 | 30 | 10 | 70 |
| Kochia | 0 | 30 | 0 | 0 | 100 | 40 | 0 | 0 | 40 | 30 | 50 |
| Pigweed | 60 | 80 | 0 | 0 | 90 | 80 | 50 | 30 | 40 | 50 | 60 |
| Ragweed | 100 | 100 | 0 | 0 | 100 | 30 | 10 | 0 | 10 | 0 | 30 |
| Ryegrass, Italian | 100 | 80 | 0 | 0 | 100 | 10 | 0 | 0 | 20 | 20 | 10 |
| Wheat | 30 | 70 | 0 | 0 | 30 | 0 | 0 | 0 | 0 | 0 | 0 |

| | Compounds | | | | | | |
|---|---|---|---|---|---|---|---|
| 1000 g ai/ha | 221 | 222 | 223 | 224 | 225 | 237 | 238 |
| Preemergence | | | | | | | |
| Barnyardgrass | 30 | 0 | 0 | 10 | 0 | 0 | 0 |
| Foxtail, Giant | 100 | 50 | 0 | 80 | 60 | 60 | 40 |
| Kochia | 60 | 0 | 0 | 20 | 10 | 30 | 30 |
| Pigweed | 100 | 100 | 100 | 100 | 80 | 90 | 100 |
| Ragweed | 80 | 50 | 20 | 70 | 40 | 20 | 90 |
| Ryegrass, Italian | 100 | 0 | 0 | 0 | 0 | 0 | 20 |

| | Compounds | | | | | | Compound |
|---|---|---|---|---|---|---|---|
| 500 g ai/ha | 149 | 150 | 151 | 168 | 206 | 125 g ai/ha | 305 |
| Preemergence | | | | | | Preemergence | |
| Barnyardgrass | 0 | 0 | 0 | 80 | 80 | Barnyardgrass | 20 |
| Foxtail, Giant | — | — | 0 | 100 | 100 | Foxtail, Giant | 30 |
| Foxtail, Green | 0 | 0 | — | — | — | Foxtail, Green | — |
| Kochia | 0 | 0 | 0 | 10 | 40 | Kochia | 30 |
| Pigweed | 20 | 0 | 0 | 100 | 100 | Pigweed | 70 |
| Ragweed | 0 | 80 | 0 | — | 40 | Ragweed | 20 |
| Ryegrass, Italian | 0 | 0 | 30 | 90 | 100 | Ryegrass, Italian | 30 |

| | Compounds | | | | | | |
|---|---|---|---|---|---|---|---|
| 125 g ai/ha | 149 | 151 | 152 | 153 | 154 | 155 | 156 |
| Preemergence | | | | | | | |
| Barnyardgrass | 0 | 0 | 10 | 90 | 90 | 100 | 80 |
| Foxtail, Giant | — | 0 | 20 | 100 | 100 | 90 | 100 |
| Foxtail, Green | 0 | — | — | — | — | — | — |
| Kochia | 0 | 0 | 10 | 90 | 90 | 90 | 100 |
| Pigweed | 0 | 0 | 30 | 100 | 90 | 100 | 80 |
| Ragweed | 0 | 0 | 40 | 100 | 80 | 90 | 100 |
| Ryegrass, Italian | 0 | 0 | 80 | 100 | 100 | 100 | 100 |
| | Compounds | | | | | | |
| 125 g ai/ha | 157 | 158 | 159 | 163 | 164 | 165 | 166 |
| Preemergence | | | | | | | |
| Barnyardgrass | 90 | 30 | 40 | 30 | 50 | 80 | 50 |
| Foxtail, Giant | 100 | 80 | 50 | 50 | 50 | 70 | 60 |
| Foxtail, Green | — | — | — | — | — | — | — |
| Kochia | 100 | 0 | 20 | 50 | 50 | 100 | 80 |
| Pigweed | 40 | 50 | 40 | 50 | 90 | 20 | 50 |
| Ragweed | 100 | 40 | 90 | 0 | 30 | 90 | 10 |
| Ryegrass, Italian | 100 | 0 | 80 | 100 | 30 | 100 | 60 |
| | Compounds | | | | | | |
| 125 g ai/ha | 167 | 168 | 173 | 174 | 175 | 176 | 177 |
| Preemergence | | | | | | | |
| Barnyardgrass | 100 | 60 | 50 | 90 | 70 | 0 | 60 |
| Foxtail, Giant | 100 | 80 | 90 | 90 | 70 | 10 | 10 |
| Foxtail, Green | — | — | — | — | — | — | — |
| Kochia | 100 | 0 | 80 | 80 | 80 | 0 | 0 |
| Pigweed | 100 | 100 | 30 | 70 | 30 | 60 | 10 |
| Ragweed | 100 | — | 90 | 90 | 100 | 20 | 20 |
| Ryegrass, Italian | 100 | 20 | 100 | 90 | 50 | 20 | 10 |
| | Compounds | | | | | | |
| 125 g ai/ha | 178 | 179 | 180 | 181 | 182 | 183 | 184 |
| Preemergence | | | | | | | |
| Barnyardgrass | 50 | 100 | 80 | 60 | 90 | 70 | 90 |
| Foxtail, Giant | 60 | 100 | 100 | 90 | 100 | 100 | 100 |
| Foxtail, Green | — | — | — | — | — | — | — |
| Kochia | 0 | 90 | 100 | 70 | 0 | 0 | 0 |
| Pigweed | 70 | 100 | 80 | 90 | 70 | 10 | 60 |
| Ragweed | 40 | 100 | — | — | — | — | — |
| Ryegrass, Italian | 70 | 100 | 100 | 90 | 100 | 80 | 100 |
| | Compounds | | | | | | |
| 125 g ai/ha | 185 | 186 | 187 | 188 | 189 | 190 | 191 |
| Preemergence | | | | | | | |
| Barnyardgrass | 70 | 90 | 100 | 100 | 90 | 50 | 90 |
| Foxtail, Giant | 100 | 100 | 100 | 100 | 90 | 70 | 90 |
| Foxtail, Green | — | — | — | — | — | — | — |
| Kochia | 50 | 0 | 90 | 100 | 40 | 0 | 60 |
| Pigweed | 100 | 100 | 90 | 90 | 100 | 90 | 100 |
| Ragweed | — | — | — | — | — | — | 80 |
| Ryegrass, Italian | 100 | 100 | 100 | 100 | 100 | 60 | 80 |
| | Compounds | | | | | | |
| 125 g ai/ha | 192 | 193 | 197 | 198 | 199 | 200 | 201 |
| Preemergence | | | | | | | |
| Barnyardgrass | 90 | 60 | 0 | 40 | 0 | 0 | 10 |
| Foxtail, Giant | 90 | 80 | 10 | 70 | 10 | 10 | 30 |
| Foxtail, Green | — | — | — | — | — | — | — |
| Kochia | 20 | 70 | 0 | 0 | 0 | 0 | 0 |
| Pigweed | 100 | 90 | 20 | 0 | 10 | 0 | 0 |
| Ragweed | 70 | 60 | 0 | 0 | 0 | 0 | 20 |
| Ryegrass, Italian | 70 | 80 | 20 | 70 | 20 | 0 | 80 |
| | Compounds | | | | | | |
| 125 g ai/ha | 202 | 203 | 204 | 205 | 206 | 207 | 208 |
| Preemergence | | | | | | | |
| Barnyardgrass | 0 | 0 | 0 | 100 | 50 | 80 | 80 |
| Foxtail, Giant | 0 | 10 | 0 | 100 | 70 | 90 | 90 |
| Foxtail, Green | — | — | — | — | — | — | — |
| Kochia | 0 | 0 | 0 | 70 | 0 | 60 | 20 |
| Pigweed | 0 | 0 | 0 | 100 | 90 | 80 | 100 |
| Ragweed | 10 | 0 | 0 | — | 20 | — | 100 |
| Ryegrass, Italian | 10 | 0 | 0 | 100 | 60 | 80 | 70 |
| | Compounds | | | | | | |
| 125 g ai/ha | 209 | 210 | 211 | 212 | 213 | 214 | 216 |
| Preemergence | | | | | | | |
| Barnyardgrass | 80 | 80 | 90 | 80 | 90 | 90 | 0 |
| Foxtail, Giant | 100 | 100 | 90 | 100 | 80 | 100 | 0 |
| Foxtail, Green | — | — | — | — | — | — | — |
| Kochia | 40 | 0 | 60 | 20 | 80 | 70 | 0 |
| Pigweed | 100 | 100 | 90 | 100 | 100 | 100 | 0 |
| Ragweed | 90 | 90 | 100 | 100 | 100 | 100 | 0 |
| Ryegrass, Italian | 30 | 20 | 80 | 30 | 100 | 100 | 10 |
| | Compounds | | | | | | |
| 125 g ai/ha | 217 | 218 | 219 | 220 | 226 | 227 | 229 |
| Preemergence | | | | | | | |
| Barnyardgrass | 0 | 0 | 0 | 90 | 0 | 20 | 30 |
| Foxtail, Giant | 0 | 0 | 0 | 100 | 10 | 30 | 30 |
| Foxtail, Green | — | — | — | — | — | — | — |
| Kochia | 20 | 0 | 0 | 60 | 40 | 10 | 0 |
| Pigweed | 0 | 0 | 10 | 100 | 70 | 10 | 40 |
| Ragweed | 0 | 0 | 0 | — | 70 | 80 | 0 |
| Ryegrass, Italian | 0 | 10 | 0 | 100 | 70 | 60 | 90 |
| | Compounds | | | | | | |
| 125 g ai/ha | 230 | 231 | 232 | 233 | 234 | 235 | 236 |
| Preemergence | | | | | | | |
| Barnyardgrass | 0 | 90 | 100 | 100 | 0 | 100 | 90 |
| Foxtail, Giant | 30 | 90 | 100 | 100 | 20 | 90 | 100 |
| Foxtail, Green | — | — | — | — | — | — | — |
| Kochia | 0 | 0 | 10 | 0 | 0 | 70 | 40 |
| Pigweed | 30 | 50 | 90 | 90 | 0 | 90 | 70 |
| Ragweed | 0 | — | — | — | 40 | — | — |
| Ryegrass, Italian | 10 | 0 | 0 | 0 | 90 | 0 | 20 |
| | Compounds | | | | | | |
| 125 g ai/ha | 239 | 240 | 241 | 242 | 243 | 244 | 245 |
| Preemergence | | | | | | | |
| Barnyardgrass | 0 | 0 | 60 | 90 | 80 | 70 | 70 |
| Foxtail, Giant | 10 | 80 | 90 | 100 | 100 | 100 | 90 |
| Foxtail, Green | — | — | — | — | — | — | — |
| Kochia | 10 | 0 | 0 | 50 | 0 | 0 | 0 |
| Pigweed | 10 | 100 | 100 | 100 | 100 | 90 | 100 |
| Ragweed | 50 | 10 | 10 | 90 | 80 | 20 | 40 |
| Ryegrass, Italian | 50 | 0 | 0 | 90 | 20 | 20 | 0 |
| | Compounds | | | | | | |
| 125 g ai/ha | 246 | 250 | 253 | 254 | 261 | 264 | 268 |
| Preemergence | | | | | | | |
| Barnyardgrass | 80 | 80 | 0 | 0 | 0 | 0 | 70 |
| Foxtail, Giant | 80 | 80 | 10 | 40 | 10 | 20 | 100 |
| Foxtail, Green | — | — | — | — | — | — | — |
| Kochia | 0 | 80 | 0 | 0 | 0 | 0 | 0 |
| Pigweed | 100 | 70 | 80 | 10 | 10 | 30 | 0 |
| Ragweed | 90 | — | 0 | 90 | 10 | 30 | 60 |
| Ryegrass, Italian | 60 | 0 | 30 | 60 | 70 | 90 | 90 |
| | Compounds | | | | | | |
| 125 g ai/ha | 269 | 270 | 271 | 272 | 273 | 277 | 278 |
| Preemergence | | | | | | | |
| Barnyardgrass | 0 | 0 | 50 | 50 | 80 | 30 | 20 |
| Foxtail, Giant | 30 | 20 | 80 | 80 | 90 | 30 | 60 |
| Foxtail, Green | — | — | — | — | — | — | — |
| Kochia | 0 | 0 | 100 | 90 | 40 | 90 | 70 |
| Pigweed | 10 | 0 | 100 | 100 | 90 | 70 | 0 |
| Ragweed | 0 | 10 | 80 | 40 | 20 | 80 | 40 |
| Ryegrass, Italian | 10 | 10 | 100 | 100 | 40 | 100 | 100 |
| | Compounds | | | | | | |
| 125 g ai/ha | 279 | 285 | 286 | 287 | 288 | 289 | 290 |
| Preemergence | | | | | | | |
| Barnyardgrass | 0 | 0 | 90 | 90 | 100 | 0 | 0 |
| Foxtail, Giant | 0 | 0 | 50 | 40 | 100 | 10 | 20 |
| Foxtail, Green | — | — | — | — | — | — | — |
| Kochia | 0 | 0 | 0 | 0 | 30 | 0 | 0 |
| Pigweed | 0 | 0 | 10 | 0 | 0 | 40 | 30 |
| Ragweed | 0 | 10 | 20 | 20 | 20 | 30 | 90 |
| Ryegrass, Italian | 0 | 10 | 10 | 90 | 100 | 30 | 40 |
| | Compounds | | | | | | |
| 125 g ai/ha | 291 | 292 | 293 | 294 | 295 | 296 | 297 |
| Preemergence | | | | | | | |
| Barnyardgrass | 100 | 100 | 0 | 100 | 0 | 100 | 0 |
| Foxtail, Giant | 100 | 100 | 100 | 100 | 100 | 100 | 0 |
| Foxtail, Green | — | — | — | — | — | — | — |
| Kochia | 90 | 100 | 0 | 50 | 70 | 20 | 0 |
| Pigweed | 80 | 100 | 70 | 80 | 100 | 100 | 0 |
| Ragweed | 100 | 100 | 100 | 100 | 100 | 100 | 0 |
| Ryegrass, Italian | 100 | 100 | 100 | 100 | 100 | 100 | 0 |
| | Compounds | | | | | | |
| 125 g ai/ha | 298 | 299 | 300 | 301 | 302 | 303 | 304 |
| Preemergence | | | | | | | |
| Barnyardgrass | 0 | 100 | 60 | 40 | 0 | 0 | 0 |
| Foxtail, Giant | 0 | 100 | 80 | 80 | 10 | 10 | 10 |
| Foxtail, Green | — | — | — | — | — | — | — |
| Kochia | 0 | 100 | 30 | 0 | 0 | 10 | 0 |
| Pigweed | 10 | 100 | 90 | 20 | 40 | 30 | 20 |
| Ragweed | 0 | 100 | 20 | 0 | 20 | 20 | 40 |
| Ryegrass, Italian | 0 | 100 | 70 | 30 | 0 | 20 | 20 |
| | Compounds | | | | | | |
| 31 p ai/ha | 152 | 153 | 154 | 155 | 156 | 157 | 158 |
| Preemergence | | | | | | | |
| Barnyardgrass | 0 | 80 | 40 | 70 | 0 | 70 | 20 |
| Foxtail, Giant | 0 | 90 | 60 | 80 | 50 | 80 | 10 |
| Kochia | 0 | 60 | 0 | 0 | 40 | 50 | 0 |
| Pigweed | 0 | 100 | 40 | 40 | 40 | 10 | 0 |
| Ragweed | 20 | 90 | 40 | 70 | 100 | 100 | 40 |
| Ryegrass Italian | 20 | 100 | 40 | 40 | 100 | 90 | 0 |
| | Compounds | | | | | | |
| 31 p ai/ha | 159 | 163 | 164 | 165 | 166 | 167 | 173 |
| Preemergence | | | | | | | |
| Barnyardgrass | 40 | 0 | 0 | 60 | 0 | 100 | 60 |
| Foxtail, Giant | 0 | 10 | 10 | 60 | 0 | 100 | 40 |
| Kochia | 0 | 0 | 0 | 80 | 0 | 70 | 60 |
| Pigweed | 30 | 30 | 70 | 0 | 0 | 100 | 30 |
| Ragweed | 70 | 0 | 0 | 50 | 0 | 80 | 30 |
| Ryegrass Italian | 0 | 70 | 0 | 80 | 10 | 100 | 30 |
| | Compounds | | | | | | |
| 31 g ai/ha | 174 | 175 | 176 | 177 | 178 | 179 | 180 |
| Preemergence | | | | | | | |
| Barnyardgrass | 70 | 50 | 0 | 40 | 30 | 90 | 0 |
| Foxtail, Giant | 70 | 30 | 0 | 0 | 10 | 100 | 70 |
| Kochia | 20 | 40 | 0 | 0 | 0 | 90 | 0 |
| Pigweed | 20 | 0 | 10 | 0 | 20 | 100 | 50 |
| Ragweed | 60 | 40 | 10 | 0 | 0 | 90 | — |
| Ryegrass, Italian | 80 | 30 | 10 | 0 | 0 | 100 | 40 |
| | Compounds | | | | | | |
| 31 g ai/ha | 181 | 182 | 183 | 184 | 185 | 186 | 187 |
| Preemergence | | | | | | | |
| Barnyardgrass | 0 | 0 | 0 | 0 | 0 | 40 | 70 |
| Foxtail, Giant | 50 | 70 | 40 | 60 | 90 | 100 | 80 |
| Kochia | 30 | 0 | 0 | 0 | 0 | 0 | 0 |
| Pigweed | 30 | 0 | 0 | 50 | 70 | 50 | 60 |
| Ragweed | — | — | — | — | — | — | — |
| Ryegrass, Italian | 0 | 30 | 0 | 0 | 10 | 40 | 40 |
| | Compounds | | | | | | |
| 31 g ai/ha | 188 | 189 | 190 | 191 | 192 | 193 | 197 |
| Preemergence | | | | | | | |
| Barnyardgrass | 30 | 20 | 30 | 30 | 30 | 30 | 0 |
| Foxtail, Giant | 90 | 20 | 60 | 70 | 60 | 30 | 0 |
| kochia | 10 | 0 | 0 | 10 | 0 | 10 | 0 |
| Pigweed | 70 | 70 | 20 | 80 | 40 | 50 | 0 |
| Ragweed | — | — | — | 50 | 30 | 30 | 0 |
| Ryegrass, Italian | 80 | 90 | 0 | 30 | 10 | 30 | 0 |
| | Compounds | | | | | | |
| 31 g ai/ha | 198 | 199 | 200 | 201 | 202 | 203 | 204 |
| Preemergence | | | | | | | |
| Barnyardgrass | 0 | 0 | 0 | 0 | 0 | 0 | 0 |
| Foxtail, Giant | 0 | 0 | 0 | 10 | 0 | 0 | 0 |
| kochia | 0 | 0 | 0 | 0 | 0 | 0 | 0 |
| Pigweed | 0 | 0 | 0 | 0 | 0 | 0 | 0 |
| Ragweed | 0 | 0 | 0 | 0 | 0 | 0 | 0 |
| Ryegrass, Italian | 0 | 0 | 0 | 10 | 0 | 0 | 0 |
| | Compounds | | | | | | |
| 31 g ai/ha | 205 | 207 | 208 | 209 | 210 | 211 | 212 |
| Preemergence | | | | | | | |
| Barnyardgrass | 70 | 20 | 0 | 50 | 30 | 50 | 40 |
| Foxtail, Giant | 80 | 30 | 50 | 80 | 90 | 70 | 80 |
| Kochia XXXX | 0 | 0 | 0 | 0 | 0 | 0 | 0 |
| Pigweed | 80 | 50 | 40 | 20 | 30 | 70 | 10 |
| Ragweed | — | — | 60 | 90 | 0 | 40 | 10 |
| Ryegrass, Italian | 20 | 40 | 10 | 10 | 0 | 30 | 0 |
| | Compounds | | | | | | |
| 31 g ai/ha | 213 | 214 | 216 | 217 | 218 | 219 | 220 |
| Preemergence | | | | | | | |
| Barnyardgrass | 70 | 70 | 50 | 0 | 0 | 0 | 0 |
| Foxtail, Giant | 60 | 80 | 0 | 0 | 0 | 0 | 20 |
| Kochia | 20 | 20 | 0 | 0 | 0 | 0 | 0 |
| Pigweed | 100 | 80 | 0 | 0 | 0 | 0 | 30 |
| Ragweed | 70 | 20 | 0 | 0 | 0 | 0 | — |
| Ryegrass, Italian | 70 | 90 | 0 | 0 | 0 | 0 | 30 |
| | Compounds | | | | | | |
| 31 p ai/ha | 226 | 227 | 229 | 230 | 231 | 232 | 233 |
| Preemergence | | | | | | | |
| Barnyardgrass | 0 | 0 | 0 | 0 | 60 | 60 | 90 |
| Foxtail, Giant | 10 | 0 | 10 | 0 | 30 | 70 | 60 |
| Kochia | 0 | 0 | 0 | 0 | — | 0 | 0 |
| Pigweed | 30 | 0 | 10 | 10 | 40 | 70 | 90 |
| Ragweed | 30 | 20 | 0 | 0 | — | — | — |
| Ryegrass, Italian | 30 | 30 | 20 | 0 | — | 0 | 0 |
| | Compounds | | | | | | |
| 31 p ai/ha | 234 | 235 | 236 | 239 | 240 | 241 | 242 |
| Preemergence | | | | | | | |
| Barnyardgrass | 0 | 90 | 90 | 0 | 0 | 10 | 60 |
| Foxtail, Giant | 0 | 30 | 80 | 0 | 10 | 80 | 80 |
| Kochia | 0 | 0 | 0 | 0 | 0 | 0 | 30 |
| Pigweed | 0 | 100 | 10 | 0 | 50 | 90 | 100 |
| Ragweed | 10 | — | — | 10 | 0 | 40 | 80 |
| Ryegrass, Italian | 40 | 0 | 0 | 10 | 0 | 0 | 10 |
| | Compounds | | | | | | |
| 31 g ai/ha | 243 | 244 | 245 | 246 | 250 | 253 | 254 |
| Preemergence | | | | | | | |
| Barnyardgrass | 20 | 40 | 30 | 60 | 70 | 0 | 0 |
| Foxtail, Giant | 80 | 90 | 60 | 80 | 40 | 0 | 0 |
| Kochia | 0 | 0 | 0 | 0 | 0 | 0 | 0 |
| Pigweed | 70 | 50 | 50 | 90 | 10 | 0 | 0 |
| Ragweed | 30 | 10 | 10 | 100 | — | 0 | 40 |
| Ryegrass, Italian | 0 | 0 | 0 | 30 | 0 | 0 | 20 |
| | Compounds | | | | | | |
| 31 g ai/ha | 261 | 264 | 268 | 269 | 270 | 271 | 272 |
| Preemergence | | | | | | | |
| Barnyardgrass | 0 | 0 | 30 | 0 | 0 | 30 | 30 |
| Foxtail, Giant | 0 | 0 | 70 | 0 | 0 | 40 | 80 |
| Kochia | 0 | 0 | 0 | 0 | 0 | 20 | 10 |
| Pigweed | 0 | 0 | 0 | 0 | 0 | 100 | 70 |
| Ragweed | 0 | 0 | 10 | 0 | 0 | 30 | 20 |
| Ryegrass, Italian | 0 | 0 | 20 | 0 | 0 | 80 | 90 |
| | Compounds | | | | | | |
| 31 g ai/ha | 273 | 277 | 278 | 279 | 285 | 286 | 287 |
| Preemergence | | | | | | | |
| Barnyardgrass | 40 | 0 | 0 | 0 | 0 | 60 | 40 |
| Foxtail, Giant | 80 | 10 | 30 | 0 | 0 | 0 | 10 |
| kochia | 0 | 0 | 0 | 0 | 0 | 0 | 0 |
| Pigweed | 90 | 10 | 0 | 0 | 0 | 0 | 0 |
| Ragweed | 20 | 20 | 10 | 0 | 0 | 0 | 0 |
| Ryegrass, Italian | 0 | 10 | 20 | 0 | 0 | 0 | 50 |
| | Compounds | | | | | | |
| 31 g ai/ha | 288 | 289 | 290 | 291 | 292 | 293 | 294 |
| Preemergence | | | | | | | |
| Barnyardgrass | 70 | 0 | 0 | 0 | 60 | 0 | 70 |
| Foxtail, Giant | 40 | 0 | 0 | 50 | 70 | 90 | 70 |
| kochia | 0 | 0 | 0 | 0 | 30 | 0 | 0 |
| Pigweed | 0 | 0 | 10 | 10 | 50 | 0 | 20 |
| Ragweed | 10 | 0 | 20 | 60 | 90 | 40 | 70 |
| Ryegrass, Italian | 70 | 0 | 0 | 10 | 80 | 30 | 60 |

| | Compounds | | | | | | | | | | |
|---|---|---|---|---|---|---|---|---|---|---|---|
| 31 g ai/ha | 295 | 296 | 297 | 298 | 299 | 300 | 301 | 302 | 303 | 304 | 305 |
| Preemergence | | | | | | | | | | | |
| Barnyardgrass | 0 | 60 | 0 | 0 | 90 | 40 | 0 | 0 | 0 | 0 | 10 |
| Foxtail, Giant | 100 | 80 | 0 | 0 | 100 | 40 | 20 | 0 | 0 | 0 | 10 |
| Kochia | 0 | 0 | 0 | 0 | 90 | 0 | 0 | 0 | 0 | 0 | 10 |
| Pigweed | 80 | 40 | 0 | 0 | 100 | 30 | 0 | 10 | 10 | 10 | 30 |
| Ragweed | 90 | 30 | 0 | 0 | 90 | 0 | 0 | 0 | 0 | 0 | 20 |
| Ryegrass, Italian | 80 | 90 | 0 | 0 | 100 | 10 | 0 | 0 | 0 | 0 | 0 |

### Test D

Plant species in the flooded paddy test selected from rice (Oryza sativa), sedge umbrella (small-flower umbrella sedge, Cyperus difformis) duck salad (Heteranthera limosa), and barnyardgrass (Echinochloa crus-galli) were grown to the 2-leaf stage for testing. At time of treatment, test pots were flooded to 3 cm above the soil surface, treated by application of test compounds directly to the paddy water, and then maintained at that water depth for the duration of the test. Treated plants and controls were maintained in a greenhouse for 13 to 15 d, after which time all species were compared to controls and visually evaluated. Plant response ratings, summarized in Table D, are based on a scale of 0 to 100 where 0 is no effect and 100 is complete control. A dash (-) response means no test result.

TABLE D

| | Compounds | | | | | | | | | | | | | |
|---|---|---|---|---|---|---|---|---|---|---|---|---|---|---|
| 250 g ai/ha | 149 | 151 | 152 | 153 | 154 | 155 | 156 | 157 | 158 | 159 | 163 | 164 | 165 | 166 |
| Flood | | | | | | | | | | | | | | |
| Barnyardgrass | 0 | 0 | 0 | 80 | 0 | 15 | 0 | 0 | 45 | 0 | 0 | 0 | 0 | 0 |
| Ducksalad | 0 | 0 | 0 | 55 | 0 | 30 | 30 | 0 | 50 | 0 | 0 | 0 | 0 | 0 |
| Rice | 0 | 0 | 0 | 40 | 0 | 0 | 0 | 0 | 15 | 0 | 0 | 0 | 0 | 0 |
| Sedge, Umbrella | 0 | 0 | 0 | 70 | 0 | 45 | 30 | 0 | 65 | 0 | 0 | 0 | 0 | 0 |
| | Compounds | | | | | | | | | | | | | |
| 250 g ai/ha | 167 | 168 | 173 | 174 | 175 | 176 | 177 | 178 | 179 | 180 | 181 | 182 | 183 | 184 |
| Flood | | | | | | | | | | | | | | |
| Barnyardgrass | 95 | 0 | 0 | 0 | 0 | 0 | 0 | 0 | 95 | 40 | 0 | 0 | 0 | 0 |
| Ducksalad | 70 | 0 | 0 | 0 | 0 | 0 | 0 | 0 | 80 | 60 | 0 | 0 | 0 | 0 |
| Rice | 65 | 0 | 0 | 0 | 0 | 0 | 0 | 0 | 70 | 0 | 0 | 0 | 0 | 0 |
| Sedge, Umbrella | 95 | 0 | 0 | 0 | 0 | 0 | 0 | 0 | 95 | 65 | 0 | 0 | 0 | 0 |
| | Compounds | | | | | | | | | | | | | |
| 250 g ai/ha | 185 | 186 | 187 | 188 | 189 | 190 | 191 | 192 | 193 | 197 | 198 | 199 | 200 | 201 |
| Flood | | | | | | | | | | | | | | |
| Barnyardgrass | 45 | 0 | 35 | 15 | 0 | 0 | 70 | 50 | 0 | 0 | 0 | 0 | 0 | 0 |
| Ducksalad | 85 | 0 | 75 | 70 | 25 | 0 | 75 | 70 | 0 | 0 | 0 | 0 | 0 | 0 |
| Rice | 60 | 0 | 15 | 0 | 15 | 0 | 60 | 0 | 0 | 0 | 0 | 0 | 0 | 0 |
| Sedge, Umbrella | 95 | 0 | 60 | 85 | 35 | 0 | 80 | 70 | 0 | 0 | 0 | 0 | 0 | 0 |
| | Compounds | | | | | | | | | | | | | |
| 250 g ai/ha | 202 | 203 | 204 | 205 | 206 | 207 | 208 | 209 | 210 | 211 | 212 | 213 | 214 | 216 |
| Flood | | | | | | | | | | | | | | |
| Barnyardgrass | 0 | 0 | 0 | 0 | 0 | 0 | 0 | 0 | 0 | 0 | 20 | 70 | 70 | 0 |
| Ducksalad | 0 | 0 | 0 | 0 | 0 | 0 | 0 | 50 | 45 | 0 | 60 | 70 | 70 | 0 |
| Rice | 0 | 0 | 0 | 0 | 0 | 0 | 0 | 0 | 0 | 0 | 0 | 40 | 45 | 0 |
| Sedge, Umbrella | 0 | 0 | 0 | 0 | 0 | 0 | 0 | 80 | 90 | 0 | 65 | 90 | 70 | 0 |
| | Compounds | | | | | | | | | | | | | |
| 250 g ai/ha | 217 | 218 | 219 | 220 | 221 | 222 | 226 | 227 | 229 | 230 | 231 | 232 | 233 | 234 |
| Flood | | | | | | | | | | | | | | |
| Barnyardgrass | 0 | 0 | 0 | 0 | 0 | 0 | 0 | 0 | 0 | 0 | 15 | 30 | 30 | 0 |
| Ducksalad | 0 | 0 | 0 | 0 | 0 | 0 | 0 | 0 | 0 | 0 | 75 | 30 | 0 | 0 |
| Rice | 0 | 0 | 0 | 0 | 0 | 0 | 0 | 0 | 0 | 0 | 0 | 0 | 0 | 0 |
| Sedge, Umbrella | 0 | 0 | 0 | 0 | 0 | 0 | 0 | 0 | 0 | 0 | 30 | 95 | 85 | 0 |

| | Compounds | | | | | | | | | | | | | | |
|---|---|---|---|---|---|---|---|---|---|---|---|---|---|---|---|
| 250 g ai/ha | 235 | 236 | 239 | 240 | 241 | 242 | 243 | 244 | 245 | 246 | 250 | 253 | 254 | 261 | 264 |
| Flood | | | | | | | | | | | | | | | |
| Barnyardgrass | 60 | 35 | 0 | 15 | 90 | 95 | 40 | 10 | 15 | 90 | 25 | 0 | 0 | 0 | 0 |
| Ducksalad | 75 | 70 | 0 | 0 | 85 | 98 | 80 | 50 | 30 | 95 | 85 | 0 | 0 | 0 | 0 |
| Rice | 0 | 0 | 0 | 0 | 85 | 85 | 45 | 15 | 25 | 90 | 10 | 0 | 0 | 0 | 0 |
| Sedge, Umbrella | 90 | 85 | 0 | 0 | 95 | 100 | 100 | 45 | 50 | 100 | 70 | 0 | 0 | 0 | 0 |
| | Compounds | | | | | | | | | | | | | | |
| 250 g ai/ha | 268 | 269 | 270 | 271 | 272 | 273 | 277 | 278 | 279 | 285 | 286 | 287 | 288 | 289 | 290 |
| Flood | | | | | | | | | | | | | | | |
| Barnyardgrass | 0 | 0 | 0 | 0 | 25 | 90 | 0 | 0 | 0 | 0 | 0 | 0 | 0 | 0 | 0 |
| Ducksalad | 0 | 0 | 0 | 0 | 60 | 95 | 0 | 0 | 0 | 0 | 0 | 0 | 0 | 0 | 0 |
| Race | 0 | 0 | 0 | 0 | 15 | 85 | 0 | 0 | 0 | 0 | 0 | 0 | 0 | 0 | 0 |
| Sedge, Umbrella | 0 | 0 | 0 | 0 | 65 | 95 | 0 | 0 | 0 | 0 | 0 | 0 | 0 | 0 | 0 |
| | Compounds | | | | | | | | | | | | | | |
| 250 g aa/ha | 291 | 292 | 293 | 294 | 295 | 296 | 297 | 298 | 299 | 300 | 301 | 302 | 303 | 304 | 305 |
| Flood | | | | | | | | | | | | | | | |
| Barnyardgrass | 0 | 0 | 0 | 0 | 60 | 0 | 0 | 0 | 85 | 15 | 0 | 0 | 0 | 0 | 0 |
| Ducksalad | 25 | 45 | 0 | 0 | 65 | 0 | 0 | 0 | 80 | 55 | 0 | 0 | 0 | 0 | 0 |
| Rice | 0 | 0 | 0 | 0 | 60 | 0 | 0 | 0 | 45 | 20 | 0 | 0 | 0 | 0 | 0 |
| Sedge, Umbrella | 45 | 55 | 0 | 0 | 70 | 0 | 0 | 0 | 90 | 45 | 0 | 0 | 0 | 0 | 0 |

What is claimed is:

1. A compound selected from Formula 1, N-oxides, salts and stereoisomers thereof 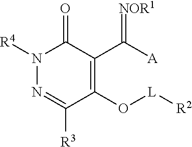
wherein
R¹ is H, C₁-C₇ alkyl, C₂-C₇ alkenyl, C₃-C₇ alkynyl, C₁-C₇ haloalkyl, C₂-C₇ haloalkenyl, C₄-C₈ alkylcycloalkyl, C₄-C₈ haloalkycycloalkyl, C₃-C₇ cycloalkyl, C₃-C₇ halocycloalkyl, C₄-C₇ cycloalkylalkyl, C₂-C₇ cyanoalkyl, C₃-C₈ alkylcarbonylalkyl, C₃-C₈ alkoxycarbonylalkyl, C₂-C₄ nitroalkyl, C₂-C₇ haloalkoxyalkyl, C₂-C₇ alkoxyalkyl, C₇-C₇ hydroxyalkyl or C₃-C₇ alkylthioalkyl; or benzyl optionally substituted by halogen, C₁-C₄ alkyl or C₁-C₄ haloalkyl;
A is selected from the group consisting of 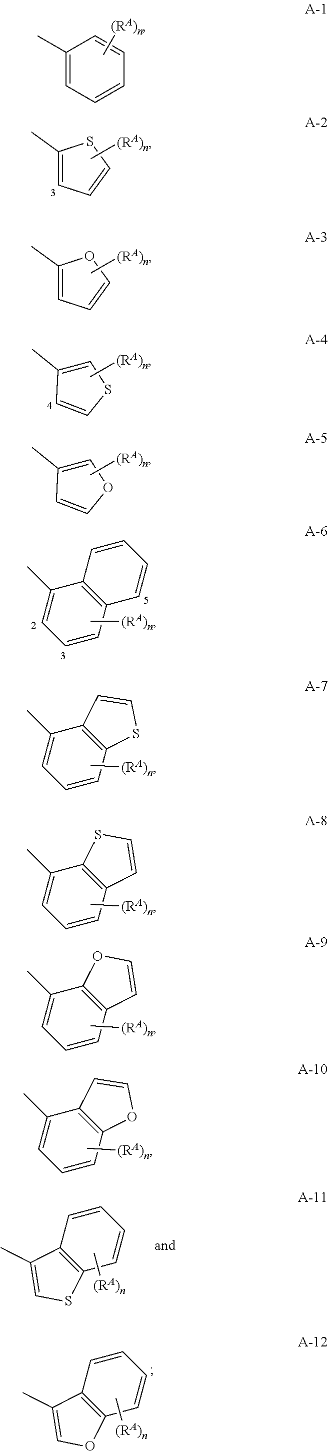
each R^{A} is independently halogen, nitro, cyano, C₁-C₅ alkyl, C₂-C₅ alkenyl, C₂-C₅ alkynyl, C₃-C₅ cycloalkyl, C₄-C₅ cycloalkylalkyl, C₁-C₅ haloalkyl, C₃-C₅ haloalkenyl, C₃-C₅ haloalkynyl, C₂-C₅ alkoxyalkyl, C₁-C₅ alkoxy, C₁-C₅ haloalkoxy, C₁-C₅ alkylthio, C₂-C₄ alkylsulfinyl, C₁-C₄ alkylsulfonyl, C₁-C₅ haloalkylthio or C₂-C₅ alkoxycarbonyl;
n is 0, 1 or 2;
L is a direct bond, C₁-C₄ alkanediyl or C₂-C₄ alkenediyl;
R² is H, C(═O)R⁵, C(═S)R⁵, CO₂R⁶, C(═O)SR⁶, S(O)₂R⁵, CONR⁷R⁸, S(O)₂N(R⁷)R⁸ or P(═O)(R⁹)R¹⁰; or C₁-C₄ alkyl, C₂-C₄ alkenyl, C₂-C₄ alkynyl, C₁-C₄ haloalkyl, C₂-C₄ haloalkenyl, C₂-C₄ haloalkynyl, C₂-C₄ alkoxyalkyl, C₃-C₆ cycloalkyl or C₄-C₇ cycloalkylalkyl; or a 5- or 6-membered heterocyclic ring optionally substituted by halogen, C₁-C₄ alkyl or C₁-C₄ haloalkyl;
R³ is H, halogen, cyano, —CHO, C₁-C₇ alkyl, C₃-C₈ alkylcarbonylalkyl, C₃-C₈ alkoxycarbonylalkyl, C₁-C₄ alkylcarbonyl, C₂-C₇ alkylcarbonyloxy, C₄-C₇ alkylcycloalkyl, C₃-C₇ alkenyl, C₃-C₇ alkynyl, C₁-C₄ alkylsulfinyl, C₁-C₄ alkylsulfonyl, C₁-C₄ alkylamino, C₂-C₈ dialkylamino, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₃-C₇ haloalkenyl, C₂-C₇ alkoxyalkyl, C₁-C₇ alkoxy, C₁-C₅ alkylthio or C₂-C₃ alkoxycarbonyl;
R⁴ is H, C₁-C₇ alkyl, C₃-C₈ alkylcarbonylalkyl, C₃-C₈ alkoxycarbonylalkyl, C₄-C₇ alkylcycloalkyl, C₃-C₇ alkenyl, C₃-C₇ alkynyl, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₃-C₇ haloalkenyl, C₂-C₇ alkoxyalkyl, C₃-C₇ alkylthioalkyl, C₁-C₇ alkoxy: or benzyl optionally substituted by halogen, C₁-C₄ alkyl or C₁-C₄ haloalkyl;
each R⁵ and R⁷ are independently H, C₁-C₇ alkyl, C₃-C₇ alkenyl, C₃-C₇ alkynyl, C₃-C₇ cycloalkyl, C₁-C₇ haloalkyl, C₃-C₇ haloalkenyl, C₂-C₇ alkoxyalkyl or C₄-C₇ cycloalkylalkyl; or phenyl, benzyl, or a 5- to 6-membered heterocyclic ring, each phenyl, benzyl or heterocyclic ring optionally substituted by halogen, C₁-C₄ alkyl or C₁-C₄ haloalkyl;
R⁶ is C₁-C₇ alkyl, C₃-C₇ alkenyl, C₃-C₇ alkynyl, C₃-C₇ cycloalkyl, C₂-C₇ haloalkyl, C₃-C₇ haloalkenyl, C₂-C₇ alkoxyalkyl or C₄-C₇ cycloalkylalkyl; or phenyl, benzyl or a 5- to 6-membered heterocyclic ring, each phenyl, benzyl or heterocyclic ring optionally substituted by halogen, C₁-C₄ alkyl or C₁-C₄ haloalkyl;
R⁸ is H, C₁-C₇ alkyl, C₂-C₇ alkenyl, C₂-C₇ alkynyl, C₃-C₇ cycloalkyl, C₄-C₇ cycloalkylalkyl, C₁-C₇ haloalkyl or C₂-C₇ alkoxyalkyl;
R⁹ is C₁-C₇ alkyl or C₁-C₇ alkoxy; and
R¹⁰ is C₁-C₇ alkyl or C₁-C₇ alkoxy.

2. The compound of claim 1 wherein
R¹ is H, C₁-C₇ alkyl, C₂-C₇ alkenyl, C₃-C₇ alkynyl, C₁-C₇ haloalkyl, C₂-C₇ haloalkenyl, C₄-C₈ alkylcycloalkyl or C₂-C₇ cyanoalkyl;
A is selected from the group consisting of A-1, A-2, A-3, A-4, A-6, A-7, A-8 and A-9;
each R^{A} is independently halogen, cyano, C₁-C₅ alkyl, C₃-C₅ cycloalkyl, C₄-C₅ cycloalkylalkyl, C₁-C₅ haloalkyl, C₂-C₅ alkoxyalkyl, C₁-C₅ alkoxy, C₁-C₅ alkylthio or C₁-C₄ alkylsulfonyl;
n is 0, 1 or 2;
L is a direct bond, C₁-C₂ alkanediyl or C₂-C₃ alkenediyl;
R² is H, C(═O)R⁵, C(═S)R⁵, CO₂R⁶, C(═O)SR⁶, CON(R⁷)R⁸ or P(═O)(R⁹)R¹⁰; or C₁-C₄ alkyl, C₂-C₄ alkenyl, C₂-C₄ alkynyl, C₁-C₄ haloalkyl, C₂-C₄ haloalkenyl, C₂-C₄ haloalkynyl or C₂-C₄ alkoxyalkyl;
R³ is H, halogen, cyano, —CHO, C₁-C₇ alkyl, C₃-C₈ alkylcarbonylalkyl, C₃-C₈ alkoxycarbonylalkyl, C₁-C₄ alkylcarbonyl, C₂-C₇ alkylcarbonyloxy, C₄-C₇ alkylcycloalkyl, C₃-C₇ alkenyl, C₃-C₇ alkynyl, C₁-C₄ alkylsulfinyl, C₁-C₄ alkylsulfonyl, C₁-C₄ alkylamino, C₂-C₈ dialkylamino, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₃-C₇ haloalkenyl, C₂-C₇ alkoxyalkyl, C₁-C₇ alkoxy or C₁-C₅ alkylthio;
R⁴ is H, C₁-C₇ alkyl, C₃-C₈ alkylcarbonylalkyl, C₃-C₈ alkoxycarbonylalkyl, C₄-C₇ alkylcycloalkyl, C₃-C₇ alkenyl, C₃-C₇ alkynyl, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₃-C₇ haloalkenyl, C₂-C₇ alkoxyalkyl, C₃-C₇ alkylthioalkyl or C₁-C₇ alkoxy; or benzyl optionally substituted by halogen, C₁-C₄ alkyl or C₁-C₄ haloalkyl;
each R⁵ and R⁷ are independently H, C₁-C₇ alkyl, C₃-C₇ alkenyl, C₃-C₇ alkynyl, C₃-C₇ cycloalkyl, C₁-C₇ haloalkyl, C₃-C₇ haloalkenyl, C₂-C₇ alkoxyalkyl or C₄-C₇ cycloalkylalkyl; or phenyl, benzyl, each phenyl, benzyl optionally substituted by halogen, C₁-C₄ alkyl or C₁-C₄ haloalkyl;
R⁶ is C₁-C₇ alkyl, C₃-C₇ alkenyl, C₃-C₇ alkynyl, C₃-C₇ cycloalkyl, C₂-C₇ haloalkyl, C₃-C₇ haloalkenyl, C₂-C₇ alkoxyalkyl or C₄-C₇ cycloalkylalkyl; or phenyl or benzyl, each phenyl or benzyl optionally substituted by halogen, C₁-C₄ alkyl or C₁-C₄ haloalkyl;
R⁸ is H, C₁-C₇ alkyl, C₃-C₇ cycloalkyl, C₄-C₇ cycloalkylalkyl or C₁-C₇ haloalkyl;
R⁹ is C₁-C₄ alkyl or C₁-C₄ alkoxy; and
R¹⁰ is C₁-C₄ alkyl or C₁-C₄ alkoxy.

3. The compound of claim 2 wherein
R¹ is H, C₁-C₇ alkyl, C₂-C₇ alkenyl, C₃-C₇ alkynyl, C₁-C₇ haloalkyl, C₂-C₇ haloalkenyl or C₄-C₈ alkylcycloalkyl;
A is selected from the group consisting of A-1, A-2, A-3, A-6, A-7 and A-8;
each R^{A} is independently halogen, C₁-C₅ alkyl, C₁-C₅ haloalkyl or C₁-C₅ alkoxy;
n is 1 or 2;
L is a direct bond, —CH₂— or —CH═CH—;
R² is H, C(═O)R⁵, CO₂R⁶, CON(R⁷)R⁸ or P(═O)(R⁹)R¹⁰; or C₁-C₄ alkyl, C₂-C₄ alkenyl, C₁-C₄ haloalkyl, C₂-C₄ haloalkenyl or C₂-C₄ alkoxyalkyl;
R³ is H, halogen, cyano, —CHO, C₁-C₇ alkyl, C₁-C₄ alkylcarbonyl, C₂-C₇ alkylcarbonyloxy, C₄-C₇ alkylcycloalkyl, C₁-C₄ alkylsulfinyl, C₁-C₄ alkylsulfonyl, C₁-C₄ alkylamino, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₂-C₇ alkoxyalkyl or C₁-C₇ alkoxy;
R⁴ is H, C₁-C₇ alkyl, C₃-C₈ alkoxycarbonylalkyl, C₄-C₇ alkylcycloalkyl, C₃-C₇ alkenyl, C₃-C₇ cycloalkyl, C₄-C₇ cycloalkylalkyl, C₂-C₃ cyanoalkyl, C₁-C₄ nitroalkyl, C₂-C₇ haloalkoxyalkyl, C₁-C₇ haloalkyl, C₂-C₇ alkoxyalkyl or C₁-C₇ alkoxy; or benzyl optionally substituted by halogen, C₁-C₄ alkyl or C₁-C₄ haloalkyl;
each R⁵ and R⁷ are independently H, C₁-C₇ alkyl, C₃-C₇ cycloalkyl or C₂-C₇ alkoxyalkyl: or phenyl, optionally substituted by halogen, C₁-C₄ alkyl or C₁-C₄ haloalkyl;
R⁶ is C₁-C₇ alkyl, C₂-C₇ haloalkyl or C₂-C₇ alkoxyalkyl; or phenyl optionally substituted by halogen, C₁-C₄ alkyl or C₁-C₄ haloalkyl;
R⁸ is H, C₁-C₇ alkyl or C₁-C₇ haloalkyl;
R⁹ is CH₃ or OCH₃; and
R¹⁰ is CH₃ or OCH₃.

4. The compound of claim 3 wherein
R¹ is C₁-C₃ alkyl, C₂-C₃ alkenyl, C₂-C₃ alkynyl or C₂-C₃ haloalkenyl;
A is selected from the group consisting of A-1, A-6, A-7 and A-8;
each R^{A} is independently F, Cl, Br, CH₃ or OCH₃;
R² is H, C(═O)R⁵, CO₂R⁶ or P(═O)(R⁹)R¹⁰; or C₁-C₄ alkyl, C₁-C₄ haloalkyl or C₂-C₄ alkoxyalkyl;
R³ is H, halogen, cyano, C₁-C₄ alkyl, C₃-C₅ cycloalkyl, C₁-C₃ haloalkyl, C₂-C₄ alkoxyalkyl or C₁-C₃ alkoxy;
R⁴ is C₁-C₄ alkyl, C₃-C₇ alkenyl, C₃-C₇ alkenyl, C₃-C₄ cycloalkyl, C₄-C₇ cycloalkylalkyl, C₂-C₃ cyanoalkyl, C₁-C₃ haloalkyl or C₂-C₄ alkoxyalkyl
R⁵ is C₁-C₇ alkyl;
R⁶ is C₁-C₇ alkyl; or phenyl optionally substituted by halogen or C₁-C₄ alkyl;
R⁹ is OCH₃; and
R¹⁰ is OCH₃.

5. The compound of claim 4 wherein
R¹ is CH₃, CH₂CH₃, i-Pr, —CH₂CH═CH₂ or —CH₂C═CH;
A is selected from the group consisting of A-1 and A-6;
each R^{A} is independently F, Cl, Br or CH₃;
R² is H, C(═O)R⁵ or CO₂R⁶; or C₂-C₄ alkoxyalkyl;
R³ is H, halogen, C₁-C₃ alkyl, cyclopropyl or C₁-C₂ haloalkyl;
R⁴ is C₁-C₃ alkyl, —CH₂CH₂C═N, C₁-C₂ haloalkyl or 2-methoxyethyl; and
R⁶ is C₁-C₇ alkyl.

6. The compound of claim 5 wherein
R¹ is CH₃, i-Pr or —CH₂C═CH;
A is A-1;
each R^{A} is independently F, Cl or Br;
R² is H, C(═O)R⁵ or CO₂R⁶;
R³ is H, Cl, Br, I, CH₃, CH₂CH₃ or cyclopropyl; and
R⁴ is CH₃, CH₂CH₃ or c-Pr.

7. The compound of claim 5 wherein
R¹ is CH₃ or i-Pr;
A is A-6;
each R^{A} is independently F, Cl or Br;
R² is H, C(═O)R⁵ or CO₂R⁶;
R³ is H, Cl, CH₃ or cyclopropyl; and
R⁴ is CH₃ or CH₂CH₃.

8. A compound of claim 1 selected from the group consisting of
4-[(E)-(3-bromo-1-naphthalenyl)(methoxyimino)methyl]-5-hydroxy-2,6-dimethyl-3(2H)-pyridazinone;
4-[(Z)-(3-bromo-1-naphthalenyl)(methoxyimino)methyl]-5-hydroxy-2,6-dimethyl-3(2H)-pyridazinone;
4-[(E)-(3-bromo-1-naphthalenyl)[(2-propyn-1-yloxy)imino]methyl]-5-hydroxy-2,6-dimethyl-3(2H)-pyridazinone;
4-[(E)-(3-bromo-1-naphthalenyl)(ethoxyimino)methyl]-5-hydroxy-2,6-dimethyl-3(2H)-pyridazinone;
4-[(Z)-(4-fluoro-1-naphthalenyl)[(2-propyn-1-yloxy)imino]methyl]-5-hydroxy-2,6-dimethyl-3(2H)-pyridazinone; and
4-[(E)-(4-fluoro-1-naphthalenyl)[(2-propyn-1-yloxy)imino]methyl]-5-hydroxy-2,6-dimethyl-3(2H)-pyridazinone.

9. A compound of claim 1 selected from the group consisting of
a mixture of E and Z isomers wherein A is A-6; n=0; R¹ is CH₃; L is a direct bond; R² is H; R³ is Cl; and R⁴ is CH₃;
a mixture of E and Z isomers wherein A is A-6; n=0; R¹ is CH₂CH₃; L is a direct bond; R² is H; R³ is Cl; and R⁴ is CH₃);
a mixture of E and Z isomers wherein A is A-6; R^{A} is 3-Br; R¹ is CH₃; L is a direct bond; R² is H; R³ is CH₃; and R⁴ is CH₃);
a mixture of E and Z isomers wherein A is A-6; R^{A} is 3-F; R¹ is CH(CH₃)₂; L is a direct bond; R² is H; R³ is CH₃; and R⁴ is CH₃); and
a mixture of E and Z isomers wherein A is A-6; R^{A} is 3-Br; R¹ is CH₂CH₃; L is a direct bond; R² is H; R³ is CH₃; and R⁴ is CH₃).

10. A herbicidal composition comprising a compound of claim 1 and at least one component selected from the group consisting of surfactants, solid diluents and liquid diluents.

11. A herbicidal composition comprising a compound of claim 1, at least one additional active ingredient selected from the group consisting of other herbicides and herbicide safeners, and at least one component selected from the group consisting of surfactants, solid diluents and liquid diluents.

12. A herbicidal mixture comprising (a) a compound of claim 1, and (b) at least one additional active ingredient selected from (b1) photosystem II inhibitors, (b2) acetohydroxy acid synthase (AHAS) inhibitors, (b3) acetyl-CoA carboxylase (ACCase) inhibitors, (b4) auxin mimics, (b5) 5-enol-pyruvylshikimate-3-phosphate (EPSP) synthase inhibitors, (b6) photosystem I electron diverters, (b7) protoporphyrinogen oxidase (PPO) inhibitors, (b8) glutamine synthetase (GS) inhibitors, (b9) very long chain fatty acid (VLCFA) elongase inhibitors, (b10) auxin transport inhibitors, (b11) phytoene desaturase (PDS) inhibitors, (b12) 4-hydroxyphenyl-pyruvate dioxygenase (HPPD) inhibitors, (b13) homogentisate solenesyltransererase (HST) inhibitors, (b14) cellulose biosynthesis inhibitors, (b15) other herbicides including mitotic disruptors, organic arsenicals, asulam, bromobutide, cinmethylin, cumyluron, dazomet, difenzoquat, dymron, etobenzanid, flurenol, fosamine, fosamine-ammonium, hydantocidin, metam, methyldymron, oleic acid, oxaziclomefone, pelargonic acid and pyributicarb, and (b16) herbicide safeners; and salts of compounds of (b1) through (b16).

13. The mixture of claim 12 comprising comprising (a) a compound selected from Formula 1, N-oxides, and salts thereof, and (b) at least one additional active ingredient selected from (b2) acetohydroxy acid synthase (AHAS) inhibitors; and (b12) 4-hydroxyphenyl-pyruvate dioxygenase (HPPD) inhibitors.

14. A method for controlling the growth of undesired vegetation comprising contacting the vegetation or its environment with a herbicidally effective amount of a compound of claim 1.
